SEQUENCE LISTING (110 sequences)

SEQ ID NO: 1 - protein, 949 aa, unidentified
  REGION 1..949: note = Bacteroides fragilis
MQKLLSLPPNLVQSFHELERVNRTDWFCTSDPVGKKLGSGGGTSWLLEECYNEYSDGATFGEWLEKEKRILLHAGGQSRRLPGYAPSGKILTPVPVFRWERGQHLGQNLLSLQLPLYEKIMSLAPDKLHTLIASGDVYIRSEKPLQSIPEADVVCYGLWVDPSLATHHGVFASDRKHPEQLDFMLQKPSLAELESLSKTHLFLMDIGIWLLSDRAVEILMKRSHKESSEELKYYDLYSDFGLALGTHPRIEDEEVNTLSVAILPLPGGEFYHYGTSKELISSTLSVQNKVYDQRRIMHRKVKPNPAMFVQNAVVRIPLCAENADLWIENSHIGPKWKIASRHIITGVPENDWSLAVPAGVCVDVVPMGDKGFVARPYGLDDVFKGDLRDSKTTLTGIPFGEWMSKRGLSYTDLKGRTDDLQAASVFPMVNSVEELGLVLRWMLSEPELEEGKNIWLRSERFSADEISAGANLKRLYAQREEFRKGNWQALAVNHEKSVFYQLDLADAAEDFVRLGLDMPELLPEDALQMSRIHNRMLRARILKLDGKDYRPEEQAAFDLLRDGLLDGISNRKSTPKLDVYSDQIVWGRSPVRIDMAGGWTDTPPYSLYSGGNVVNLAIELNGQPPLQVYVKPCKDFHIVLRSIDMGAMEIVSTFDELQDYKKIGSPFSIPKAALSLAGFAPAFSAVSYASLEEQLKDFGAGIEVTLLAAIPAGSGLGTSSILASTVLGAINDFCGLAWDKNEICQRTLVLEQLLTTGGGWQDQYGGVLQGVKLLQTEAGFAQSPLVRWLPDHLFTHPEYKDCHLLYYTGITRTAKGILAEIVSSMFLNSSLHLNLLSEMKAHALDMNEAIQRGSFVEFGRLVGKTWEQNKALDSGTNPPAVEAIIDLIKDYTLGYKLPGAGGGGYLYMVAKDPQAAVRIRKILTENAPNPRARFVEMTLSDKGFQVSRS

SEQ ID NO: 2 - DNA, 2850 bp, unidentified
  misc_feature 1..2850: note = Bacteroides fragilis
  source 1..2850: mol_type = genomic DNA
atgcaaaaactactatctttaccgcccaatctggttcagtcttttcatgaactggagagggtgaaccgtaccgattggttttgtacttccgacccggtaggtaagaaacttggttccggtggtggaacatcctggttgcttgaagaatgttataatgaatattcagatggtgctacttttggagagtggcttgaaaaagaaaaaagaattcttcttcatgcgggtgggcaaagccgtcgtttacccggctatgcaccttctggaaagattctcactccggttcctgtgttccggtgggagagagggcaacatctgggacaaaatctgctttctctgcaacttcccctatatgaaaaaatcatgtctttggctccggataaactccatacactgattgcgagtggtgatgtctatattcgttcggagaaacctttgcagagtattcccgaagcggatgtggtttgttatggactgtgggtagatccgtctctggctacccatcatggcgtgtttgcttccgatcgcaaacatcccgaacaactcgactttatgcttcagaagccttcgttggcagaattggaatctttatcgaagacccatttgttcctgatggacatcggtatatggcttttgagtgaccgtgccgtagaaatcttgatgaaacgttctcataaagaaagctctgaagaactaaagtattatgatctttattccgattttggattagctttgggaactcatccccgtattgaagacgaagaggtcaatacgctatccgttgctattctgcctttgccgggaggagagttctatcattacgggaccagtaaagaactgatatcttcaactctttccgtacagaataaggtttacgatcagcgtcgtatcatgcaccgtaaagtaaagcccaatccggctatgtttgtccaaaatgctgtagtgcggatacctctttgtgccgagaatgctgatttatggatcgagaacagtcatatcggaccaaagtggaagattgcttcacgacatattattaccggggttccggaaaatgactggtcattggctgtgcctgccggagtgtgtgtagatgtggttccgatgggtgataagggctttgttgcccgtccatacggcctggacgatgttttcaaaggagatttgagagattccaaaacaaccctgacgggtattccttttggtgaatggatgtccaaacgcggtttgtcatatacagatttgaaaggacgtacggacgatttacaggcagcttccgtattccctatggttaattctgtagaagagttgggattggtgttgaggtggatgttgtccgaacccgaactggaggaaggaaagaatatctggttacgttccgaacgtttttctgcggacgaaatttcggcaggtgccaatctgaagcgtttgtatgcacaacgtgaagagttcagaaaaggaaactggcaagcattggccgttaatcatgaaaaaagtgttttctatcaacttgatttggccgatgcagctgaagattttgtacgtcttggtttggatatgcctgaattattgcctgaggatgctctgcagatgtcacgcatccataaccggatgttgcgtgcgcgtattttgaaattagacgggaaagattatcgtccggaagaacaggctgcttttgatttgcttcgtgacggcttgctggacgggatcagtaatcgtaagagtaccccaaaattggatgtatattccgatcagattgtttggggacgtagtcccgtgcgcatcgatatggcaggtggatggaccgatactcctccttattcactttattcgggaggaaatgtggtgaatctggctattgagttgaacggacaacctcccttacaggtctatgtgaagccgtgtaaagatttccatatcgtcctgcgttctatcgatatgggtgctatggaaatagtatctacgtttgatgaattgcaagattataagaagatcggttcacctttctctattccgaaagccgctctgtcattggcaggctttgcacctgcgttttctgctgtatcttatgcttcattagaagaacagcttaaagatttcggtgcaggtattgaagtgactttattggctgctattcctgccggttccggtttgggcaccagttccattctggcttctaccgtacttggtgccattaacgatttctgtggtttagcctgggataaaaatgagatttgtcaacgtactcttgtccttgaacaattgctgactaccggtggtggatggcaggatcagtatggaggtgtgttgcagggtgtgaagcttcttcagaccgaggccggctttgctcaaagtccattggtgcgttggctacccgatcatttatttacgcatcctgaatacaaagactgtcacttgctttattataccggtataactcgtacggcaaaagggatcttggcagaaatagtcagttccatgttcctcaattcatcgttgcatctcaatttactctcggaaatgaaggcgcatgcattggatatgaatgaagctatacagcgtggaagttttgttgagtttggccgtttggtaggaaaaacctgggaacaaaacaaagcattggatagcggaacaaatcctccggctgtggaggcaattatcgatctgataaaagattataccttgggatataaattgccgggagccggtggtggcgggtacttatatatggtagcgaaagatccgcaagctgctgttcgtattcgtaagatactgacagaaaacgctccgaatccgcgggcacgttttgttgaaatgacgttatctgataagggattccaagtatcacgatcatga

SEQ ID NO: 3 - protein, 377 aa, Arabidopsis thaliana
MGTTNGTDYGAYTYKELEREQYWPSENLKISITGAGGFIASHIARRLKHEGHYVIASDWKKNEHMTEDMFCDEFHLVDLRVMENCLKVTEGVDHVFNLAADMGGMGFIQSNHSVIMYNNTMISFNMIEAARINGIKRFFYASSACIYPEFKQLETTNVSLKESDAWPAEPQDAYGLEKLATEELCKHYNKDFGIECRIGRFHNIYGPFGTWKGGREKAPAAFCRKAQTSTDRFEMWGDGLQTRSFTFIDECVEGVLRLTKSDFREPVNIGSDEMVSMNEMAEMVLSFEEKKLPIHHIPGPEGVRGRNSDNNLIKEKLGWAPNMRLKEGLRITYFWIKEQIEKEKAKGSDVSLYGSSKVVGTQAPVQLGSLRAADGKE

SEQ ID NO: 4 - DNA, 1134 bp, Arabidopsis thaliana
  source 1..1134: mol_type = genomic DNA
atgggcacgactaacggcaccgactatggagcgtacacgtacaaagaactggaacgcgaacaatactggccatccgagaatttgaaaatcagtattacgggcgcgggcggcttcattgctagccacatcgcacgccgcctgaaacacgaaggtcactatgtgattgcaagcgattggaagaagaacgagcacatgaccgaagatatgttttgcgatgaatttcatttagtggacctgcgtgtaatggagaattgcttaaaagtgactgagggtgtggatcacgtgttcaatctcgccgcggatatgggcggcatgggctttattcaaagtaaccatagcgtgattatgtacaacaacacgatgattagctttaacatgatcgaggccgcgcgcatcaatggtatcaaacggttcttctatgccagctcggcgtgcatttaccctgaatttaaacagctggaaaccaccaatgtgtccttgaaagaatctgatgcgtggccggcagaaccgcaagacgcgtacggcctggaaaagctggcgactgaagaactatgcaagcactacaataaagattttggtatcgaatgccgcattggccggttccacaacatttatggtccttttgggacgtggaaaggcggacgtgagaaggcgccagccgcgttttgtcgcaaagcgcagacttctacagatcggtttgagatgtggggtgatggtttgcagacccgctcattcacttttatcgacgagtgtgtggaaggagtgctgcgcctgaccaaatcggacttccgcgagcccgttaatatcggttctgacgagatggtgtcgatgaacgaaatggcggaaatggtactgagttttgaagaaaagaaattacctatccatcacattcccggccctgagggagtacggggtcgcaactcagataataacctgatcaaagagaaactgggctgggctccaaacatgcgcctcaaagaaggcctgcgtatcacctacttttggataaaagaacaaatagagaaagaaaaggcgaaaggtagtgatgtctcgttgtatggatcatcgaaagtggtgggtacgcaagccccggttcagctcggcagcctgcgcgcggcagacggaaaagaataa

SEQ ID NO: 5 - protein, 373 aa, Arabidopsis thaliana
MASENNGSRSDSESITAPKADSTVVEPRKIALITGITGQDGSYLTEFLLGKGYEVHGLIRRSSNFNTQRINHIYIDPHNVNKALMKLHYADLTDASSLRRWIDVIKPDEVYNLAAQSHVAVSFEIPDYTADVVATGALRLLEAVRSHTIDSGRTVKYYQAGSSEMFGSTPPPQSETTPFHPRSPYAASKCAAHWYTVNYREAYGLFACNGILFNHESPRRGENFVTRKITRALGRIKVGLQTKLFLGNLQASRDWGFAGDYVEAMWLMLQQEKPDDYVVATEEGHTVEEFLDVSFGYLGLNWKDYVEIDQRYFRPAEVDNLQGDASKAKEVLGWKPQVGFEKLVKMMVDEDLELAKREKVLVDAGYMDAKQQP

SEQ ID NO: 6 - DNA, 1122 bp, Arabidopsis thaliana
  source 1..1122: mol_type = genomic DNA
atggcaagtgagaacaatggttcacgttctgactctgaaagcatcacggctcctaaagcggacagcaccgttgtggaaccacggaaaatcgctctaatcaccggcatcacgggtcaggacggtagttacttgactgaatttctactaggcaaaggttacgaagtgcatggcctgatccgtaggagtagcaattttaacacgcagcggatcaatcatatctatattgatccacacaacgtgaacaaagctttaatgaaactccattacgcggatctcactgacgcctcttcgttgcgtcgctggatcgacgtcattaaacctgacgaagtgtataacctggcggcacagtctcatgtggccgtttcattcgaaatacctgattatacggcggacgtggttgccaccggtgccttaagactgctcgaggcggttcgctcccataccattgattccgggcgcacggtaaaatattatcaggcaggaagcagcgaaatgtttggaagtacgccgccccctcagtctgagacaaccccgtttcacccgcgcagtccgtatgcggcatctaaatgtgccgcacattggtatacagtcaattatcgtgaggcttatggcttgtttgcatgcaatggcattctgttcaatcatgaaagcccgcgcagaggcgaaaattttgttacccgcaaaattacgcgtgccctgggccgtattaaagtaggtctgcaaactaaactgtttcttggcaacctccaggctagccgtgactggggatttgccggtgattatgtcgaagccatgtggctcatgttacagcaggagaaaccggacgattatgttgttgcgacagaagaaggacacacagtggaggaatttttggatgtatcgttcggctatttaggtctaaactggaaagattacgttgagattgatcaacgctacttccggccggcggaagtggacaacctgcaaggagatgcctccaaggcaaaagaagtactgggttggaaaccgcaggtgggcttcgagaaacttgtcaaaatgatggtggatgaagatctggaattagctaaacgcgagaaggtactggtagatgcaggatacatggatgcgaagcagcaaccgtaa

SEQ ID NO: 7 - protein, 321 aa, Escherichia coli
MSKQRVFIAGHRGMVGSAIRRQLEQRGDVELVLRTRDELNLLDSRAVHDFFASERIDQVYLAAAKVGGIVANNTYPADFIYQNMMIESNIIHAAHQNDVNKLLFLGSSCIYPKLAKQPMAESELLQGTLEPTNEPYAIAKIAGIKLCESYNRQYGRDYRSVMPTNLYGPHDNFHPSNSHVIPALLRRFHEATAQNAPDVVVWGSGTPMREFLHVDDMAAASIHVMELAHEVWLENTQPMLSHINVGTGVDCTIRELAQTIAKVVGYKGRVVFDASKPDGTPRKLLDVTRLHQLGWYHEISLEAGLASTYQWFLENQDRFRG

SEQ ID NO: 8 - DNA, 966 bp, Escherichia coli
  source 1..966: mol_type = genomic DNA
atgagcaaacagcgcgtgtttattgccggccatcgtggtatggttggtagcgccattcgtcgccagctggaacagcgtggtgatgtggagctggtgctgcgtacccgcgacgaactgaatttattagatagccgcgccgttcacgactttttcgccagcgaacgcatcgaccaagtttatctggccgccgcaaaagtgggcggtatcgttgccaacaacacctatccggccgactttatctatcagaatatgatgattgaaagcaacatcatccatgccgcccaccagaacgacgtgaacaaactgctgtttttaggtagcagctgcatctacccgaagctggccaaacagccgatggccgaaagcgaactgctgcaaggtacactggaaccgaccaacgaaccttacgcaattgccaagatcgccggcattaagctgtgtgagagctacaaccgccagtacggtcgcgattatcgcagcgttatgccgaccaatttatatggcccgcatgataactttcacccgagtaacagccacgttattccggctttattacgccgtttccacgaagcaaccgcccagaacgccccggatgttgttgtttggggcagcggtacccctatgcgcgagtttttacacgttgatgatatggcagcagccagcatccatgttatggaactggcccatgaagtgtggctggagaacacacagccgatgctgagccatatcaatgtgggcactggtgtggattgcaccattcgtgaactggcccagaccatcgcaaaagtggtgggctacaaaggtcgcgtggtgtttgatgccagcaaaccggatggcacaccgcgcaaactgctggacgtgacccgtttacatcagctgggctggtaccacgaaatcagtttagaggctggtttagccagcacctaccagtggtttttagaaaatcaagatcgctttcgcggttga

SEQ ID NO: 9 - protein, 351 aa, Homo sapiens
MRNVALITGITGQDGSYLAEFLLEKGYEVHGIVRRSSSFNTGRIEHLYKNPQAHIEGNMKLHYGDLTDSTCLVKIINEVKPTEIYNLGAQSHVKISFDLAEYTADVDGVGTLRLLDAVKTCGLINSVKFYQASTSELYGKVQEIPQKETTPFYPRSPYGAAKLYAYWIVVNFREAYNLFAVNGILFNHESPRRGANFVTRKISRSVAKIYLGQLECFSLGNLDAKRDWGHAKDYVEAMWLMLQNDEPEDFVIATGEVHSVREFVEKSFLHIGKTIVWEGKNENEVGRCKETGKVHVTVDLKYYRPTEVDFLQGDCTKAKQKLNWKPRVAFDELVREMVHADVELMRTNPNA

SEQ ID NO: 10 - DNA, 1056 bp, Homo sapiens
  source 1..1056: mol_type = genomic DNA
atgcgaaacgtggccttgatcaccggtattaccggccaggatggctcatatctggcagaatttctgcttgaaaaaggctatgaggttcatggcatcgtgcgccgcagcagtagttttaataccggccgcattgaacatctgtataaaaacccacaagcacacatcgaaggaaatatgaaactgcattatggcgatttgacagactcaacgtgtctggttaagataataaacgaagtgaagcctaccgaaatttacaacctgggtgcgcagtctcatgtgaaaattagcttcgatttggccgaatataccgcggatgtcgatggtgtgggtacgttacgactgttggacgctgttaaaacctgcgggctgatcaacagcgtgaaattttatcaggctagcacgagtgagctctatggaaaggtccaggagattccccagaaggaaacgacgcctttctatccacgcagcccgtatggggcagcaaaactttatgcctattggatcgtagtgaactttcgcgaagcttataatctttttgcggttaatggcatactgtttaaccacgagtcgccacgacgcggcgcaaacttcgtgacccgtaaaataagtcgtagcgtcgcgaagatctatctgggtcagctcgaatgtttcagccttggcaacctggatgcgaaacgtgattggggacacgcgaaagattatgtcgaagccatgtggctgatgttacaaaacgatgaacctgaggacttcgttatcgccacgggtgaagtgcatagcgtacgcgaatttgtcgaaaaaagcttcctccatataggtaagaccatcgtgtgggaaggcaaaaatgagaacgaggttggtcgctgcaaagaaaccggcaaagttcacgttacggttgatctcaaatactacagacccaccgaagtggactttctgcaaggcgattgtaccaaagccaaacagaaactaaattggaaacctcgcgttgccttcgacgaactcgtccgtgaaatggtccatgcagatgtcgaactgatgagaacaaaccctaacgcgtaa

SEQ ID NO: 11 - protein, 373 aa, Escherichia coli [sequence omitted]

SEQ ID NO: 12 - DNA, 1122 bp, Escherichia coli
  source 1..1122: mol_type = genomic DNA
atgagcaaagttgctttaatcaccggtgtgaccggccaagatggcagctatttagccgagtttctgctggagaaaggctacgaagtgcatggtattaagcgtcgcgccagcagcttcaataccgaacgtgtggatcatatctatcaagatccgcacacttgtaacccgaaattccatctgcactatggcgatctgagcgataccagtaatttaacccgcattctgcgcgaagttcagccggatgaggtgtacaatctgggcgccatgagtcatgtggccgtgagctttgaaagcccggaatacaccgccgatgttgatgcaatgggcactttacgtttactggaagccattcgctttttaggtctggagaagaaaactcgcttctaccaagctagcacaagcgaactgtatggtctggtgcaagaaatcccgcagaaagaaactaccccgttttatccgcgtagtccgtatgcagtggccaagctgtatgcctactggatcaccgtgaactaccgtgagagctatggcatgtatgcttgtaacggcattttatttaaccatgagagcccgcgtcgcggcgagacatttgttacccgcaaaattacccgcgcaatcgccaatatcgcacaaggtttagagagttgtttatatctgggcaatatggactctttacgtgactggggccatgccaaagattacgtgaagatgcagtggatgatgctgcagcaagaacagccggaagatttcgtgattgccaccggcgttcagtacagcgttcgtcagttcgtggaaatggccgccgcccagctgggcattaaactgcgtttcgaaggtaccggcgtggaggagaaaggtattgtggttagcgtgaccggccatgatgccccgggcgttaaaccgggcgatgttatcatcgccgtggatccgcgctattttcgcccggccgaagttgaaacactgctgggcgatcctaccaaagcccacgaaaagctgggttggaagcccgaaattactttacgcgaaatggttagcgagatggttgccaatgatctggaagccgccaaaaagcactctttactgaaaagccatggctacgatgtggccattgcactggaaagctga

SEQ ID NO: 13 - protein, 342 aa, Yersinia pseudotuberculosis
MNNVLITGFTGQVGSQLADYILENTDDHVIGMMRWQESMDNIYHLTDRINKKDRISIQYADLNDLMSLYNLIDTVRPKFIFHLAAQSFPRTSFDIPIETLQTNIIGTANLLECIRKLKQQDGYDPVVHVCSSSEVYGRAKVGEALNEDTQFHGASPYSISKIGTDYLGQFYGEAYGIRTFVTRMGTHTGPRRSDVFFESTVAKQIALIEAGHQEPKLKVGNLASVRTFQDARDAVRAYYLLALESGKGNIPNGEVFNIAGDEAFKLPEVIELLLSFSTRNDIEVVTDTDRLRPIDADYQMFDSTKIKSYINWKPEIKAADMFRDLLQHWRNEIASGRIPLNR

SEQ ID NO: 14 - DNA, 1029 bp, Yersinia pseudotuberculosis
  source 1..1029: mol_type = genomic DNA
atgaacaatgttctgattacgggtttcaccgggcaggtaggttcgcagcttgccgattacattctggaaaacaccgacgatcatgtgatcgggatgatgcgctggcaggagagcatggacaatatttatcatttaaccgaccgcatcaacaaaaaagatcggatttcaatccaatacgcggatctgaatgaccttatgtctctgtataatctgatagacacggtccggccgaaattcattttccatttagcggcacagagctttccgcgcacgtcctttgacatcccaatcgaaaccctgcaaacgaatattatcggcactgcgaacctgttggagtgtattcgcaaactgaaacagcaagacgggtacgacccggttgttcatgtctgtagctccagcgaagtgtatgggcgcgcgaaagtgggtgaggccttaaatgaagatacgcaatttcacggcgccagcccgtattccattagcaagattggcacggattatcttggtcagttttatggcgaagcgtacggcattcgcacgtttgttaccaggatgggcacccatacgggtccgcgtcgctcggacgtgtttttcgaaagcaccgttgccaaacagatcgccctgatcgaggcgggccatcaagagccaaagttaaaagtcggcaatttggcctcggtacgcacgtttcaagatgcccgcgatgccgtgcgcgcttactatctcctggcccttgaatccggaaaaggcaatattccgaacggtgaggtctttaacatcgcgggggacgaagcgttcaaactgccggaagtcattgaactgctgctgtccttctcgactcgtaatgatattgaggttgttaccgataccgatcgcttacgtccaatcgacgccgattatcagatgtttgactcgaccaaaatcaaatcatatatcaattggaaaccggaaatcaaggcagcggacatgtttcgcgatctactgcaacattggcgcaatgagatcgccagtggtcgtattccgcttaatcgctaa

SEQ ID NO: 15 - protein, 277 aa, Yersinia pseudotuberculosis
MTKVFILGSNGYIGNNLMESLCDNIEVITVGRSNADIYINLESDDFQSLLNKVEFKDTVIFLSAISSPDECNNNYDYSYKINVKNTISLISLLLAKNVRVMFSSSDAVFGATQNLCDENSEKKPFGKYGEMKSEVEDYFTLEDDFFVVRFSYVLGRNDKFSMMIKEFYEQGKILDVFDGFERNVISINDVTAGIKNIICDWDSIKTRIVNFSGNELVSRQDIVNALVKEKYLNLKYKFTAAPESFWVGRPKKIHTKSNYLESILNRKLESYLEVIKE

SEQ ID NO: 16 - DNA, 834 bp, Yersinia pseudotuberculosis
  source 1..834: mol_type = genomic DNA
atgacgaaagttttcattctgggctcaaatggttacataggtaataacctgatggagtcgctgtgtgataatattgaggtgatcacggtcggtcgttcaaacgctgatatatacattaaccttgaatccgacgatttccagtctctgctgaacaaagtagagtttaaagatacagtgatcttcctgagcgcgatcagtagcccggacgaatgcaataataactatgattatagctataaaattaatgtgaaaaataccataagcctgattagcctcttactagctaaaaacgttcgcgtgatgttctcaagcagcgacgcggtatttggcgctacgcaaaatctgtgcgatgaaaattccgaaaaaaaaccctttggaaagtatggcgaaatgaaaagcgaagttgaagattatttcacccttgaggatgatttctttgtggtccgcttcagctatgtgctggggcgaaacgataaatttagcatgatgatcaaagagttttacgaacagggtaaaatactggatgtgtttgatggctttgaacgtaacgtgattagcataaatgacgtgacagcggggatcaaaaacatcatttgtgactgggattctatcaaaactcgtatcgtcaatttttccggcaacgaattagtttctcgccaggacatcgttaatgcgctggtgaaggaaaaatacctgaacctcaaatacaaatttaccgccgcccccgagtcgttctgggttggccgtcccaaaaagattcacaccaaaagcaattacctggaatcgattttaaaccgtaaactggaaagttacctggaggtcatcaaagagtaa

SEQ ID NO: 17 - protein, 313 aa, Campylobacter jejuni
MSKKVLITGGAGYIGSVLTPILLEKGYEVCVIDNLMFDQISLLSCFHNKNFTFINGDAMDENLIRQEVAKADIIIPLAALVGAPLCKRNPKLAKMINYEAVKMISDFASPSQIFIYPNTNSGYGIGEKDAMCTEESPLRPISEYGIDKVHAEQYLLDKGNCVTFRLATVFGISPRMRLDLLVNDFTYRAYRDKFIVLFEEHFRRNYIHVRDVVKGFIHGIENYDKMKGQAYNMGLSSANLTKRQLAETIKKYIPDFYIHSANIGEDPDKRDYLVSNTKLEATGWKPDNTLEDGIKELLRAFKMMKVNRFANFN

SEQ ID NO: 18 - DNA, 942 bp, Campylobacter jejuni
  source 1..942: mol_type = genomic DNA
atgtccaaaaaggtgctgatcaccggcggcgcgggctatatcggaagtgtcctgaccccgatcctgttagaaaaaggctatgaagtctgtgttatcgacaatctgatgtttgaccagatttctctgctttcctgttttcataataagaatttcacgttcataaacggggatgcgatggatgaaaatctgattcgccaggaagtagccaaagccgatattatcattccgctggcggcactggtcggggcgcctctgtgtaaacgcaacccgaaactggctaaaatgatcaactacgaggcagttaagatgattagcgattttgcctccccatcgcagatctttatttacccaaacaccaatagcggttacgggatcggcgagaaagatgcgatgtgcaccgaagaatcgccgctgcgtccgatttccgagtatgggatcgataaagtgcatgctgaacagtacctgctggataaaggtaactgcgtgacctttcgtttagcaacagtctttggaatttcaccgcgtatgcgccttgatctgctcgtgaatgattttacataccgcgcttatcgtgacaaatttatcgttttattcgaagagcactttcgccgcaactatattcacgttcgtgatgtcgtgaaaggcttcatccatgggatagagaactatgataaaatgaaaggccaagcgtacaacatgggtctgagctcggccaacctaaccaagcgccaactggccgaaaccattaagaaatatattccagacttctacatccattcagcgaacattggagaagatccggataaacgcgactatctggtttcgaatacgaagttggaagccaccggttggaaacctgataatactcttgaggatggcatcaaagaactgttacgtgcttttaaaatgatgaaggttaaccgctttgcgaattttaattaa

SEQ ID NO: 19 - protein, 378 aa, Oryza sativa
MGSSEKNGTAYGEYTYAELEREQYWPSEKLRISITGAGGFIGSHIARRLKSEGHYIIASDWKKNEHMTEDMFCHEFHLVDLRVMDNCLKVTNGVDHVFNLAADMGGMGFIQSNHSVIMYNNTMISFNMLEAARINGVKRFFYASSACIYPEFKQLETNVSLKESDAWPAEPQDAYGLEKLATEELCKHYTKDFGIECRVGRFHNIYGPFGTWKGGREKAPAAFCRKAQTSTDRFEMWGDGLQTRSFTFIDECVEGVLRLTKSDFREPVNIGSDEMVSMNEMAEIILSFEDRELPIHHIPGPEGVRGRNSDNTLIKEKLGWAPTMKLKDGLRFTYFWIKEQIEKEKTQGVDIAGYGSSKVVSTQAPVQLGSLRAADGKE

SEQ ID NO: 20 - DNA, 1161 bp, Oryza sativa
  source 1..1161: mol_type = genomic DNA
atgggctccagtgagaagaacggaactgcctatggcgaatatacatacgctgagttagaacgcgagcagtattggccatcggagaaattacggatttcaattaccggggccggcggcttcatcggctcccacatcgcgcgacgactgaagagtgaaggtcattatattatcgcctcggactggaagaagaacgaacacatgaccgaggacatgttttgtcacgagttccatctggtggaccttcgagtgatggataactgtttaaaagtgacgaatggcgttgaccatgtatttaatctggcagccgatatgggcgggatgggctttattcaatcgaaccattcggtgattatgtataataacacgatgatttcgttcaacatgttagaagcggcccgtattaacggcgttaaacgtttcttctatgcatcttcagcttgcatttatccagagttcaaacagcttgaaaccaatgtgtctctgaaggaatctgatgcgtggcccgcagaaccacaggacgcatacggcttagaaaagctggcgaccgaggaactctgtaaacactacaccaaagatttcggcattgagtgccgcgtaggtcgctttcataacatttatgggccatttggcacctggaaaggtggtcgcgaaaaggcgccagcggccttctgtcgaaaagcacagacatccaccgaccgtttcgaaatgtggggtgatggcttgcaaacacggtcttttacattcattgacgaatgcgttgagggtgttctgagattgacaaaatcggattttcgtgagcctgttaacattggcagcgacgagatggtgagtatgaacgaaatggccgagatcattctgtcttttgaagatcgcgaactgcctattcaccatattccgggaccggaaggtgtacgtggccgcaattcggacaatactctgatcaaagaaaagctgggctgggctccgaccatgaaattaaaagacgggctccgtttcacttacttctggattaaagagcagattgagaaagagaaaacgcaaggggttgacattgccggttacggcagcagtaaagttgttagtacccaggccccggtgcaacttggttctctgcgcgcagcagacgggaaagaaagcttgcatcaccatcaccatcactaa

SEQ ID NO: 21 - protein, 298 aa, unidentified
  REGION 1..298: note = Halorubrum sp. T3
MVSIILRGGLGNQLFQYATGRAHSLRTNSTLFVNLSKLDSNLGPDVAKRSLHLEAFDLPVEYVDNETSHSFGRTIRRRIPQVVASINQLLATHLFKLYVEDQSLTFDPNVPNLPGNVTLDGYWQSERYFTEFTETLRREITVRNPVSGENQRWYDLISDTGSVSVHVRRGDYVDLGWALPPSYYRNALNQIQDETDVTDLFFFSDNIDWIRTNQKDLVPDHSDTNVHYVECNDGETAHEDLRLMRACDHHIVANSSFSWWGAWLDNSETKIVIAPDYWVHDPVNHLDIIPDRWDTVSW

SEQ ID NO: 22 - DNA, 897 bp, unidentified
  misc_feature 1..897: note = Halorubrum sp. T3
  source 1..897: mol_type = genomic DNA
atggtctcgataatcctacgcggtggactcggcaaccaactattccagtacgcgacgggacgcgcacactcactccgaactaattctactctttttgtaaacctctctaaacttgactcgaaccttggccccgacgtagcgaaacgatcgctacatcttgaggcgttcgatcttccagttgaatatgtagataatgagacaagccacagttttggcaggacgatacgcagacggatcccgcaggtcgtcgcaagtataaaccagttactagcgacacatctcttcaaattgtacgtcgaagatcagtcactgacgttcgatccgaatgtccctaatctacctggaaacgtcacactcgacggttactggcaatccgaacgctattttacagagtttaccgagacgcttcggcgtgaaattacggttcgtaatcctgtgtctggtgaaaaccaacggtggtacgacctcatctccgatactggctcagtaagtgtacacgtccgtcgtggagactacgttgatctcggctgggcacttccaccgtcctactacagaaatgccctcaatcagattcaggatgaaactgacgtgacagatctgttttttttctccgacaacattgactggattcgtaccaaccagaaagaccttgtgccggatcacagcgataccaacgtacactacgtcgagtgtaacgatggagaaacggcccacgaggatctccgtctgatgcgagcctgtgatcaccatatcgtcgccaacagcagcttcagttggtggggtgcgtggctggataattctgagacgaaaattgtcatcgctcccgactattgggttcatgacccggtcaatcatctcgatattattcccgatcgatgggataccgtcagttggtag

SEQ ID NO: 23 - protein, 291 aa, unidentified
  REGION 1..291: note = Pisciglobus halotolerans
MIYTRITSGLGNQMFQYAIAYSYSRKYDMPLILDLTNFKISKKRTYQLDKFKLNDYKKITFKNAPLEIKIFWLVEVLNMISIKLRKKEMKRKNNYNLKSTQFICEKYKEKYNINFDLTNKSLYLSGFWQSPLYFENYRDELIQQFSPNYVLSNKLKEYETKIINCRSVSVHIRRGDFLQHGLFKDVDYQKKAITYLEKKLDNPIFFFFSDDIEWTKEKFKNQKNCFFVSSDSKNSGIEEMYLMSKCENNIIANSTFSWWGAWLNQNQNKIVIAPSTGFGNKDILPKSWYTI

SEQ ID NO: 24 - DNA, 876 bp, unidentified
  misc_feature 1..876: note = Pisciglobus halotolerans
  source 1..876: mol_type = genomic DNA
atgatatatacacgaataacgagcggtttagggaaccaaatgtttcagtatgctattgcgtattcgtattctaggaaatatgatatgccacttattcttgatcttacaaattttaaaatttcaaaaaagagaacctatcaattagataaattcaaacttaatgattataaaaagataacatttaaaaatgctccattagaaataaaaatattttggttggtagaggttttaaacatgatttctattaaactaaggaaaaaagaaatgaaaagaaaaaataattataacttgaaatcaactcaatttatatgcgagaaatataaagaaaaatataacataaactttgatctcacaaataaatcactttatttatctggattttggcaaagtcctttatattttgaaaactatagagatgaattaatacaacagttttctcctaattatgttttatcaaataagttaaaagaatatgaaactaagataataaactgtagaagcgtttctgttcatattagaagaggagattttttacaacatggtttatttaaagatgtagattaccaaaagaaagctataacttatttagaaaagaaattagataaccctatttttttctttttttcagacgatattgaatggacaaaagaaaaatttaaaaatcaaaaaaattgtttttttgtatcttcagattcaaaaaattctggtatagaagaaatgtatcttatgtctaagtgtgagaacaatatcattgcaaatagtacttttagttggtggggagcatggttaaatcaaaaccaaaataaaattgtaatagcaccaagcactggttttggtaataaagatatattaccaaaatcttggtatacaatttag

SEQ ID NO: 25 - protein, 297 aa, unidentified
  REGION 1..297: note = Anaerovibrio sp. RM50
MLVVSMGCGLGNQMFEYAFYKHLCKKYTSEIIKLDIRHAFPFAHNGIELFDIFDLSGEVASKQEVLFLTSGYGLHGVGFEYKTIFHRIGEKVRKLFSLTPQTMKIQDDYTEYYNEFFNVMPGKSVYYLGVFANYHYFKEIQYDIKNIYKFPTIDDLKNKRYAEKMENCNSVSIHVRRGDYVSEGVKLTPLSFYRKAILKIEEKVKNAHFFVFADDVEYARSLFPDNDHYTFVEGNNGKNSFRDMQLMSLCKHNITANSTFSFWGAFLNSNPSKIVIAPNLPYTGAKYPFVCDDWVLI

SEQ ID NO: 26 - DNA, 894 bp, unidentified
  misc_feature 1..894: note = Anaerovibrio sp. RM50
  source 1..894: mol_type = genomic DNA
atgcttgttgttagtatggggtgtggtttggggaatcagatgtttgaatatgcattttataagcatttatgtaaaaaatatacaagcgagataattaaacttgatataagacacgcatttccgtttgctcataatggaattgagctatttgatatttttgatttatctggagaagttgcgagtaagcaagaagttctgtttttgacgtcagggtatggcctacatggtgttgggtttgaatataaaactatttttcacagaataggagaaaaagtaagaaaacttttctcgttgacaccacaaactatgaaaattcaagatgattatacagagtattataatgaattttttaatgtgatgcccggtaaatcggtgtactatctaggtgttttcgcaaattaccattattttaaggagatacaatatgatataaaaaatatatacaaatttcctactatagatgatctgaaaaacaaaagatatgcagaaaaaatggaaaattgtaattcagtatctattcacgttagaagaggagattatgtaagcgaaggagtaaagcttacgcccttatcattttatagaaaagctattttaaagattgaagaaaaggtaaaaaatgctcatttttttgtcttcgcagatgatgtagagtatgctcgttcgctttttcctgataatgatcattatacgtttgtagaaggaaataatggcaagaatagttttcgcgatatgcaacttatgagtttatgtaagcataatatcacagcaaacagtacgtttagcttttggggagcatttttaaattcaaatcctagtaaaatagttatagcgcccaacttgccatatacaggtgcaaaatatccatttgtatgtgatgattgggtgttgatatag

SEQ ID NO: 27 - protein, 299 aa, unidentified
  REGION 1..299: note = Ferrovum sp. 37-45-19
MIITRLIGGLGNQIFQYAVGRAVAARTNTPLLLDASGFPGYELRRYELDGFNVRAELVSAAQLARVGVTASAPHSLLERIKLRFFSQSTQKLPLREPILREASFTYDTRIEYVQAPIYLDGYWQSERYFSAIRMQLLQELTLKNEWGVGNEDMFAQIQAAGLGAVSLHVRRGDYVTNSHTATYHGVCSLDYYRAAVAYIAERVAAPHFFIFSDDHDWVSTNLQTGFPTTFVSVNSADHGIYDMMLMKTCRHHVIANSSFSWWGAWLNPYQDKIVVAPQRWFSGASHDISDLIPASWIRI

SEQ ID NO: 28 - DNA, 900 bp, unidentified
  misc_feature 1..900: note = Ferrovum sp. 37-45-19
  source 1..900: mol_type = genomic DNA
atgatcattactcgtctaattggtggtctcggcaatcaaatattccaatatgcagtgggtcgcgccgtcgccgcgcgcacgaacacgcctctgctgctggacgcttccggttttccgggttatgaattgcggcgttacgagctcgatggtttcaacgtccgcgccgaactggtctcggctgcgcaactggcccgcgttggggtaaccgccagcgctccccactctttgctggagcgaatcaagctccgttttttctctcaatccacgcagaagctacctctgcgggagccgatcctgcgcgaagccagctttacctacgatacccgcattgaatacgtacaggcaccgatctatctggatggatattggcagagcgagcgttatttctcggctatccgcatgcagctgctgcaggagctaactctcaaaaacgagtggggagtaggaaacgaagatatgtttgctcagatccaggctgccggactcggcgccgtgtcgctgcatgtccgccggggcgattatgtgacaaattcccacacggctacttatcacggagtatgctcgctggattactaccgtgcggcagtggcttacatcgccgaacgcgtggcagcgccgcattttttcatcttttccgatgaccacgactgggtcagcaccaatctgcagaccggattcccgaccacttttgtctccgttaattccgctgaccatggcatctacgacatgatgctgatgaagacctgccgtcatcacgtaatcgccaatagctccttcagctggtggggcgcctggttgaatccttatcaagacaagatcgtggttgcgccgcaacgctggtttagcggcgcatcgcacgacataagtgacctcattccggcttcttggatccgaatatga

SEQ ID NO: 29 - protein, 307 aa, unidentified
  REGION 1..307: note = Lachnospiraceae bacterium XBB2008
MIILQMSGGLGNQMFQYALYLKLKKLGREVKFDDETSYELDNARPVQLAVFDITYPRATRQEVTDMRDSSPAWKDRIRRKLKGRNLKQYTEANYSYDEHVFELDDTYLRGYFQTEKYFSDIRDEIYKTYTMRKDLITEQTTQYEEDILSHENSVSIHIRRGDYMTIEGGEIYAGICTDEFYDSAIKYVLERHPDAVFYLFTNDSSWAEYFCNIHSDVNIHVVEGNTEYFGYLDMYLMSRCKHHIVANSSFSWWGAWLGRDADGMVIAPDPWFNCSNCADIHTDRMILIDPKGELLTDDKGVRNESEE

SEQ ID NO: 30 - DNA, 924 bp, unidentified
  misc_feature 1..924: note = Lachnospiraceae bacterium XBB2008
  source 1..924: mol_type = genomic DNA
atgatcatattacagatgagcggcggactcgggaatcagatgttccagtacgctttatatctgaaacttaagaagctcggcagagaagtcaaattcgatgatgagacgagctatgaacttgataatgcgagaccggtacagcttgccgtttttgacataacctatcctcgtgcgacgagacaggaagtcaccgacatgcgcgattcttcccccgcatggaaggacaggatcagacgtaagttaaaaggccggaacctgaagcagtacaccgaagcaaactacagttacgatgaacatgtattcgagctggacgatacgtatcttcggggatattttcagaccgagaagtatttttccgatatcagggatgagatctacaagacatacacgatgcgtaaggatctgatcaccgaacagactacgcagtatgaggaagacatattaagtcatgaaaacagtgtgagcatccatatacgccgcggcgattacatgaccatagagggcggagagatatatgccggcatctgtacggacgaattttatgactcagccataaagtatgttcttgagagacatccggatgctgtattttatctttttaccaatgacagttcatgggcggagtatttctgtaacatacattccgatgtgaacattcatgtcgtcgaaggcaataccgaatatttcggatacctggacatgtacctgatgagcaggtgtaagcatcatatcgtggcaaacagttctttttcatggtggggagcatggctcggcagggatgcggacggtatggtcatagcaccggatccgtggtttaactgcagcaactgtgcggacatccacaccgacaggatgatcctgatcgatcccaagggtgagctgttgacagatgataagggcgtaagaaatgagtcagaagaataa

SEQ ID NO: 31 - protein, 281 aa, unidentified
  REGION 1..281: note = Phaeobacter sp. CECT 7735
MIIARLFGGLGNQMFQYAAGKSLAERLGAELALDFRIIDERGTRRLTDVFDLDIVPATNLPATKHENLLRYGLWRAFGQSPKFRRETGLGYNAAFAEWSDDTYLHGYWQSEQYFSAISDHLRRVFQAVPAPSKENGAIADDIRDCSAISLHVRRGDYLALGAHGVCDEAYYNAALSHIAPQLNQDPRVFVFSDDPQWAKDNLPLPFEKIVVDLNGPTTDYEDLRLMSLCDHNIIANSSFSWWGAWLNANPDKIVTAPANWFADAKLDNPDILPEGWQRITP

SEQ ID NO: 32 - DNA, 846 bp, unidentified
  misc_feature 1..846: note = Phaeobacter sp. CECT 7735
  source 1..846: mol_type = genomic DNA
atgatcattgcaagactgttcgggggtctgggaaaccagatgttccaatatgccgcaggaaagtcactcgctgaacgattgggtgctgagcttgcactcgattttagaataattgatgaacgtggcacccgccgcctgacagacgtgtttgacctcgacattgtgccggcaacaaaccttcccgccaccaaacatgaaaatcttctgagatatgggctatggcgtgcattcggtcagtccccaaaatttcgacgcgagacaggtcttggatacaatgccgccttcgcggaatggagcgacgacacttatctgcatggctattggcagtcagagcagtatttttcggcaatctccgaccatttacgccgcgtgtttcaagcggtgcctgcaccgtcgaaagagaatggtgcaattgcagatgacattcgcgactgcagcgcgatctcgctgcatgtgcgccgcggggactaccttgcccttggggcgcatggcgtctgtgatgaagcctattacaatgcggcgttgtctcatatcgcaccccaattgaaccaagatccacgtgtctttgtgttttccgacgatccgcaatgggccaaagacaaccttcccctgccgtttgaaaagattgtcgtcgatctgaacggcccgacaaccgactatgaagacctgcgattgatgagcctgtgcgaccacaacatcatcgcaaacagttcattttcctggtggggcgcatggttaaacgcaaaccctgacaagattgtcaccgcgccagcaaactggttcgcggatgcaaagttggacaaccccgacattctccctgaaggctggcaaaggatcaccccctga

SEQ ID NO: 33 - protein, 293 aa, unidentified
  REGION 1..293: note = Runella sp. YX9
MYFQKKMIIVKLSGGLGNQLFQYALGRQLSIVNHTDLKMDTTNFSQPSGGTTRTFALGSFNIHAAQANKDEIKLLAGEPNRIFQRVRRKIGLMPIHYFKEPHFHFYQPVLSLQDGVYLDGYWQSEKYFAEIADRIREDLKPVGSFSNQYETFKQSIKQSVSVSVHIRRGDYTTTSKANRYLKPCEALYYQTAVEYLTKRISNLVFFVFSDDIEWAKAHIHFGFPMQYVEGNSAQEDLLLIASCQHHIIANSTFSWWGAWLNPHPDKIVIAPQKWFSTERFDTKDLLPESWILL

SEQ ID NO: 34 - DNA, 885 bp, unidentified
  misc_feature 1..885: note = Runella sp. YX9
  source 1..885: mol_type = genomic DNA
atggcttacttccagaaaaagatgatcatcgttaaactgagtggcggtctgggcaatcagctgtttcagtatgccctgggtcgtcagctgagcattgttaatcataccgatctgaaaatggataccaccaattttagccagccgagcggtggcaccacccgtacctttgcactgggcagctttaatattcatgccgcacaggccaataaggatgaaattaagctgctggcaggcgaaccgaatcgcatttttcagcgtgttcgtcgcaaaattggcctgatgccgattcattattttaaagaaccgcattttcacttctaccagccggtgctgagtctgcaggatggcgtgtatctggatggctattggcagagtgaaaaatattttgccgaaattgccgatcgcattcgcgaagatctgaaaccggtgggtagttttagcaatcagtatgaaacctttaagcagagcattaagcagagcgttagcgttagcgtgcatattcgtcgtggtgactataccaccaccagtaaagccaatcgctatctgaaaccgtgtgaagcactgtattatcagaccgcagttgaatatctgaccaaacgtattagcaatctggttttctttgtgtttagtgatgatattgagtgggccaaagcccatattcattttggttttccgatgcagtatgttgaaggcaatagtgcccaggaagatctgctgctgattgcaagctgccagcatcatattattgccaatagtacctttagctggtggggtgcatggctgaatccgcatccggataaaattgtgattgccccgcagaaatggtttagtaccgaacgttttgataccaaagatctgctgccggaaagctggattctgctgtaa

SEQ ID NO: 35 - protein, 288 aa, Pseudomonas asplenii
MIISNIIGGLGNQMFQYAMARSLSLELKSDLLLDISSYDSYPLHQGYELDRVFKVRSSLAKVEDVKSVLGWQQNLFIHRVLRRPQFSWLRKKSLAIEPFFQYWEGVNFLPKNCYLFGYWQSEKYFNKFSEVIRQDFSFDSNMSEENSFYSERIRKSNSVSVHIRRGDYLNNSVYASCSLEYYRSAIAHVSARSGNPVFFVFSDDIEWVKDNLEFEAESYFVAHNKAGESYNDMRLMSYCKHHVIANSSFSWWGAWLNPSPEKIVIAPKQWFTDGTNTKDLIPSEWMVL

SEQ ID NO: 36 - DNA, 870 bp, Pseudomonas asplenii
  source 1..870: mol_type = genomic DNA
atggctatcatcagtaacatcatcggcggtctgggtaatcagatgtttcagtatgcaatggctcgtagtctgagtctggaactgaaaagcgatctgctgctggatattagcagttatgatagctatccgctgcatcagggctatgaactggatcgtgtttttaaagttcgtagtagcctggccaaagtggaagatgtgaaaagtgtgctgggctggcagcagaatctgtttattcatcgcgtgctgcgtcgtccgcagtttagctggctgcgtaaaaaatctctggccattgaaccgtttttccagtattgggaaggcgttaattttctgccgaaaaattgttatctgttcggttattggcagagcgaaaaatattttaataagttcagcgaggttattcgtcaggattttagttttgatagtaacatgagtgaggaaaatagtttttacagtgaacgtattcgcaaaagcaatagcgtgagtgttcatattcgtcgtggtgactatctgaataatagcgtttatgccagttgtagtctggaatattatcgtagtgccattgcacatgtgagcgcccgcagcggtaatccggtgtttttcgtttttagtgatgatattgagtgggtgaaagataatctggaatttgaagcagaaagttatttcgttgcccataataaggcaggcgaaagttataatgatatgcgtctgatgagttattgtaaacatcatgtgattgccaatagtagctttagctggtggggtgcctggctgaatccgagcccggaaaaaattgttattgcaccgaaacagtggtttaccgatggcaccaataccaaagatctgattccgagcgaatggatggttctgtaa

SEQ ID NO: 37 - protein, 278 aa, unidentified
  REGION 1..278: note = Clostridiales
MVIIKMMGGLGNQMFQYALYKAFEQKHIDVYADLAWYKNKSVKFELYNFGIKINVASEKDINRLSDCQADFVSRIRRKIFGKKKSFVSEKNDSCYENDILRMDNVYLSGYWQTEKYFSNTREKLLEDYSFALVNSQVSEWEDSIRNKNSVSIHIRRGDYLQGELYGGICTSLYYAEAIEYIKMRVPNAKFFVFSDDVEWVKQQEDFKGFVIVDRNEYSSALSDMYLMSLCKHNIIANSSFSWWAAWLNRNEEKIVIAPRRWLNGKCTPDIWCKKWIRI

SEQ ID NO: 38 - DNA, 837 bp, unidentified
  misc_feature 1..837: note = Clostridiales
  source 1..837: mol_type = genomic DNA
atggttattatcaagatgatgggtggtctgggcaatcagatgtttcagtatgccctgtataaagcatttgaacagaaacatatcgacgtgtatgccgatctggcatggtataaaaataagagcgtgaaatttgagctgtataattttggcattaagatcaatgtggccagtgaaaaagatattaatcgtctgagcgattgccaggcagattttgttagtcgcattcgccgcaaaatttttggcaaaaagaaaagtttcgtgagtgaaaagaatgatagttgttatgaaaacgacatcctgcgtatggataatgtgtatctgagcggttattggcagaccgaaaaatattttagcaatacccgtgaaaagctgctggaagattatagctttgcactggttaatagtcaggtgagcgaatgggaagatagcattcgtaataagaatagtgttagcattcacattcgccgtggtgactatctgcagggcgaactgtatggcggcatttgtaccagtctgtattatgccgaagccattgaatatattaagatgcgcgttccgaatgccaaatttttcgtttttagtgatgacgtggaatgggtgaaacagcaggaagattttaaaggttttgtgattgttgaccgtaatgaatatagcagtgcactgagtgatatgtatctgatgagcctgtgcaaacataatattattgccaatagtagcttcagctggtgggcagcatggctgaatcgtaatgaagaaaaaattgttatcgcgccgcgccgttggctgaatggcaaatgtaccccggatatttggtgcaaaaaatggattcgcatttaa

SEQ ID NO: 39 - protein, 293 aa, unidentified
  REGION 1..293: note = Thermosynechococcus elongatus
MIIVRLCGGLGNQMFQYAAGLAAAHRIGSEVKFDTHWFDATCLHQGLELRRVFGLELPEPSSKDLRKVLGACVHPAVRRLLSRRLLRALRPKSLVIQPHFHYWTGFEHLTDNVYLEGYWQSERYFSNIADIIRQQFRFVEPLDPHNAALMDEMQSGVSVSLHIRRGDYFNNPQMRRVHGVDLSEYYPAAVATMIEKTNAERFYVFSDDPQWVLEHLKLPVSYTVVDHNRGAASYRDMQLMSACRHHIIANSTFSWWGAWLNPRPDKVVIAPRHWFNVDVFDTRDLYCPEWIVL

SEQ ID NO: 40 - DNA, 885 bp, unidentified
  misc_feature 1..885: note = Thermosynechococcus elongatus
  source 1..885: mol_type = genomic DNA
atggctatcatcgtgcgtctgtgcggtggtctgggtaatcagatgtttcagtatgccgcaggtctggcagccgcacatcgcattggtagtgaagtgaaatttgatacccattggtttgatgcaacctgtctgcatcagggcctggaactgcgtcgtgtgtttggtctggaactgccggaaccgagcagcaaagatctgcgcaaagttctgggtgcatgcgtgcatccggcagttcgccgtctgctgagtcgccgtctgttacgtgcactgcgtccgaaaagtctggttattcagccgcattttcattattggaccggttttgaacatctgaccgataatgtttatctggaaggttattggcagagcgaacgctattttagcaatattgcagatattatccgccagcagtttcgctttgtggaaccgctggaccctcataatgccgccctgatggatgaaatgcagagtggcgttagtgtgagcctgcatattcgccgtggtgactattttaataatccgcagatgcgccgtgttcatggcgttgatctgagtgaatattatccggccgcagtggccaccatgattgaaaaaaccaatgccgaacgtttttatgtgtttagtgatgatccgcagtgggttctggaacatctgaaactgccggttagctataccgtggttgatcataatcgtggtgccgcaagttatcgcgatatgcagctgatgagtgcatgtcgccatcatattattgcaaatagcacctttagttggtggggtgcatggctgaatccgcgcccggataaagtggttattgccccgcgtcattggtttaatgtggatgtttttgatacccgtgatctgtattgcccggaatggattgtgctgtaa

SEQ ID NO: 41 - protein, 294 aa, unidentified
  REGION 1..294: note = Porphyromonadaceae bacterium H1
MIISKLKGGLGNQLFQYAIGRKMALEQGVELKLELSFFERQNNKTQARDFGLSCFNIDASIASSEDIRMILGPHFLRPLKRRLSKMGIPLFRWNYVRENSWAYHPEILKKKAPLILDGYWQSAAYFESIRDVLLSDFELKAECVSDKLRLLQKQITTESSVALHVRRGDYVTNPIVAKEFGICSESYYEEAVSYMKALEGEPVFFVFSDDIDWCKKHFGEKAGTFVFVSGNQDYEDLMLMSACKHQIIANSSFSWWSAWLNKNPEKKVIAPKIWFADTQMYKTEHIVPQEWIRI

SEQ ID NO: 42 - DNA, 888 bp, unidentified
  misc_feature 1..888: note = Porphyromonadaceae bacterium H1
  source 1..888: mol_type = genomic DNA
atggctatcatcagcaaactgaaaggtggcctgggcaatcagctgtttcagtatgccattggccgcaaaatggccctggaacagggtgttgaactgaaactggaactgagtttctttgaacgtcagaataataagacccaggcccgtgattttggtctgagttgttttaatattgacgccagcattgcaagtagcgaagatattcgtatgattctgggcccgcattttctgcgtccgctgaaacgtcgcctgagcaaaatgggcattccgctgtttcgttggaattatgttcgcgaaaatagttgggcctatcatccggaaattctgaaaaagaaagcaccgctgattctggatggttattggcagagtgcagcctattttgaaagcattcgtgatgttctgctgagcgattttgaactgaaagcagaatgcgttagtgataaactgcgtctgctgcagaaacagattaccaccgaaagtagcgtggccctgcatgtgcgccgcggtgactatgttaccaatccgattgttgcaaaagaatttggtatttgcagtgaaagttactatgaagaagcagttagttatatgaaggcactggaaggtgaaccggttttctttgtgtttagcgatgatattgattggtgtaaaaagcatttcggcgaaaaagcaggtacctttgtgtttgtgagcggtaatcaggattatgaagatctgatgctgatgagcgcatgtaaacatcagattattgcaaatagtagcttcagctggtggagcgcctggctgaataagaatccggaaaagaaagttattgcaccgaaaatttggtttgcagatacccagatgtataaaaccgaacatattgttccgcaggaatggattcgcatttaa

SEQ ID NO: 43 - protein, 304 aa, unidentified
  REGION 1..304: note = Bradyrhizobium
MITVSLIGGLGNQMFQYAAGKALAERHGVPLVLDLSGFRDYAVRSYLLDRLHVPEAGGALGQAESFQKFAARFARAKWKGRIDRLLGQVGLPKIVASSQEYREPHFHYDPAFEALGPSAVLFGYFQSERYFGSISESLSDWFSAREPFGDTAADMLARIETSPLAISVHVRRGDYLNPGTAEFHGILGESYYRQALGRLERLCGQDSELFVFSDDPPAAEKVLDFASRSRLVHVRGDPERPWEDMALMARCHHHIIANSSFSWWGAWLNRSPHKHVVAPRAWFAPAELEKTNTADLYPAEWILV

SEQ ID NO: 44 - DNA, 918 bp, unidentified
  misc_feature 1..918: note = Bradyrhizobium
  source 1..918: mol_type = genomic DNA
atggctatcaccgttagtctgattggcggcctgggcaatcagatgtttcagtatgcagccggcaaagccctggcagaacgtcatggtgtgccgctggttctggatctgagtggctttcgtgattatgcagtgcgcagctatctgctggatcgcctgcatgttccggaagccggcggcgctctgggccaagcagaaagctttcagaaatttgccgcacgttttgcccgcgcaaaatggaaaggtcgcattgatcgtctgctgggccaggtgggtctgccgaaaattgttgcaagcagccaggaatatcgcgaaccgcattttcattatgatccggcatttgaagcactgggtccgagcgccgtgctgtttggctattttcagagcgaacgttattttggtagcattagtgaaagcctgagtgattggtttagcgcccgtgaaccgtttggtgacaccgccgccgatatgctggcccgtattgaaaccagcccgctggccattagcgtgcatgttcgccgcggtgactatctgaatccgggcaccgccgaatttcatggtattctgggtgaaagctattatcgccaggcactgggtcgcctggaacgcctgtgcggtcaggatagtgaactgtttgtgtttagtgatgatccgccggccgcagaaaaagtgctggattttgccagtcgcagccgtctggttcatgttcgcggcgatccggaacgcccgtgggaagatatggcactgatggcccgctgccatcatcatattattgcaaatagtagtttcagctggtggggcgcctggctgaatcgtagtccgcataaacatgttgtggcaccgcgtgcatggtttgccccggcagaactggaaaaaaccaataccgcagatctgtatccggccgaatggattctggtttaa

SEQ ID NO: 45 - protein, 315 aa, unidentified
  REGION 1..315: note = Candidatus Brocadia sapporoensis
MQLKRWPQLKPTDAAVFGSGKQTIMIIVKLMGGLGNQMFQYAAGRRLAEKLGVKLKLDIEMFKDNTLRKYELGAFNIQECFAAVEEIERLTVVKRGIVEKALDRVFKRPIRRPGGYVAEKYFVFDPSILQLPDQVYLDGYWQSEKYFAEIETIIREEFTIKYPQTDKNKVLSDSIKSGNSVTVHVRRGDYVNNPETNSLHGVCGIDYYQRCIDFIITKIANPHFFFFSDDPEWVKNNLKIKYESTVVEHNGAEKCYEDLRLLSQGKYHIIANSTFSWWGAWLNKNPEKMVVAPEKWFKKEDVNTKGFIPEDWIRL

SEQ ID NO: 46 - DNA, 951 bp, unidentified
  misc_feature 1..951: note = Candidatus Brocadia sapporoensis
  source 1..951: mol_type = genomic DNA
atggctcagctgaaacgttggccgcagctgaaaccgaccgatgcagccgtgtttggcagtggcaaacagaccattatgattattgttaaactgatgggtggtctgggcaatcagatgtttcagtatgccgccggccgccgtctggccgaaaaactgggtgttaaactgaaactggatattgaaatgttcaaggataacaccctgcgcaaatatgaactgggcgcattcaatattcaggaatgttttgccgcagttgaagaaattgaacgtctgaccgttgttaaacgcggtattgttgaaaaagccctggatcgcgtttttaaacgcccgattcgccgtccgggtggttatgttgccgaaaaatattttgttttcgacccgagtattctgcagctgccggatcaggtttatctggatggctattggcagagtgaaaaatatttcgcagaaattgaaaccatcatccgcgaagaattcactattaagtatccgcagaccgataaaaataaggttctgagcgatagtattaagagcggcaatagcgtgaccgtgcatgtgcgtcgtggtgactatgttaataatccggaaaccaatagcctgcatggcgtgtgcggtattgattattatcagcgctgtattgatttcattatcaccaaaattgcgaacccgcatttctttttctttagtgatgatccggaatgggttaaaaataatctgaaaattaagtacgagagcaccgtggtggaacataatggcgcagaaaaatgctatgaagatctgcgtctgctgagtcagggtaaatatcatattattgccaatagcaccttcagttggtggggtgcatggctgaataagaatccggaaaaaatggttgttgccccggaaaaatggtttaaaaaagaagatgtgaacaccaaaggctttattccggaagattggattcgtctgtaa

SEQ ID NO: 47 - protein, 273 aa, unidentified
  REGION 1..273: note = Lacinutrix sp. Hel_I_90
MIVIKLIGGLGNQMFQYATAKAIALHKNTTLKLDVSAFENYDLHDYSLDHFNITAKKYQQPPKWLKKIQNKLKPKTYYNEESFRYNSFLFDSNAKTILLNGYFQSEQYFLKYREEIIKDFSITSPLKPETKALLQKVHKTNAVSIHIRRGDFLKHDVHNTFKEEYYKKAMKTIESKIDNPTYYLFSDDMPWVKLNFKSNFKTVYVDFNDAQTAFEDLVLMSNCKHNIIANSSFSWWAAWLNTNPSKIVIAPEQWFNGNKYDYTDVVPETWVKI

SEQ ID NO: 48 - DNA, 825 bp, unidentified
  misc_feature 1..825: note = Lacinutrix sp. Hel_I_90
  source 1..825: mol_type = genomic DNA
atggctatcgtgattaagctgattggtggtctgggtaatcagatgtttcagtatgccaccgccaaagcaattgccctgcataaaaataccaccctgaaactggatgttagtgcctttgaaaattatgatctgcatgattatagcctggatcattttaatatcaccgcaaaaaagtaccagcagccgccgaaatggctgaaaaagattcagaataagctgaaaccgaaaacctattataacgaagaaagttttcgctataacagttttctgtttgatagcaatgccaaaaccattctgctgaatggttattttcagagcgaacagtattttctgaaatatcgtgaagaaatcatcaaggatttcagtattaccagcccgctgaaaccggaaaccaaagcactgctgcagaaagtgcataaaaccaatgccgttagcattcatattcgccgtggcgattttctgaaacatgatgttcataataccttcaaagaggaatattacaagaaggccatgaaaaccattgaaagcaaaattgataacccgacctattatctgtttagtgatgatatgccgtgggttaaactgaattttaaaagcaatttcaagaccgtgtacgtggattttaatgatgcccagaccgcatttgaagatctggtgctgatgagcaattgtaaacataatattatcgccaacagcagttttagctggtgggccgcctggctgaataccaatccgagcaaaattgttattgcaccggaacagtggtttaatggtaataagtatgattacaccgacgttgtgccggaaacctgggttaaaatttaa

SEQ ID NO: 49 - protein, 277 aa, unidentified
  REGION 1..277: note = Butyrivibrio proteoclasticus
MIIIKFCGALGNQLFQYALYEKMRILGKDVKADISAFGDGNEKRFFYLDELGIEFNIASADEIAEYLNRKTIRFVPGFLQHRHYYFEKKPYVYNKKILSYDDCYLEGYWQNYRYFDDIKDELLKHMKFPCLPLEQKKLAEKMENENSVAVHVRMGDYLNLQDLYGGICDADYYDRAFSYIEGNISNPVYYGFSDDVDKASALLAKHKINWIDYNSEKGAIYDLILMSKCKNNIIANSSFSWWGAYLEYNNGKVVVSPNRWMNCFENSNIAYWGWISL

SEQ ID NO: 50 - DNA, 837 bp, unidentified
  misc_feature 1..837: note = Butyrivibrio proteoclasticus
  source 1..837: mol_type = genomic DNA
atggctatcatcatcaagttctgtggtgccctgggtaatcagctgtttcagtatgccctgtatgaaaaaatgcgtattctgggcaaagatgtgaaagcagatattagcgcctttggcgatggtaatgaaaaacgtttcttttatctggatgagctgggtattgaattcaatattgccagcgcagatgaaattgcagaatatctgaatcgtaaaaccattcgttttgttccgggttttctgcagcatcgccattattattttgaaaagaaaccgtatgtgtacaacaaaaagattctgagttacgatgattgctatctggaaggctattggcagaattatcgttattttgatgacattaaggacgaactgctgaaacatatgaaatttccgtgcctgccgctggaacagaaaaaactggccgaaaaaatggaaaatgaaaatagcgtggcagttcatgttcgtatgggcgattatctgaatctgcaggatctgtatggtggtatttgcgatgcagattattatgatcgtgcattttcatatatcgagggtaatattagcaacccggtttattatggttttagcgatgatgtggataaagcaagcgcactgctggcaaaacataaaattaattggattgactacaacagcgaaaaaggtgcaatctatgatctgattctgatgagtaaatgtaagaataacatcatcgccaatagcagctttagctggtggggtgcatatctggaatataataatggtaaagtggtggtgagtccgaatcgctggatgaattgctttgaaaatagcaatatcgcctattggggctggattagcctgtaa

SEQ ID NO: 51 - protein, 300 aa, unidentified
  REGION 1..300: note = Gramella sp. MAR_2010_147
MSKKKPVIIEILGGIGNQMFQFALAKILAEKNDSELFIDTNFYKETSQNLKNFPRYFSVGIFDLQFKLATEKEKIFFKHPSLKNRLNRKLGLNYPKVFKEKSFNFDPELLTMKAPIFLKGYFQSYKYFAGTESKIRQLYEFPDEKLDSRNEEIKNRIITKTSVSVHIRRGDYVENRKTQDFHGNCSVEYYKKAVEYLSATIKDFNLVFFSDDIAWVQNQFKDLPYEKKFVTGNLYENSWKDMYLMSLCDHNIIANSSFSWWAAWLNKNPEKKVVAPKKWFADMDQEQKSLDLLPPDWVRI

SEQ ID NO: 52 - DNA, 906 bp, unidentified
  misc_feature 1..906: note = Gramella sp. MAR_2010_147
  source 1..906: mol_type = genomic DNA
atggctagcaaaaagaagccggttattattgaaattctgggtggcattggcaatcagatgtttcagtttgccctggccaaaattctggcagaaaagaatgatagtgaactgtttattgacaccaatttttacaaggaaaccagccagaatctgaaaaattttccgcgttattttagcgtgggtatttttgatctgcagtttaaactggcaaccgaaaaagaaaaaatctttttcaagcacccgagcctgaaaaatcgtctgaatcgtaaactgggcctgaattatccgaaagtgtttaaagaaaagagctttaatttcgacccggaactgctgaccatgaaagccccgatttttctgaaaggctattttcagagctataaatatttcgcaggtaccgaaagtaaaattcgtcagctgtatgaatttccggatgaaaaactggatagccgcaatgaagaaattaagaatcgcattattaccaagaccagtgttagcgttcatattcgtcgtggcgattatgttgaaaatcgcaaaacccaggattttcatggtaattgcagtgtggaatattataaaaaggcagttgaatacctgagcgcaaccattaaggattttaatctggttttctttagcgatgatatcgcatgggttcagaatcagtttaaagatctgccgtatgaaaagaaattcgtgaccggtaatctgtatgaaaatagttggaaagatatgtacctgatgagtctgtgcgatcataatattattgcaaatagtagcttcagctggtgggcagcatggctgaataagaatccggaaaagaaagttgttgccccgaaaaaatggtttgcagatatggatcaggaacagaaaagcctggatctgctgccgccggattgggttcgtatttaa

SEQ ID NO: 53 - protein, 267 aa, unidentified
  REGION 1..267: note = Polaribacter glomeratus
MIVVRIIGGLGNQMFQYAFAKSLQQKGYQVKIDITKFKTYKLHGGYQLDKFKIDLETATTLENIISRLGFRRSTKERSLLFNKKFLEVPKREYIKGYFQTEKYFEDIKAILLKQFVVKNEISSSTLKYLKEITIQQNACSLHIRRGDYVSDKKANSVHGTCDLAYYKEAIKVMKNKFNDTHFFIFSDDIAWVKQNLKVKNTTYIDHEVIPHEDIHLMSLCKHNITANSSFSWWGAWLNQHSNKVVIAPKQWYLNKENEIASKDWIKI

SEQ ID NO: 54 - DNA, 807 bp, unidentified
  misc_feature 1..807: note = Polaribacter glomeratus
  source 1..807: mol_type = genomic DNA
atggctatcgtggtgcgcattattggcggcctgggtaatcagatgtttcagtatgcctttgccaaaagtctgcagcagaaaggttatcaggttaaaattgatatcaccaaattcaagacctacaaactgcatggtggttatcagctggataaattcaaaattgatctggaaaccgccaccaccctggaaaatattattagtcgcctgggttttcgccgtagtaccaaagaacgcagtctgctgtttaataagaaatttctggaagtgccgaaacgtgaatatattaagggttattttcagaccgaaaagtattttgaagatattaaggccatcctgctgaaacagtttgtggtgaaaaatgaaattagcagcagcaccctgaaatatctgaaagaaattaccattcagcagaatgcctgtagtctgcatattcgtcgcggtgactatgtgagcgataaaaaagccaatagtgtgcatggcacctgtgatctggcatattataaagaagcaattaaggttatgaagaacaagtttaacgacacccatttctttattttcagtgatgatatcgcctgggtgaaacagaatctgaaagtgaaaaataccacctatatcgatcatgaagttattccgcatgaagatattcatctgatgagcctgtgcaaacataatattaccgccaatagcagttttagttggtggggtgcatggctgaatcagcatagcaataaggtggttattgccccgaaacagtggtatctgaataaggaaaatgaaattgcaagcaaagactggattaagatttaa

SEQ ID NO: 55 - protein, 287 aa, unidentified
  REGION 1..287: note = Rhizobiales bacterium
MIVTRIVGGLGNQMFQYAVGRALSAKTGQEFKLDLSEMDRYKVHALQLDQFNIKGVRAGRHEIPFRPRKSFFGKILTALKNRNRIPQVFETTPSFDPSVLQRKGSCYLSGYWQSEKYFSDCSELIRADFSLKGPMSDERQAVLSQIRDAEAPVSVHVRRGDYVTNTTANSIHGTCEPEWYRQAMRKISDRTGDPTFFVFSDDPMWARSNLPTYEKMVFVEPRADGKDAEDMHLMSSCQSHIIANSTFSWWGAWLNPRQDKRVIAPARWFRAEDRDSTDLVPAQWERL

SEQ ID NO: 56 - DNA, 867 bp, unidentified
  misc_feature 1..867: note = Rhizobiales bacterium
  source 1..867: mol_type = genomic DNA
atggctatcgttacccgtattgtgggtggcctgggtaatcagatgtttcagtatgcagttggccgtgccctgagtgcaaaaaccggtcaggaattcaaactggatctgagcgaaatggatcgctataaagttcatgcactgcagctggatcagtttaatattaagggtgttcgcgccggccgtcatgaaattccgtttcgtccgcgcaaaagtttctttggcaaaattctgaccgcactgaaaaatcgcaatcgtattccgcaggtttttgaaaccaccccgagctttgatccgagcgtgctgcagcgtaaaggtagctgttatctgagtggttattggcagagcgaaaaatattttagcgattgtagcgaactgattcgtgcagattttagcctgaaaggtccgatgagcgatgaacgtcaggcagtgctgagtcagattcgtgatgcagaagcaccggtgagcgttcatgttcgccgcggcgattatgttaccaataccaccgccaatagcattcatggcacctgtgaaccggaatggtatcgtcaggccatgcgcaaaattagtgatcgtaccggtgacccgacctttttcgtttttagcgatgatccgatgtgggcacgcagcaatctgccgacctatgaaaaaatggtttttgtggaaccgcgtgccgatggtaaagatgccgaagatatgcatctgatgagcagctgccagagtcatattattgcaaatagcacctttagttggtggggtgcatggctgaatccgcgccaggataaacgcgtgattgcaccggcacgctggtttcgcgcagaagatcgcgatagcaccgatctggttccggcccagtgggaacgtctgtaa

SEQ ID NO: 57 - protein, 287 aa, unidentified
  REGION 1..287: note = Hoeflea sp. BAL378
MIITHINGGLGNQMFQYAAGRALALRHGEELRLDTREFDGKVQFGFGLDHFAIAARPGAPAELPPERRRDRLRYLAWRGFRLSPRLVRENGLGYNPGFAEIGDGAYLKGYWQSERYFRDVEATIRRDFTIITPPDPVNRAILDDLAASPAVSLHIRRGDYVVDPRTNATHGTCSMDYYARAVDLIAERMAETPVVYAFSDDPAWVRDNLELPCEIRVMDHNDSARNYEDLRLMSACRHHVIANSSFSWWGAWLNPSADKIVVSPARWFADPKLVNEDIWPTSWIRLS

SEQ ID NO: 58 - DNA, 867 bp, unidentified
  misc_feature 1..867: note = Hoeflea sp. BAL378
  source 1..867: mol_type = genomic DNA
atggctatcatcacccatattaacggcggtctgggcaatcagatgtttcagtatgccgccggtcgtgcactggccctgcgtcatggtgaagaactgcgtctggatacccgcgaatttgatggcaaagtgcagtttggttttggtctggatcattttgccattgccgcacgcccgggtgccccggcagaattaccgcctgaacgtcgtcgcgatcgcctgcgctatctggcctggcgtggctttcgcctgagtccgcgtctggtgcgtgaaaatggtctgggctataatccgggttttgccgaaattggtgacggcgcatatctgaaaggttattggcagagtgaacgctattttcgcgatgttgaagcaaccattcgtcgtgattttaccattattaccccgccggaccctgtgaatcgcgccattctggatgatctggccgccagtccggcagtgagcctgcatattcgtcgtggcgattatgttgtggaccctcgtaccaatgccacccacggtacctgtagcatggattattatgcccgcgcagttgatctgattgcagaacgtatggcagaaaccccggtggtgtatgcattttcagatgatccggcctgggtgcgcgataatctggaactgccgtgcgaaattcgcgttatggatcataatgatagcgcacgcaattatgaagatctgcgcctgatgagtgcctgccgtcatcatgttattgccaatagtagctttagctggtggggcgcatggctgaatccgagcgccgataaaattgtggttagtccggcccgttggtttgccgatccgaaactggttaatgaagatatttggccgaccagttggattcgtctgagttaa

SEQ ID NO: 59 - protein, 289 aa, unidentified
  REGION 1..289: note = Acetatifactor muris
MVIVRVQGGLGNQMFQYGFAKYQELSNEEVYLDITDYQTHIHHYGFELEKVFSNLTYKTIDGERLNKVRANPNMLLNRMLNKVLNIQIVRGSEFREQPAVSVSKRYTYNKDIYFNGFWANNEYVDAVKDTLKKDFTFKYILEGRNRELMDFLQGKISVGVHVRRGDYLQEKELRDVCDPDYYRKAFEIFMKRDVKTVFIIFSDDIPWVRKNFHFSKNMVFVDWNSGGEKSHVDMQMMSLCNHNIIANSTFSWWGAWLNANKDKCVVAPRYWRNNSKNESLIYPKNWMLL

SEQ ID NO: 60 - DNA, 870 bp, unidentified
  misc_feature 1..870: note = Acetatifactor muris
  source 1..870: mol_type = genomic DNA
atggttatcgttcgtgtgcagggcggtctgggtaatcagatgtttcagtatggttttgcaaaatatcaggaactgagtaatgaagaagtttatctggatattaccgattatcagacccatattcatcattatggttttgaactggaaaaggtgtttagtaatctgacctataaaaccattgacggtgaacgtctgaataaggttcgcgcaaatccgaatatgctgctgaatcgcatgctgaataaggtgctgaatattcagattgtgcgtggtagtgaatttcgcgaacagccggcagtgagcgttagcaaacgctatacctataataaggatatctatttcaacggcttctgggccaataatgaatatgtggatgcagtgaaagataccctgaaaaaagattttaccttcaaatacatcctggaaggccgcaatcgtgaactgatggattttctgcagggcaaaattagtgtgggtgtgcatgtgcgtcgcggcgattatctgcaggaaaaagaactgcgcgatgtttgtgatccggattattatcgcaaagcatttgaaattttcatgaagcgcgatgttaaaaccgtttttattattttcagcgacgatattccgtgggtgcgcaaaaattttcattttagcaaaaacatggtgttcgttgattggaatagcggcggcgaaaaaagccatgttgatatgcagatgatgagcctgtgtaatcataatattatcgcaaatagcaccttcagctggtggggtgcatggctgaatgccaataaggataaatgtgtggttgcaccgcgttattggcgtaataatagcaaaaatgaaagcctgatctatccgaaaaattggatgctgctgtaa

SEQ ID NO: 61 - protein, 298 aa, Helicobacter pylori
MAFKVVQICGGLGNQMFQYAFAKSLQKHLNTPVLLDITSFDWSNRKMQLELFPIDLPYASAKEIAIAKMQHLPKLVRDTLKCMGFDRVSQEIVFEYEPGLLKPSRLTYFYGYFQDPRYFDAISPLIKQTFTLPPPENGNNKKKEEEYHRKLALILAAKNSVFVHVRRGDYVGIGCQLGIDYQKKALEYIAKRVPNMELFVFCEDLKFTQNLDLGYPFMDMTTRDKEEEAYWDMLLMQSCKHGIIANSTYSWWAAYLINNPEKIIIGPKHWLFGHENILCKEWVKIESHFEVKSKKYNA

SEQ ID NO: 62 - DNA, 894 bp, Helicobacter pylori
  source 1..894: mol_type = genomic DNA
atggcattcaaggtggtgcagatttgtggcggtctgggcaaccagatgttccagtatgccttcgccaagagtctgcagaagcatctgaacaccccggtgctgctggatattaccagttttgattggagcaatcgcaagatgcagctggagctgttccctattgatctgccgtatgccagcgccaaagagatcgccatcgccaaaatgcagcatctgccgaaactggtgcgcgatactttaaaatgcatgggctttgatcgtgtgagccaagaaatcgtgtttgagtatgagccgggtctgctgaaaccgagccgtttaacctatttctacggctatttccaagatccgcgctacttcgatgccatcagcccgctgattaagcagaccttcactttaccgccgccggagaatggcaacaataagaaaaaagaggaagaatatcatcgcaagctggctttaattctggcagccaaaaacagcgtgttcgtgcatgttcgccgcggtgactatgtgggtatcggctgccagctgggcatcgactatcagaagaaggctttagaatatatcgcaaaacgcgtgccgaacatggagctgtttgtgttttgcgaggatttaaaatttacccagaatttagatttaggctacccgtttatggacatgaccacccgtgataaagaagaagaagcctattgggacatgctgctgatgcagagctgcaagcacggcatcattgccaacagcacctatagctggtgggccgcatatttaattaacaacccggaaaagatcatcatcggcccgaagcattggctgttcggccatgagaacattttatgcaaggaatgggttaaaattgagagccatttcgaagtgaaaagcaaaaagtataacgcc

SEQ ID NO: 63 - protein, 531 aa, Oryctolagus cuniculus
MSKSHSEAGSAFIQTQQLHAAMADTFLEHMCRLDIDSAPITARNTGIICTIGPASRSVETLKEMIKSGMNVARMNFSHGTHEYHAETIKNVRTATESFASDPILYRPVAVALDTKGPEIRTGLIKGSGTAEVELKKGATLKITLDNAYMEKCDENILWLDYKNICKVVDVGSKVYVDDGLISLQVKQKGPDFLVTEVENGGFLGSKKGVNLPGAAVDLPAVSEKDIQDLKFGVEQDVDMVFASFIRKAADVHEVRKILGEKGKNIKIISKIENHEGVRRFDEILEASDGIMVARGDLGIEIPAEKVFLAQKMIIGRCNRAGKPVICATQMLESMIKKPRPTRAEGSDVANAVLDGADCIMLSGETAKGDYPLEAVRMQHLIAREAEAAMFHRKLFEELARSSSHSTDLMEAMAMGSVEASYKCLAAALIVLTESGRSAHQVARYRPRAPIIAVTRNHQTARQAHLYRGIFPVVCKDPVQEAWAEDVDLRVNLAMNVGKARGFFKKGDVVIVLTGWRPGSGFTNTMRVVPVP

SEQ ID NO: 64 - protein, 381 aa, Oryctolagus cuniculus
MPFGNTHNKYKLNYKSEEEYPDLSKHNNHMAKVLTPDLYKKLRDKETPSGFTLDDVIQTGVDNPGHPFIMTVGCVAGDEESYTVFKDLFDPIIQDRHGGFKPTDKHKTDLNHENLKGGDDLDPHYVLSSRVRTGRSIKGYTLPPHCSRGERRAVEKLSVEALNSLTGEFKGKYYPLKSMTEQEQQQLIDDHFLFDKPVSPLLLASGMARDWPDARGIWHNDNKSFLVWVNEEDHLRVISMEKGGNMKEVFRRFCVGLQKIEEIFKKAGHPFMWNEHLGYVLTCPSNLGTGLRGGVHVKLAHLSKHPKFEEILTRLRLQKRGTGGVDTAAVGSVFDISNADRLGSSEVEQVQLVVDGVKLMVEMEKKLEKGQSIDDMIPAQK

SEQ ID NO: 65 - protein, 396 aa, Geobacter sulfurreducens
MAKVLAVNAGSSSLKFQLFDMPAETVLTKGIVERIGFDDAIFTIVVNGEKQREVTSIPNHAVAVKLLLDKLIRYGIIRSFDEIDGIGHRVVHGGEKFSDSVLITDEVIKQIEEVSELAPLHNPANLVGIRAFQEVLPNVPAVAVFDTAFHQTMPEQSFLYSLPYEYYTKFGIRKYGFHGTSHKYVTQRAAELLGRPIEQLRLISCHLGNGASIAAVEGGKSIDTSMGFTPLAGVAMGTRSGNIDPALIPYIMEKTGMTVNEVIEVLNKKSGMLGISGISSDLRDLEKAAAEGNERAELALEVFANRIHKYIGSYAARMCGVDAIIFTAGIGENSEVVRAKVLRGLEFMGVYWDPILNKVRGKEAFISYPHSPVKVLVIPTNEEVMIARDVMRLANL

SEQ ID NO: 66 - DNA, 1191 bp, Geobacter sulfurreducens
  source 1..1191: mol_type = genomic DNA
atggcaaaagtcctggcggtcaatgcggggtcgagcagtttgaaattccagctcttcgacatgccggcggaaactgtgctgaccaaagggattgtggaacgaatcggcttcgacgatgctatttttacgattgtggtgaacggcgaaaaacagcgtgaagtcacaagcataccaaatcacgcggttgccgtcaaactgctgctggacaaattaattcgctatgggattattcgtagcttcgatgaaattgatggcatcggccaccgcgtggtgcacgggggagaaaaattcagcgattctgtacttatcacagatgaagtaatcaaacagattgaagaagtctcggaactcgctccgttacataacccggcaaacctggtaggaatccgcgcgttccaggaggtgcttcccaacgtcccggcggtcgcggtttttgacacggcgtttcaccagaccatgccggagcaaagcttcttgtattctttgccgtatgagtattatacaaaatttggtatccgcaaatacggtttccacggcacatcccataaatatgtgacccaacgtgcggctgagttgttggggcgtcctatcgaacagctgagactcatcagttgtcacctggggaacggcgcatctattgcggctgtagaaggcggtaaatccatagacacgtctatgggtttcactccgctggctggtgtggccatgggtacgcgctcgggaaatatcgaccccgcccttatcccctacattatggaaaagaccggcatgacggtgaacgaagttattgaggtcctgaataaaaagtcgggcatgctcggcatatccggtattagctcggatctccgagatctggagaaagcggcggcggaaggtaatgaacgcgcggaactggcgttagaggtttttgcgaatcgcattcataagtatattggtagctatgcggcacgaatgtgtggtgtcgatgctattatttttacggccggcattggtgaaaattctgaagtggtacgagccaaggtgttacgtggtctggagtttatgggcgtatattgggacccgatactgaataaagtacgcggtaaagaagcgtttatcagttatccgcatagccctgtcaaagtcttggttatcccaacgaacgaagaagtcatgattgcgcgcgatgttatgcggttagcgaatttataa

SEQ ID NO: 67 - protein, 759 aa, Escherichia coli
MDDQLKQSALDFHEFPVPGKIQVSPTKPLATQRDLALAYSPGVAAPCLEIEKDPLKAYKYTARGNLVAVISNGTAVLGLGNIGALAGKPVMEGKGVLFKKFAGIDVFDIEVDELDPDKFIEVVAALEPTFGGINLEDIKAPECFYIEQKLRERMNIPVFHDDQHGTAIISTAAILNGLRVVEKNISDVRMVVSGAGAAAIACMNLLVALGLQKHNIVVCDSKGVIYQGREPNMAETKAAYAVVDDGKRTLDDVIEGADIFLGCSGPKVLTQEMVKKMARAPMILALANPEPEILPPLAKEVRPDAIICTGRSDYPNQVNNVLCFPFIFRGALDVGATAINEEMKLAAVRAIAELAHAEQSEVVASAYGDQDLSFGPEYIIPKPFDPRLIVKIAPAVAKAAMESGVATRPIADFDVYIDKLTEFVYKTNLFMKPIFSQARKAPKRVVLPEGEEARVLHATQELVTLGLAKPILIGRPNVIEMRIQKLGLQIKAGVDFEIVNNESDPRFKEYWTEYFQIMKRRGVTQEQAQRALISNPTVIGAIMVQRGEADAMICGTVGDYHEHFSVVKNVFGYRDGVHTAGAMNALLLPSGNTFIADTYVNDEPDAEELAEITLMAAETVRRFGIEPRVALLSHSNFGSSDCPSSSKMRQALELVRERAPELMIDGEMHGDAALVEAIRNDRMPDSSLKGSANILVMPNMEAARISYNLLRVSSSEGVTVGPVLMGVAKPVHVLTPIASVRRIVNMVALAVVEAQTQPL

SEQ ID NO: 68 - DNA, 2280 bp, Escherichia coli
  source 1..2280: mol_type = genomic DNA
atggatgaccagttaaaacaaagtgcacttgatttccatgaatttccagttccagggaaaatccaggtttctccaaccaagcctctggcaacacagcgcgatctggcgctggcctactcaccaggcgttgccgcaccttgtcttgaaatcgaaaaagacccgttaaaagcctacaaatataccgcccgaggtaacctggtggcggtgatctctaacggtacggcggtgctggggttaggcaacattggcgcgctggcaggcaaaccggtgatggaaggcaagggcgttctgtttaagaaattcgccgggattgatgtatttgacattgaagttgacgaactcgacccggacaaatttattgaagttgtcgccgcgctcgaaccaaccttcggcggcatcaacctcgaagatattaaagcgccagaatgtttctatattgaacagaaactgcgcgagcggatgaatattccggtattccacgacgatcagcacggcacggcaattatcagcactgccgccatcctcaacggcttgcgcgtggtggagaaaaacatctccgacgtgcggatggtggtttccggcgcgggtgccgcagcaatcgcctgtatgaacctgctggtagcgctgggtctgcaaaaacataacatcgtggtttgcgattcaaaaggcgttatctatcagggccgtgagccaaacatggcggaaaccaaagccgcgtatgcggtggtggatgacggcaaacgtaccctcgatgatgtgattgaaggcgcggatattttcctgggctgttccggcccgaaagtgctgacccaggaaatggtgaagaaaatggctcgtgcgccaatgatcctggcgctggcgaacccggaaccggaaattctgccgccgctggcgaaagaagtgcgtccggatgccatcatttgcaccggtcgttctgactatccgaaccaggtgaacaacgtcctgtgcttcccgttcatcttccgtggcgcgctggacgttggcgcaaccgccatcaacgaagagatgaaactggcggcggtacgtgcgattgcagaactcgcccatgcggaacagagcgaagtggtggcttcagcgtatggcgatcaggatctgagctttggtccggaatacatcattccaaaaccgtttgatccgcgcttgatcgttaagatcgctcctgcggtcgctaaagccgcgatggagtcgggcgtggcgactcgtccgattgctgatttcgacgtctacatcgacaagctgactgagttcgtttacaaaaccaacctgtttatgaagccgattttctcccaggctcgcaaagcgccgaagcgcgttgttctgccggaaggggaagaggcgcgcgttctgcatgccactcaggaactggtaacgctgggactggcgaaaccgatccttatcggtcgtccgaacgtgatcgaaatgcgcattcagaaactgggcttgcagatcaaagcgggcgttgattttgagatcgtcaataacgaatccgatccgcgctttaaagagtactggaccgaatacttccagatcatgaagcgtcgcggcgtcactcaggaacaggcgcagcgggcgctgatcagtaacccgacagtgatcggcgcgatcatggttcagcgtggggaagccgatgcaatgatttgcggtacggtgggtgattatcatgaacattttagcgtggtgaaaaatgtctttggttatcgcgatggcgttcacaccgcaggtgccatgaacgcgctgctgctgccgagtggtaacacctttattgccgatacctatgttaatgatgaaccggatgcagaagagctggcggagatcaccttgatggcggcagaaactgtccgtcgttttggtattgagccgcgcgttgctttgttgtcgcactccaactttggttcttctgactgcccgtcgtcgagcaaaatgcgtcaggcgctggaactggtcagggaacgtgcaccagaactgatgattgatggtgaaatgcacggcgatgcagcgctggtggaagcgattcgcaacgaccgtatgccggacagctctttgaaaggttccgccaatattctggtgatgccgaacatggaagctgcccgcattagttacaacttactgcgtgtttccagctcggaaggtgtgactgtcggcccggtgctgatgggtgtggcgaaaccggttcacgtgttaacgccgatcgcatcggtgcgtcgtatcgtcaacatggtggcgctggccgtggtagaagcgcaaacccaaccgctgtaa

SEQ ID NO: 69 - protein, 364 aa, Candida boidinii
MKIVLVLYDAGKHAADEEKLYGCTENKLGIANWLKDQGHELITTSDKEGGNSVLDQHIPDADIIITTPFHPAYITKERIDKAKKLKLVVVAGVGSDHIDLDYINQTGKKISVLEVTGSNVVSVAEHVVMTMLVLVRNFVPAHEQIINHDWEVAAIAKDAYDIEGKTIATIGAGRIGYRVLERLVPFNPKELLYYQHQALPKDAEEKVGARRVENIEELVAQADIVTVNAPLHAGTKGLINKELLSKFKKGAWLVNTARGAICVAEDVAAALESGQLRGYGGDVWFPQPAPKDHPWRDMRNKYGAGNAMTPHYSGTTLDAQTRYAQGTKNILESFFTGKFDYRPQDIILLNGEYVTKAYGKHDKK

SEQ ID NO: 70 - DNA, 1095 bp, Candida boidinii
  source 1..1095: mol_type = genomic DNA
atgaagatcgttttagtcttatatgatgctggtaaacacgctgccgatgaagaaaaattatacggttgtactgaaaacaaattaggtattgccaattggttgaaagatcaaggacatgaattaatcaccacgtctgataaagaaggcggaaacagtgtgttggatcaacatataccagatgccgatattatcattacaactcctttccatcctgcttatatcactaaggaaagaatcgacaaggctaaaaaattgaaattagttgttgtcgctggtgtcggttctgatcatattgatttggattatatcaaccaaaccggtaagaaaatctccgttttggaagttaccggttctaatgttgtctctgttgcagaacacgttgtcatgaccatgcttgtcttggttagaaattttgttccagctcacgaacaaatcattaaccacgattgggaggttgctgctatcgctaaggatgcttacgatatcgaaggtaaaactatcgccaccattggtgccggtagaattggttacagagtcttggaaagattagtcccattcaatcctaaagaattattatactaccagcatcaagctttaccaaaagatgctgaagaaaaagttggtgctagaagggttgaaaatattgaagaattggttgcccaagctgatatagttacagttaatgctccattacacgctggtacaaaaggtttaattaacaaggaattattgtctaaattcaagaaaggtgcttggttagtcaatactgcaagaggtgccatttgtgttgccgaagatgttgctgcagctttagaatctggtcaattaagaggttatggtggtgatgtttggttcccacaaccagctccaaaagatcacccatggagagatatgagaaacaaatatggtgctggtaacgccatgactcctcattactctggtactactttagatgctcaaactagatacgctcaaggtactaaaaatatcttggagtcattctttactggtaagtttgattacagaccacaagatatcatcttattaaacggtgaatacgttaccaaagcttacggtaaacacgataagaaataa

SEQ ID NO: 71 - protein, 336 aa, Pseudomonas stutzeri
MLPKLVITHRVHEEILQLLAPHCELITNQTDSTLTREEILRRCRDAQAMMAFMPDRVDADFLQACPELRVIGCALKGFDNFDVDACTARGVWLTFVPDLLTVPTAELAIGLAVGLGRHLRAADAFVRSGKFRGWQPRFYGTGLDNATVGFLGMGAIGLAMADRLQGWGATLQYHARKALDTQTEQRLGLRQVACSELFASSDFILLALPLNADTLHLVNAELLALVRPGALLVNPCRGSVVDEAAVLAALERGQLGGYAADVFEMEDWARADRPQQIDPALLAHPNTLFTPHIGSAVRAVRLEIERCAAQNILQALAGERPINAVNRLPKANPAAD

SEQ ID NO: 72 - DNA, 1014 bp, Pseudomonas stutzeri
  source 1..1014: mol_type = genomic DNA
atgctgccgaaactcgttataactcaccgagtacacgaagagatcctgcaactgctggcgccacattgcgagctgatcaccaaccagaccgacagcacgctgacgcgcgaggaaattctgcgccgctgccgcgatgctcaggcgatgatggcgttcatgcccgatcgggtcgatgcagactttcttcaagcctgccctgagctgcgtgtaatcggctgcgcgctcaagggcttcgacaatttcgatgtggacgcctgtactgcccgcggggtctggctgaccttcgtgcctgatctgttgacggtcccgactgccgagctggcgatcggactggcggtggggctggggcggcatctgcgggcagcagatgcgttcgtccgctctggcaagttccggggctggcaaccacggttctacggcacggggctggataacgctacggtcggcttccttggcatgggcgccatcggactggccatggctgatcgcttgcagggatggggcgcgaccctgcagtaccacgcgcggaaggctctggatacacaaaccgagcaacggctcggcctgcgccaggtggcgtgcagcgaactcttcgccagctcggacttcatcctgctggcgcttcccttgaatgccgataccctgcatctggtcaacgccgagctgcttgccctcgtacggccgggcgctctgcttgtaaacccctgtcgtggttcggtagtggatgaagccgccgtgctcgcggcgcttgagcgaggccagctcggcgggtatgcggcggatgtattcgaaatggaagattgggctcgcgcggaccggccgcagcagatcgatcctgcgctgctcgcgcatccgaatacgctgttcactccgcacatagggtcggcagtgcgcgcggtgcgcctggagattgaacgttgtgcagcgcagaacatcctccaggcattggcaggtgagcgcccaatcaacgctgtgaaccgtctgcccaaggccaaccctgccgcagattgataa

SEQ ID NO: 73 - protein, 261 aa, Bacillus subtilis
MYPDLKGKVVAITGAASGLGKAMAIRFGKEQAKVVINYYSNKQDPNEVKEEVIKAGGEAVVVQGDVTKEEDVKNIVQTAIKEFGTLDIMINNAGLENPVPSHEMPLKDWDKVIGTNLTGAFLGSREAIKYFVENDIKGNVINMSSVHEVIPWPLFVHYAASKGGIKLMTETLALEYAPKGIRVNNIGPGAINTPINAEKFADPKQKADVESMIPMGYIGEPEEIAAVAAWLASKEASYVTGITLFADGGMTQYPSFQAGRG

SEQ ID NO: 74 - DNA, 786 bp, Bacillus subtilis
  source 1..786: mol_type = genomic DNA
atgtatcctgatctcaagggaaaagttgtagccattacaggtgcagccagtggacttggaaaagctatggcgattagattcgggaaagaacaagcaaaggtcgtcatcaactattattctaataagcaggaccccaacgaagtaaaagaagaagtaatcaaagcaggaggtgaagccgttgtggttcagggagatgttaccaaagaagaggatgtcaagaatatagttcagaccgcgattaaggaatttggaacgttagatattatgattaataatgcaggtttggaaaaccccgtaccttctcacgaaatgccattgaaggattgggataaggtaataggaacgaatctaaccggagcgttcttaggcagcagagaagccatcaagtattttgtcgagaacgatataaaaggaaatgttattaacatgtcatccgtccatgaggttattccatggccacttttcgttcattacgctgctagtaaaggtggtatcaaattaatgacagaaactttggctctggaatatgcaccaaaaggtattagagttaacaacattggaccaggcgctattaatactcccataaatgctgagaaatttgccgacccaaaacaaaaagctgatgttgaatcaatgatacccatgggatatattggagagcctgaggaaatagccgctgttgctgcatggcttgcttccaaggaagcttcttatgtgactgggatcactcttttcgcagacggaggaatgacgcaatatccatcctttcaggccgggcggggctaa

SEQ ID NO: 75 - protein, 373 aa, synthetic construct
  REGION 1..373: note = Arabidopsis thaliana GMD mutant
MASENNGSRSDSESITAPKADSTVVEPRKIALITGITGQDGSYLTEFLLGKGYEVHGLIRRSSNFNTQRINHIYIDPANVNKALMKLHYADLTDASSLRRWIDVIKPDEVYNLAAQSHVAVSFEIPDYTADVVATGALRLLEAVRSHTIDSGRTVKYYQAGSSEMFGSTPPPQSETTPFHPRSPYAASKCAAHWYTVNYREAYGLFACNGILFNHESPRRGENFVTRKITRALGRIKVGLQTKLFLGNLQASRDWGFAGDYVEAMWLMLQQEKPDDYVVATEEGHTVEEFLDVSFGYLGLNWKDYVEIDQRYFRPAEVDNLQGDASKAKEVLGWKPQVGFEKLVKMMVDEDLELAKREKVLVDAGYMDAKQQP

SEQ ID NO: 76 - DNA, 1122 bp, synthetic construct
  misc_feature 1..1122: note = Arabidopsis thaliana GMD mutant
  source 1..1122: mol_type = other DNA
atggcaagtgagaacaatggttcacgttctgactctgaaagcatcacggctcctaaagcggacagcaccgttgtggaaccacggaaaatcgctctaatcaccggcatcacgggtcaggacggtagttacttgactgaatttctactaggcaaaggttacgaagtgcatggcctgatccgtaggagtagcaattttaacacgcagcggatcaatcatatctatattgatccacacaacgtgaacaaagctttaatgaaactccatgccgcggatctcactgacgcctcttcgttgcgtcgctggatcgacgtcattaaacctgacgaagtgtataacctggcggcacagtctcatgtggccgtttcattcgaaatacctgattatacggcggacgtggttgccaccggtgccttaagactgctcgaggcggttcgctcccataccattgattccgggcgcacggtaaaatattatcaggcaggaagcagcgaaatgtttggaagtacgccgccccctcagtctgagacaaccccgtttcacccgcgcagtccgtatgcggcatctaaatgtgccgcacattggtatacagtcaattatcgtgaggcttatggcttgtttgcatgcaatggcattctgttcaatcatgaaagcccgcgcagaggcgaaaattttgttacccgcaaaattacgcgtgccctgggccgtattaaagtaggtctgcaaactaaactgtttcttggcaacctccaggctagccgtgactggggatttgccggtgattatgtcgaagccatgtggctcatgttacagcaggagaaaccggacgattatgttgttgcgacagaagaaggacacacagtggaggaatttttggatgtatcgttcggctatttaggtctaaactggaaagattacgttgagattgatcaacgctacttccggccggcggaagtggacaacctgcaaggagatgcctccaaggcaaaagaagtactgggttggaaaccgcaggtgggcttcgagaaacttgtcaaaatgatggtggatgaagatctggaattagctaaacgcgagaaggtactggtagatgcaggatacatggatgcgaagcagcaaccgtaa

SEQ ID NO: 77 - protein, 373 aa, synthetic construct
  REGION 1..373: note = Arabidopsis thaliana GMD mutant
MASENNGSRSDSESITAPKADSTVVEPRKIALITGITGQDGSYLTEFLLGKGYEVHGLIRRSSNFNTQRINHIYIDPHNVNKALMKLHYADLTDASSLRRWIDVIKPDEVYNLAAQSHVAVSFEIPDYTADVVATGALRLLEAVRSHTIDSGRTVKYYQAGSSEMFGSTPPPQSETTPFHPRSPYAASKCAAHWYTVNYREAYGLFACNGILFNHESPRRGENFVTRAITRALGRIKVGLQTKLFLGNLQASRDWGFAGDYVEAMWLMLQQEKPDDYVVATEEGHTVEEFLDVSFGYLGLNWKDYVEIDQRYFRPAEVDNLQGDASKAKEVLGWKPQVGFEKLVKMMVDEDLELAKREKVLVDAGYMDAKQQP

SEQ ID NO: 78 - DNA, 1122 bp, synthetic construct
  misc_feature 1..1122: note = Arabidopsis thaliana GMD mutant
  source 1..1122: mol_type = other DNA
atggcaagtgagaacaatggttcacgttctgactctgaaagcatcacggctcctaaagcggacagcaccgttgtggaaccacggaaaatcgctctaatcaccggcatcacgggtcaggacggtagttacttgactgaatttctactaggcaaaggttacgaagtgcatggcctgatccgtaggagtagcaattttaacacgcagcggatcaatcatatctatattgatccacacaacgtgaacaaagctttaatgaaactccattacgcggatctcactgacgcctcttcgttgcgtcgctggatcgacgtcattaaacctgacgaagtgtataacctggcggcacagtctcatgtggccgtttcattcgaaatacctgattatacggcggacgtggttgccaccggtgccttaagactgctcgaggcggttcgctcccataccattgattccgggcgcacggtaaaatattatcaggcaggaagcagcgaaatgtttggaagtacgccgccccctcagtctgagacaaccccgtttcacccgcgcagtccgtatgcggcatctaaatgtgccgcacattggtatacagtcaattatcgtgaggcttatggcttgtttgcatgcaatggcattctgttcaatcatgaaagcccgcgcagaggcgaaaattttgttacccgcgcaattacgcgtgccctgggccgtattaaagtaggtctgcaaactaaactgtttcttggcaacctccaggctagccgtgactggggatttgccggtgattatgtcgaagccatgtggctcatgttacagcaggagaaaccggacgattatgttgttgcgacagaagaaggacacacagtggaggaatttttggatgtatcgttcggctatttaggtctaaactggaaagattacgttgagattgatcaacgctacttccggccggcggaagtggacaacctgcaaggagatgcctccaaggcaaaagaagtactgggttggaaaccgcaggtgggcttcgagaaacttgtcaaaatgatggtggatgaagatctggaattagctaaacgcgagaaggtactggtagatgcaggatacatggatgcgaagcagcaaccgtaa

SEQ ID NO: 79 - protein, 373 aa, synthetic construct
  REGION 1..373: note = Arabidopsis thaliana GMD mutant
MASENNGSRSDSESITAPKADSTVVEPRKIALITGITGQDGSYLTEFLLGKGYEVHGLIRRSSNFNTQRINHIYIDPHNVNKALMKLHYADLTDASSLRRWIDVIKPDEVYNLAAQSHVAVSFEIPDYTADVVATGALRLLEAVRSHTIDSGRTVKYYQAGSSEMFGSTPPPQSETTPFHPRSPYAASKCAAHWYTVNYREAYGLFACNGILFNHESPRRGENFVTRKITAALGRIKVGLQTKLFLGNLQASRDWGFAGDYVEAMWLMLQQEKPDDYVVATEEGHTVEEFLDVSFGYLGLNWKDYVEIDQRYFRPAEVDNLQGDASKAKEVLGWKPQVGFEKLVKMMVDEDLELAKREKVLVDAGYMDAKQQP

SEQ ID NO: 80 - DNA, 1122 bp, synthetic construct
  misc_feature 1..1122: note = Arabidopsis thaliana GMD mutant
  source 1..1122: mol_type = other DNA
atggcaagtgagaacaatggttcacgttctgactctgaaagcatcacggctcctaaagcggacagcaccgttgtggaaccacggaaaatcgctctaatcaccggcatcacgggtcaggacggtagttacttgactgaatttctactaggcaaaggttacgaagtgcatggcctgatccgtaggagtagcaattttaacacgcagcggatcaatcatatctatattgatccacacaacgtgaacaaagctttaatgaaactccattacgcggatctcactgacgcctcttcgttgcgtcgctggatcgacgtcattaaacctgacgaagtgtataacctggcggcacagtctcatgtggccgtttcattcgaaatacctgattatacggcggacgtggttgccaccggtgccttaagactgctcgaggcggttcgctcccataccattgattccgggcgcacggtaaaatattatcaggcaggaagcagcgaaatgtttggaagtacgccgccccctcagtctgagacaaccccgtttcacccgcgcagtccgtatgcggcatctaaatgtgccgcacattggtatacagtcaattatcgtgaggcttatggcttgtttgcatgcaatggcattctgttcaatcatgaaagcccgcgcagaggcgaaaattttgttacccgcaaaattacggctgccctgggccgtattaaagtaggtctgcaaactaaactgtttcttggcaacctccaggctagccgtgactggggatttgccggtgattatgtcgaagccatgtggctcatgttacagcaggagaaaccggacgattatgttgttgcgacagaagaaggacacacagtggaggaatttttggatgtatcgttcggctatttaggtctaaactggaaagattacgttgagattgatcaacgctacttccggccggcggaagtggacaacctgcaaggagatgcctccaaggcaaaagaagtactgggttggaaaccgcaggtgggcttcgagaaacttgtcaaaatgatggtggatgaagatctggaattagctaaacgcgagaaggtactggtagatgcaggatacatggatgcgaagcagcaaccgtaa

SEQ ID NO: 81 - protein, 351 aa, synthetic construct
  REGION 1..351: note = Homo sapiens GMD mutant
MRNVALITGITGQDGSYLAEFLLEKGYEVHGIVRRSSSFNTGRIEHLYKNPQAAIEGNMKLHYGDLTDSTCLVKIINEVKPTEIYNLGAQSHVKISFDLAEYTADVDGVGTLRLLDAVKTCGLINSVKFYQASTSELYGKVQEIPQKETTPFYPRSPYGAAKLYAYWIVVNFREAYNLFAVNGILFNHESPRRGANFVTRKISRSVAKIYLGQLECFSLGNLDAKRDWGHAKDYVEAMWLMLQNDEPEDFVIATGEVHSVREFVEKSFLHIGKTIVWEGKNENEVGRCKETGKVHVTVDLKYYRPTEVDFLQGDCTKAKQKLNWKPRVAFDELVREMVHADVELMRTNPNA

SEQ ID NO: 82 - DNA, 1056 bp, synthetic construct
  misc_feature 1..1056: note = Homo sapiens GMD mutant
  source 1..1056: mol_type = other DNA
atgcgaaacgttgccttgatcaccggtattaccggccaggatggctcatatctggcagaatttctgcttgaaaaaggctatgaggttcatggcatcgtgcgccgcagcagtagttttaataccggccgcattgaacatctgtataaaaacccacaagcagccatcgaaggaaatatgaaactgcattatggcgatttgacagactcaacgtgtctggttaagataataaacgaagtgaagcctaccgaaatttacaacctgggtgcgcagtctcatgtgaaaattagcttcgatttggccgaatataccgcggatgtcgatggtgtgggtacgttacgactgttggacgctgttaaaacctgcgggctgatcaacagcgtgaaattttatcaggctagcacgagtgagctctatggaaaggtccaggagattccccagaaggaaacgacgcctttctatccacgcagcccgtatggggcagcaaaactttatgcctattggatcgtagtgaactttcgcgaagcttataatctttttgcggttaatggcatactgtttaaccacgagtcgccacgacgcggcgcaaacttcgtgacccgtaaaataagtcgtagcgtcgcgaagatctatctgggtcagctcgaatgtttcagccttggcaacctggatgcgaaacgtgattggggacacgcgaaagattatgtcgaagccatgtggctgatgttacaaaacgatgaacctgaggacttcgttatcgccacgggtgaagtgcatagcgtacgcgaatttgtcgaaaaaagcttcctccatataggtaagaccatcgtgtgggaaggcaaaaatgagaacgaggttggtcgctgcaaagaaaccggcaaagttcacgttacggttgatctcaaatactacagacccaccgaagtggactttctgcaaggcgattgtaccaaagccaaacagaaactaaattggaaacctcgcgttgccttcgacgaactcgtccgtgaaatggtccatgcagatgtcgaactgatgagaacaaaccctaacgcgtga

SEQ ID NO: 83 - protein, 351 aa, synthetic construct
  REGION 1..351: note = Homo sapiens GMD mutant
MRNVALITGITGQDGSYLAEFLLEKGYEVHGIVRRSSSFNTGRIEHLYKNPQAHIEGNMKLHYGDLTDSTCLVKIINEVKPTEIYNLGAQSHVKISFDLAEYTADVDGVGTLRLLDAVKTCGLINSVKFYQASTSELYGKVQEIPQKETTPFYPRSPYGAAKLYAYWIVVNFREAYNLFAVNGILFNHESPRRGANFVTRAISRSVAKIYLGQLECFSLGNLDAKRDWGHAKDYVEAMWLMLQNDEPEDFVIATGEVHSVREFVEKSFLHIGKTIVWEGKNENEVGRCKETGKVHVTVDLKYYRPTEVDFLQGDCTKAKQKLNWKPRVAFDELVREMVHADVELMRTNPNA

SEQ ID NO: 84 - DNA, 1056 bp, synthetic construct
  misc_feature 1..1056: note = Homo sapiens GMD mutant
  source 1..1056: mol_type = other DNA
atgcgaaacgtggccttgatcaccggtattaccggccaggatggctcatatctggcagaatttctgcttgaaaaaggctatgaggttcatggcatcgtgcgccgcagcagtagttttaataccggccgcattgaacatctgtataaaaacccacaagcacacatcgaaggaaatatgaaactgcattatggcgatttgacagactcaacgtgtctggttaagataataaacgaagtgaagcctaccgaaatttacaacctgggtgcgcagtctcatgtgaaaattagcttcgatttggccgaatataccgcggatgtcgatggtgtgggtacgttacgactgttggacgctgttaaaacctgcgggctgatcaacagcgtgaaattttatcaggctagcacgagtgagctctatggaaaggtccaggagattccccagaaggaaacgacgcctttctatccacgcagcccgtatggggcagcaaaactttatgcctattggatcgtagtgaactttcgcgaagcttataatctttttgcggttaatggcatactgtttaaccacgagtcgccacgacgcggcgcaaacttcgtgacccgtgcaataagtcgtagcgtcgcgaagatctatctgggtcagctcgaatgtttcagccttggcaacctggatgcgaaacgtgattggggacacgcgaaagattatgtcgaagccatgtggctgatgttacaaaacgatgaacctgaggacttcgttatcgccacgggtgaagtgcatagcgtacgcgaatttgtcgaaaaaagcttcctccatataggtaagaccatcgtgtgggaaggcaaaaatgagaacgaggttggtcgctgcaaagaaaccggcaaagttcacgttacggttgatctcaaatactacagacccaccgaagtggactttctgcaaggcgattgtaccaaagccaaacagaaactaaattggaaacctcgcgttgccttcgacgaactcgtccgtgaaatggtccatgcagatgtcgaactgatgagaacaaaccctaacgcgtga

SEQ ID NO: 85 - protein, 351 aa, synthetic construct
  REGION 1..351: note = Homo sapiens GMD mutant
MRNVALITGITGQDGSYLAEFLLEKGYEVHGIVRRSSSFNTGRIEHLYKNPQAHIEGNMKLHYGDLTDSTCLVKIINEVKPTEIYNLGAQSHVKISFDLAEYTADVDGVGTLRLLDAVKTCGLINSVKFYQASTSELYGKVQEIPQKETTPFYPRSPYGAAKLYAYWIVVNFREAYNLFAVNGILFNHESPRRGANFVTRKISASVAKIYLGQLECFSLGNLDAKRDWGHAKDYVEAMWLMLQNDEPEDFVIATGEVHSVREFVEKSFLHIGKTIVWEGKNENEVGRCKETGKVHVTVDLKYYRPTEVDFLQGDCTKAKQKLNWKPRVAFDELVREMVHADVELMRTNPNA

SEQ ID NO: 86 - DNA, 1056 bp, synthetic construct
  misc_feature 1..1056: note = Homo sapiens GMD mutant
  source 1..1056: mol_type = other DNA
atgcgaaacgtggccttgatcaccggtattaccggccaggatggctcatatctggcagaatttctgcttgaaaaaggctatgaggttcatggcatcgtgcgccgcagcagtagttttaataccggccgcattgaacatctgtataaaaacccacaagcacacatcgaaggaaatatgaaactgcattatggcgatttgacagactcaacgtgtctggttaagataataaacgaagtgaagcctaccgaaatttacaacctgggtgcgcagtctcatgtgaaaattagcttcgatttggccgaatataccgcggatgtcgatggtgtgggtacgttacgactgttggacgctgttaaaacctgcgggctgatcaacagcgtgaaattttatcaggctagcacgagtgagctctatggaaaggtccaggagattccccagaaggaaacgacgcctttctatccacgcagcccgtatggggcagcaaaactttatgcctattggatcgtagtgaactttcgcgaagcttataatctttttgcggttaatggcatactgtttaaccacgagtcgccacgacgcggcgcaaacttcgtgacccgtaaaataagtgctagcgtcgcgaagatctatctgggtcagctcgaatgtttcagccttggcaacctggatgcgaaacgtgattggggacacgcgaaagattatgtcgaagccatgtggctgatgttacaaaacgatgaacctgaggacttcgttatcgccacgggtgaagtgcatagcgtacgcgaatttgtcgaaaaaagcttcctccatataggtaagaccatcgtgtgggaaggcaaaaatgagaacgaggttggtcgctgcaaagaaaccggcaaagttcacgttacggttgatctcaaatactacagacccaccgaagtggactttctgcaaggcgattgtaccaaagccaaacagaaactaaattggaaacctcgcgttgccttcgacgaactcgtccgtgaaatggtccatgcagatgtcgaactgatgagaacaaaccctaacgcgtga

SEQ ID NO: 87 - protein, 300 aa, synthetic construct
  REGION 1..300: note = ASR1
MAFKVVQICGGLGNQMFQYAFAKSLQKHLNIPVLLDVTSFDWSNRKLQLELFPIDLPYASAKEIAMAKMQHLPKLVRDALKRMGFDRVSQEIVFEYEPKLLKPNRLTYFHGYFQDPRYFDGISPLIKQTFTLPPPPPENGNNKKKEEEYQRKLSLILAAKNSVFVHIRRGDYVGIGCQLGIDYQKKAVEYMAKRVPNMELFVFCEDLEFTQNLDLGYPFMDMTTRDKEEEAYWDMMLMQSCKHGIIANSTYSWWAAYLINNPEKIIIGPKHWLFGHENILCKDWVKIESHFEVKSEKYNA

SEQ ID NO: 88 - DNA, 900 bp, synthetic construct
  misc_feature 1..900: note = ASR1
  source 1..900: mol_type = other DNA
atggcgtttaaagtcgtccagatttgtggaggcttaggtaatcaaatgtttcagtatgcttttgctaagtcactgcaaaaacaccttaacattcctgtgcttctggacgttacctcgtttgattggtcgaatcgcaaattacagctggagttgtttccaattgacttgccgtatgcctcagccaaagaaatcgcaatggcgaaaatgcagcatcttccgaaactggtgcgcgatgcgctgaaacgcatgggattcgatcgcgtgtcccaggaaatcgtctttgaatatgaaccaaagctcctgaaaccaaaccgcttgacctactttcatggctactttcaggacccccgctatttcgacggcatctctcccttaattaaacagaccttcacactccctcctccgccgcctgaaaacgggaataataaaaagaaagaggaggaatatcaacgcaaactgagtctgattctggcggcgaaaaactctgttttcgtccacatccgtcgcggcgattacgtcggtattggttgccagttgggcattgattaccagaaaaaagcggtggaatatatggcgaaacgagtgccgaatatggaactatttgtgttttgtgaggatctggagttcacgcagaacctagacttggggtatccatttatggatatgaccacgcgggacaaggaagaggaagcctactgggatatgatgctgatgcagtcatgcaagcacggtattatcgccaatagcacctactcgtggtgggccgcctacttaattaacaatcctgagaagattattattggtccgaaacactggttatttggccacgaaaacatcctctgcaaggattgggttaaaattgaatcgcactttgaagtcaaatctgaaaaatacaacgca

SEQ ID NO: 89 - protein, 293 aa, synthetic construct
  REGION 1..293: note = ASR2
MIIIRMSGGLGNQMFQYALYLKLKAMGKEVKIDDITEYEGDNARPIMLDVFGIDYDRATKEEVTELTDGSMDFLSRIRRKLFGRKSKEYREKSCNFDPQVLEMDPAYLEGYFQSEKYFQDVREQVRKAFRFRGIESGSIPLSEKTRELQKQIEDSESVSIHIRRGDYLENGHGEVYGGICTDAYYKKAIEYMKEKFPDAKFYIFSNDTEWAKQHFKGENFVVVEGSTENTGYLDMFLMSKCRHHIIANSSFSWWGAWLNENPEKIVIAPSKWLNNRECKDIYTERMIRINPEV

SEQ ID NO: 90 - DNA, 879 bp, synthetic construct
  misc_feature 1..879: note = ASR2
  source 1..879: mol_type = other DNA
atgattatcattcgcatgagcgggggtctgggcaatcagatgttccagtatgccctctatctgaagctgaaagcgatgggcaaggaagtaaaaatcgatgatataaccgaatacgagggcgataatgctcgcccgataatgctggacgtgtttggaatcgattatgatcgtgcgaccaaagaagaagttaccgaactcaccgacggttctatggactttctgtcgcgcatccgccgtaaacttttcggccgcaaatcgaaagaataccgtgaaaaaagctgcaattttgacccgcaagttttggagatggacccggcgtacctggagggctatttccagagcgaaaaatattttcaagatgtgcgcgaacaggttcgaaaagcgttccgatttcgtggtattgaatcagggtccattccgctgtcagaaaaaacccgcgaattgcagaaacagatcgaagatagcgagtccgttagcattcatatccgtcgtggtgactatctggagaacggccacggcgaagtgtacggcggaatctgcaccgatgcctattacaaaaaagccatcgaatacatgaaggagaaattccctgatgccaaattttacatttttagcaatgatacggagtgggcaaaacaacatttcaagggagagaactttgtggtggttgagggctccactgaaaatactggttatcttgatatgttcctgatgagcaaatgtcgccaccacatcattgcgaatagttcgtttagctggtggggggcgtggttgaacgaaaacccggaaaaaatcgtgattgccccgagcaaatggctgaataaccgtgaatgtaaagacatctataccgaacgcatgatccgtatcaaccccgaggtg

SEQ ID NO: 91 - protein, 291 aa, synthetic construct
  REGION 1..291: note = ASR3
MIIIRIMGGLGNQMFQYALYRKLKSMGKEVKLDISWYDDHNQTHRSFELDVFGIDYDVASKEEISKFSNRSANFLSRIRRKLFGRKNKIYKEEDFNYDPEILELDDVYLEGYWQSEKYFEDIREQLRKEFTFPEELNEKNRELLEQMENENSVSIHIRRGDYLNNENADVYGGICTDDYYKKAIEYIRERIPDPKFYIFSDDIEWAKQQFKGDDFTIVDWNNGKDSYYDMYLMSKCKHNIIANSTFSWWGAWLNQNPEKIVISPKKWLNNHETSDIVCESWIRIDGQGEIR

SEQ ID NO: 92 - DNA, 873 bp, synthetic construct
  misc_feature 1..873: note = ASR3
  source 1..873: mol_type = other DNA
atgatcatcattcgcattatgggcggcctgggtaatcagatgtttcaatacgcgctgtatcgcaaactgaaatcgatgggaaaagaagtgaaactggacatcagttggtacgatgatcataatcaaactcaccgcagctttgaactcgacgtctttggtattgattatgatgtggcatccaaagaggaaattagcaagttttccaaccgctccgcgaatttcctgagtagaattaggcgaaaactgtttggccgaaaaaacaaaatttataaagaggaggactttaactacgatccagaaatccttgaattagatgatgtttatctggagggctattggcaaagtgagaagtatttcgaagatattcgcgaacaactgcgtaaagagtttacctttcccgaagagctgaacgaaaagaatcgtgagctgctggaacaaatggaaaacgaaaactcggtatcgattcacattcgtcgcggagattatctgaacaacgagaacgcagatgtatatggtggcatctgcacagatgattactataaaaaagctatcgaatatattcgtgagcgcattcccgatccaaagttttatatattctcagatgacatcgaatgggcaaaacaacagtttaaaggtgatgacttcaccatcgtagattggaacaatggcaaagacagctattatgatatgtatctgatgtcaaagtgtaaacacaacatcattgctaattccaccttttcctggtggggcgcctggctgaatcaaaatcccgagaaaatcgtgatttcccctaagaaatggcttaacaaccatgaaacctcagacatagtatgcgaaagttggattaggattgacggtcaaggtgaaattcgc

SEQ ID NO: 93 - protein, 291 aa, synthetic construct
  REGION 1..291: note = ASR4
MIIVRLTGGLGNQMFQYAMGRRLAEKHNTELKLDISGFENYKLRKYSLNHFNIQENFATPEEISRLTSVKQGRIEKLLRRILRKRPKKPNTYIREKHFHFDPEILNLPDNVYLDGYWQSEKYFKDIEDIIRREFTIKNPQTGKNKEIAEQIQSCNSVSLHVRRGDYVTNPTTNQVHGVCGLDYYQRCVDYIAKKVENPHFFVFSDDPEWVKENLKIDYPTTFVDHNGADKDYEDLRLMSQCKHHIIANSTFSWWGAWLNSNPDKIVIAPKKWFNTSDMDTKDLIPENWIKL

SEQ ID NO: 94 - DNA, 873 bp, synthetic construct
  misc_feature 1..873: note = ASR4
  source 1..873: mol_type = other DNA
atgattattgtccggcttacgggcggcttaggcaaccaaatgtttcagtacgcaatggggcgccgcttagctgaaaaacataataccgagctgaaattagacatcagcgggtttgaaaactataaactgcgtaaatacagcttgaatcactttaatattcaggaaaattttgccacaccggaagagatttcgcggctgacatcagttaaacagggccgtattgaaaagttgttgcgcaggattctgaggaagcgcccaaaaaaaccgaatacgtatatccgcgagaaacacttccactttgatcctgaaattctgaacctcccggacaacgtttacttggacggttactggcagagtgagaaatactttaaggacattgaggacatcattcgccgtgagtttaccataaaaaatccgcagaccggcaaaaacaaagagatcgcggaacagatccagagttgcaatagtgtctcactgcatgttcgtcgcggtgattacgttacgaaccccactaccaaccaagtccacggcgtctgtgggctagattactatcaacgttgcgtggattatatcgcaaaaaaggttgaaaacccacacttctttgtttttagcgatgatcccgagtgggtgaaagaaaaccttaaaatcgattatcctactaccttcgtggaccacaacggtgcggataaagactatgaagatttacgtctgatgtcacaatgcaaacatcatatcattgcaaactctacctttagttggtggggtgcctggctcaattctaaccctgacaaaattgtgattgcgccgaagaagtggttcaacactagcgatatggataccaaagatttgattccagagaattggatcaaacta

SEQ ID NO: 95 - protein, 273 aa, synthetic construct
  REGION 1..273: note = ASR5
MIVVKLIGGLGNQMFQYAAAKALALEKNQKLRLDVSAFESYKLHNYGLNHFNITAKIYKKENKWLRKIKSFFKKNTYYKEQDFGYNPDLFDLKADNIFLEGYFQSEKYFLKYEKEIRKDFEIISPLKKQTKEMIEQIQSVNSVSIHIRRGDYLTNPIHNTSKEEYYKKAMEFIESKIENPVFFVFSDDMDWVKENFKTNHETVFVDFNDASTNFEDLKLMSSCKHNIIANSSFSWWGAWLNKNPNKIVIAPKQWFNDDSINTSDIIPESWIKI

SEQ ID NO: 96 - DNA, 819 bp, synthetic construct
  misc_feature 1..819: note = ASR5
  source 1..819: mol_type = other DNA
atgatcgttgtaaaactgattggtggtcttggcaaccagatgttccagtacgcggcggcgaaagctctggcgctcgaaaaaaaccagaagctgcgtcttgatgtcagtgctttcgaatcatacaaactgcacaattatggactgaatcatttcaacataaccgccaaaatctataaaaaagaaaataagtggttacgcaaaatcaaaagtttcttcaaaaagaatacctactacaaagaacaagactttggctataacccggatctgtttgatttgaaagcggacaatatttttctggagggttatttccaaagcgagaaatattttctaaagtacgaaaaagaaatacgtaaagatttcgagatcatctcaccattaaaaaaacagaccaaagaaatgattgaacaaattcagtctgtgaatagtgtctcgatacatataaggcgcggtgattatctgaccaatccgattcataatacgtcaaaagaagaatactataagaaggcaatggagtttattgaatccaaaattgaaaacccggtattcttcgtgtttagtgatgacatggactgggtcaaagaaaactttaaaacgaaccatgagactgtgttcgtagatttcaatgatgccagcaccaactttgaggacctaaagctgatgtcctcatgtaaacacaatattattgcgaacagctcttttagctggtggggtgcttggctgaataaaaatccgaacaaaattgttatcgcgccaaaacagtggtttaacgacgatagcattaatacttcagacatcatcccggagtcctggattaaaata

SEQ ID NO: 97 - protein, 300 aa, synthetic construct
  REGION 1..300: note = ASR6
MAFKVVQICGGLGNQMFQYAFAKSLQKHLNIPVLLDVTSFDSSNRKLQLELFPIDLPYASAKEIAMAKMQHLPKLVRDALKRMGFDRVSQEIVFEYEPKLLKPNRLTYFHGYFQDPRYFDGISPLIKQTFTLPPPPPENGNNKKKEEEYQRKLSLILAAKNSVFVHIRRGDYVGIGCQLGIDYQKKAVEYMAKRVPNMELFVFCEDLEFTQNLDLGYPFMDMTTRDKEEEAYWDMMLMQSCKHGIIANSTYSWWAAYLINNPEKIIIGPKHWLFGHENILCKDWVKIESHFEVKSEKYNA

SEQ ID NO: 98 - DNA, 900 bp, synthetic construct
  misc_feature 1..900: note = ASR6
  source 1..900: mol_type = other DNA
atggcgtttaaagtggttcagatttgcggcggcttaggtaatcagatgttccagtatgcttttgcgaaaagcctgcaaaaacatctgaatattcctgtccttttagacgtcacgagctttgactcctctaatagaaaactccaattagaactgttcccaattgatctgccgtatgcaagtgcaaaagagattgcgatggcaaaaatgcagcacctcccaaaactggttcgagatgccttaaagcgaatgggattcgaccgcgtcagccaggagattgtttttgaatacgaacctaaacttcttaagccaaaccgcctgacgtacttccacggttactttcaagatccgcgctatttcgacggaatcagtccgctgatcaagcagacgttcaccttgccgccgccgccccctgaaaacggtaataataagaaaaaagaagaggaatatcagcggaagctgagcttgatcctggcagccaaaaacagtgtctttgtgcacattcgtcgcggcgactatgtgggcattggttgtcaattggggattgattaccagaaaaaagcggtcgagtacatggcgaaacgagtgcccaatatggagctgtttgttttctgcgaggacttagaatttacccagaatttggatctgggctatccgtttatggacatgacgacacgcgataaagaagaggaagcctactgggatatgatgctgatgcagagctgcaagcacggtattatcgctaactcaacatattcctggtgggccgcatatctgattaataaccccgaaaagattatcatcggaccaaaacactggctcttcggtcacgaaaatatcctgtgcaaagattgggtaaagattgaaagccactttgaagtgaaaagcgaaaaatataacgcc

SEQ ID NO: 99 - protein, 308 aa, synthetic construct
  REGION 1..308: note = ASR7
MIIIRMSGGLGNQMFQYALYLKLKSMGKEVKIDDITAYEGDNARPIMLDVFGIDYDRATKEEITEMTDSSMDFLSRIRRKLFGRKSKEYREKDFNFDPQVLEMDPAYLEGYFQSEKYFQDVREQVRKAFRFRKGSVPKELSEQTKELQKQIENSNSVSIHIRRGDYLENSHGEIYGGICTDAYYKKAIEYMKEKFPDAKFYIFSNDTEWAKQHFKGENFVIVEGSTENTGYLDMYLMSKCKHHIIANSSFSWWGAWLNDNPEKIVIAPSKWLNNRECKDIYTDRMIRIDAKGEVRSDDYGVRTNSTVK

SEQ ID NO: 100 - DNA, 924 bp, synthetic construct
  misc_feature 1..924: note = ASR7
  source 1..924: mol_type = other DNA
atgattatcatccgcatgagcggcggactgggcaaccaaatgttccagtatgccttgtatctgaaactgaaaagtatgggtaaagaagtgaaaatcgatgatataacagcctatgaaggggataacgcccgcccgatcatgctggacgttttcggcatcgattatgaccgtgctacgaaagaggagattaccgaaatgaccgattcctcgatggattttctgtcacgcattcgtcgcaaactgtttggacgtaaaagtaaagaatatcgcgaaaaagatttcaatttcgatccgcaggtcctggagatggacccggcgtacttggaaggctacttccagtccgagaaatactttcaggatgtgcgcgaacaggtccgcaaggcgttccggttccgcaagggaagcgtaccgaaagaattgtccgaacagaccaaggaactgcaaaaacagattgaaaactcgaactcagtgtcaattcatatccgtcgcggcgactatctggaaaactcacacggtgagatttatggggggatttgcaccgatgcttactataaaaaagcgattgaatacatgaaagaaaaattcccggatgccaaattctatattttcagcaacgacactgaatgggccaagcagcattttaaaggcgaaaactttgtcatcgttgagggctcaactgaaaataccgggtacttagacatgtatctgatgtccaaatgtaaacaccacattattgcaaactctagctttagctggtggggtgcctggctgaacgataacccggaaaaaattgtaatcgccccgtcaaaatggttaaacaatcgcgagtgcaaggacatttatactgaccgcatgattcgtatagatgcaaaaggcgaagtccgtagcgatgattatggggttcgtacgaacagcacggtgaaa

SEQ ID NO: 101 - protein, 303 aa, synthetic construct
  REGION 1..303: note = ASR8
MIIIRIMGGLGNQMFQYALYRKLKSMGKEVKLDISWYDDHNTHRSFELDVFGIEYDVASKKEISKFSNRSSNFLSRIRRKLFGKKNKIYQEEDFNYDPEILEMDDVYLEGYWQSEKYFEDIREQLRKEFTFPKEMNKQNKELLEQMENENSVSIHIRRGDYLNKENASIYGGICTDDYYKKAIEYIREKVSNPKFYIFSDDIEWAKQHFKGDDMTIVDWNNGKDSYYDMYLMSSCKHNIIANSTFSWWGAWLNQNPEKIVIAPKKWLNNHETSDIVCDNWIRIDGNGEIRSEEYGVRTGSTVK

SEQ ID NO: 102 - DNA, 909 bp, synthetic construct
  misc_feature 1..909: note = ASR8
  source 1..909: mol_type = other DNA
atgattattatccgcattatggggggcttgggcaaccagatgttccaatatgctctgtatcgcaaactaaagtcaatgggtaaagaggttaaattggatatttcgtggtatgacgatcataatacccatcgctcatttgaattagatgtttttggcattgaatatgacgtcgcatccaaaaaagaaatctcgaaattctctaaccgctcaagcaactttttgtctcgaatccgccggaagttgttcggaaaaaagaataaaatctatcaggaggaggacttcaactatgacccggagatcctggaaatggatgatgtgtacctggaagggtactggcagtcggaaaaatattttgaggatattcgtgaacagttacgtaaagaatttaccttcccgaaagagatgaacaaacagaacaaggaactgctggaacagatggaaaacgaaaattccgtgtccatccatattcgtcgtggagattatttaaacaaagaaaacgcaagcatttatggaggaatctgcaccgatgattattataaaaaggcaattgagtatattcgcgagaaagttagtaacccgaagttctatattttttcggatgatatagagtgggcaaaacagcatttcaaaggggacgatatgaccatcgtggactggaataacggcaaagattcctattacgatatgtacctgatgtcgagttgtaaacacaacattattgccaactccacgttttcatggtggggcgcctggctgaaccaaaacccggaaaagattgtgatcgctccgaaaaaatggcttaacaatcatgaaactagcgatattgtttgcgataactggattcgtatcgatggtaatggagaaattcggtcggaggaatatggggtccgcaccggaagcaccgtgaaa

SEQ ID NO: 103 - protein, 291 aa, synthetic construct
  REGION 1..291: note = ASR9
MIIVRLTGGLGNQMFQYAMGRRLAEKHNTELKLDISAFENYKLRKYSLHHFNIQENFATPEEISRLTSVKQNKIEKLLHKILRKKPKKSNTYIKEKHFHFDPNILNLPDNVYLDGYWQSEKYFKDIEDIIRKEFTIKYPQTGKNKEIAEKIQSCNSVSIHIRRGDYVTNPTTNQVHGVCGLDYYQRCIDYIAKKVENPHFFVFSDDPEWVKENLKIQYPTTYVDHNNTDKDYEDLRLMSQCKHHIIANSTFSWWGAWLNSNPDKIVIAPKKWFNTSDYNTKDLIPENWIKL

SEQ ID NO: 104 - DNA, 873 bp, synthetic construct
  misc_feature 1..873: note = ASR9
  source 1..873: mol_type = other DNA
atgattattgtccgactcaccggcggtctgggcaatcaaatgttccaatatgcaatgggtcgccgtttagcggaaaaacacaatacagaactcaaactggacattagcgcgttcgagaattataaactgcgaaagtatagtctgcaccattttaatatccaagaaaattttgcaaccccagaagagattagtcgtttaacgagcgtaaaacaaaacaagatcgaaaaactgttgcacaaaatccttcgcaagaaaccgaaaaaatcaaacacctacattaaggagaaacattttcattttgatccgaatatactgaatctgccggataatgtatacttagatggatactggcaaagcgaaaaatacttcaaggatattgaagatattattcgtaaagaatttacaatcaaatatccacagacgggtaaaaacaaggaaattgcggagaaaattcagtcttgcaactctgtaagtatacacattcgtcgcggtgattatgtaaccaacccgaccactaaccaggttcatggtgtttgtggcctggattattatcagaggtgcatcgactatattgcgaaaaaggtggagaacccgcacttttttgttttctctgatgatcctgaatgggtaaaagaaaatcttaaaatccagtatccaaccacgtatgtggaccataataacacagataaagattacgaagatttgcgtctgatgtcgcagtgtaaacaccacatcatcgcgaactctacctttagctggtggggtgcctggctgaatagtaatccagataaaatagtgattgctccgaaaaaatggtttaatacgagcgactacaataccaaagacttaatacctgaaaattggatcaaactg

SEQ ID NO: 105 - protein, 273 aa, synthetic construct
  REGION 1..273: note = ASR10
MIVVKLIGGLGNQMFQYAAAKALALEKNQKLRLDVSAFETYKLHNYGLNHFNITAKIYKKENKWLRKIKSFFKKNTYYKEQDFGYNPDLFNLKADNIFLEGYFQSEKYFLKYEKEIRKDFEIISPLKKQTKEMIEKIQSVNSVSIHIRRGDYLTNPIHNTSKEEYYKKAMKFIESKIENPVFFVFSDDMDWVKENFKTNHETVFVDFNDASTNFEDIKLMSSCKHNIIANSSFSWWGAWLNQNPNKIVIAPKQWFNDDSINTSDIIPESWIKI

SEQ ID NO: 106 - DNA, 819 bp, synthetic construct
  misc_feature 1..819: note = ASR10
  source 1..819: mol_type = other DNA
atgatcgtcgttaaacttatcggtggtctggggaaccaaatgtttcagtatgccgcggcgaaggctctggcgctcgaaaaaaaccaaaaactgcgcttggacgttagtgcatttgaaacttataaattacacaactatggcctcaatcatttcaatatcacggcgaaaatttacaaaaaggaaaacaagtggttacgcaaaataaaatcattctttaaaaaaaacacctattataaagagcaggacttcggatacaatcctgacctgtttaacttgaaagctgataacatctttcttgaagggtatttccaatcggaaaaatatttcctcaaatatgaaaaagagattcgaaaagacttcgaaattattagtcctctgaaaaaacaaacgaaagaaatgatcgaaaaaatccaatccgtgaactctgtctctatccatatccgtcgcggcgactacctcacgaatcccatacataacacctccaaggaggaatactataaaaaagcaatgaaatttattgagtcgaaaatcgaaaaccccgtgttctttgtattttcggatgatatggactgggtgaaagaaaactttaaaacgaaccatgagactgtattcgtggatttcaatgatgcgagcacaaatttcgaagatattaagctgatgtcatcgtgtaaacacaatatcattgcgaacagttccttctcttggtggggggcctggctgaatcagaatccaaataaaattgtgatcgctccgaagcaatggtttaatgatgattcgattaatacctcggatattattcctgagagttggatcaaaatc

SEQ ID NO: 107 - protein, 290 aa, synthetic construct
  REGION 1..290: note = ASR11
MIIIRMSGGLGNQMFQYALYRKLKAMGKEVKIDDVTGYEDDNQRPIMLDVFGIDYDRATKEEVTELTDSSMDFLSRIRRKLFGRKSKEYREEDCNFDPQVLEMDDAYLEGYFQSEKYFQDVREQLRKEFRFRSGSVPLSEKTRELQKQIENSNSVSIHIRRGDYLENGHAEVYGGICTDDYYKKAIEYMKEKFPDAKFYIFSNDVEWAKQHFKGENFVVVEGSEENTGYLDMFLMSKCRHHIIANSSFSWWGAWLNENPEKIVIAPSKWLNNRECKDIYTERMIRISAEV

SEQ ID NO: 108 - DNA, 870 bp, synthetic construct
  misc_feature 1..870: note = ASR11
  source 1..870: mol_type = other DNA
atgatcattattcgcatgtcaggcgggctgggcaaccagatgtttcagtatgccctctatcgcaagttgaaagctatgggcaaagaggttaaaattgacgacgtaacgggatatgaagatgacaatcaacgtccgatcatgctggacgtgtttggtatcgattacgaccgtgcgaccaaagaagaagtgaccgaactcaccgactcctcaatggactttctgtcccgtatccgccgtaagctgtttggccgcaaatctaaagaatatcgtgaagaagattgtaattttgatccgcaggtgcttgaaatggatgacgcatacctggagggttatttccagagcgaaaaatactttcaggatgttagggaacagctgcgcaaagagtttcgatttcgttcaggttcagtgccgctgtcggaaaagacgcgggaattacagaaacagattgagaacagcaactctgtgagtatccatatcagacgtggtgactacctggaaaatggtcatgcagaagtttatggtggcatctgtacggacgactactataaaaaagccatcgaatacatgaaagagaaattcccggatgcgaagttctacattttttctaatgatgtcgaatgggctaagcagcattttaaaggcgaaaattttgtggttgtggaaggttcggaagaaaataccggctatttagatatgtttcttatgagcaagtgtcgccatcatataattgccaactctagttttagctggtggggcgcatggctcaatgaaaacccagaaaagattgtaatcgcgccgtctaaatggctgaacaaccgtgaatgcaaagatatttataccgaacgtatgattcgtatttccgcagaagta

SEQ ID NO: 109 - protein, 307 aa, synthetic construct
  REGION 1..307: note = ASR12
MIIIRMSGGLGNQMFQYALYRKLKSMGKEVKIDDITGYEDDNQRSIMLDVFGIDYDKATKEEITKLTDSSMDFLSRIRRKLFGRKSKEYQEEDFNFDPQVLEMDDAYLEGYFQSEKYFQDVREQLRKEFTFRKNSVPELSEQTKELRKQIENSNSVSIHIRRGDYLENSHAEIYGGICTDDYYKKAIEYMKEKFPDAKFYIFSNDIEWAKQHFKGENFVIVDASEENTGYADMYLMSKCKHHIIANSSFSWWGAWLNDNPEKIVIAPSKWLNNKECKDIYTDRMIKIDAKGEVRSEDYGVRTNSTVK

SEQ ID NO: 110 - DNA, 921 bp, synthetic construct
  misc_feature 1..921: note = ASR12
  source 1..921: mol_type = other DNA
atgattattatacgtatgagtggcggcctgggtaatcaaatgtttcagtatgccctgtaccgcaaattgaaatcgatggggaaagaggtgaaaatagacgacatcaccgggtatgaggacgataaccagcgttctatcatgctcgatgtgtttgggattgattacgacaaagcaaccaaagaagagataaccaagctgaccgacagtagcatggactttctgtctcgcattcgtcgcaaactgtttggccgcaaatcgaaggagtaccaggaagaagattttaattttgacccacaagtcctggaaatggatgatgcctacctcgaagggtacttccaaagtgaaaagtatttccaggatgtgcgggagcagctgcgaaaagaatttacctttcgaaaaaacagcgtgccggaactgtcggaacagacgaaagaactgcgcaaacaaattgaaaatagcaacagcgtgtcgattcacattcgccgtggtgactatttggaaaactcccacgccgagatttatggcggtatttgtactgacgattactacaagaaagcgattgagtacatgaaagagaaattcccggatgcaaagttttacattttctcgaatgatattgaatgggcgaaacagcactttaaaggggagaattttgtaattgttgacgcatcagaagagaacactggctatgcggatatgtacctgatgagcaaatgcaaacaccacattattgccaattcctccttctcgtggtggggtgcctggctgaacgataacccggaaaaaatcgtgattgctccgagtaaatggctcaataataaagagtgcaaagatatttacaccgaccgcatgattaaaattgacgccaaaggtgaggtccgttcagaggattacggcgtacgtaccaactctaccgtgaaa

(19) United States
(12) Patent Application Publication
(10) Pub. No.: US 2024/0093255 A1
(43) Pub. Date: Mar. 21, 2024

(54) BIOSYNTHETIC PRODUCTION OF 2-FUCOSYLLACTOSE

(71) Applicant: Conagen Inc., Bedford, MA (US)

(72) Inventors: Guohong Mao, Burlington, MA (US); Meaghan Valliere, Lexington, MA (US); Johnson Wu, Bedford, MA (US); Oliver Yu, Lexington, MA (US); Sean Robert Johnson, Arlington, MA (US)

(73) Assignee: Conagen Inc., Bedford, MA (US)

(21) Appl. No.: 18/170,576

(22) Filed: Feb. 17, 2023

Related U.S. Application Data
(63) Continuation of application No. PCT/US21/46659, filed on Aug. 19, 2021.
(60) Provisional application No. 63/067,858, filed on Aug. 19, 2020.
(60) Provisional application No. 63/199,978, filed on Feb. 5, 2021.

(51) Int. Cl. C12P 19/18; C12N 9/10; C12N 9/88; C12P 19/04

(52) U.S. Cl. CPC C12P 19/18; C12N 9/1051; C12N 9/88; C12P 19/04; C12Y 204/01047; C12Y 204/01069

(57) ABSTRACT

The present invention provides a novel biosynthetic production process which converts L-galactose into 2′-fucosyllactose via four enzymatically catalyzed reaction steps. The present process is designed such that co-factors required by the process are regenerated within the four reaction steps, hence making the process cost-effective and efficient. The process can be performed in vitro in a cell-free system. The present invention also provides mutant enzymes that can be used to increase production levels of 2′-fucosyllactose, whether using the novel pathway described herein or the mannose-dependent pathway known in the art.

### RELATED APPLICATIONS

This application is a continuation of International Patent Application No. PCT/US2021/046659, filed Aug. 19, 2021, entitled “BIOSYNTHETIC PRODUCTION OF 2-FUCOSYLLACTOSE”, which claims the benefit under 35 U.S.C. § 119(e) to U.S. Provisional Application No. 63/067,858, filed Aug. 19, 2020, entitled “BIOSYNTHETIC PRODUCTION OF 2-FUCOSYLLACTOSE,” and to U.S. Provisional Application No. 63/199,978, filed Feb. 5, 2021, entitled “BIOSYNTHETIC PRODUCTION OF 2-FUCOSYLLACTOSE,” the entire contents of each of which is incorporated herein by reference.

### FIELD OF THE INVENTION

The field of the invention relates to the production of 2′-fucosyllactose. More specifically, the present disclosure provides a novel biosynthetic pathway which converts L-galactose into 2′-fucosyllactose via four enzymatically catalyzed reaction steps that include the regeneration of various co-factors used in the pathway.

### REFERENCE TO AN ELECTRONIC SEQUENCE LISTING

The contents of the electronic sequence listing (C149770041US03-SEQ-ZJG.xml; Size: 197,436 bytes; and Date of Creation: Feb. 13, 2023) is herein incorporated by reference in its entirety.

### BACKGROUND OF THE INVENTION

Human milk oligosaccharides (HMOs) are the third most abundant solid component of human milk after lactose and lipids. However, they are not found in comparable abundances in other natural sources, including cow milk, sheep milk, or goat milk. Comparing to formula-fed infants, breast-fed infants have lower incidences of diarrhea, respiratory diseases, and otitis media, and appear to develop better. Clinical data show that many benefits of human milk can be attributed to HMOs.

Trisaccharide 2′-fucosyllactose (2′-FL, the chemical structure of which is illustrated in FIG. 1) is one of the most abundant and clinically demonstrated HMOs, making 2′-FL a potential nutritional supplement and therapeutic agent. In particular, there is immense interest in incorporating 2′-FL as a functional additive in infant formula. However, the limited availability of human milk and the complexity of the chemical synthesis of 2′-FL pose limits to supply and cost efficiency. In recent years, industry and academia have explored producing 2′-FL via biosynthesis by utilizing engineered microbial strains (mostly E. coli strains) for fermentative production.

In a typical biosynthetic process, microbial strains were engineered to overexpress α-1,2-fucosyltransferase (FutC), which catalyzes the production of 2′-FL from lactose and GDP-L-fucose. Two major approaches have been adopted to engineer a GDP-L-fucose synthesis pathway in 2′-FL production. One approach (the “salvage pathway” illustrated in FIG. 2) requires only a bi-functional enzyme, FKP, to convert L-fucose to GDP-L-fucose directly. The other approach (the “De novo synthesis” illustrated in FIG. 2) uses glucose to synthesize GDP-L-fucose via a 7-step process.

Nonetheless, there are concerns with regulators and consumers that fermentatively produced food products, especially those intended to be used in baby foods and formula, are susceptible to endotoxin and phage contamination. In addition, there is a need in the art for novel methods to produce 2′-FL that involve fewer steps and are more cost-effective.

### SUMMARY OF THE INVENTION

In one aspect, the present disclosure provides a novel biosynthetic production process which converts L-galactose into 2′-fucosyllactose via four enzymatically catalyzed reaction steps (FIG. 3). The present process is designed such that co-factors required by the process are regenerated within the four reaction steps, hence making the process cost-effective and efficient. The process can be performed in vitro in a cell-free system.

In one embodiment, the present disclosure provides a method for producing 2′-fucosyllactose, where the method includes (a) incubating GDP-L-galactose with a dehydratase and a reductase in the presence of NADPH and/or NADP⁺ for a sufficient time to convert the GDP-L-galactose into GDP-L-fucose; and (b) incubating the GDP-L-fucose with lactose and an α-1,2-fucosyltransferase for a sufficient time to convert the GDP-L-fucose and lactose into 2′-fucosyllactose and GDP.

In some embodiments, the dehydratase can be a GDP-mannose-4,6-dehydratase. Suitable dehydratases include an enzyme comprising an amino acid sequence having at least 60%, at least 70%, at least 80%, at least 85%, at least 90%, at least 95%, at least 98%, or at least 99% sequence identity to SEQ ID NO: 5, SEQ ID NO: 9, SEQ ID NO: 11, or SEQ ID NO: 13. In particular embodiments, the dehydratase used in the present method comprises the amino acid sequence set forth in SEQ ID NO: 5, SEQ ID NO: 9, SEQ ID NO: 11, or SEQ ID NO: 13.

In some embodiments, the reductase used in the present method can be a GDP-4-keto-6-deoxy-mannose reductase. Suitable reductases include an enzyme comprising an amino acid sequence having at least 60%, at least 70%, at least 80%, at least 85%, at least 90%, at least 95%, at least 98%, or at least 99% sequence identity to SEQ ID NO: 7, SEQ ID NO: 15, or SEQ ID NO: 17. In particular embodiments, the reductase used in the present method comprises the amino acid sequence set forth in SEQ ID NO: 7, SEQ ID NO: 15, or SEQ ID NO: 17.

Suitable α-1,2-fucosyltransferases for use in the present method include those enzymes comprising an amino acid sequence having at least 60%, at least 70%, at least 80%, at least 85%, at least 90%, at least 95%, at least 98%, or at least 99% sequence identity to any one of SEQ ID NOs: 21, 23, 25, 27, 29, 31, 33, 35, 37, 39, 41, 43, 45, 47, 49, 51, 53, 55, 57, 59, and 61. In certain embodiments, the α-1,2-fucosyltransferase used in the present method comprises the amino acid sequence set forth in any one of SEQ ID NOs: 21, 23, 25, 27, 29, 31, 33, 35, 37, 39, 41, 43, 45, 47, 49, 51, 53, 55, 57, 59, and 61. In particular embodiments, the α-1,2-fucosyltransferase used in the present method comprises the amino acid sequence set forth in SEQ ID NO: 23, SEQ ID NO: 29, SEQ ID NO: 39, SEQ ID NO: 45, SEQ ID NO: 55, or SEQ ID NO: 61.

In some embodiments, the present method further includes incubating the GDP-L-galactose in the presence of a first regenerating enzyme and a first substrate for said first regenerating enzyme, wherein said first regenerating enzyme catalyzes a reaction involving the first substrate that uses NADP⁺ as a co-factor, thereby regenerating NADPH. For example, the first regenerating enzyme and the first substrate can be selected from the group consisting of (a) a malate dehydrogenase and a malate, (b) a formate dehydrogenase and a formate, (c) a phosphite dehydrogenase and a phosphite, and (d) a glucose dehydrogenase and glucose.

In some embodiments, the GDP-L-galactose used in the present method is generated in situ. In certain embodiments, the GDP-L-galactose used in the present method can be generated from GDP-mannose. In such embodiments, the present method can further include incubating GDP-mannose with a GDP-mannose-3,5-epimerase for a sufficient time to convert the GDP-mannose into GDP-L-galactose. For example, the GDP-mannose-3,5-epimerase can be an enzyme comprising an amino acid sequence having at least 60%, at least 70%, at least 80%, at least 85%, at least 90%, at least 95%, at least 98%, or at least 99% sequence identity to SEQ ID NO: 3 or SEQ ID NO: 19. In particular embodiments, the GDP-mannose-3,5-epimerase can comprise the amino acid sequence set forth in SEQ ID NO: 3 or SEQ ID NO: 19.

Alternatively, the GDP-L-galactose used in the present method can be generated from L-galactose. In such embodiments, the present method can further include incubating L-galactose with a fucokinase/guanylyltransferase in the presence of ATP and GTP for a sufficient time to convert the L-galactose into GDP-L-galactose. For example, the fucokinase/guanylyltransferase can be an enzyme comprising an amino acid sequence having at least 60%, at least 70%, at least 80%, at least 85%, at least 90%, at least 95%, at least 98%, or at least 99% sequence identity to SEQ ID NO: 1. In particular embodiments, the fucokinase/guanylyltransferase can comprise the amino acid sequence set forth in SEQ ID NO: 1.

In preferred embodiments, the present method further includes incubating the L-galactose in the presence of a second regenerating enzyme and a second substrate for said second regenerating enzyme, wherein said second regenerating enzyme catalyzes a reaction involving the second substrate that uses ADP as a co-factor, thereby regenerating ATP. In further preferred embodiments, the present method can further include incubating the L-galactose in the presence of a third regenerating enzyme and a third substrate for said third regenerating enzyme, wherein said third regenerating enzyme catalyzes a reaction involving the third substrate that uses GDP as a co-factor, thereby regenerating GTP. As noted above, GDP is produced as a by-product in the bioconversion of 2′-fucosyllactose from GDP-L-fucose.

Respectively, the second regenerating enzyme and the second substrate, and the third regenerating enzyme and the third substrate, independently can be selected from the group consisting of (a) a pyruvate kinase and a phospho(enol)pyruvate (PEP), (b) a creatine kinase and a creatine phosphate, (c) an acetate kinase and an acetyl phosphate, (d) a polyphosphate kinase and a polyphosphate, and (e) a polyphosphate:AMP phosphotransferase, an adenylate kinase and an adenosine monophosphate.

In another aspect, the present disclosure relates to identifying new enzymes that can be used to convert GDP-L-fucose into 2′-fucosyllactose. Such enzymes can be an enzyme comprising an amino acid sequence having at least 60%, at least 70%, at least 80%, at least 85%, at least 90%, at least 95%, at least 98%, or at least 99% sequence identity to any one of the SEQ ID NOs: 21, 23, 25, 27, 29, 31, 33, 35, 37, 39, 41, 43, 45, 47, 49, 51, 53, 55, 57, 59, and 61. In certain embodiments, the enzyme can comprise the amino acid sequence set forth in any one of SEQ ID NOs: 21, 23, 25, 27, 29, 31, 33, 35, 37, 39, 41, 43, 45, 47, 49, 51, 53, 55, 57, 59, and 61. In particular embodiments, the enzyme can comprise the amino acid sequence set forth in SEQ ID NO: 29, SEQ ID NO: 61, SEQ ID NO: 39, SEQ ID NO: 45, SEQ ID NO: 55, or SEQ ID NO: 23.

Accordingly, the present disclosure also relates to a method for producing 2′-fucosyllactose, where the method involves incubating GDP-L-fucose with lactose and an α-1,2-fucosyltransferase for a sufficient time to convert said GDP-L-fucose and lactose into 2′-fucosyllactose, wherein the α-1,2-fucosyltransferase is an enzyme comprising an amino acid sequence having at least 60%, at least 70%, at least 80%, at least 85%, at least 90%, at least 95%, at least 98%, or at least 99% sequence identity to any one of SEQ ID NOs: 21, 23, 25, 27, 29, 31, 33, 35, 37, 39, 41, 43, 45, 47, 49, 51, 53, 55, 57, 59, and 61. In certain embodiments, the α-1,2-fucosyltransferase can comprise the amino acid sequence set forth in any one of SEQ ID NOs: 21, 23, 25, 27, 29, 31, 33, 35, 37, 39, 41, 43, 45, 47, 49, 51, 53, 55, 57, 59, and 61. In particular embodiments, the α-1,2-fucosyltransferase used in the present method can comprise the amino acid sequence set forth in SEQ ID NO: 29, SEQ ID NO: 61, SEQ ID NO: 39, SEQ ID NO: 45, SEQ ID NO: 55, or SEQ ID NO: 23.

In another aspect, the present disclosure relates to a method for producing 2′-fucosyllactose from L-galactose. The method can include (a) providing a reaction mixture comprising (i) a fucokinase/guanylyltransferase, (ii) a dehydratase, (iii) a reductase, (iv) an ca-1,2-fucosyltransferase, (v) ATP, (vi) GTP, (vii) NADP⁺, and (viii) NADPH; (b) adding L-galactose to the reaction mixture; and (c) incubating the reaction mixture for a sufficient time to produce 2′-fucosyllactose. The reaction mixture can further include (ix) a first regenerating enzyme and a first substrate for said first regenerating enzyme, wherein said first regenerating enzyme is capable of catalyzing a first regeneration reaction involving the first substrate that uses NADP⁺ as a co-factor, thereby regenerating NADPH; (x) a second regenerating enzyme and a second substrate for said second regenerating enzyme, wherein said second regenerating enzyme is capable of catalyzing a second regeneration reaction involving the second substrate that uses ADP as a co-factor, thereby regenerating ATP; and (xi) a third regenerating enzyme and a third substrate for said third regenerating enzyme, wherein said third regenerating enzyme is capable of catalyzing a third regeneration reaction involving the third substrate that uses GDP as a co-factor, thereby regenerating GTP.

In yet another aspect, the present disclosure relates to mutant enzymes that can be used to increase production levels of 2′-fucosyllactose. These mutant enzymes can include mutant dehydratases and mutant α-1,2-fucosyltransferases.

In the de novo pathway described in FIG. 2 (left), the conversion of GDP-D-mannose to GDP-4-keto-6-deoxymannose is catalyzed by GDP-mannose-4,6-dehydratase (GMD). The resulting GDP-4-keto-6-deoxymannose is converted to GDP-L-fucose by a bifunctional 3,5-epimerase-4-reductase (e.g., WcaG from E. coli) enzyme. Yet, it has been well-established that GDP-L-fucose acts as a negative feedback to the activity of GMD enzymes (FIG. 13). The inhibition is characterized as allosteric inhibition with human and A. thaliana GMD. Therefore, it is beneficial to generate mutant enzymes targeting the GDP-L-fucose allosteric binding pocket in A. thaliana GMD (At GMD, SEQ ID NO: 5) and human GMD (Hs GMD, SEQ ID NO: 9). Accordingly, in one embodiment, the present disclosure relates to a mutant At GMD comprising an amino acid sequence of SEQ ID NO: 79, SEQ ID NO: 77, or SEQ ID NO: 75. In another embodiment, the present disclosure relates to a mutant Hs GMD comprising an amino acid sequence of SEQ ID NO: 85, SEQ ID NO: 83, or SEQ ID NO: 81.

In another embodiment, the present disclosure relates to a mutant enzyme having improved α-1,2-fucosyltransferase activity. Accordingly, in one embodiment, such mutant ca-1,2-fucosyltransferase can be a polypeptide comprising an amino acid sequence having at least 60%, at least 70%, at least 80%, at least 85%, at least 90%, at least 95%, at least 98%, or at least 99% sequence identity to SEQ ID NO: 109, SEQ ID NO: 107, SEQ ID NO: 89, SEQ ID NO: 91, SEQ ID NO: 93, SEQ ID NO: 95, SEQ ID NO: 99, SEQ ID NO: 101, SEQ ID NO: 103, or SEQ ID NO: 105.

In one aspect, the present disclosure relates to a method for producing 2′-fucosyllactose, said method includes providing the following enzymes in a culture medium comprising L-galactose, said enzymes comprise: (i) a fucokinase comprising an amino acid sequence having at least 90% sequence identity to SEQ ID NO: 1; (ii) a dehydratase comprising an amino acid having at least 90% sequence identity to SEQ ID NO: 5, SEQ ID NO: 79, SEQ ID NO: 77, or SEQ ID NO: 75; (iii) a reductase comprising an amino acid having at least 90% sequence identity to SEQ ID NO: 7; and (iv) an α-1,2-fucosyltransferase comprising an amino acid sequence having at least 70%, at least 80%, at least 85%, at least 90%, at least 95%, at least 98%, or at least 99% sequence identity to SEQ ID NO: 109; and incubating L-galactose with said enzymes for a sufficient time to produce 2′-fucosyllactose.

In another aspect, the present disclosure relates to a method for producing 2′-fucosyllactose, said method includes providing the following enzymes in a culture medium comprising GDP-mannose, said enzymes comprise: (i) an epimerase comprising an amino acid sequence having at least 90% sequence identity to SEQ ID NO: 3; (ii) a dehydratase comprising an amino acid having at least 90% sequence identity to SEQ ID NO: 5, SEQ ID NO: 79, SEQ ID NO: 77, or SEQ ID NO: 75; (iii) a reductase comprising an amino acid having at least 90% sequence identity to SEQ ID NO: 7; and (iv) an α-1,2-fucosyltransferase comprising an amino acid sequence having at least 70%, at least 80%, at least 85%, at least 90%, at least 95%, at least 98%, or at least 99% sequence identity to SEQ ID NO: 109; and incubating GDP-mannose with said enzymes for a sufficient time to produce 2′-fucosyllactose.

The present disclosure also encompasses nucleic acid constructs comprising a nucleic acid sequence that encodes at least a mutant dehydratase and/or a mutant α-1,2-fucosyltransferase described herein, as well as a microorganism comprising said nucleic acid construct(s). Said microorganism or host cell can be induced to express the mutant dehydratase and/or mutant α-1,2-fucosyltransferase. To facilitate protein purification after expression, the nucleic acid sequence that encodes the present mutant dehydratase and/or a mutant α-1,2-fucosyltransferase can include a polyhistidine tag. The most common polyhistidine tag are formed of six histidine (6×His tag) residues, which are added at the N-terminus preceded by methionine or C-terminus before a stop codon, in the coding sequence of the protein of interest.

The present disclosure also relates to an engineered microorganism for enhanced production of 2′-fucosyllactose, where such microorganism includes at least the following heterologous genes for producing 2′-fucosyllactose: (i) a first heterologous gene that encodes a mutant dehydratase for producing GDP-L-fucose, said mutant dehydratase being a polypeptide comprising an amino sequence selected from SEQ ID NO: 79, SEQ ID NO: 77, SEQ ID NO: 75, SEQ ID NO: 85, SEQ ID NO: 83, and SEQ ID NO: 81; and (ii) a second heterologous gene that encodes a mutant α-1,2-fucosyltransferase for converting GDP-L-fucose to 2′-fucosyllactose, said mutant α-1,2-fucosyltransferase being a polypeptide comprising an amino acid sequence having at least 70%, at least 80%, at least 85%, at least 90%, at least 95%, at least 98%, or at least 99% sequence identity to SEQ ID NO: 109. The microorganism can further include a heterologous gene for exporting 2′-fucosyllactose extracellularly.

Although many aspects of the present disclosure relate to producing 2′-fucosyllactose enzymatically, the present teaching also encompasses producing 2′-fucosyllactose via fermentation, in particular, via culturing a microorganism that has been engineered to include (i) a first heterologous gene that encodes a mutant dehydratase for producing GDP-L-fucose, said mutant dehydratase being a polypeptide comprising an amino sequence selected from SEQ ID NO: 79, SEQ ID NO: 77, SEQ ID NO: 75, SEQ ID NO: 85, SEQ ID NO: 83, and SEQ ID NO: 81; and (ii) a second heterologous gene that encodes a mutant α-1,2-fucosyltransferase for converting GDP-L-fucose to 2′-fucosyllactose, said mutant α-1,2-fucosyltransferase being a polypeptide comprising an amino acid sequence having at least 70%, at least 80%, at least 85%, at least 90%, at least 95%, at least 98%, or at least 99% sequence identity to SEQ ID NO: 109. The microorganism can be cultured in culture medium including at least one carbon source. The method can include separating the culture medium from the microorganism, then isolating 2′-fucosyllactose from the culture medium.

Some aspects of the present disclosure provide methods for producing 2′-fucosyllactose comprising: incubating GDP-L-fucose with an α-1,2-fucosyltransferase in a culture medium comprising lactose for a sufficient time to convert said GDP-L-fucose and lactose into 2′-fucosyllactose and GDP; wherein said α-1,2-fucosyltransferase is selected from the group consisting of a polypeptide comprising an amino acid sequence having at least 70% (e.g., at least 70%, at least 75%, at least 80%, at least 85%, at least 90%, at least 95%, or at least 99%) sequence identity to SEQ ID NO: 109, SEQ ID NO: 29, SEQ ID NO: 107, SEQ ID NO: 89, SEQ ID NO: 91, SEQ ID NO: 93, SEQ ID NO: 95, SEQ ID NO: 99, SEQ ID NO: 101, SEQ ID NO: 103, or SEQ ID NO: 105. In some embodiments, the α-1,2-fucosyltransferase is a polypeptide comprising the amino acid sequence of any one of SEQ ID NO: 109, SEQ ID NO: 29, SEQ ID NO: 107, SEQ ID NO: 89, SEQ ID NO: 91, SEQ ID NO: 93, SEQ ID NO: 95, SEQ ID NO: 99, SEQ ID NO: 101, SEQ ID NO: 103, or SEQ ID NO: 105.

In some embodiments, the GDP-L-fucose is generated in situ in the culture medium from GDP-mannose or GDP-L-galactose in a reaction catalyzed by a dehydratase enzyme.

In some embodiments, the dehydratase enzyme is a polypeptide comprising an amino acid sequence having at least 70% (e.g., at least 70%, at least 75%, at least 80%, at least 85%, at least 90%, at least 95%, or at least 99%) sequence identity to SEQ ID NO: 5 or SEQ ID NO: 9. In some embodiments, the dehydratase enzyme is a polypeptide comprising the amino acid sequence of SEQ ID NO: 5 or SEQ ID NO: 9.

In some embodiments, the dehydratase enzyme is a polypeptide comprising an amino acid sequence having at least 70% (e.g., at least 70%, at least 75%, at least 80%, at least 85%, at least 90%, at least 95%, or at least 99%) sequence identity to SEQ ID NO: 79, SEQ ID NO: 77, or SEQ ID NO: 75. In some embodiments, the dehydratase enzyme is a polypeptide comprising the amino acid sequence of SEQ ID NO: 79, SEQ ID NO: 77, or SEQ ID NO: 75.

In some embodiments, the dehydratase enzyme is a polypeptide comprising an amino acid sequence having at least 70% (e.g., at least 70%, at least 75%, at least 80%, at least 85%, at least 90%, at least 95%, or at least 99%) sequence identity to SEQ ID NO: 85, SEQ ID NO: 83, or SEQ ID NO: 81. In some embodiments, the dehydratase enzyme is a polypeptide comprising the amino acid sequence of SEQ ID NO: 85, SEQ ID NO: 83, or SEQ ID NO: 81.

Further provided herein are methods for producing 2′-fucosyllactose, the method comprising:
incubating GDP-mannose and/or GDP-L-galactose with a dehydratase and a reductase in the presence of NADPH and/or NADP+ in a culture medium for a sufficient time to convert said GDP-mannose and/or GDP-L-galactose into GDP-L-fucose; and
incubating said GDP-L-fucose with an α-1,2-fucosyltransferase and lactose for a sufficient time to convert said GDP-L-fucose and lactose into 2′-fucosyllactose and GDP; wherein said dehydratase is selected from the group consisting of: a polypeptide comprising an amino acid sequence having at least 70% (e.g., at least 70%, at least 75%, at least 80%, at least 85%, at least 90%, at least 95%, or at least 99%) sequence identity to SEQ ID NO: 79, SEQ ID NO: 77, SEQ ID NO: 75, or SEQ ID NO: 5; and a polypeptide comprising an amino acid sequence having at least 70% (e.g., at least 70%, at least 75%, at least 80%, at least 85%, at least 90%, at least 95%, or at least 99%) sequence identity to SEQ ID NO: 85, SEQ ID NO: 83, SEQ ID NO: 81, or SEQ ID NO: 9.

In some embodiments, the dehydratase is a polypeptide comprising the amino acid of any one of SEQ ID NO: 79, SEQ ID NO: 77, or SEQ ID NO: 75. In some embodiments, the dehydratase is a polypeptide comprising the amino acid of any one of SEQ ID NO: 85, SEQ ID NO: 83, SEQ ID NO: 81, or SEQ ID NO: 9.

In some embodiments, the α-1,2-fucosyltransferase is a polypeptide comprising an amino acid sequence having at least 70% (e.g., at least 70%, at least 75%, at least 80%, at least 85%, at least 90%, at least 95%, or at least 99%) sequence identity to SEQ ID NO: 109, SEQ ID NO: 29, SEQ ID NO: 107, SEQ ID NO: 89, SEQ ID NO: 91, SEQ ID NO: 93, SEQ ID NO: 95, SEQ ID NO: 99, SEQ ID NO: 101, SEQ ID NO: 103, or SEQ ID NO: 105. In some embodiments, the α-1,2-fucosyltransferase is a polypeptide comprising the amino acid sequence of SEQ ID NO: 109, SEQ ID NO: 29, SEQ ID NO: 107, SEQ ID NO: 89, SEQ ID NO: 91, SEQ ID NO: 93, SEQ ID NO: 95, SEQ ID NO: 99, SEQ ID NO: 101, SEQ ID NO: 103, or SEQ ID NO: 105.

In some embodiments, the reductase is a polypeptide comprising an amino acid having at least 70% (e.g., at least 70%, at least 75%, at least 80%, at least 85%, at least 90%, at least 95%, or at least 99%) sequence identity to SEQ ID NO: 7, SEQ ID NO: 15, or SEQ ID NO: 17. In some embodiments, the reductase is a polypeptide comprising the amino acid of SEQ ID NO: 7, SEQ ID NO: 15, or SEQ ID NO: 17.

An engineered microorganism for enhanced production of 2′-fucosyllactose, said microorganism comprising at least the following heterologous genes for producing 2′-fucosyllactose:
a first heterologous gene that encodes a mutant dehydratase for producing GDP-L-fucose, said mutant dehydratase being a polypeptide comprising an amino sequence having at least 70% (e.g., at least 70%, at least 75%, at least 80%, at least 85%, at least 90%, at least 95%, or at least 99%) sequence identity to SEQ ID NO: 79, SEQ ID NO: 77, SEQ ID NO: 75, SEQ ID NO: 85, SEQ ID NO: 83, or SEQ ID NO: 81; and
a second heterologous gene that encodes a mutant α-1,2-fucosyltransferase for converting GDP-L-fucose to 2′-fucosyllactose, said mutant α-1,2-fucosyltransferase being a polypeptide comprising an amino acid sequence having at least 70% (e.g., at least 70%, at least 75%, at least 80%, at least 85%, at least 90%, at least 95%, or at least 99%) sequence identity to SEQ ID NO: 109.

In some embodiments, the mutant dehydratase is a polypeptide comprising the amino sequence of SEQ ID NO: 79, SEQ ID NO: 77, SEQ ID NO: 75, SEQ ID NO: 85, SEQ ID NO: 83, or SEQ ID NO: 81. In some embodiments, the mutant α-1,2-fucosyltransferase comprises the amino acid sequence of SEQ ID NO: 109.

In some embodiments, the microorganism further comprises a heterologous gene for exporting 2′-fucosyllactose extracellularly.

Other aspects of the present disclosure provide methods for producing 2′-fucosyllactose comprising culturing the microorganism described herein in a culture medium comprising at least one carbon source. In some embodiments, the method further comprises separating the culture medium from the microorganism. In some embodiments, the method further comprises isolating 2′-fucosyllactose from the culture medium.

Also provided herein are mutant dehydratases for producing GDP-L-fucose, said mutant dehydratase being a polypeptide comprising an amino sequence having at least 70% (e.g., at least 70%, at least 75%, at least 80%, at least 85%, at least 90%, at least 95%, or at least 99%) sequence identity to SEQ ID NO: 79, SEQ ID NO: 77, SEQ ID NO: 75, SEQ ID NO: 85, SEQ ID NO: 83, or SEQ ID NO: 81. In some embodiments, the mutant dehydratase comprises the amino acid sequence of SEQ ID NO: 79, SEQ ID NO: 77, SEQ ID NO: 75, SEQ ID NO: 85, SEQ ID NO: 83, or SEQ ID NO: 81

Further provided herein are mutant α-1,2-fucosyltransferases for producing 2′-fucosyllactose, said mutant α-1,2-fucosyltransferase being a polypeptide comprising an amino acid sequence having at least 70% (e.g., at least 70%, at least 75%, at least 80%, at least 85%, at least 90%, at least 95%, or at least 99%) sequence identity to SEQ ID NO: 109, SEQ ID NO: 29, SEQ ID NO: 107, SEQ ID NO: 89, SEQ ID NO: 91, SEQ ID NO: 93, SEQ ID NO: 95, SEQ ID NO: 99, SEQ ID NO: 101, SEQ ID NO: 103, or SEQ ID NO: 105. In some embodiments, the mutant α-1,2-fucosyltransferase comprises the amino acid sequence of SEQ ID NO: 109, SEQ ID NO: 29, SEQ ID NO: 107, SEQ ID NO: 89, SEQ ID NO: 91, SEQ ID NO: 93, SEQ ID NO: 95, SEQ ID NO: 99, SEQ ID NO: 101, SEQ ID NO: 103, or SEQ ID NO: 105.

Nucleic acid constructs comprising a nucleic acid sequences that encodes at least one of the mutant enzymes described herein, and microorganisms comprising such nucleic acid constructs are also provided.

Further provided herein are method for producing 2′-fucosyllactose, the method comprising: incubating GDP-L-galactose with a dehydratase and a reductase in the presence of NADPH and/or NADP+ for a sufficient time to convert said GDP-L-galactose into GDP-L-fucose; and incubating said GDP-L-fucose with lactose and an α-1,2-fucosyltransferase for a sufficient time to convert said GDP-L-fucose and lactose into 2′-fucosyllactose and GDP.

In some embodiments, the GDP-L-galactose is further incubated in the presence of a first regenerating enzyme and a first substrate for said first regenerating enzyme, wherein said first regenerating enzyme catalyzes a reaction involving the first substrate that uses NADP+ as a co-factor, thereby regenerating NADPH.

In some embodiments, the dehydratase is a GDP-mannose-4,6-dehydratase.

In some embodiments, the dehydratase is an enzyme comprising an amino acid sequence having at least 70% (e.g., at least 70%, at least 75%, at least 80%, at least 85%, at least 90%, at least 95%, or at least 99%) sequence identity to SEQ ID NO: 5, SEQ ID NO: 9, SEQ ID NO: 11, or SEQ ID NO: 13. In some embodiments, the dehydratase is an enzyme comprising the amino acid sequence of SEQ ID NO: 5, SEQ ID NO: 9, SEQ ID NO: 11, or SEQ ID NO: 13.

In some embodiments, the reductase is a GDP-4-keto-6-deoxy-mannose reductase. In some embodiments, the dehydratase is an enzyme comprising an amino acid sequence having at least 70% (e.g., at least 70%, at least 75%, at least 80%, at least 85%, at least 90%, at least 95%, or at least 99%) sequence identity to SEQ ID NO: 7, SEQ ID NO: 15, or SEQ ID NO: 17. In some embodiments, the dehydratase is an enzyme comprising the amino acid sequence of SEQ ID NO: 7, SEQ ID NO: 15, or SEQ ID NO: 17.

In some embodiments, the method further comprises incubating GDP-mannose with a GDP-mannose-3,5-epimerase for a sufficient time to convert said GDP-mannose into GDP-L-galactose. In some embodiments, the GDP-mannose-3,5-epimerase is an enzyme comprising an amino acid sequence having at least 70% (e.g., at least 70%, at least 75%, at least 80%, at least 85%, at least 90%, at least 95%, or at least 99%) sequence identity to SEQ ID NO: 3 or SEQ ID NO: 19. In some embodiments, the GDP-mannose-3,5-epimerase is an enzyme comprising the amino acid sequence of SEQ ID NO: 3 or SEQ ID NO: 19.

In some embodiments, the method further comprises incubating L-galactose with a fucokinase/guanylyltransferase in the presence of ATP and GTP for a sufficient time to convert said L-galactose into GDP-L-galactose.

In some embodiments, the fucokinase/guanylyltransferase is an enzyme comprising an amino acid sequence having at least 70% (e.g., at least 70%, at least 75%, at least 80%, at least 85%, at least 90%, at least 95%, or at least 99%) sequence identity to SEQ ID NO: 1. In some embodiments, the fucokinase/guanylyltransferase is an enzyme comprising the amino acid sequence having at least 70% (e.g., at least 70%, at least 75%, at least 80%, at least 85%, at least 90%, at least 95%, or at least 99%) sequence identity to SEQ ID NO: 1.

In some embodiments, the L-galactose is further incubated in the presence of a second regenerating enzyme and a second substrate for said second regenerating enzyme, wherein said second regenerating enzyme catalyzes a reaction involving the second substrate that uses ADP as a co-factor, thereby regenerating ATP.

In some embodiments, the L-galactose is further incubated in the presence of a third regenerating enzyme and a third substrate for said third regenerating enzyme, wherein said third regenerating enzyme catalyzes a reaction involving the third substrate that uses GDP as a co-factor, thereby regenerating GTP.

In some embodiments, the first regenerating enzyme and the first substrate is selected from the group consisting of (a) a malate dehydrogenase and a malate, (b) a formate dehydrogenase and a formate, (c) a phosphite dehydrogenase and a phosphite, and (d) a glucose dehydrogenase and glucose.

In some embodiments, the second regenerating enzyme and the second substrate is selected from the group consisting of (a) a pyruvate kinase and a phospho(enol)pyruvate (PEP), (b) a creatine kinase and a creatine phosphate, (c) an acetate kinase and an acetyl phosphate, (d) a polyphosphate kinase and a polyphosphate, and (e) a polyphosphate:AMP phosphotransferase, an adenylate kinase and an adenosine monophosphate.

In some embodiments, the third regenerating enzyme and the third substrate is selected from the group consisting of (a) a pyruvate kinase and a phospho(enol)pyruvate (PEP), (b) a creatine kinase and a creatine phosphate, (c) an acetate kinase and an acetyl phosphate, (d) a polyphosphate kinase and a polyphosphate, and (e) a polyphosphate:AMP phosphotransferase, an adenylate kinase and an adenosine monophosphate.

In some embodiments, the α-1,2-fucosyltransferase is an enzyme comprising an amino acid sequence having at least 70% (e.g., at least 70%, at least 75%, at least 80%, at least 85%, at least 90%, at least 95%, or at least 99%) sequence identity to any one of SEQ ID NOs: 21, 23, 25, 27, 29, 31, 33, 35, 37, 39, 41, 43, 45, 47, 49, 51, 53, 55, 57, 59, and 61. In some embodiments, the α-1,2-fucosyltransferase is an enzyme comprising the amino acid sequence of any one of SEQ ID NOs: 21, 23, 25, 27, 29, 31, 33, 35, 37, 39, 41, 43, 45, 47, 49, 51, 53, 55, 57, 59, and 61.

Also provided herein are methods for producing 2′-fucosyllactose, the method comprising incubating GDP-L-fucose with lactose and an α-1,2-fucosyltransferase for a sufficient time to convert said GDP-L-fucose and lactose into 2′-fucosyllactose, wherein the α-1,2-fucosyltransferase is an enzyme comprising an amino acid sequence having at least 70% (e.g., at least 70%, at least 75%, at least 80%, at least 85%, at least 90%, at least 95%, or at least 99%) sequence identity to any one of SEQ ID NOs: 21, 23, 25, 27, 29, 31, 33, 35, 37, 39, 41, 43, 45, 47, 49, 51, 53, 55, 57, 59, and 61. In some embodiments, the α-1,2-fucosyltransferase is an enzyme comprising the amino acid sequence of SEQ ID NOs: 21, 23, 25, 27, 29, 31, 33, 35, 37, 39, 41, 43, 45, 47, 49, 51, 53, 55, 57, 59, and 61.

Other aspects of the present disclosure provide methods for producing 2′-fucosyllactose from L-galactose, the method comprising:
(a) providing a reaction mixture comprising (i) a fucokinase/guanylyltransferase, (ii) a dehydratase, (iii) a reductase, (iv) an α-1,2-fucosyltransferase, (v) ATP, (vi) GTP, (vii) NADP+, and (viii) NADPH;
(b) adding L-galactose to the reaction mixture; and
(c) incubating said reaction mixture for a sufficient time to produce 2′-fucosyllactose;
wherein the reaction mixture further comprises: (ix) a first regenerating enzyme and a first substrate for said first regenerating enzyme, wherein said first regenerating enzyme is capable of catalyzing a first regeneration reaction involving the first substrate that uses NADP+ as a co-factor, thereby regenerating NADPH; (x) a second regenerating enzyme and a second substrate for said second regenerating enzyme, wherein said second regenerating enzyme is capable of catalyzing a second regeneration reaction involving the second substrate that uses ADP as a co-factor, thereby regenerating ATP; and (xi) a third regenerating enzyme and a third substrate for said third regenerating enzyme, wherein said third regenerating enzyme is capable of catalyzing a third regeneration reaction involving the third substrate that uses GDP as a co-factor, thereby regenerating GTP.

In some embodiments, the fucokinase/guanylyltransferase is an enzyme comprising an amino acid sequence having at least 70% (e.g., at least 70%, at least 75%, at least 80%, at least 85%, at least 90%, at least 95%, or at least 99%) sequence identity to SEQ ID NO: 1. In some embodiments, the fucokinase/guanylyltransferase is an enzyme comprising the amino acid sequence of SEQ ID NO: 1.

In some embodiments, the dehydratase is a GDP-mannose-4,6-dehydratase. In some embodiments, the dehydratase is an enzyme comprising an amino acid sequence having at least 70% (e.g., at least 70%, at least 75%, at least 80%, at least 85%, at least 90%, at least 95%, or at least 99%) sequence identity to SEQ ID NO: 5, SEQ ID NO: 9, SEQ ID NO: 11, or SEQ ID NO: 13. In some embodiments, the dehydratase is a GDP-mannose-4,6-dehydratase. In some embodiments, the dehydratase is an enzyme comprising the amino acid of SEQ ID NO: 5, SEQ ID NO: 9, SEQ ID NO: 11, or SEQ ID NO: 13.

In some embodiments, the reductase is a GDP-4-keto-6-deoxy-mannose reductase. In some embodiments, the reductase is an enzyme comprising an amino acid sequence having at least 70% (e.g., at least 70%, at least 75%, at least 80%, at least 85%, at least 90%, at least 95%, or at least 99%) sequence identity to SEQ ID NO: 7, SEQ ID NO: 15, or SEQ ID NO: 17. In some embodiments, the reductase is a GDP-4-keto-6-deoxy-mannose reductase. In some embodiments, the reductase is an enzyme comprising the amino acid of SEQ ID NO: 7, SEQ ID NO: 15, or SEQ ID NO: 17.

In some embodiments, the alpha-1,2-fucosyltransferase is an enzyme comprising an amino acid sequence having at least 70% (e.g., at least 70%, at least 75%, at least 80%, at least 85%, at least 90%, at least 95%, or at least 99%) sequence identity to any one of SEQ ID NOs: 21, 23, 25, 27, 29, 31, 33, 35, 37, 39, 41, 43, 45, 47, 49, 51, 53, 55, 57, 59, and 61. In some embodiments, the alpha-1,2-fucosyltransferase is an enzyme comprising the amino acid sequence of any one of SEQ ID NOs: 21, 23, 25, 27, 29, 31, 33, 35, 37, 39, 41, 43, 45, 47, 49, 51, 53, 55, 57, 59, and 61.

In some embodiments, the first regenerating enzyme and the first substrate is selected from the group consisting of (a) a malate dehydrogenase and a malate, (b) a formate dehydrogenase and a formate, (c) a phosphite dehydrogenase and a phosphite, and (d) a glucose dehydrogenase and glucose.

In some embodiments, the second regenerating enzyme and the second substrate is selected from the group consisting of (a) a pyruvate kinase and a phospho(enol)pyruvate (PEP), (b) a creatine kinase and a creatine phosphate, (c) an acetate kinase and an acetyl phosphate, (d) a polyphosphate kinase and a polyphosphate, and (e) a polyphosphate:AMP phosphotransferase, an adenylate kinase and an adenosine monophosphate.

In some embodiments, the third regenerating enzyme and the third substrate is selected from the group consisting of (a) a pyruvate kinase and a phospho(enol)pyruvate (PEP), (b) a creatine kinase and a creatine phosphate, (c) an acetate kinase and an acetyl phosphate, (d) a polyphosphate kinase and a polyphosphate, and (e) a polyphosphate:AMP phosphotransferase, an adenylate kinase and an adenosine monophosphate.

While the disclosure is susceptible to various modifications and alternative forms, specific embodiments thereof are shown by way of example in the drawing and will herein be described in detail. It should be understood, however, that the drawings and detailed description presented herein are not intended to limit the disclosure to the particular embodiment disclosed, but on the contrary, the intention is to cover all modifications, equivalents, and alternatives falling within the spirit and scope of the present disclosure as defined by the appended claims.

Other features and advantages of this invention will become apparent in the following detailed description of preferred embodiments of this invention, taken with reference to the accompanying drawings.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1 shows the chemical structure of 2′-fucosyllactose (2′-FL).

FIG. 2 shows two prior art biosynthetic pathways for producing 2′-fucosyllactose. GDP-L-fucose, a critical intermediate, is either synthesized from L-fucose via a fucose-dependent salvage pathway, or from D-glucose via a 7-step GDP-mannose-dependent de novo pathway. Glk: glucokinase; Pgi: phosphalucoisomerase; ManA: mannose 6-phosphate isomerase; ManB: phosphomannomutase; ManC: α-D-mannose 1-phosphate guanylytransferase; Gmd: GDP-mannose 6-dehydrogenase; WcaG: GDP-L-fucose synthase; Fkp: phosphofructokinase; FutC: α-1,2-fucosyltransferase.

FIG. 3 shows the novel biosynthetic pathway for the production of 2′-FL from L-galactose according to the present disclosure. L-galactose can be converted to GDP-L-galactose by Fkp enzyme. The produced GDP-L-galactose can be converted to GDP-L-fucose by two enzymes, dehydratase and reductase. Subsequently, GDP-L-fucose and lactose can be converted to 2′-fucosyllactose (2′-FL) by an α-1,2-fucosyltransferase (FutC). Alternatively, GDP-L-galactose also can be produced from GDP-D-mannose by GDP-mannose 3′, 5′-epimerase (GME). There are three co-factor regeneration systems that can be combined with the bioconversion process: (1) ATP regeneration; (2) GTP recycling system; and (3) NADPH regeneration system.

FIGS. 4A-4E show LC-MS spectra confirming the conversion of L-galactose to GDP-L-galactose catalyzed by FKP: (FIG. 4A) HPLC-UV chromatogram (254 nm) obtained from a sample without FKP (“No FKP”); (FIG. 4B) extracted total-ion-current (TIC) chromatogram for the GDP-L-galactose ion (604.05) from the same sample without FKP (“No FKP”); (FIG. 4C) HPLC-UV chromatogram obtained from a sample with FKP (“With FKP”); (FIG. 4D) extracted TIC chromatogram for the GDP-L-galactose ion (604.05) from the same sample with FKP (“With FKP”); (FIG. 4E) mass spectrum obtained from the sample “with FKP” from the 5.7-5.9 minute region.

FIGS. 5A-5D show HPLC-UV chromatograms confirming the conversion of L-galactose to GDP-L-galactose via FKP combined with ATP regeneration system. (FIG. 5A) Full HPLC-UV chromatogram (254 nm) obtained from a sample without FKP (“No FKP”); (FIG. 5B) a magnified view of a partial UV chromatogram (254 nm) obtained from the same sample without FKP (“No FKP”) from the 6.5 to 9.5 minute region; (FIG. 5C) full UV chromatogram (254 nm) obtained from a sample with FKP (“With FKP”); (FIG. 5D) a magnified view of a partial UV chromatogram obtained from the same sample with FKP (“With FKP”) from the 6.5-9.5 minute region.

FIGS. 6A-6E show HPLC-UV chromatograms confirming the conversion of GDP-D-mannose to GDP-L-galactose by At GME. (FIG. 6A) Full UV chromatogram (254 nm) obtained from a sample without At GME (“No GME”); (FIG. 6B) a magnified view of a partial UV chromatogram obtained from the same sample without GME (“No GME”) from the 6-8 minute region; (FIG. 6C) full UV chromatogram (254 nm) obtained from a sample with GME (“With GME”); (FIG. 6D) a magnified view of a partial UV chromatogram obtained from the same sample with GME (“With GME”) from the 6-8-minute region; (FIG. 6E) UV chromatogram (254 nm) of the product of the FKP reaction described in FIGS. 5A-5D within the 6-8 minute region.

FIGS. 7A-7D show HPLC-UV chromatograms confirming the conversion of GDP-L-galactose to GDP-L-fucose. Full UV (254 nm) chromatograms were obtained from (FIG. 7A) a control sample with no enzymes (“No Enzymes”); (FIG. 7B) a sample under the test reaction (“Test”); (FIG. 7C) a 1 mM GDP-L-fucose standard. (FIG. 7D) A magnified and superimposed view of the HPLC-UV chromatogram of the GDP-L-fucose standard over the UV chromatograms of the “No Enzymes” control and the “Test” reaction within the 8.2-9.5 minute region.

FIGS. 8A-8G show LC-MS spectra confirming GDP-L-fucose production from GDP-L-galactose. Full UV (254 nm) chromatograms were obtained from (FIG. 8A) a control sample with no enzymes (“No Enzymes”); (FIG. 8B) a sample under the test reaction (“Test”); (FIG. 8C) a control sample with no dehydratase (“No Dehydratase”); (FIG. 8D) a 1 mM GDP-L-fucose standard; and (FIG. 8E) a control sample with no reductase (“No Reductase”). (FIG. 8F) A magnified and superimposed view of the HPLC-UV chromatogram of the GDP-L-fucose standard over the UV chromatograms of the “No Enzymes” control, the “No Dehydratase” control, the “No Reductase” control, and the “Test” reaction within the 10-10.8 minute region. (FIG. 8G) A mass spectrum showing a 10.4 minute peak obtained from the sample from the “Test” reaction.

FIGS. 9A-9G show LC-MS spectra confirming the bioconversion of GDP-L-galactose to 2′-FL. Extracted TIC chromatograms for the [M-H]⁻ ion of 2′-FL were obtained from (FIG. 9A) a 2′-FL standard; (FIG. 9B) a control with no Gmd (“No Gmd”); (FIG. 9C) the “Test” reaction sample; (FIG. 9D) a negative control with no substrate (“No GDP-L-Gal”); (FIG. 9E) a negative control with no WcaG enzyme (“No WcaG”); and (FIG. 9F) a negative control with no FutC enzyme (“No FutC”). (FIG. 9G) The mass spectrum for the 18.9 minute peak from the Test reaction sample.

FIGS. 10A-10G show HPLC chromatograms confirming 2′-FL production by various FutC candidate enzymes. The refractive index unit (RIU) trace is shown for each of the (FIG. 10A) 2′-FL standard, (FIG. 10B) FutC 2, (FIG. 10C) FutC 5, (FIG. 10D) FutC 10, (FIG. 10E) FutC 13, (FIG. 10F) FutC 18, and (FIG. 10G) FutC 21. Arrow indicates the peak of 2′-FL.

FIGS. 11A-11E illustrate various NTP regeneration systems according to the present teachings. (FIG. 11A) Pyruvate kinase (PK) system; (FIG. 11B) creatine kinase system (CPK); (FIG. 11C) acetate kinase system (AckA); (FIG. 11D) polyphosphate kinase system (PPK); and (FIG. 11E) polyphosphate:AMP phosphotransferase/adenylate kinase system (PAP/ADK).

FIGS. 12A-12D illustrate various NADPH regeneration systems according to the present disclosure. (FIG. 12A) NADP-dependent malic enzyme (MaeB) system; (FIG. 12B) formate dehydrogenase (FDH) system; (FIG. 12C) phosphite dehydrogenase (PTDH) system; and (FIG. 12D) glucose dehydrogenase (GDH) system.

FIG. 13 illustrates how GDP-L-fucose is a negative feedback to wild-type GMD enzymes known to catalyze the conversion of GDP-mannose to GDP-4-keto-6-deoxy-D-mannose in the de novo pathway.

FIG. 14 illustrates how GDP-L-fucose, similarly, acts as a negative feedback to wild-type GMD that can be used to catalyze the conversion of GDP-L-galactose to GDP-4-keto-6-deoxy-L-galactose in the novel pathway according to the present teachings.

FIG. 15 illustrates how GDP-L-fucose, similarly, acts as a negative feedback to wild-type GMD that can be used to catalyze the conversion of GDP-L-galactose to GDP-4-keto-6-deoxy-L-galactose, where GDP-L-galactose can be derived from GDP-mannose using a GDP-mannose 3′, 5′-epimerase (GME).

FIGS. 16A-16B show GDP-L-fucose inhibition data for At GMD and Hs GMD. (FIG. 16A) The relative activity of the At GMD mutants (At M2, At M3 and At M4), At GMD WT (At WT) and Ec GMD WT (Ec WT) at 0, 70 and 350 μM. (FIG. 16B) The relative activity of H GMD mutants (H M2, H M3 and H M4) compared to H GMD WT (H WT) and Ec GMD WT (Ec WT).

FIGS. 17A-17B show FutC Activity: (FIG. 17A) activity screen for the ASR library; (FIG. 17B) activity of various FutC enzymes compared to H. pylori FutC and the parent enzyme (FutC 5) for the ASR12 construct.

FIG. 18 illustrates the present novel biosynthesis pathway of 2′-FL production from L-galactose.

FIG. 19 shows the production of 2′-FL over time using the novel in vitro pathway that converts L-galactose into 2′-FL. The ASR12 data are represented by circles, Hp FutC data are represented by squares, ASR11 data are represented by triangles, and FutC 5 data are represented by diamonds.

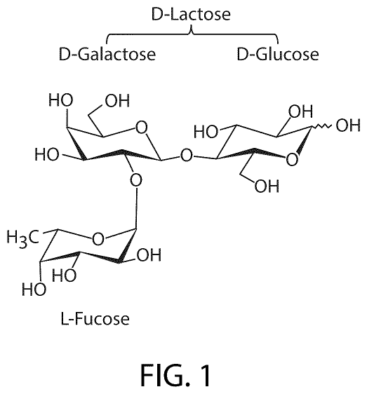

### DETAILED DESCRIPTION

The present disclosure provides a novel multi-enzyme pathway for 2′-FL biosynthesis that has the following advantages: (1) it uses L-galactose instead of L-fucose as the starting substrate; (2) it is a 4-step process compared to the 8-step process required by the de novo mannose-dependent pathway (7 steps to synthesize GDP-fucose, and an 8^{th} step to convert GDP-fucose into 2′-FL); and (3) the 4-step pathway includes a GTP-regeneration process, an ATP regeneration process, and an NAD(P)⁺/NAD(P)H recycling mechanism, hence significantly reducing the need and the associated costs for cofactors. In addition, the present pathway can be performed cell-free in vitro, which brings forth the following additional advantages comparing to fermentative production: (1) it is a non-chemical and non-GMO process that uses all-natural biomolecules such as enzymes, sugars, and co-factors to synthesize 2′-FL; (2) as a cell-free process, it eliminates possibility for endotoxin production and phage contamination, which are two major concerns for E. coli and other bacterial fermentation; (3) enzymes can be expressed in preferred organisms to ensure that all enzymes are in the most active form, and the process can be performed under preferred condition without interference by other processes; and (4) a cell-free process leads to simpler product purification steps.

Referring to FIG. 3, the present disclosure provides a biosynthetic process for preparing 2′-FL which consists of four steps: (1) first, L-galactose is converted into GDP-L-galactose via a reaction catalyzed by a fucokinase/guanylyltransferase in the presence of ATP and GTP; (2) second, GDP-L-galactose is converted into GDP-4-keto-6-deoxygalactose via a reaction catalyzed by either a dehydratase (e.g., a GDP-mannose 4,6-dehydratase) in the presence of NAD(P)⁺ as a co-factor which is reduced into NAD(P)H; (3) GDP-4-keto-6-deoxyglucose is converted into GDP-L-fucose via a reaction catalyzed by a reductase (e.g., a GDP-4-keto-6-deoxy-mannose reductase) in the presence of NADPH as a co-factor which is oxidized to NADP⁺; and (4) the final reaction step utilizes an alpha-1,2-fucosyltransferase (futC) enzyme to convert GDP-fucose into 2′-FL in the presence of lactose, producing GDP as a side product. With continued reference to FIG. 3, the reaction system can include an ATP regeneration system, a GTP regeneration system, and an NADPH regeneration system so that only small amounts of these co-factors are needed to initiate the process, which makes the present process much more cost-effective than existing methods.

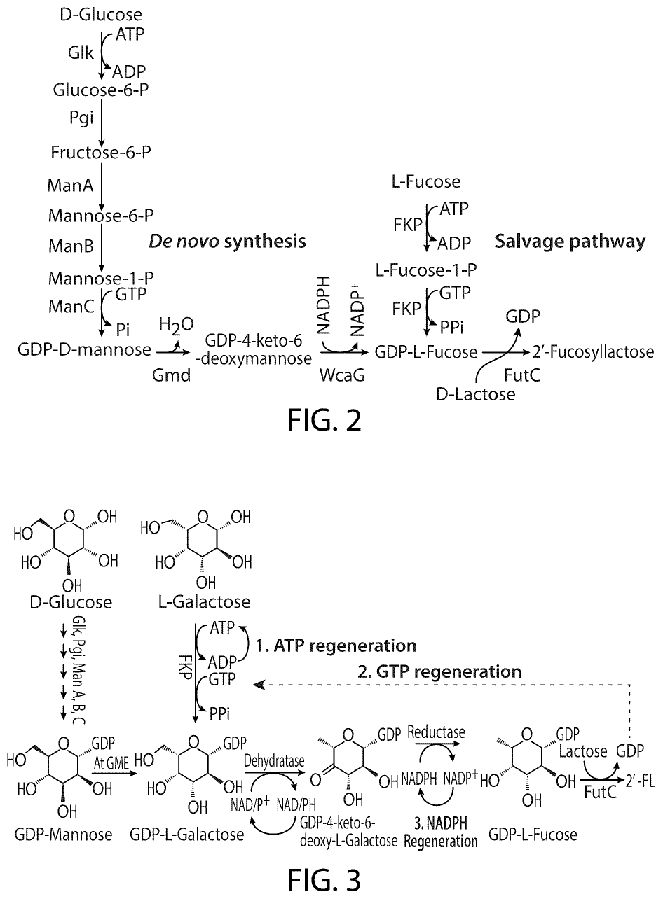

Alternatively, the present method can be a modification of the de novo synthesis pathway (FIG. 2). Starting from D-glucose, the first five reaction steps can be performed to provide GDP-mannose. The GDP mannose can be converted into GDP-L-galactose in a reaction catalyzed by a GDP-mannose-3,5-epimerase. Steps 2 to 4 in the above 4-step method can then be performed to provide 2′-FL.

Each step of the present process will be discussed in more details below.

Synthesis of GDP-L-Galactose

FKP naturally catalyzes the conversion of L-fucose into GDP-L-fucose. It has been reported that FKP also could generate GDP-L-galactose from L-galactose (Ohashi et al. 2017). Accordingly, the first step of the present method can include incubating L-galactose with a fucokinase/guanylyltransferase in the presence of ATP and GTP for a sufficient time to convert the L-galactose into GDP-L-galactose. For example, the fucokinase/guanylyltransferase can be an enzyme comprising an amino acid sequence having at least 60%, at least 70%, at least 80%, at least 85%, at least 90%, at least 95%, at least 98%, or at least 99% sequence identity to SEQ ID NO: 1. In particular embodiments, the fucokinase/guanylyltransferase can comprise the amino acid sequence set forth in SEQ ID NO: 1.

Alternatively, the GDP-L-galactose used in the present method can be generated from GDP-mannose. The present method therefore can include a first step comprising incubating GDP-mannose with a GDP-mannose-3,5-epimerase for a sufficient time to convert the GDP-mannose into GDP-L-galactose. For example, the GDP-mannose-3,5-epimerase can be an enzyme comprising an amino acid sequence having at least 60%, at least 70%, at least 80%, at least 85%, at least 90%, at least 95%, at least 98%, or at least 99% sequence identity to SEQ ID NO: 3 or SEQ ID NO: 19. In particular embodiments, the GDP-mannose-3,5-epimerase can comprise the amino acid sequence set forth in SEQ ID NO: 3 or SEQ ID NO: 19.

Synthesis of GDP-L-Fucose

Without wishing to be bound by any particular theory, the inventors believe that enzymes capable of converting GDP-mannose to GDP-4-keto-6-deoxymannose also can convert GDP-L-galactose into GDP-4-keto-6-deoxy-L-galactose.

While a GDP-mannose 4,6-dehydratase (GMD) normally uses GDP-mannose as substrate to produce GDP-4-keto-6-deoxymannose, the inventors have shown in Example 3 below that a GDP-mannose 4,6-dehydratase also can use GDP-L-galactose as substrate to produce GDP-4-keto-6-deoxy-L-galactose. Accordingly, suitable enzymes for catalyzing the conversion of GDP-L-galactose into GDP-4-keto-6-deoxy-L-galactose can include GDP-mannose 4,6-dehydratases having the amino acid sequence set forth in SEQ ID NO: 5, SEQ ID NO: 9, SEQ ID NO: 11, and SEQ ID NO: 13. In some embodiments, suitable dehydratases can include functional fragments or homologs of a polypeptide having the amino acid sequence set forth in SEQ ID NO: 5, SEQ ID NO: 9, SEQ ID NO: 11, or SEQ ID NO: 13.

In preferred embodiments, the GMD is a mutant that obstructs or otherwise inhibits GDP-L-fucose allosteric binding by the GMD. The GMD mutant can be a mutant At GMD comprising an amino acid sequence of SEQ ID NO: 79, SEQ ID NO: 77, or SEQ ID NO: 75. Alternatively, the GMD mutant can be a mutant Hs GMD comprising an amino acid sequence of SEQ ID NO: 85, SEQ ID NO: 83, or SEQ ID NO: 81.

Next, a reductase is used to convert GDP-4-keto-6-deoxy-L-galactose into GDP-L-fucose. Suitable enzymes for catalyzing the conversion of GDP-4-keto-6-deoxy-L-galactose into GDP-L-fucose can include reductases known to have activity as GDP-4-keto-6-deoxy-mannose reductases. For example, reductases having the amino acid sequence set forth in SEQ ID NO: 7, SEQ ID NO: 15, or SEQ ID NO: 17 can be used. In some embodiments, suitable dehydratases can include functional fragments or homologs of a polypeptide having the amino acid sequence set forth in SEQ ID NO: 7, SEQ ID NO: 15, or SEQ ID NO: 17.

Synthesis of 2′-Fucosyllactose

The last step of the present method involves the conversion of GDP-L-fucose into 2′-fucosyllactose. The reaction is catalyzed by an alpha-1,2-fucosyltransferase (futC). Exemplary enzymes that can function as futCs include those listed in Table 3. Additional exemplary enzymes that can function as futCs include those listed in Table 5 (ASR1 to ASR 12). In preferred embodiments, the α-1,2-fucosyltransferase is selected from the group consisting of a polypeptide comprising an amino acid sequence having at least 60%, at least 70%, at least 80%, at least 85%, at least 90%, at least 95%, at least 98%, or at least 99% sequence identity to SEQ ID NO: 109, SEQ ID NO: 29, SEQ ID NO: 107, SEQ ID NO: 89, SEQ ID NO: 91, SEQ ID NO: 93, SEQ ID NO: 95, SEQ ID NO: 99, SEQ ID NO: 101, SEQ ID NO: 103, or SEQ ID NO: 105.

Co-Factors Regeneration in the Bioconversion of 2′-Fucosyllactose

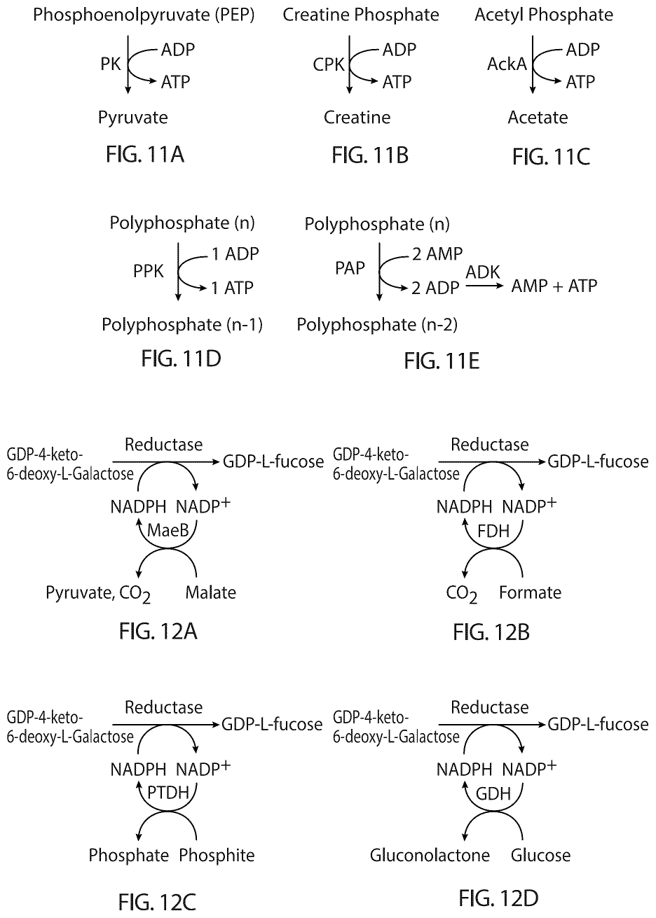

ATP and GTP are essential for FKP activity and GDP-L-galactose production. The present method includes NTP regeneration systems that help to provide a sustainable cost-effective reaction system. NTP regeneration systems require a high energy phosphate donor to add a phosphate onto the NDP. Suitable systems include: phospho(enol)pyruvate (PEP) and pyruvate kinase (A), creatine phosphate and creatine kinase (B), acetyl phosphate and acetate kinase (C), polyphosphate and polyphosphate kinase (D) and polyphosphate:AMP phosphotransferase, adenylate kinase and adenosine monophosphate (E) (FIG. 11).

In addition, NADPH is a critical co-factor for the reductase activity necessary for GDP-L-fucose production. In the course of the reductase-catalyzed reaction, NADPH is oxidized to NADP⁺. By incorporating an NADP⁺-dependent oxidation reaction as part of the GDP-L-fucose synthesis disclosed herein, NADPH can be regenerated. Exemplary NADP⁺-dependent oxidation reactions include the oxidation of malate into pyruvate, the oxidation of formate into CO₂, the oxidation of phosphite into phosphate and the oxidation of glucose into gluconolactone (FIG. 12). By including a donor substrate (malate, formate, phosphite or glucose) and the corresponding dehydrogenase (malate dehydrogenase (MaeB, SEQ ID NO: 67), formate dehydrogenase (FDH, SEQ ID NO: 69), phosphite dehydrogenase (PTDH, SEQ ID NO: 71) and glucose dehydrogenase (GDH, SEQ ID NO: 73), respectively), NADPH can be continuously regenerated, further improving GDP-L-fucose and 2′-FL production.

Unless specified otherwise, the percent identity of two polypeptide or polynucleotide sequences refers to the percentage of identical amino acid residues or nucleotides across the entire length of the shorter of the two sequences.

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred materials and methods are described below.

The disclosure will be more fully understood upon consideration of the following non-limiting Examples. It should be understood that these Examples, while indicating preferred embodiments of the subject technology, are given by way of illustration only. From the above discussion and these Examples, one skilled in the art can ascertain the essential characteristics of the subject technology, and without departing from the spirit and scope thereof, can make various changes and modifications of the subject technology to adapt it to various uses and conditions.

### EXAMPLES

### Example 1: Screening of Candidate Enzymes

Gene candidates were selected based on bioinformatic analysis. The following enzymes were screened for the desired activity: fucokinase/guanylyltransferase (FKP) from Bacteroides fragilis (SEQ ID NO: 1), GDP-mannose-3,5-epimerase from Arabidopsis thaliana (At GME) (SEQ ID NO: 3) and Oryza sativa (Os GME) (SEQ ID NO: 19), GDP-mannose-4,6-dehydratase from Escherichia coli (Ec GMD)(SEQ ID NO: 11), Homo sapiens (Hs GMD) (SEQ ID NO: 9), Arabidopsis thaliana (At GMD) (SEQ ID NO: 5), and Yersinia pseudotuberculosis (Yp DmhA) (SEQ ID NO: 13), GDP-L-fucose synthase (GFS) or GDP-4-keto-6-deoxy-mannose reductase from Escherichia coli (WcaG) (SEQ ID NO: 7), Campylobacter jejuni (MlghC) (SEQ ID NO: 17) and Yersinia pseudotuberculosis (DmhB) (SEQ ID NO: 15), and 21 α-1,2-fucosyltransferases (FutC 1-21) (odd-numbered SEQ ID NO: 21-61, the source organism for each of which is listed in Table 2).

Full length DNA fragments of all candidate genes were commercially synthesized. Almost all codons of the cDNA were changed to those preferred for E. coli (Twist Bioscience, CA). The synthesized DNA was cloned into a bacterial expression vector (pET21 or pET28) to generate the expression construct.

Each expression construct was transformed into E. coli T7 Express or BL21 (DE3) cell, which was subsequently grown in LB media containing 50 μg/mL ampicillin or 50 μg/mL kanamycin at 37° C. until reaching an OD600 of 0.4-0.8. Protein expression was induced by addition of 1 mM isopropyl β-D-1-thiogalactopyranoside (IPTG) and the culture was further grown at 16° C. for 16 hr. Cells were harvested by centrifugation (3,000×g; 10 min; 4° C.). The cell pellets were collected and were either used immediately or stored at −80° C.

The cells were resuspended in lysis buffer (50 mM Tris-HCl pH 8.0, 150 mM NaCl, 20 mM imidazole). After sonication, the lysate was clarified by centrifugation at 16,000×g for 15 minutes. The clarified lysate was loaded onto an equilibrated (equilibration buffer: 50 mM Tris-HCl, pH 8.0, 20 mM imidazole, 150 mM NaCl, 20% glycerol) talon metal affinity column (Takara Bio). After loading of the protein sample, the column was washed with an equilibration buffer to remove unbound contaminant proteins. The His-tagged recombinant polypeptides were eluted by equilibration buffer containing 250 mM imidazole. The proteins were used for activity assays or aliquoted and stored at −80 until needed.

All samples were analyzed by suitable HPLC and LC-MS methods.

For the LC-MS detection of GDP-L-fucose and GDP-L-galactose, the samples were quenched by heating at 99° C. for 10 minutes, and the proteins were removed by centrifugation. The column was a Luna, C18(2) HST, 2.0 mm×100 mm with 2.5 μm particle size and 100 Å pore size from Phenomenex. Mobile phase A was 10 mM triethylammonium acetate pH 7.0, and mobile phase B was 10 mM triethylamine acetate pH 7.0 (90%) and 10% acetonitrile. The column compartment was set at 25° C., and the flow rate was 0.3 mL/min. The pump method was: 0 min 1% B, 1 min 1% B, 8 min 5% B, 8.1 min 20% B, 10 min 20% B, 10.1 min 1% B and hold until 15 mins. The UV detector was set to 254 nm. The spray voltage was set to 2.7 kV, the capillary temperature was 300° C., the sheath gas was 40, the auxiliary gas was 8, the spare gas was 2, the max spray current was 100, the probe heater temperature was 320° C. and the S-Lens RF level was 60.

For the HPLC detection of GDP-L-fucose, GDP-mannose, GDP-L-gulose, and GDP-L-galactose, the samples were prepared as described above. The column was a Luna 5 μm C18(2) 100 Å, 4.6×250 mm from Phenomenex. Mobile phase A was 10 mM triethylammonium acetate pH 7.0 and mobile phase B was 10 mM triethylamine acetate pH 7.0 (90%) and 10% acetonitrile. The column compartment was set at 25° C., and the flow rate was 1.3 mL/min. The pump method was: 0 min 1% B, 1 min 1% B, 8 min 5% B, 8.1 min 20% B, 10 min 20% B, 10.1 min 1% B and hold until 15 mins. The UV detector was set to 254 nm.

The following method was used for the LC-MS detection of 2′-FL. The samples were prepared as described above. The analytes were separated using a Thermo Fisher, Hypercarb column, 2.1×100 mm, 3 um particle size. Mobile phase A was 0.1% formic acid in water and mobile phase B was 0.1% formic acid in acetonitrile. The column compartment was set to 25° C., and the flow rate was set to 0.2 mL/min. The full run time was 30 minutes. The pump method was: 0 min 0% B, 21 min 12% B, 22 min 0% B, 30 min 0% B. The spray voltage was 3.5 kV, the capillary temperature was 300° C., the sheath gas was 50, the auxiliary gas was 10, the spare gas was 2, the max spray current was 100, the probe heater temperature was 370° C. and the S-Lens RF level was 45.

For HPLC detection of 2′-FL, the samples were prepared as described above. The HPLC instrument method was optimized with isocratic elution of the analytes with distilled water, using an Aminex HPX-87H, 300×7.8 mm (BioRad) column and a flow rate of 0.6 mL/min and total run time of 12 min. The column compartment was set to 50° C. 2′-FL was monitored using a Refractomax 520.

### Example 2: Identification of Enzymes for GDP-L-Galactose Synthesis

The first step in the novel pathway according to the present disclosure is the production of GDP-L-galactose. FIG. 3 outlines two methods for GDP-L-galactose production according to the present disclosure.

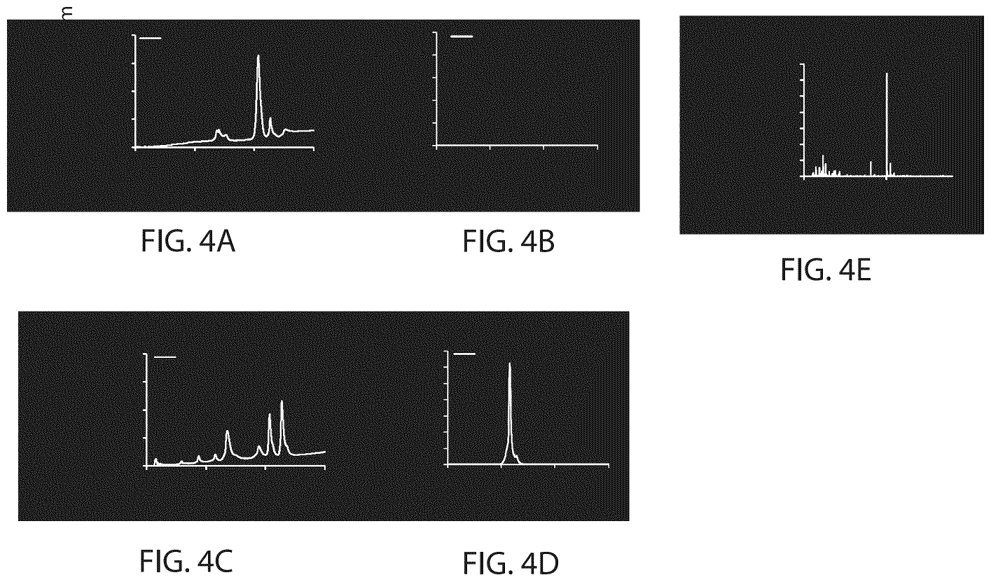

FKP naturally catalyzes the conversion of L-fucose into GDP-L-fucose. It has been reported that FKP also could generate GDP-L-galactose from L-galactose (Ohashi et al. 2017). FKP was cloned into pET21, expressed and purified as described in Example 1. The activity of FKP towards L-galactose was assayed under the following conditions: 2 mM L-galactose, 2 mM ATP, 2 mM GTP, 4 mM MgCl₂, 50 mM Tris-HCl, pH 7.5 and with or without 0.25 g/L FKP. The samples were incubated at room temperature overnight, and quenched by heating at 99° C. for 10 minutes, and analyzed using LC-MS. The LC-MS results are shown in FIG. 4.

Referring to FIG. 4, several new peaks were observed from the reaction with the FKP addition (C), compared to the No FKP reaction (A). The m/z for GDP-L-galactose was extracted to identify the peak of GDP-L-galactose in the UV spectrum. The extracted chromatogram (D) shows GDP-L-galactose elutes at ˜5.8 min, and this peak was not present in the No FKP control. The mass spectrum for the 5.8 min peak in the HPLC chromatogram is shown in FIG. 4 (E). The most abundant ion was 604.07, which is the [M-H]⁻ for GDP-L-galactose. This demonstrates that FKP catalyzes the conversion of L-galactose to GDP-L-galactose.

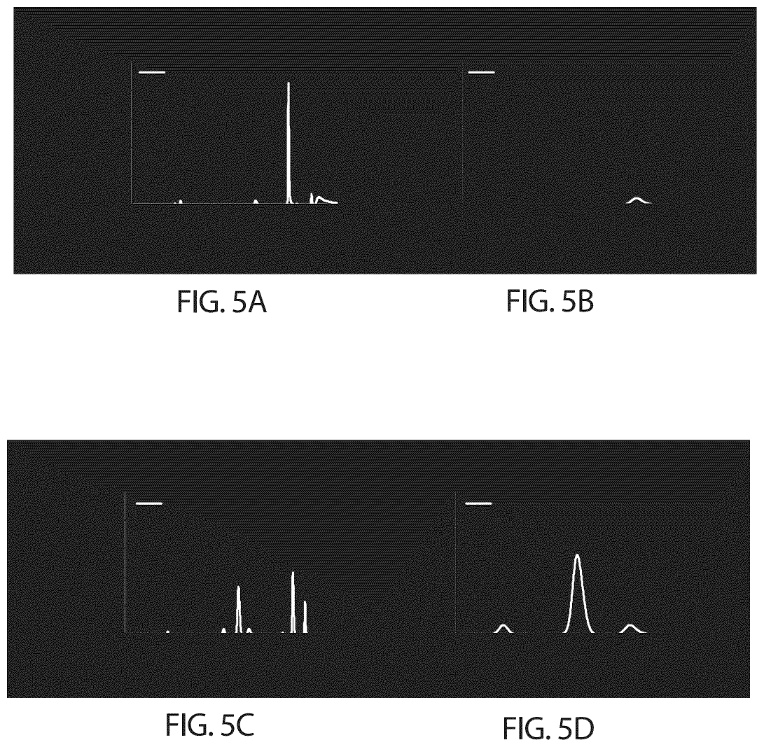

Further work was conducted to optimize the FKP reaction with L-galactose as the substrate, by varying reaction temperature, substrate concentrations, and adding an ATP/GTP regeneration system. The reaction conditions were: 50 mM Tris-HCl pH 7.5, 5 mM MgCl₂, 5 mM L-galactose, 2 mM ATP, 2 mM GTP, 10 mM PEP, 1 U pyruvate kinase, and with or without 0.8 mg/mL FKP. The reaction was incubated at 37° C. for 44 hours. The reaction was quenched by heating at 99° C. for 10 minutes. The samples were analyzed using HPLC (FIG. 5).

As shown in FIG. 5, GDP-L-galactose (retention time: 8.4 min) can be produced in the reaction with FKP (D) compared to the reaction without FKP (B). The final titer was 2.5 g/L. This data demonstrates the production of GDP-L-galactose from L-galactose using the FKP production method.

In addition to generating GDP-L-galactose from L-galactose, GDP-L-galactose also can be generated from GDP-D-mannose according to the present teachings.

After enzymatic screening of various potential GDP-mannose-3,5-epimerase candidate enzymes, At GME (SEQ ID NO: 3) was found to show higher activity than Os GME (SEQ ID NO: 19). The reaction conditions were: 2 mM GDP-D-mannose, 1 mM NAD⁺, 50 mM Tris-HCl, pH 8.0 and with or without 0.33 mg/mL At GME. The reactions were incubated at room temperature for 16 hours, quenched by heating at 99° C. for 10 mins, and analyzed using HPLC.

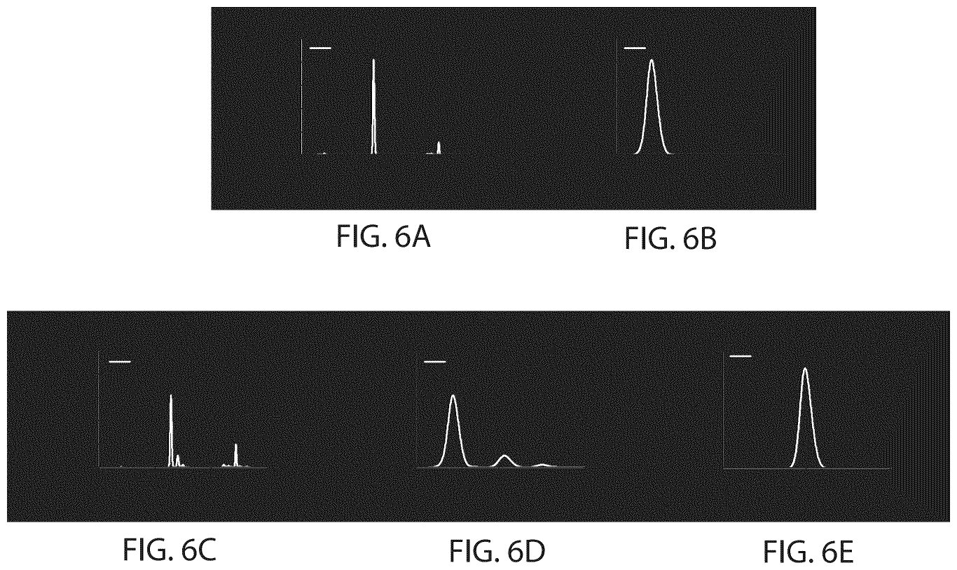

FIG. 6 shows HPLC data confirming the conversion of GDP-D-mannose to GDP-L-galactose. In the reaction with At GME, a decrease in GDP-mannose was observed, and two new peaks were formed (D) compared to the negative control (B). The two new peaks were identified to be GDP-L-galactose and GDP-L-gulose. Specifically, the GDP-L-galactose peak was identified based on the product from the FKP reaction (E).

In summary, these data show two methods for GDP-L-galactose production. The FKP approach reached higher titers.

### Example 3: Identification of Enzymes for GDP-L-Fucose Synthesis

With the production of GDP-L-galactose demonstrated, the next step was to generate GDP-L-fucose, which requires a dehydratase and a reductase. There is an expansive list of GDP-mannose-4,6-dehydratases that will dehydrate GDP-D-mannose. However, there has been no report of GDP-L-galactose-4,6-dehydratases.

Four enzymes were screened: At Gmd (SEQ ID NO: 5), Hs Gmd (SEQ ID NO: 9), Ec Gmd (SEQ ID NO: 11) and Yp DmhA (SEQ ID NO: 13), while Ec WcaG (SEQ ID NO: 7) is known to be a reductase. Among the potential dehydratases, At Gmd showed the highest initial activity. The reaction conditions are shown in Table 1. The reactions were incubated for 16 hours at 37° C., and then quenched by heating at 99° C. for 10 minutes. Samples were analyzed by both HPLC and LC-MS methods.

TABLE 1
GDP-L-fucose reaction conditions
GDP-L-Fucose Reaction Conditions

| Reaction | No | No | No | |
|---|---|---|---|---|
| Component | Enzymes | Reductase | Dehydratase | Test |
| GDP-L-Gal (mM) | 5 | 5 | 5 | 5 |
| NADP⁺ (mM) | 0.5 | 0.5 | 0.5 | 0.5 |
| NADPH (mM) | 2 | 2 | 2 | 2 |
| Tris-HCl pH 7.5 | 50 | 50 | 50 | 50 |
| (mM) | | | | |
| Ec WcaG (g/L) | 0 | 0 | 3.2 | 3.2 |
| At Gmd (g/L) | 0 | 2.4 | 0 | 2.4 |

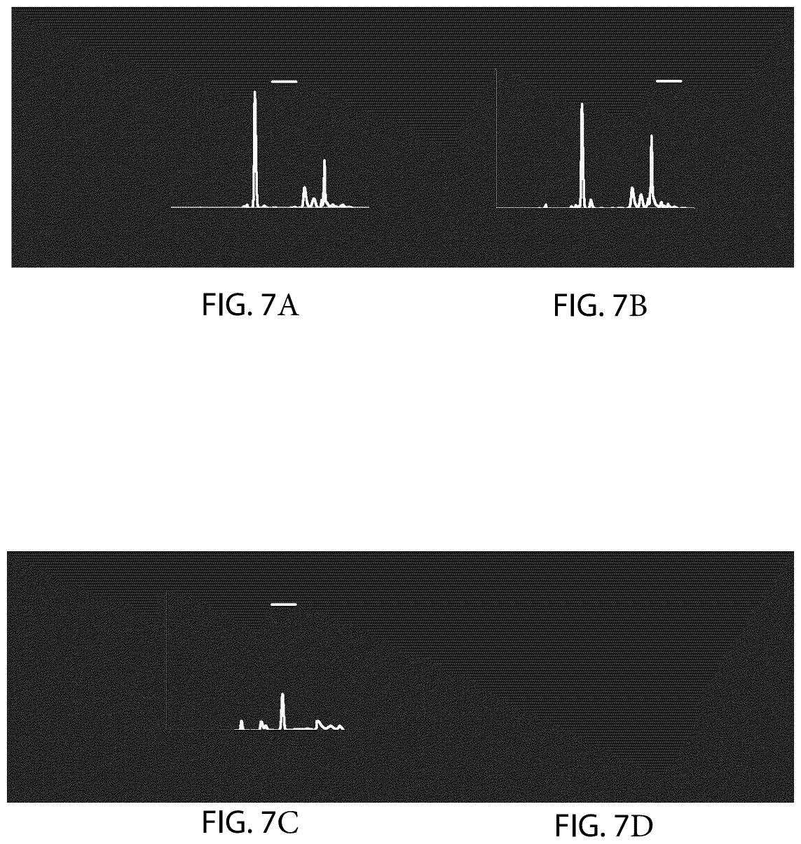

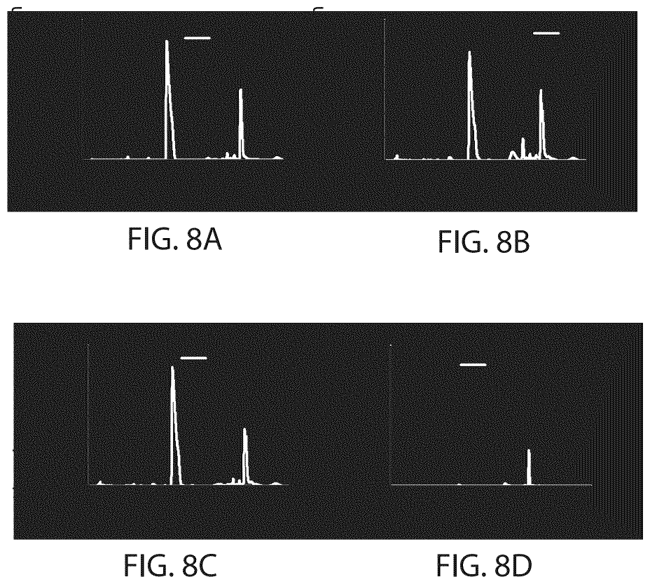

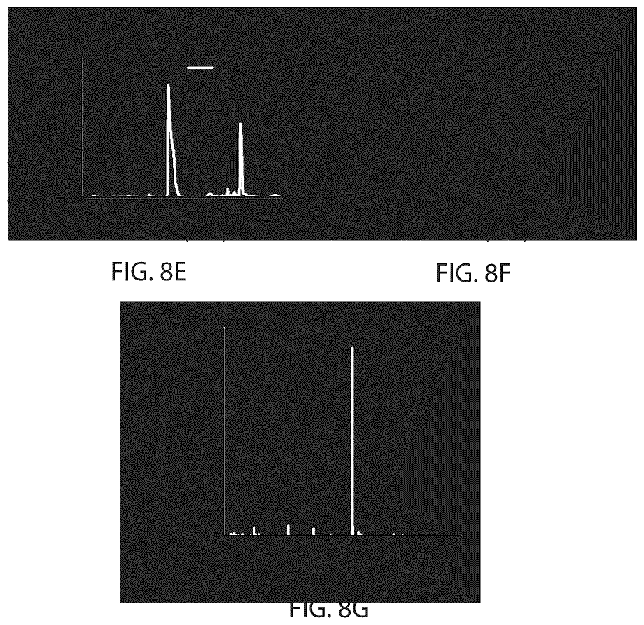

FIG. 7 shows the HPLC data. There is a small peak in the Test reaction that co-elutes with GDP-L-fucose standard (B). To confirm that this peak was GDP-L-fucose, we analyzed the samples using LC-MS. The LC-MS data is shown in FIG. 8. FIG. 8 shows the full UV 254 nm trace for the (A) No Enzymes, (B) Test, (C) No Dehydratase, (D) 1 mM GDP-L-fucose and (E) No Reductase samples, respectively. By comparing the “Test” reaction sample (B) against the standard (D), there is a peak in the test condition that elutes at a similar retention time (˜10.4 min) to GDP-L-fucose. When superimposing the various chromatograms over one another (F), there is a peak in the Test reaction sample that co-elutes with GDP-L-fucose, which is not present in the negative controls. The mass spectrum for 10.4-minute peak in the Test reaction sample is shown in FIG. 8 (E). The most abundant ion corresponds to the [M-H]⁻ 588.07 m/z for GDP-L-fucose. The above data demonstrate that At Gmd has dehydratase activity towards GDP-L-galactose which allows for GDP-L-fucose production.

### Example 4: Bioconversion of GDP-L-Galactose/L-Galactose to 2′-FL

To complete the novel pathway from GDP-L-galactose to 2′-FL, a series of reactions were set up to produce 2′-FL using GDP-L-galactose as substrate. The reaction conditions are shown in Table 2. The reactions were incubated at 37° C. for 16 hours, and then quenched by heating at 99° C. for 10 minutes. The samples were analyzed using LC-MS.

TABLE 2
Reaction conditions for 2′-FL Production

| Reaction Component | No Dehydratase | No Reductase | No FutC | No Substrate | Test |
|---|---|---|---|---|---|
| GDP-L-Gal (mM) | 5 | 5 | 5 | 0 | 5 |
| NADP⁺ (mM) | 0.25 | 0.25 | 0.25 | 0.25 | 0.25 |
| NADPH (mM) | 1 | 1 | 1 | 1 | 1 |
| Lactose (mM) | 40 | 40 | 40 | 40 | 40 |
| At Gmd (g/L) | 0 | 0.6 | 0.6 | 0.6 | 0.6 |
| Ec WcaG (g/L) | 0.75 | 0 | 0.75 | 0.75 | 0.75 |
| FutC-21 (g/L) | 0.72 | 0.72 | 0 | 0.72 | 0.72 |
| Tris-HCl pH 7.5 (mM) | 50 | 50 | 50 | 50 | 50 |

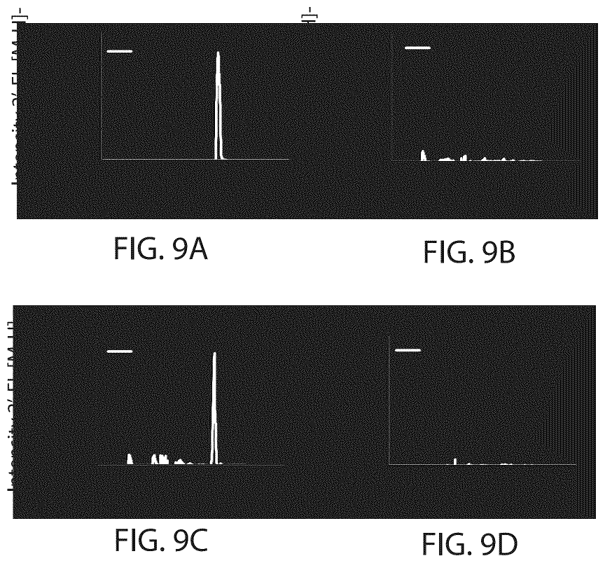

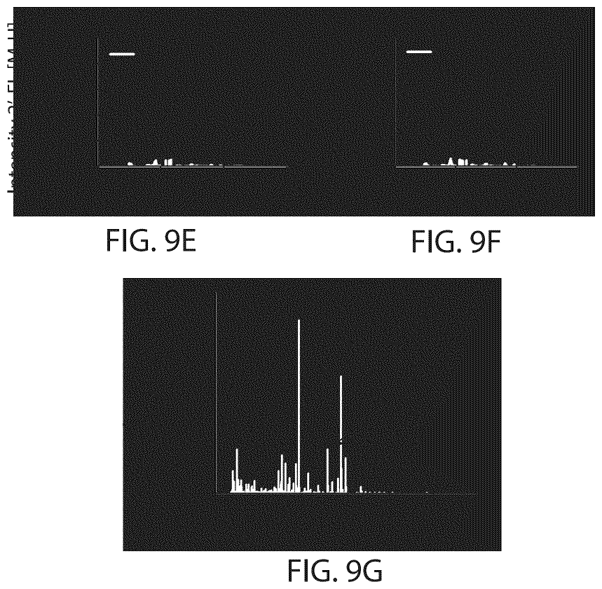

Four negative controls were set up as follows: No Gmd, No WcaG, No FutC and No GDP-L-galactose, and one test reaction sample with the substrate and all enzymes present. The LC-MS data is shown in FIG. 9.

The [M-H]⁻ ion of 2′-FL was extracted from each of the chromatograms obtained from the test reaction sample and each of the four negative controls. For the negative controls, no significant 2′-FL signal was observed (B), (D), (E) and (F).

From the Test reaction sample, a peak was observed at 18.9-minutes (C), which is the retention time of 2′-FL (A). The mass spectrum for the 18.9-minute peak in the Test reaction is shown in FIG. 9 (G), where the [M-H]⁻ ion for 2′-FL and the [M+FA-H]⁻ formic acid adduct are observed at 487.17 and 533.17 m/z, respectively. Together, these data confirm that GDP-L-galactose was indeed converted into 2′-FL, thereby completing the novel pathway.

### Example 5: Identification of Novel FutC Enzymes for 2′-FL Production

The final step in the novel path to 2′-FL requires an α-1,2-fucosyltransferase. Various FutC candidates were screened for soluble expression in E. coli and fucosyltransferase activity. The full list of FutC candidates is provided in Table 3. The candidate enzymes show different solubilities and enzymatic activity for 2′-FL synthesis. For the candidates that have highly soluble expression, their activity was tested in vitro using the de novo synthesis pathway. The reaction conditions were: 50 mM Tris-HCl pH 8.0, 2 mM NADPH, 0.1 mM NADP⁺, 1 mM GDP-mannose, 80 mM lactose, 0.9 mg/mL Ec Gmd (SEQ 11), 0.2 mg/mL Ec WcaG (SEQ ID NO: 7) and 0.2 mg/mL of the fucosyltransferase candidates. The reactions were incubated at room temperature for 16 hours. The reactions were quenched by heating at 99° C. for 10 minutes, and then analyzed by HPLC.

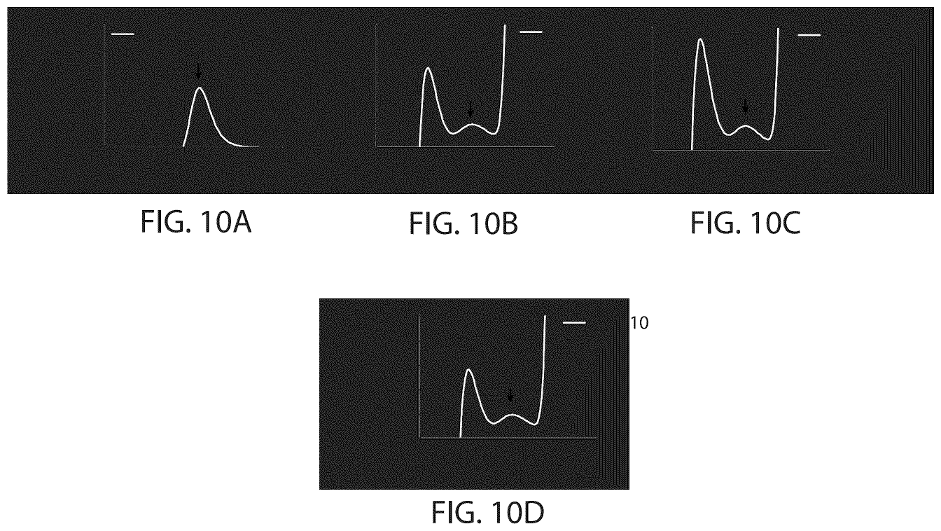

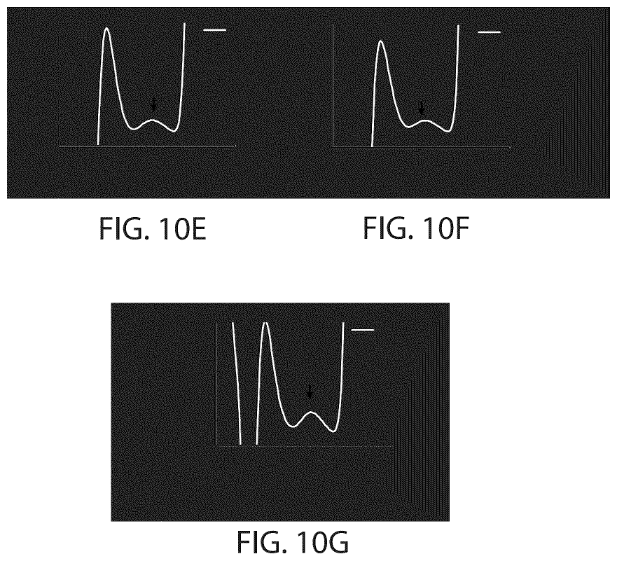

FIG. 10 shows a focused μRIU trace from 6-8 minutes for five novel FutC candidates, a 2′-FL standard and FutC from Helicobacter pylori (FutC 21, SEQ ID NO: 61). The five novel FutC candidates were FutC2 (from Pisciglobus halotolerans, SEQ ID NO: 23), FutC5 (from Lachnospiraceae bacterium XBB2008, SEQ ID NO: 29), FutC10 (from Thermosynechococcus elongatus, SEQ ID NO: 39), FutC13 (from Candidatus Brocadia sapporoensis, SEQ ID NO: 45), and FutC 18 (from Rhizobiales bacterium, SEQ ID NO: 55). All of the FutC candidates tested show a peak that elutes at the same retention time (B), (C), (D), (E), (F), as the 2′-FL standard (A). These data conclude that the 5 novel FutC candidates tested (SEQ ID NO: 23, SEQ ID NO: 29, SEQ ID NO: 39, SEQ ID NO: 45, and SEQ ID NO: 55) have α-1,2-fucosyltransferase activity for 2′-FL production.

TABLE 3
Novel FutC Candidates

| FutC | NCBI Accession No. | Species | Amino acid | DNA |
|---|---|---|---|---|
| 1 | WP_082224228.1 | Halorubrum sp. T3 | SEQ 21 | SEQ 22 |
| 2 | WP_092093466.1 | Pisciglobus halotolerans | SEQ 23 | SEQ 24 |
| 3 | WP_027405947.1 | Anaerovibrio sp. RM50 | SEQ 25 | SEQ 26 |
| 4 | OYV93441.1 | Ferrovum sp. 37-45-19 | SEQ 27 | SEQ 28 |
| 5 | WP_089855777.1 | Lachnospiraceae bacterium XBB2008 | SEQ 29 | SEQ 30 |
| 6 | WP_058309396.1 | Phaeobacter sp. CECT 7735 | SEQ 31 | SEQ 32 |
| 7 | WP_114459232.1 | Runella sp. YX9 | SEQ 33 | SEQ 34 |
| 8 | WP_090203298.1 | Pseudomonas asplenii | SEQ 35 | SEQ 36 |
| 9 | WP_080637051.1 | Clostridiales | SEQ 37 | SEQ 38 |
| 10 | WP_011058149.1 | Thermosynechococcus elongatus | SEQ 39 | SEQ 40 |
| 11 | WP_068906291.1 | Porphyromonadaceae bacterium H1 | SEQ 41 | SEQ 42 |
| 12 | WP_024582763.1 | Bradyrhizobium | SEQ 43 | SEQ 44 |
| 13 | WP_080324832.1 | Candidatus Brocadia sapporoensis | SEQ 45 | SEQ 46 |
| 14 | WP_044399014.1 | Lacinutrix sp. Hel_I_90 | SEQ 47 | SEQ 48 |
| 15 | WP_052301780.1 | Butyrivibrio proteoclasticus | SEQ 49 | SEQ 50 |
| 16 | WP_089665267.1 | Gramella sp. MAR_2010_147 | SEQ 51 | SEQ 52 |
| 17 | WP_105019998.1 | Polaribacter glomeratus | SEQ 53 | SEQ 54 |
| 18 | WP_112956699.1 | Rhizobiales bacterium | SEQ 55 | SEQ 56 |
| 19 | WP_035531266.1 | Hoeflea sp. BAL378 | SEQ 57 | SEQ 58 |
| 20 | WP_103238280.1 | Acetatifactor muris | SEQ 59 | SEQ 60 |
| 21 | ABO61750.1 | Helicobacter pylori | SEQ 61 | SEQ 62 |

### Example 6: Regeneration of Co-Factors in the Bioconversion of 2′-FL

ATP and GTP are essential for FKP activity and GDP-L-galactose production; however, they are expensive co-factors. In order to build a sustainable cost-effective system, the present disclosure provides a bioproduction method of 2′-FL that includes ATP and GTP regeneration systems. NTP regeneration systems require a high energy phosphate donor to add a phosphate onto the NDP.

FIG. 11 illustrate several systems that can accomplish this objective. These systems include: phospho(enol)pyruvate (PEP) and pyruvate kinase (A), creatine phosphate and creatine kinase (B), acetyl phosphate and acetate kinase (C), polyphosphate and polyphosphate kinase (D) and polyphosphate:AMP phosphotransferase, adenylate kinase and adenosine monophosphate (E).

In addition, NADPH is a critical co-factor for the reductase activity necessary for GDP-L-fucose production. In the course of the reductase (WcaG) catalyzed reaction, NADPH is oxidized to NADP⁺. By incorporating an NADP⁺-dependent oxidation reaction as part of the GDP-L-fucose synthesis disclosed herein, NADPH can be regenerated. Exemplary NADP⁺-dependent oxidation reactions include the oxidation of malate into pyruvate, the oxidation of formate into CO₂, the oxidation of phosphite into phosphate and the oxidation of glucose into gluconolactone (FIG. 12). By including a donor substrate (malate, formate, phosphite or glucose) and the corresponding dehydrogenase (malate dehydrogenase (MaeB, SEQ ID NO: 67), formate dehydrogenase (FDH, SEQ ID NO: 69), phosphite dehydrogenase (PTDH, SEQ ID NO: 71) and glucose dehydrogenase (GDH, SEQ ID NO: 73), respectively), NADPH can be continuously regenerated, further improving GDP-L-fucose and 2′-FL production.

### Example 7: Screening of Candidate Mutant Enzymes

Full-length DNA fragments of all candidate genes were commercially synthesized. Almost all codons of the cDNA were changed to those preferred for E. coli (Twist Bioscience, CA). The synthesized DNA was cloned into a bacterial expression vector (pET21 or pET28) to generate the expression construct.

Each expression construct was transformed into E. coli T7 Express or BL21 (DE3) cell, which was subsequently grown in LB media containing 50 μg/mL ampicillin or 50 μg/mL kanamycin at 37° C. until reaching an OD₆₀₀ of 0.4-0.8. Protein expression was induced by adding 1 mM isopropyl β-D-1-thiogalactopyranoside (IPTG) and the culture was further grown at 16° C. for 16 hr. Cells were harvested by centrifugation (3,000×g; 10 min; 4° C.). The cell pellets were collected and were either used immediately or stored at −80° C.

The cells were resuspended in lysis buffer (50 mM Tris-HCl pH 8.0, 150 mM NaCl, 20 mM imidazole). After sonication, the lysate was clarified by centrifugation at 16,000×g for 15 minutes. The clarified lysate was loaded onto an equilibrated (equilibration buffer: 50 mM Tris-HCl, pH 8.0, 20 mM imidazole, 150 mM NaCl, 20% glycerol) talon metal affinity column (Takara Bio). After loading of protein sample, the column was washed with equilibration buffer to remove unbound contaminant proteins. The His-tagged recombinant polypeptides were eluted by equilibration buffer containing 250 mM imidazole. The proteins were used for activity assays or aliquoted and stored at −80 until needed.

All samples were analyzed by use of suitable HPLC and LC MS methods.

For the LC-MS detection of GDP-L-fucose and GDP-L-galactose, the samples were quenched by heating at 99° C. for 10 minutes, and the proteins were removed by centrifugation. The column was a Luna, C18(2) HST, 2.0 mm×100 mm with 2.5 μm particle size and 100 Å pore size from Phenomenex. Mobile phase A was 10 mM triethylammonium acetate pH 7.0 and mobile phase B was 10 mM triethylamine acetate pH 7.0 (90%) and 10% acetonitrile. The column compartment was set at 25° C., and the flow rate was 0.3 mL/min. The pump method was: 0 min 1% B, 1 min 1% B, 8 min 5% B, 8.1 min 20% B, 10 min 20% B, 10.1 min 1% B and hold until 15 mins. The UV detector was set to 254 nm. The spray voltage was set to 2.7 kV, the capillary temperature was 300° C., the sheath gas was 40, the auxiliary gas was 8, the spare gas was 2, the max spray current was 100, the probe heater temperature was 320° C. and the S-Lens RF level was 60.

For the HPLC detection of GDP-L-fucose, GDP-mannose, GDP-L-gulose and GDP-L-galactose, the samples were prepared as described above. The column was a Luna 5 μm C18(2) 100 Å, 4.6×250 mm from Phenomenex. Mobile phase A was 10 mM triethylammonium acetate pH 7.0 and mobile phase B was 10 mM triethylamine acetate pH 7.0 (90%) and 10% acetonitrile. The column compartment was set at 25° C., and the flow rate was 1.3 mL/min. The pump method was: 0 min 1% B, 1 min 1% B, 8 min 5% B, 8.1 min 20% B, 10 min 20% B, 10.1 min 1% B and hold until 15 mins. The UV detector was set to 254 nm.

The following method was used for the LC-MS detection of 2′-FL. The samples were prepared as described above. The analytes were separated using a Thermo Fisher, Hypercarb column, 2.1×100 mm, 3 um particle size. Mobile phase A was 0.1% formic acid in water and mobile phase B was 0.1% formic acid in acetonitrile. The column compartment was set to 25° C., and the flow rate was set to 0.2 mL/min. The full run time was 30 minutes. The pump method was: 0 min 0% B, 21 min 12% B, 22 min 0% B, 30 min 0% B. The spray voltage was 3.5 kV, the capillary temperature was 300° C., the sheath gas was 50, the auxiliary gas was 10, the spare gas was 2, the max spray current was 100, the probe heater temperature was 370° C. and the S-Lens RF level was 45.

For HPLC detection of 2′-FL, the samples were prepared as described above. The HPLC instrument method was optimized with isocratic elution of the analytes with distilled water, using an Aminex HPX-87H, 300×7.8 mm (BioRad) column and a flow rate of 0.6 mL/min and total run time of 12 min. The column compartment was set to 50° C. 2′-FL was monitored using a Refractomax 520.

### Example 8: Identification of Novel GDP-Mannose-4,6-Dehydratase Enzymes for GDP-L-Fucose and 2′-FL Production

In the de novo pathway, the conversion of GDP-D-mannose to GDP-4-keto-6-deoxymannose is catalyzed by GDP-mannose-4,6-dehydratase (GMD). The resulting GDP-4-keto-6-deoxymannose is converted to GDP-L-fucose by a bifunctional 3,5-epimerase-4-reductase (e.g., WcaG from E. coli) enzyme.

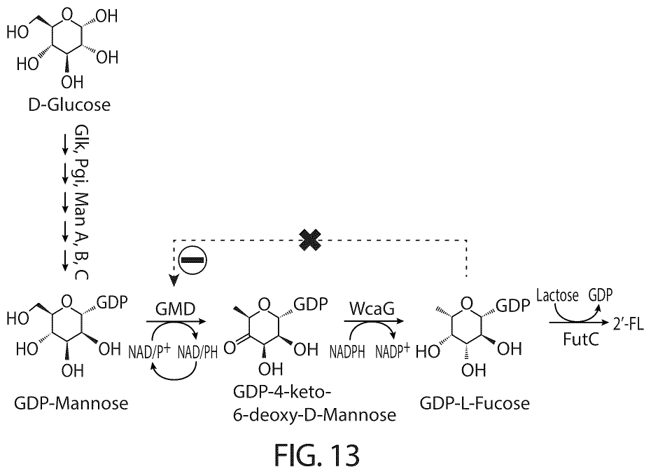

It has been well-established that GDP-L-fucose acts as a negative feedback to the activity of GMD enzymes (FIG. 13). The inhibition is characterized as competitive inhibition in E. coli, and allosteric inhibition with human and A. thaliana GMD. See Somoza, J. R. et al., “Structural and Kinetic Analysis of Escherichia Coli GDP-Mannose 4,6 Dehydratase Provides Insights into the Enzyme's Catalytic Mechanism and Regulation by GDP-L-fucose,” Structure, 8(2): 123-125 (2000); and Pfeiffer, M. et al., “A Parsimonious Mechanism of Sugar Dehydration by Human GDP-Mannose-4,6-Dehydratase,” ACS Catalysis, 9(4): 2962-2968 (2019).

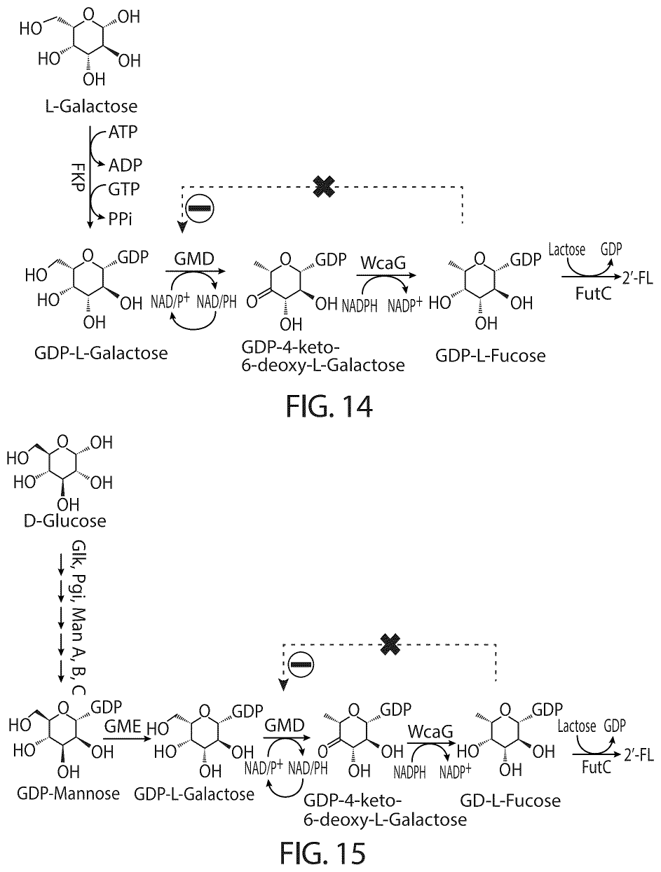

In order to drive the production of 2′-FL, it would be beneficial to generate a large pool of GDP-L-fucose. The challenge posed by the GDP-L-fucose negative feedback is present regardless of whether the de novo pathway is used as described above, or the novel pathways according to the present teachings are used. Referring to FIG. 14, it can be seen that after L-galactose is converted to GDP-L-galactose, a GMD is used to convert GDP-L-galactose to GDP-4-keto-6-deoxy-L-galactose, which is converted to GDP-L-fucose by a GDP-L-fucose synthase. Similarly, referring to FIG. 15, in the modified de novo pathway according to the present teachings, after GDP-mannose is converted to GDP-L-galactose by a GDP-mannose 3′, 5′-epimerase (GME) enzyme, a GMD is used to convert GDP-L-galactose to GDP-4-keto-6-deoxy-L-galactose, which is converted to GDP-L-fucose by a GDP-L-fucose synthase.

To alleviate GDP-L-fucose inhibition that is present in all three pathways, a series of mutations (Table 4) were generated, targeting the GDP-L-fucose allosteric binding pocket in A. thaliana GMD (At GMD, SEQ ID NO: 5) and human GMD (Hs GMD, SEQ ID NO: 9).

TABLE 4
List of GMD enzymes and mutants

| | | Amino acid | |
|---|---|---|---|
| GMD enzyme | Source Organism | sequence | DNA sequence |
| At GMD | Arabidopsis thaliana | 5 | 6 |
| Hs GMD | Homo sapiens | 9 | 10 |
| Ec GMD | Escherichia coli | 11 | 12 |
| At GMD M2 | Arabidopsis thaliana | 75 | 76 |
| At GMD M3 | Arabidopsis thaliana | 77 | 78 |
| At GMD M4 | Arabidopsis thaliana | 79 | 80 |
| Hs GMD M2 | Homo sapiens | 81 | 82 |
| Hs GMD M3 | Homo sapiens | 83 | 84 |
| Hs GMD M4 | Homo sapiens | 85 | 86 |

To test for inhibition, the mutants were expressed and purified as described in Example 7, and assayed at a range of GDP-L-fucose concentrations. The assay conditions were as follows: 50 mM Tris pH 7.5, 1 mM GDP-mannose, 0.5 mM NADP⁺, 0.5 mg/mL dehydratase (GMD) enzyme, and 0 μM, 70 μM or 350 μM GDP-L-fucose. The reactions were quenched at 99° C. for 10 minutes, and the enzymes' respective activities were analyzed using the nucleotide sugar LC-MS method by GDP-4-keto-6-deoxymannose detection.

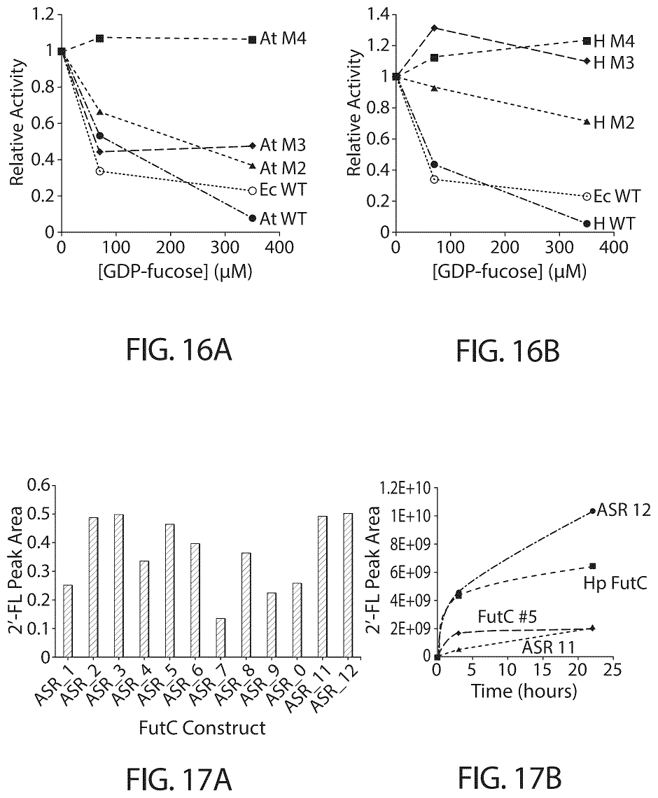

The relative activity for the GMD mutants was plotted as a function of GDP-L-fucose concentration (FIG. 16). Referring to Panel A of FIG. 16, it can be seen that both At GMD wild type (At WT) and Ec GMD wild type (Ec WT) were inhibited by GDP-L-fucose, with a 95% (At WT) and 80% (Ec WT) decrease in activity at 350 μM GDP-L-fucose.

By comparison, the At GMD mutants show marked improvements in their activity at 350 μM GDP-L-fucose, especially with At GMD M4 (At M4, SEQ ID NO: 79) retaining a surprising 100% activity. The other two mutants At GMD M3 (At M3, SEQ ID NO: 77) and At GMD M2 (At M2, SEQ ID NO: 75) retained 50% activity and 40% activity, respectively.

Similarly, referring to Panel B of FIG. 16, the Hs GMD wild type enzyme (H WT) was severely inhibited at 350 μM GDP-L-fucose, retaining less than 5% of its activity. Again, it can be seen that the Hs GMD mutants show marked improvements in their activity at 350 μM GDP-L-fucose, especially with both Hs GMD M4 (H M4, SEQ ID NO: 85) and Hs GMD M3 (H M3, SEQ ID NO: 83) surprisingly retaining 100% activity. In addition, the third mutant Hs GMD M2 (H M2, SEQ ID NO: 81) also was able to retain 80% activity.

The above data show that the present GMD mutants can be used to improve the yield of 2′-FL production by increasing the GDP-L-fucose pool.

### Example 9: Identification of Novel Alpha-1,2-Fucosyltransferase from Ancestral Sequence Reconstruction

One of the major limitations for 2′-FL production is FutC activity. After screening various FutC candidates as described in Example 5, the inventors sought to identify mutant FutC candidates with even higher activity and improved solubility using bioinformatics. Based on ancestral sequence reconstruction (ASR) analysis, a series of ASR mutants were designed (Table 5) and screened for their solubility and activity.

TABLE 5
List of FutC mutants

| FutC enzyme | Amino acid sequence | DNA sequence |
|---|---|---|
| FutC 5 | 29 | 30 |
| FutC 21 | 61 | 62 |
| ASR 1 | 87 | 88 |
| ASR 2 | 89 | 90 |
| ASR 3 | 91 | 92 |
| ASR 4 | 93 | 94 |
| ASR 5 | 95 | 96 |
| ASR 6 | 97 | 98 |
| ASR 7 | 99 | 100 |
| ASR 8 | 101 | 102 |
| ASR 9 | 103 | 104 |
| ASR 10 | 105 | 106 |
| ASR 11 | 107 | 108 |
| ASR 12 | 109 | 110 |

The enzymes listed in Table 5 were expressed in E. coli, and the clarified lysate was normalized based on the OD600 standard curve and used to screen for activity. To the clarified lysate was added 50 mM Tris pH 7.5, 1 mM GDP-L-fucose, and 80 mM lactose, and the reactions were quenched by heating at 99° C. for 10 minutes, and analyzed using the 2′-FL HPLC method.

Referring to Panel A of FIG. 17, ASR12 showed both improved solubility and activity compared to the rest of the library. An assay was performed to compare the activity of the ASR11 and ASR12 enzymes to the activity of the parent construct, FutC #5 (SEQ ID NO: 29) and Helicobacter pylori FutC (HpFutC, SEQ ID NO: 61), an enzyme commonly used for 2′-FL production. Specifically, the assay conditions were: 2 mM GDP-L-fucose, 40 mM lactose, 50 mM Tris pH 7.5, and 0.5 mg/mL FutC.

Referring to Panel B of FIG. 17, it can be seen that ASR 12 (SEQ ID NO: 109) outperformed Hp FutC as well as the parent construct, and can be used to improve overall titers of 2′-FL using the biosynthetic pathways described herein.

### Example 10: Conversion of L-Galactose to 2′-FL Using an In Vitro Enzyme Cascade

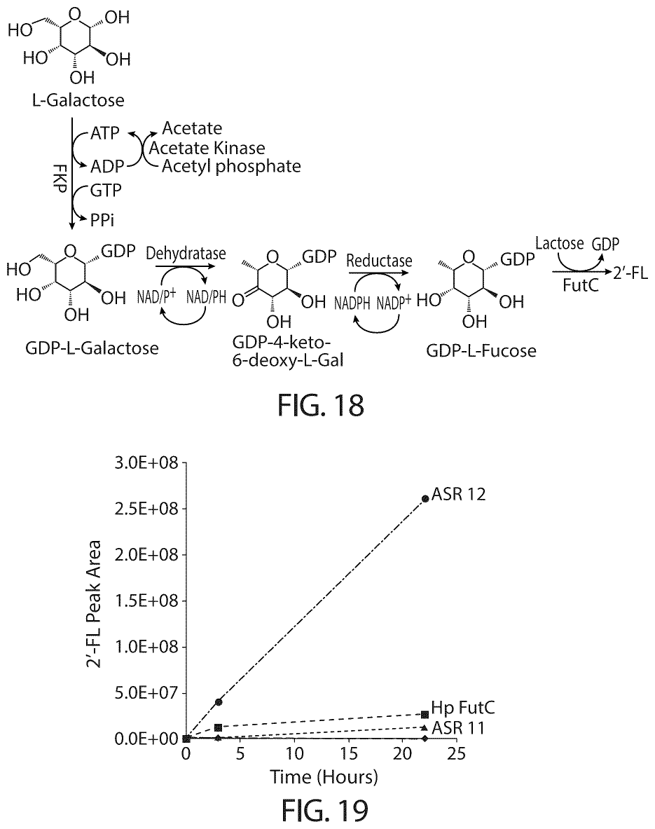

As described herein, the inventors have developed a single-pot bioconversion process that can be used to produce 2′-FL from L-galactose. Referring to FIG. 18, the present enzymatic process uses 4 main enzymes, which can be complemented by an ATP recycling system, a GTP regeneration system, and/or an NADPH regeneration system.

FIG. 18 illustrates the 4-enzyme in vitro pathway according to the present teachings. In the first step, L-galactose is converted to GDP-L-galactose using a phosphofructokinase (FKP) enzyme (e.g., SEQ ID NO: 1). In the second step, a dehydratase (e.g., a GMD and preferably, the mutant M4 from At GMD (SEQ ID NO: 79 and SEQ ID NO: 5) is used to convert GDP-L-galactose into GDP-4-keto-6-deoxy-L-galactose. In the third step, a GDP-L-fucose synthase (e.g., a reductase such as WcaG, SEQ ID NO: 7) is used to convert GDP-4-keto-6-deoxy-L-galactose to GDP-L-fucose. In the final step, a FutC (e.g., FutC 5 and preferably ASR 12, SEQ ID NO: 29 and SEQ ID NO: 109) is used to produce 2′-FL from GDP-L-fucose. Also included is an acetate kinase for ATP recycling.

To demonstrate this enzymatic process, all required enzymes were expressed and purified as previously described. The reaction conditions were: 50 mM Tris pH 7.5, 10 mM L-galactose, 2 mM ATP, 2 mM GTP, 25 mM acetyl phosphate, 5 mM magnesium chloride, 5 mM potassium chloride, 0.15 mM NADP⁺, 0.5 mM NADPH, 40 mM lactose, 0.23 g/L phosphofructokinase (FKP, SEQ ID NO: 1), 0.06 g/L an acetate kinase for the ATP recycling system (Gs Ack, SEQ ID NO: 65), 0.3 g/L At GMD (SEQ ID NO: 79), 0.75 g/L WcaG (SEQ ID NO: 7) and 0.2 g/L ASR 12 (SEQ ID NO: 109). The reactions were quenched by heating at 99° C. for 3 hours and 22 hours and analyzed using the 2′-FL LC-MS method. The results are shown in FIG. 19.

As predicted, 2′-FL production was significantly higher using the ASR12 FutC, compared to the parent enzyme (FutC #5) and the commonly used Hp FutC. The inventors henceforth have demonstrated a novel process for producing 2′-FL in vitro with high product yield.

Sequences of Interest:
FKP: AA
(SEQ ID NO: 1)
MQKLLSLPPNLVQSFHELERVNRTDWFCTSDPVGKKLGSGGGTSWLLE
ECYNEYSDGATFGEWLEKEKRILLHAGGQSRRLPGYAPSGKILTPVPV
FRWERGQHLGQNLLSLQLPLYEKIMSLAPDKLHTLIASGDVYIRSEKP
LQSIPEADVVCYGLWVDPSLATHHGVFASDRKHPEQLDFMLQKPSLAE
LESLSKTHLFLMDIGIWLLSDRAVEILMKRSHKESSEELKYYDLYSDF
GLALGTHPRIEDEEVNTLSVAILPLPGGEFYHYGTSKELISSTLSVQN
KVYDQRRIMHRKVKPNPAMFVQNAVVRIPLCAENADLWIENSHIGPKW
KIASRHIITGVPENDWSLAVPAGVCVDVVPMGDKGFVARPYGLDDVFK
GDLRDSKTTLTGIPFGEWMSKRGLSYTDLKGRTDDLQAASVFPMVNSV
EELGLVLRWMLSEPELEEGKNIWLRSERFSADEISAGANLKRLYAQRE
EFRKGNWQALAVNHEKSVFYQLDLADAAEDFVRLGLDMPELLPEDALQ
MSRIHNRMLRARILKLDGKDYRPEEQAAFDLLRDGLLDGISNRKSTPK
LDVYSDQIVWGRSPVRIDMAGGWTDTPPYSLYSGGNVVNLAIELNGQP
PLQVYVKPCKDFHIVLRSIDMGAMEIVSTFDELQDYKKIGSPFSIPKA
ALSLAGFAPAFSAVSYASLEEQLKDFGAGIEVTLLAAIPAGSGLGTSS
ILASTVLGAINDFCGLAWDKNEICQRTLVLEQLLTTGGGWQDQYGGVL
QGVKLLQTEAGFAQSPLVRWLPDHLFTHPEYKDCHLLYYTGITRTAKG
ILAEIVSSMFLNSSLHLNLLSEMKAHALDMNEAIQRGSFVEFGRLVGK
TWEQNKALDSGTNPPAVEAIIDLIKDYTLGYKLPGAGGGGYLYMVAKD
PQAAVRIRKILTENAPNPRARFVEMTLSDKGFQVSRS
FKP: DNA
(SEQ ID NO: 2)
ATGCAAAAACTACTATCTTTACCGCCCAATCTGGTTCAGTCTTTTCAT
GAACTGGAGAGGGTGAACCGTACCGATTGGTTTTGTACTTCCGACCCG
GTAGGTAAGAAACTTGGTTCCGGTGGTGGAACATCCTGGTTGCTTGAA
GAATGTTATAATGAATATTCAGATGGTGCTACTTTTGGAGAGTGGCTT
GAAAAAGAAAAAAGAATTCTTCTTCATGCGGGTGGGCAAAGCCGTCGT
TTACCCGGCTATGCACCTTCTGGAAAGATTCTCACTCCGGTTCCTGTG
TTCCGGTGGGAGAGAGGGCAACATCTGGGACAAAATCTGCTTTCTCTG
CAACTTCCCCTATATGAAAAAATCATGTCTTTGGCTCCGGATAAACTC
CATACACTGATTGCGAGTGGTGATGTCTATATTCGTTCGGAGAAACCT
TTGCAGAGTATTCCCGAAGCGGATGTGGTTTGTTATGGACTGTGGGTA
GATCCGTCTCTGGCTACCCATCATGGCGTGTTTGCTTCCGATCGCAAA
CATCCCGAACAACTCGACTTTATGCTTCAGAAGCCTTCGTTGGCAGAA
TTGGAATCTTTATCGAAGACCCATTTGTTCCTGATGGACATCGGTATA
TGGCTTTTGAGTGACCGTGCCGTAGAAATCTTGATGAAACGTTCTCAT
AAAGAAAGCTCTGAAGAACTAAAGTATTATGATCTTTATTCCGATTTT
GGATTAGCTTTGGGAACTCATCCCCGTATTGAAGACGAAGAGGTCAAT
ACGCTATCCGTTGCTATTCTGCCTTTGCCGGGAGGAGAGTTCTATCAT
TACGGGACCAGTAAAGAACTGATATCTTCAACTCTTTCCGTACAGAAT
AAGGTTTACGATCAGCGTCGTATCATGCACCGTAAAGTAAAGCCCAAT
CCGGCTATGTTTGTCCAAAATGCTGTAGTGCGGATACCTCTTTGTGCC
GAGAATGCTGATTTATGGATCGAGAACAGTCATATCGGACCAAAGTGG
AAGATTGCTTCACGACATATTATTACCGGGGTTCCGGAAAATGACTGG
TCATTGGCTGTGCCTGCCGGAGTGTGTGTAGATGTGGTTCCGATGGGT
GATAAGGGCTTTGTTGCCCGTCCATACGGCCTGGACGATGTTTTCAAA
GGAGATTTGAGAGATTCCAAAACAACCCTGACGGGTATTCCTTTTGGT
GAATGGATGTCCAAACGCGGTTTGTCATATACAGATTTGAAAGGACGT
ACGGACGATTTACAGGCAGCTTCCGTATTCCCTATGGTTAATTCTGTA
GAAGAGTTGGGATTGGTGTTGAGGTGGATGTTGTCCGAACCCGAACTG
GAGGAAGGAAAGAATATCTGGTTACGTTCCGAACGTTTTTCTGCGGAC
GAAATTTCGGCAGGTGCCAATCTGAAGCGTTTGTATGCACAACGTGAA
GAGTTCAGAAAAGGAAACTGGCAAGCATTGGCCGTTAATCATGAAAAA
AGTGTTTTCTATCAACTTGATTTGGCCGATGCAGCTGAAGATTTTGTA
CGTCTTGGTTTGGATATGCCTGAATTATTGCCTGAGGATGCTCTGCAG
ATGTCACGCATCCATAACCGGATGTTGCGTGCGCGTATTTTGAAATTA
GACGGGAAAGATTATCGTCCGGAAGAACAGGCTGCTTTTGATTTGCTT
CGTGACGGCTTGCTGGACGGGATCAGTAATCGTAAGAGTACCCCAAAA
TTGGATGTATATTCCGATCAGATTGTTTGGGGACGTAGTCCCGTGCGC
ATCGATATGGCAGGTGGATGGACCGATACTCCTCCTTATTCACTTTAT
TCGGGAGGAAATGTGGTGAATCTGGCTATTGAGTTGAACGGACAACCT
CCCTTACAGGTCTATGTGAAGCCGTGTAAAGATTTCCATATCGTCCTG
CGTTCTATCGATATGGGTGCTATGGAAATAGTATCTACGTTTGATGAA
TTGCAAGATTATAAGAAGATCGGTTCACCTTTCTCTATTCCGAAAGCC
GCTCTGTCATTGGCAGGCTTTGCACCTGCGTTTTCTGCTGTATCTTAT
GCTTCATTAGAAGAACAGCTTAAAGATTTCGGTGCAGGTATTGAAGTG
ACTTTATTGGCTGCTATTCCTGCCGGTTCCGGTTTGGGCACCAGTTCC
ATTCTGGCTTCTACCGTACTTGGTGCCATTAACGATTTCTGTGGTTTA
GCCTGGGATAAAAATGAGATTTGTCAACGTACTCTTGTCCTTGAACAA
TTGCTGACTACCGGTGGTGGATGGCAGGATCAGTATGGAGGTGTGTTG
CAGGGTGTGAAGCTTCTTCAGACCGAGGCCGGCTTTGCTCAAAGTCCA
TTGGTGCGTTGGCTACCCGATCATTTATTTACGCATCCTGAATACAAA
GACTGTCACTTGCTTTATTATACCGGTATAACTCGTACGGCAAAAGGG
ATCTTGGCAGAAATAGTCAGTTCCATGTTCCTCAATTCATCGTTGCAT
CTCAATTTACTCTCGGAAATGAAGGCGCATGCATTGGATATGAATGAA
GCTATACAGCGTGGAAGTTTTGTTGAGTTTGGCCGTTTGGTAGGAAAA
ACCTGGGAACAAAACAAAGCATTGGATAGCGGAACAAATCCTCCGGCT
GTGGAGGCAATTATCGATCTGATAAAAGATTATACCTTGGGATATAAA
TTGCCGGGAGCCGGTGGTGGCGGGTACTTATATATGGTAGCGAAAGAT
CCGCAAGCTGCTGTTCGTATTCGTAAGATACTGACAGAAAACGCTCCG
AATCCGCGGGCACGTTTTGTTGAAATGACGTTATCTGATAAGGGATTC
CAAGTATCACGATCATGA
At GME AA
(SEQ ID NO: 3)
MGTTNGTDYGAYTYKELEREQYWPSENLKISITGAGGFIASHIARRLK
HEGHYVIASDWKKNEHMTEDMFCDEFHLVDLRVMENCLKVTEGVDHVF
NLAADMGGMGFIQSNHSVIMYNNTMISFNMIEAARINGIKRFFYASSA
CIYPEFKQLETTNVSLKESDAWPAEPQDAYGLEKLATEELCKHYNKDF
GIECRIGRFHNIYGPFGTWKGGREKAPAAFCRKAQTSTDRFEMWGDGL
QTRSFTFIDECVEGVLRLTKSDFREPVNIGSDEMVSMNEMAEMVLSFE
EKKLPIHHIPGPEGVRGRNSDNNLIKEKLGWAPNMRLKEGLRITYFWI
KEQIEKEKAKGSDVSLYGSSKVVGTQAPVQLGSLRAADGKE
At GME DNA
(SEQ ID NO: 4)
ATGGGCACGACTAACGGCACCGACTATGGAGCGTACACGTACAAAGAA
CTGGAACGCGAACAATACTGGCCATCCGAGAATTTGAAAATCAGTATT
ACGGGCGCGGGCGGCTTCATTGCTAGCCACATCGCACGCCGCCTGAAA
CACGAAGGTCACTATGTGATTGCAAGCGATTGGAAGAAGAACGAGCAC
ATGACCGAAGATATGTTTTGCGATGAATTTCATTTAGTGGACCTGCGT
GTAATGGAGAATTGCTTAAAAGTGACTGAGGGTGTGGATCACGTGTTC
AATCTCGCCGCGGATATGGGCGGCATGGGCTTTATTCAAAGTAACCAT
AGCGTGATTATGTACAACAACACGATGATTAGCTTTAACATGATCGAG
GCCGCGCGCATCAATGGTATCAAACGGTTCTTCTATGCCAGCTCGGCG
TGCATTTACCCTGAATTTAAACAGCTGGAAACCACCAATGTGTCCTTG
AAAGAATCTGATGCGTGGCCGGCAGAACCGCAAGACGCGTACGGCCTG
GAAAAGCTGGCGACTGAAGAACTATGCAAGCACTACAATAAAGATTTT
GGTATCGAATGCCGCATTGGCCGGTTCCACAACATTTATGGTCCTTTT
GGGACGTGGAAAGGCGGACGTGAGAAGGCGCCAGCCGCGTTTTGTCGC
AAAGCGCAGACTTCTACAGATCGGTTTGAGATGTGGGGTGATGGTTTG
CAGACCCGCTCATTCACTTTTATCGACGAGTGTGTGGAAGGAGTGCTG
CGCCTGACCAAATCGGACTTCCGCGAGCCCGTTAATATCGGTTCTGAC
GAGATGGTGTCGATGAACGAAATGGCGGAAATGGTACTGAGTTTTGAA
GAAAAGAAATTACCTATCCATCACATTCCCGGCCCTGAGGGAGTACGG
GGTCGCAACTCAGATAATAACCTGATCAAAGAGAAACTGGGCTGGGCT
CCAAACATGCGCCTCAAAGAAGGCCTGCGTATCACCTACTTTTGGATA
AAAGAACAAATAGAGAAAGAAAAGGCGAAAGGTAGTGATGTCTCGTTG
TATGGATCATCGAAAGTGGTGGGTACGCAAGCCCCGGTTCAGCTCGGC
AGCCTGCGCGCGGCAGACGGAAAAGAATAA
At Gmd AA
(SEQ ID NO: 5)
MASENNGSRSDSESITAPKADSTVVEPRKIALITGITGQDGSYLTEFL
LGKGYEVHGLIRRSSNFNTQRINHIYIDPHNVNKALMKLHYADLTDAS
SLRRWIDVIKPDEVYNLAAQSHVAVSFEIPDYTADVVATGALRLLEAV
RSHTIDSGRTVKYYQAGSSEMFGSTPPPQSETTPFHPRSPYAASKCAA
HWYTVNYREAYGLFACNGILFNHESPRRGENFVTRKITRALGRIKVGL
QTKLFLGNLQASRDWGFAGDYVEAMWLMLQQEKPDDYVVATEEGHTVE
EFLDVSFGYLGLNWKDYVEIDQRYFRPAEVDNLQGDASKAKEVLGWKP
QVGFEKLVKMMVDEDLELAKREKVLVDAGYMDAKQQP
At Gmd DNA
(SEQ ID NO: 6)
ATGGCAAGTGAGAACAATGGTTCACGTTCTGACTCTGAAAGCATCACG
GCTCCTAAAGCGGACAGCACCGTTGTGGAACCACGGAAAATCGCTCTA
ATCACCGGCATCACGGGTCAGGACGGTAGTTACTTGACTGAATTTCTA
CTAGGCAAAGGTTACGAAGTGCATGGCCTGATCCGTAGGAGTAGCAAT
TTTAACACGCAGCGGATCAATCATATCTATATTGATCCACACAACGTG
AACAAAGCTTTAATGAAACTCCATTACGCGGATCTCACTGACGCCTCT
TCGTTGCGTCGCTGGATCGACGTCATTAAACCTGACGAAGTGTATAAC
CTGGCGGCACAGTCTCATGTGGCCGTTTCATTCGAAATACCTGATTAT
ACGGCGGACGTGGTTGCCACCGGTGCCTTAAGACTGCTCGAGGCGGTT
CGCTCCCATACCATTGATTCCGGGCGCACGGTAAAATATTATCAGGCA
GGAAGCAGCGAAATGTTTGGAAGTACGCCGCCCCCTCAGTCTGAGACA
ACCCCGTTTCACCCGCGCAGTCCGTATGCGGCATCTAAATGTGCCGCA
CATTGGTATACAGTCAATTATCGTGAGGCTTATGGCTTGTTTGCATGC
AATGGCATTCTGTTCAATCATGAAAGCCCGCGCAGAGGCGAAAATTTT
GTTACCCGCAAAATTACGCGTGCCCTGGGCCGTATTAAAGTAGGTCTG
CAAACTAAACTGTTTCTTGGCAACCTCCAGGCTAGCCGTGACTGGGGA
TTTGCCGGTGATTATGTCGAAGCCATGTGGCTCATGTTACAGCAGGAG
AAACCGGACGATTATGTTGTTGCGACAGAAGAAGGACACACAGTGGAG
GAATTTTTGGATGTATCGTTCGGCTATTTAGGTCTAAACTGGAAAGAT
TACGTTGAGATTGATCAACGCTACTTCCGGCCGGCGGAAGTGGACAAC
CTGCAAGGAGATGCCTCCAAGGCAAAAGAAGTACTGGGTTGGAAACCG
CAGGTGGGCTTCGAGAAACTTGTCAAAATGATGGTGGATGAAGATCTG
GAATTAGCTAAACGCGAGAAGGTACTGGTAGATGCAGGATACATGGAT
GCGAAGCAGCAACCGTAA
E. coli WcaG: AA
(SEQ ID NO: 7)
MSKQRVFIAGHRGMVGSAIRRQLEQRGDVELVLRTRDELNLLDSRAVH
DFFASERIDQVYLAAAKVGGIVANNTYPADFIYQNMMIESNIIHAAHQ
NDVNKLLFLGSSCIYPKLAKQPMAESELLQGTLEPTNEPYAIAKIAGI
KLCESYNRQYGRDYRSVMPTNLYGPHDNFHPSNSHVIPALLRRFHEAT
AQNAPDVVVWGSGTPMREFLHVDDMAAASIHVMELAHEVWLENTQPML
SHINVGTGVDCTIRELAQTIAKVVGYKGRVVFDASKPDGTPRKLLDVT
RLHQLGWYHEISLEAGLASTYQWFLENQDRFRG
E. coli WcaG: DNA
(SEQ ID NO: 8)
ATGAGCAAACAGCGCGTGTTTATTGCCGGCCATCGTGGTATGGTTGGT
AGCGCCATTCGTCGCCAGCTGGAACAGCGTGGTGATGTGGAGCTGGTG
CTGCGTACCCGCGACGAACTGAATTTATTAGATAGCCGCGCCGTTCAC
GACTTTTTCGCCAGCGAACGCATCGACCAAGTTTATCTGGCCGCCGCA
AAAGTGGGCGGTATCGTTGCCAACAACACCTATCCGGCCGACTTTATC
TATCAGAATATGATGATTGAAAGCAACATCATCCATGCCGCCCACCAG
AACGACGTGAACAAACTGCTGTTTTTAGGTAGCAGCTGCATCTACCCG
AAGCTGGCCAAACAGCCGATGGCCGAAAGCGAACTGCTGCAAGGTACA
CTGGAACCGACCAACGAACCTTACGCAATTGCCAAGATCGCCGGCATT
AAGCTGTGTGAGAGCTACAACCGCCAGTACGGTCGCGATTATCGCAGC
GTTATGCCGACCAATTTATATGGCCCGCATGATAACTTTCACCCGAGT
AACAGCCACGTTATTCCGGCTTTATTACGCCGTTTCCACGAAGCAACC
GCCCAGAACGCCCCGGATGTTGTTGTTTGGGGCAGCGGTACCCCTATG
CGCGAGTTTTTACACGTTGATGATATGGCAGCAGCCAGCATCCATGTT
ATGGAACTGGCCCATGAAGTGTGGCTGGAGAACACACAGCCGATGCTG
AGCCATATCAATGTGGGCACTGGTGTGGATTGCACCATTCGTGAACTG
GCCCAGACCATCGCAAAAGTGGTGGGCTACAAAGGTCGCGTGGTGTTT
GATGCCAGCAAACCGGATGGCACACCGCGCAAACTGCTGGACGTGACC
CGTTTACATCAGCTGGGCTGGTACCACGAAATCAGTTTAGAGGCTGGT
TTAGCCAGCACCTACCAGTGGTTTTTAGAAAATCAAGATCGCTTTCGC
GGTTGA
Hs Gmd: AA
(SEQ ID NO: 9)
MRNVALITGITGQDGSYLAEFLLEKGYEVHGIVRRSSSFNTGRIEHLY
KNPQAHIEGNMKLHYGDLTDSTCLVKIINEVKPTEIYNLGAQSHVKIS
FDLAEYTADVDGVGTLRLLDAVKTCGLINSVKFYQASTSELYGKVQEI
PQKETTPFYPRSPYGAAKLYAYWIVVNFREAYNLFAVNGILFNHESPR
RGANFVTRKISRSVAKIYLGQLECFSLGNLDAKRDWGHAKDYVEAMWL
MLQNDEPEDFVIATGEVHSVREFVEKSFLHIGKTIVWEGKNENEVGRC
KETGKVHVTVDLKYYRPTEVDFLQGDCTKAKQKLNWKPRVAFDELVRE
MVHADVELMRTNPNA
Hs Gmd: DNA
(SEQ ID NO: 10)
ATGCGAAACGTGGCCTTGATCACCGGTATTACCGGCCAGGATGGCTCA
TATCTGGCAGAATTTCTGCTTGAAAAAGGCTATGAGGTTCATGGCATC
GTGCGCCGCAGCAGTAGTTTTAATACCGGCCGCATTGAACATCTGTAT
AAAAACCCACAAGCACACATCGAAGGAAATATGAAACTGCATTATGGC
GATTTGACAGACTCAACGTGTCTGGTTAAGATAATAAACGAAGTGAAG
CCTACCGAAATTTACAACCTGGGTGCGCAGTCTCATGTGAAAATTAGC
TTCGATTTGGCCGAATATACCGCGGATGTCGATGGTGTGGGTACGTTA
CGACTGTTGGACGCTGTTAAAACCTGCGGGCTGATCAACAGCGTGAAA
TTTTATCAGGCTAGCACGAGTGAGCTCTATGGAAAGGTCCAGGAGATT
CCCCAGAAGGAAACGACGCCTTTCTATCCACGCAGCCCGTATGGGGCA
GCAAAACTTTATGCCTATTGGATCGTAGTGAACTTTCGCGAAGCTTAT
AATCTTTTTGCGGTTAATGGCATACTGTTTAACCACGAGTCGCCACGA
CGCGGCGCAAACTTCGTGACCCGTAAAATAAGTCGTAGCGTCGCGAAG
ATCTATCTGGGTCAGCTCGAATGTTTCAGCCTTGGCAACCTGGATGCG
AAACGTGATTGGGGACACGCGAAAGATTATGTCGAAGCCATGTGGCTG
ATGTTACAAAACGATGAACCTGAGGACTTCGTTATCGCCACGGGTGAA
GTGCATAGCGTACGCGAATTTGTCGAAAAAAGCTTCCTCCATATAGGT
AAGACCATCGTGTGGGAAGGCAAAAATGAGAACGAGGTTGGTCGCTGC
AAAGAAACCGGCAAAGTTCACGTTACGGTTGATCTCAAATACTACAGA
CCCACCGAAGTGGACTTTCTGCAAGGCGATTGTACCAAAGCCAAACAG
AAACTAAATTGGAAACCTCGCGTTGCCTTCGACGAACTCGTCCGTGAA
ATGGTCCATGCAGATGTCGAACTGATGAGAACAAACCCTAACGCGTAA
Ec Gmd: AA
(SEQ ID NO: 11)
MSKVALITGVTGQDGSYLAEFLLEKGYEVHGIKRRASSFNTERVDHIY
QDPHTCNPKFHLHYGDLSDTSNLTRILREVQPDEVYNLGAMSHVAVSF
ESPEYTADVDAMGTLRLLEAIRFLGLEKKTRFYQASTSELYGLVQEIP
QKETTPFYPRSPYAVAKLYAYWITVNYRESYGMYACNGILFNHESPRR
GETFVTRKITRAIANIAQGLESCLYLGNMDSLRDWGHAKDYVKMQWMM
LQQEQPEDFVIATGVQYSVRQFVEMAAAQLGIKLRFEGTGVEEKGIVV
SVTGHDAPGVKPGDVIIAVDPRYFRPAEVETLLGDPTKAHEKLGWKPE
ITLREMVSEMVANDLEAAKKHSLLKSHGYDVAIALES
Ec Gmd: DNA
(SEQ ID NO: 12)
ATGAGCAAAGTTGCTTTAATCACCGGTGTGACCGGCCAAGATGGCAGC
TATTTAGCCGAGTTTCTGCTGGAGAAAGGCTACGAAGTGCATGGTATT
AAGCGTCGCGCCAGCAGCTTCAATACCGAACGTGTGGATCATATCTAT
CAAGATCCGCACACTTGTAACCCGAAATTCCATCTGCACTATGGCGAT
CTGAGCGATACCAGTAATTTAACCCGCATTCTGCGCGAAGTTCAGCCG
GATGAGGTGTACAATCTGGGCGCCATGAGTCATGTGGCCGTGAGCTTT
GAAAGCCCGGAATACACCGCCGATGTTGATGCAATGGGCACTTTACGT
TTACTGGAAGCCATTCGCTTTTTAGGTCTGGAGAAGAAAACTCGCTTC
TACCAAGCTAGCACAAGCGAACTGTATGGTCTGGTGCAAGAAATCCCG
CAGAAAGAAACTACCCCGTTTTATCCGCGTAGTCCGTATGCAGTGGCC
AAGCTGTATGCCTACTGGATCACCGTGAACTACCGTGAGAGCTATGGC
ATGTATGCTTGTAACGGCATTTTATTTAACCATGAGAGCCCGCGTCGC
GGCGAGACATTTGTTACCCGCAAAATTACCCGCGCAATCGCCAATATC
GCACAAGGTTTAGAGAGTTGTTTATATCTGGGCAATATGGACTCTTTA
CGTGACTGGGGCCATGCCAAAGATTACGTGAAGATGCAGTGGATGATG
CTGCAGCAAGAACAGCCGGAAGATTTCGTGATTGCCACCGGCGTTCAG
TACAGCGTTCGTCAGTTCGTGGAAATGGCCGCCGCCCAGCTGGGCATT
AAACTGCGTTTCGAAGGTACCGGCGTGGAGGAGAAAGGTATTGTGGTT
AGCGTGACCGGCCATGATGCCCCGGGCGTTAAACCGGGCGATGTTATC
ATCGCCGTGGATCCGCGCTATTTTCGCCCGGCCGAAGTTGAAACACTG
CTGGGCGATCCTACCAAAGCCCACGAAAAGCTGGGTTGGAAGCCCGAA
ATTACTTTACGCGAAATGGTTAGCGAGATGGTTGCCAATGATCTGGAA
GCCGCCAAAAAGCACTCTTTACTGAAAAGCCATGGCTACGATGTGGCC
ATTGCACTGGAAAGCTGA
Yp DmhA: AA
(SEQ ID NO: 13)
MNNVLITGFTGQVGSQLADYILENTDDHVIGMMRWQESMDNIYHLTDR
INKKDRISIQYADLNDLMSLYNLIDTVRPKFIFHLAAQSFPRTSFDIP
IETLQTNIIGTANLLECIRKLKQQDGYDPVVHVCSSSEVYGRAKVGEA
LNEDTQFHGASPYSISKIGTDYLGQFYGEAYGIRTFVTRMGTHTGPRR
SDVFFESTVAKQIALIEAGHQEPKLKVGNLASVRTFQDARDAVRAYYL
LALESGKGNIPNGEVENIAGDEAFKLPEVIELLLSFSTRNDIEVVTDT
DRLRPIDADYQMFDSTKIKSYINWKPEIKAADMFRDLLQHWRNEIASG
RIPLNR
Yp DmhA: DNA
(SEQ ID NO: 14)
ATGAACAATGTTCTGATTACGGGTTTCACCGGGCAGGTAGGTTCGCAG
CTTGCCGATTACATTCTGGAAAACACCGACGATCATGTGATCGGGATG
ATGCGCTGGCAGGAGAGCATGGACAATATTTATCATTTAACCGACCGC
ATCAACAAAAAAGATCGGATTTCAATCCAATACGCGGATCTGAATGAC
CTTATGTCTCTGTATAATCTGATAGACACGGTCCGGCCGAAATTCATT
TTCCATTTAGCGGCACAGAGCTTTCCGCGCACGTCCTTTGACATCCCA
ATCGAAACCCTGCAAACGAATATTATCGGCACTGCGAACCTGTTGGAG
TGTATTCGCAAACTGAAACAGCAAGACGGGTACGACCCGGTTGTTCAT
GTCTGTAGCTCCAGCGAAGTGTATGGGCGCGCGAAAGTGGGTGAGGCC
TTAAATGAAGATACGCAATTTCACGGCGCCAGCCCGTATTCCATTAGC
AAGATTGGCACGGATTATCTTGGTCAGTTTTATGGCGAAGCGTACGGC
ATTCGCACGTTTGTTACCAGGATGGGCACCCATACGGGTCCGCGTCGC
TCGGACGTGTTTTTCGAAAGCACCGTTGCCAAACAGATCGCCCTGATC
GAGGCGGGCCATCAAGAGCCAAAGTTAAAAGTCGGCAATTTGGCCTCG
GTACGCACGTTTCAAGATGCCCGCGATGCCGTGCGCGCTTACTATCTC
CTGGCCCTTGAATCCGGAAAAGGCAATATTCCGAACGGTGAGGTCTTT
AACATCGCGGGGGACGAAGCGTTCAAACTGCCGGAAGTCATTGAACTG
CTGCTGTCCTTCTCGACTCGTAATGATATTGAGGTTGTTACCGATACC
GATCGCTTACGTCCAATCGACGCCGATTATCAGATGTTTGACTCGACC
AAAATCAAATCATATATCAATTGGAAACCGGAAATCAAGGCAGCGGAC
ATGTTTCGCGATCTACTGCAACATTGGCGCAATGAGATCGCCAGTGGT
CGTATTCCGCTTAATCGCTAA
Yp DmhB: AA
(SEQ ID NO: 15)
MTKVFILGSNGYIGNNLMESLCDNIEVITVGRSNADIYINLESDDFQS
LLNKVEFKDTVIFLSAISSPDECNNNYDYSYKINVKNTISLISLLLAK
NVRVMFSSSDAVFGATQNLCDENSEKKPFGKYGEMKSEVEDYFTLEDD
FFVVRFSYVLGRNDKFSMMIKEFYEQGKILDVFDGFERNVISINDVTA
GIKNIICDWDSIKTRIVNFSGNELVSRQDIVNALVKEKYLNLKYKFTA
APESFWVGRPKKIHTKSNYLESILNRKLESYLEVIKE
Yp DmhB: DNA
(SEQ ID NO: 16)
ATGACGAAAGTTTTCATTCTGGGCTCAAATGGTTACATAGGTAATAAC
CTGATGGAGTCGCTGTGTGATAATATTGAGGTGATCACGGTCGGTCGT
TCAAACGCTGATATATACATTAACCTTGAATCCGACGATTTCCAGTCT
CTGCTGAACAAAGTAGAGTTTAAAGATACAGTGATCTTCCTGAGCGCG
ATCAGTAGCCCGGACGAATGCAATAATAACTATGATTATAGCTATAAA
ATTAATGTGAAAAATACCATAAGCCTGATTAGCCTCTTACTAGCTAAA
AACGTTCGCGTGATGTTCTCAAGCAGCGACGCGGTATTTGGCGCTACG
CAAAATCTGTGCGATGAAAATTCCGAAAAAAAACCCTTTGGAAAGTAT
GGCGAAATGAAAAGCGAAGTTGAAGATTATTTCACCCTTGAGGATGAT
TTCTTTGTGGTCCGCTTCAGCTATGTGCTGGGGCGAAACGATAAATTT
AGCATGATGATCAAAGAGTTTTACGAACAGGGTAAAATACTGGATGTG
TTTGATGGCTTTGAACGTAACGTGATTAGCATAAATGACGTGACAGCG
GGGATCAAAAACATCATTTGTGACTGGGATTCTATCAAAACTCGTATC
GTCAATTTTTCCGGCAACGAATTAGTTTCTCGCCAGGACATCGTTAAT
GCGCTGGTGAAGGAAAAATACCTGAACCTCAAATACAAATTTACCGCC
GCCCCCGAGTCGTTCTGGGTTGGCCGTCCCAAAAAGATTCACACCAAA
AGCAATTACCTGGAATCGATTTTAAACCGTAAACTGGAAAGTTACCTG
GAGGTCATCAAAGAGTAA
Cp MlghC: AA
(SEQ ID NO: 17)
MSKKVLITGGAGYIGSVLTPILLEKGYEVCVIDNLMFDQISLLSCFHN
KNFTFINGDAMDENLIRQEVAKADIIIPLAALVGAPLCKRNPKLAKMI
NYEAVKMISDFASPSQIFIYPNTNSGYGIGEKDAMCTEESPLRPISEY
GIDKVHAEQYLLDKGNCVTFRLATVFGISPRMRLDLLVNDFTYRAYRD
KFIVLFEEHFRRNYIHVRDVVKGFIHGIENYDKMKGQAYNMGLSSANL
TKRQLAETIKKYIPDFYIHSANIGEDPDKRDYLVSNTKLEATGWKPDN
TLEDGIKELLRAFKMMKVNRFANFN
Cp MIghC: DNA
(SEQ ID NO: 18)
ATGTCCAAAAAGGTGCTGATCACCGGCGGCGCGGGCTATATCGGAAGT
GTCCTGACCCCGATCCTGTTAGAAAAAGGCTATGAAGTCTGTGTTATC
GACAATCTGATGTTTGACCAGATTTCTCTGCTTTCCTGTTTTCATAAT
AAGAATTTCACGTTCATAAACGGGGATGCGATGGATGAAAATCTGATT
CGCCAGGAAGTAGCCAAAGCCGATATTATCATTCCGCTGGCGGCACTG
GTCGGGGCGCCTCTGTGTAAACGCAACCCGAAACTGGCTAAAATGATC
AACTACGAGGCAGTTAAGATGATTAGCGATTTTGCCTCCCCATCGCAG
ATCTTTATTTACCCAAACACCAATAGCGGTTACGGGATCGGCGAGAAA
GATGCGATGTGCACCGAAGAATCGCCGCTGCGTCCGATTTCCGAGTAT
GGGATCGATAAAGTGCATGCTGAACAGTACCTGCTGGATAAAGGTAAC
TGCGTGACCTTTCGTTTAGCAACAGTCTTTGGAATTTCACCGCGTATG
CGCCTTGATCTGCTCGTGAATGATTTTACATACCGCGCTTATCGTGAC
AAATTTATCGTTTTATTCGAAGAGCACTTTCGCCGCAACTATATTCAC
GTTCGTGATGTCGTGAAAGGCTTCATCCATGGGATAGAGAACTATGAT
AAAATGAAAGGCCAAGCGTACAACATGGGTCTGAGCTCGGCCAACCTA
ACCAAGCGCCAACTGGCCGAAACCATTAAGAAATATATTCCAGACTTC
TACATCCATTCAGCGAACATTGGAGAAGATCCGGATAAACGCGACTAT
CTGGTTTCGAATACGAAGTTGGAAGCCACCGGTTGGAAACCTGATAAT
ACTCTTGAGGATGGCATCAAAGAACTGTTACGTGCTTTTAAAATGATG
AAGGTTAACCGCTTTGCGAATTTTAATTAA
Os GME: AA
(SEQ ID NO: 19)
MGSSEKNGTAYGEYTYAELEREQYWPSEKLRISITGAGGFIGSHIARR
LKSEGHYIIASDWKKNEHMTEDMFCHEFHLVDLRVMDNCLKVTNGVDH
VFNLAADMGGMGFIQSNHSVIMYNNTMISFNMLEAARINGVKRFFYAS
SACIYPEFKQLETNVSLKESDAWPAEPQDAYGLEKLATEELCKHYTKD
FGIECRVGRFHNIYGPFGTWKGGREKAPAAFCRKAQTSTDRFEMWGDG
LQTRSFTFIDECVEGVLRLTKSDFREPVNIGSDEMVSMNEMAEIILSF
EDRELPIHHIPGPEGVRGRNSDNTLIKEKLGWAPTMKLKDGLRFTYFW
IKEQIEKEKTQGVDIAGYGSSKVVSTQAPVQLGSLRAADGKE
Os GME: DNA
(SEQ ID NO: 20)
ATGGGCTCCAGTGAGAAGAACGGAACTGCCTATGGCGAATATACATAC
GCTGAGTTAGAACGCGAGCAGTATTGGCCATCGGAGAAATTACGGATT
TCAATTACCGGGGCCGGCGGCTTCATCGGCTCCCACATCGCGCGACGA
CTGAAGAGTGAAGGTCATTATATTATCGCCTCGGACTGGAAGAAGAAC
GAACACATGACCGAGGACATGTTTTGTCACGAGTTCCATCTGGTGGAC
CTTCGAGTGATGGATAACTGTTTAAAAGTGACGAATGGCGTTGACCAT
GTATTTAATCTGGCAGCCGATATGGGCGGGATGGGCTTTATTCAATCG
AACCATTCGGTGATTATGTATAATAACACGATGATTTCGTTCAACATG
TTAGAAGCGGCCCGTATTAACGGCGTTAAACGTTTCTTCTATGCATCT
TCAGCTTGCATTTATCCAGAGTTCAAACAGCTTGAAACCAATGTGTCT
CTGAAGGAATCTGATGCGTGGCCCGCAGAACCACAGGACGCATACGGC
TTAGAAAAGCTGGCGACCGAGGAACTCTGTAAACACTACACCAAAGAT
TTCGGCATTGAGTGCCGCGTAGGTCGCTTTCATAACATTTATGGGCCA
TTTGGCACCTGGAAAGGTGGTCGCGAAAAGGCGCCAGCGGCCTTCTGT
CGAAAAGCACAGACATCCACCGACCGTTTCGAAATGTGGGGTGATGGC
TTGCAAACACGGTCTTTTACATTCATTGACGAATGCGTTGAGGGTGTT
CTGAGATTGACAAAATCGGATTTTCGTGAGCCTGTTAACATTGGCAGC
GACGAGATGGTGAGTATGAACGAAATGGCCGAGATCATTCTGTCTTTT
GAAGATCGCGAACTGCCTATTCACCATATTCCGGGACCGGAAGGTGTA
CGTGGCCGCAATTCGGACAATACTCTGATCAAAGAAAAGCTGGGCTGG
GCTCCGACCATGAAATTAAAAGACGGGCTCCGTTTCACTTACTTCTGG
ATTAAAGAGCAGATTGAGAAAGAGAAAACGCAAGGGGTTGACATTGCC
GGTTACGGCAGCAGTAAAGTTGTTAGTACCCAGGCCCCGGTGCAACTT
GGTTCTCTGCGCGCAGCAGACGGGAAAGAATAA
FutC 1: AA
(SEQ ID NO: 21)
MVSIILRGGLGNQLFQYATGRAHSLRTNSTLFVNLSKLDSNLGPDVAK
RSLHLEAFDLPVEYVDNETSHSFGRTIRRRIPQVVASINQLLATHLFK
LYVEDQSLTFDPNVPNLPGNVTLDGYWQSERYFTEFTETLRREITVRN
PVSGENQRWYDLISDTGSVSVHVRRGDYVDLGWALPPSYYRNALNQIQ
DETDVTDLFFFSDNIDWIRTNQKDLVPDHSDTNVHYVECNDGETAHED
LRLMRACDHHIVANSSFSWWGAWLDNSETKIVIAPDYWVHDPVNHLDI
IPDRWDTVSW
FutC 1: DNA
(SEQ ID NO: 22)
ATGGTCTCGATAATCCTACGCGGTGGACTCGGCAACCAACTATTCCAG
TACGCGACGGGACGCGCACACTCACTCCGAACTAATTCTACTCTTTTT
GTAAACCTCTCTAAACTTGACTCGAACCTTGGCCCCGACGTAGCGAAA
CGATCGCTACATCTTGAGGCGTTCGATCTTCCAGTTGAATATGTAGAT
AATGAGACAAGCCACAGTTTTGGCAGGACGATACGCAGACGGATCCCG
CAGGTCGTCGCAAGTATAAACCAGTTACTAGCGACACATCTCTTCAAA
TTGTACGTCGAAGATCAGTCACTGACGTTCGATCCGAATGTCCCTAAT
CTACCTGGAAACGTCACACTCGACGGTTACTGGCAATCCGAACGCTAT
TTTACAGAGTTTACCGAGACGCTTCGGCGTGAAATTACGGTTCGTAAT
CCTGTGTCTGGTGAAAACCAACGGTGGTACGACCTCATCTCCGATACT
GGCTCAGTAAGTGTACACGTCCGTCGTGGAGACTACGTTGATCTCGGC
TGGGCACTTCCACCGTCCTACTACAGAAATGCCCTCAATCAGATTCAG
GATGAAACTGACGTGACAGATCTGTTTTTTTTCTCCGACAACATTGAC
TGGATTCGTACCAACCAGAAAGACCTTGTGCCGGATCACAGCGATACC
AACGTACACTACGTCGAGTGTAACGATGGAGAAACGGCCCACGAGGAT
CTCCGTCTGATGCGAGCCTGTGATCACCATATCGTCGCCAACAGCAGC
TTCAGTTGGTGGGGTGCGTGGCTGGATAATTCTGAGACGAAAATTGTC
ATCGCTCCCGACTATTGGGTTCATGACCCGGTCAATCATCTCGATATT
ATTCCCGATCGATGGGATACCGTCAGTTGGTAG
FutC 2: AA
(SEQ ID NO: 23)
MIYTRITSGLGNQMFQYAIAYSYSRKYDMPLILDLTNFKISKKRTYQL
DKFKLNDYKKITFKNAPLEIKIFWLVEVLNMISIKLRKKEMKRKNNYN
LKSTQFICEKYKEKYNINFDLINKSLYLSGFWQSPLYFENYRDELIQQ
FSPNYVLSNKLKEYETKIINCRSVSVHIRRGDFLQHGLFKDVDYQKKA
ITYLEKKLDNPIFFFFSDDIEWTKEKFKNQKNCFFVSSDSKNSGIEEM
YLMSKCENNIIANSTFSWWGAWLNQNQNKIVIAPSTGFGNKDILPKSW
YTI
FutC 2: DNA
(SEQ ID NO: 24)
ATGATATATACACGAATAACGAGCGGTTTAGGGAACCAAATGTTTCAG
TATGCTATTGCGTATTCGTATTCTAGGAAATATGATATGCCACTTATT
CTTGATCTTACAAATTTTAAAATTTCAAAAAAGAGAACCTATCAATTA
GATAAATTCAAACTTAATGATTATAAAAAGATAACATTTAAAAATGCT
CCATTAGAAATAAAAATATTTTGGTTGGTAGAGGTTTTAAACATGATT
TCTATTAAACTAAGGAAAAAAGAAATGAAAAGAAAAAATAATTATAAC
TTGAAATCAACTCAATTTATATGCGAGAAATATAAAGAAAAATATAAC
ATAAACTTTGATCTCACAAATAAATCACTTTATTTATCTGGATTTTGG
CAAAGTCCTTTATATTTTGAAAACTATAGAGATGAATTAATACAACAG
TTTTCTCCTAATTATGTTTTATCAAATAAGTTAAAAGAATATGAAACT
AAGATAATAAACTGTAGAAGCGTTTCTGTTCATATTAGAAGAGGAGAT
TTTTTACAACATGGTTTATTTAAAGATGTAGATTACCAAAAGAAAGCT
ATAACTTATTTAGAAAAGAAATTAGATAACCCTATTTTTTTCTTTTTT
TCAGACGATATTGAATGGACAAAAGAAAAATTTAAAAATCAAAAAAAT
TGTTTTTTTGTATCTTCAGATTCAAAAAATTCTGGTATAGAAGAAATG
TATCTTATGTCTAAGTGTGAGAACAATATCATTGCAAATAGTACTTTT
AGTTGGTGGGGAGCATGGTTAAATCAAAACCAAAATAAAATTGTAATA
GCACCAAGCACTGGTTTTGGTAATAAAGATATATTACCAAAATCTTGG
TATACAATTTAG
FutC 3: AA
(SEQ ID NO: 25)
MLVVSMGCGLGNQMFEYAFYKHLCKKYTSEIIKLDIRHAFPFAHNGIE
LFDIFDLSGEVASKQEVLFLTSGYGLHGVGFEYKTIFHRIGEKVRKLF
SLTPQTMKIQDDYTEYYNEFFNVMPGKSVYYLGVFANYHYFKEIQYDI
KNIYKFPTIDDLKNKRYAEKMENCNSVSIHVRRGDYVSEGVKLTPLSF
YRKAILKIEEKVKNAHFFVFADDVEYARSLFPDNDHYTFVEGNNGKNS
FRDMQLMSLCKHNITANSTFSFWGAFLNSNPSKIVIAPNLPYTGAKYP
FVCDDWVLI
FutC 3: DNA
(SEQ ID NO: 26)
ATGCTTGTTGTTAGTATGGGGTGTGGTTTGGGGAATCAGATGTTTGAA
TATGCATTTTATAAGCATTTATGTAAAAAATATACAAGCGAGATAATT
AAACTTGATATAAGACACGCATTTCCGTTTGCTCATAATGGAATTGAG
CTATTTGATATTTTTGATTTATCTGGAGAAGTTGCGAGTAAGCAAGAA
GTTCTGTTTTTGACGTCAGGGTATGGCCTACATGGTGTTGGGTTTGAA
TATAAAACTATTTTTCACAGAATAGGAGAAAAAGTAAGAAAACTTTTC
TCGTTGACACCACAAACTATGAAAATTCAAGATGATTATACAGAGTAT
TATAATGAATTTTTTAATGTGATGCCCGGTAAATCGGTGTACTATCTA
GGTGTTTTCGCAAATTACCATTATTTTAAGGAGATACAATATGATATA
AAAAATATATACAAATTTCCTACTATAGATGATCTGAAAAACAAAAGA
TATGCAGAAAAAATGGAAAATTGTAATTCAGTATCTATTCACGTTAGA
AGAGGAGATTATGTAAGCGAAGGAGTAAAGCTTACGCCCTTATCATTT
TATAGAAAAGCTATTTTAAAGATTGAAGAAAAGGTAAAAAATGCTCAT
TTTTTTGTCTTCGCAGATGATGTAGAGTATGCTCGTTCGCTTTTTCCT
GATAATGATCATTATACGTTTGTAGAAGGAAATAATGGCAAGAATAGT
TTTCGCGATATGCAACTTATGAGTTTATGTAAGCATAATATCACAGCA
AACAGTACGTTTAGCTTTTGGGGAGCATTTTTAAATTCAAATCCTAGT
AAAATAGTTATAGCGCCCAACTTGCCATATACAGGTGCAAAATATCCA
TTTGTATGTGATGATTGGGTGTTGATATAG
FutC 4: AA
(SEQ ID NO: 27)
MIITRLIGGLGNQIFQYAVGRAVAARTNTPLLLDASGFPGYELRRYEL
DGENVRAELVSAAQLARVGVTASAPHSLLERIKLRFFSQSTQKLPLRE
PILREASFTYDTRIEYVQAPIYLDGYWQSERYFSAIRMQLLQELTLKN
EWGVGNEDMFAQIQAAGLGAVSLHVRRGDYVINSHTATYHGVCSLDYY
RAAVAYIAERVAAPHFFIFSDDHDWVSTNLQTGFPTTFVSVNSADHGI
YDMMLMKTCRHHVIANSSFSWWGAWLNPYQDKIVVAPQRWFSGASHDI
SDLIPASWIRI
FutC 4: DNA
(SEQ ID NO: 28)
ATGATCATTACTCGTCTAATTGGTGGTCTCGGCAATCAAATATTCCAA
TATGCAGTGGGTCGCGCCGTCGCCGCGCGCACGAACACGCCTCTGCTG
CTGGACGCTTCCGGTTTTCCGGGTTATGAATTGCGGCGTTACGAGCTC
GATGGTTTCAACGTCCGCGCCGAACTGGTCTCGGCTGCGCAACTGGCC
CGCGTTGGGGTAACCGCCAGCGCTCCCCACTCTTTGCTGGAGCGAATC
AAGCTCCGTTTTTTCTCTCAATCCACGCAGAAGCTACCTCTGCGGGAG
CCGATCCTGCGCGAAGCCAGCTTTACCTACGATACCCGCATTGAATAC
GTACAGGCACCGATCTATCTGGATGGATATTGGCAGAGCGAGCGTTAT
TTCTCGGCTATCCGCATGCAGCTGCTGCAGGAGCTAACTCTCAAAAAC
GAGTGGGGAGTAGGAAACGAAGATATGTTTGCTCAGATCCAGGCTGCC
GGACTCGGCGCCGTGTCGCTGCATGTCCGCCGGGGCGATTATGTGACA
AATTCCCACACGGCTACTTATCACGGAGTATGCTCGCTGGATTACTAC
CGTGCGGCAGTGGCTTACATCGCCGAACGCGTGGCAGCGCCGCATTTT
TTCATCTTTTCCGATGACCACGACTGGGTCAGCACCAATCTGCAGACC
GGATTCCCGACCACTTTTGTCTCCGTTAATTCCGCTGACCATGGCATC
TACGACATGATGCTGATGAAGACCTGCCGTCATCACGTAATCGCCAAT
AGCTCCTTCAGCTGGTGGGGCGCCTGGTTGAATCCTTATCAAGACAAG
ATCGTGGTTGCGCCGCAACGCTGGTTTAGCGGCGCATCGCACGACATA
AGTGACCTCATTCCGGCTTCTTGGATCCGAATATGA
FutC 5: AA
(SEQ ID NO: 29)
MIILQMSGGLGNQMFQYALYLKLKKLGREVKFDDETSYELDNARPVQL
AVFDITYPRATRQEVTDMRDSSPAWKDRIRRKLKGRNLKQYTEANYSY
DEHVFELDDTYLRGYFQTEKYFSDIRDEIYKTYTMRKDLITEQTTQYE
EDILSHENSVSIHIRRGDYMTIEGGEIYAGICTDEFYDSAIKYVLERH
PDAVFYLFTNDSSWAEYFCNIHSDVNIHVVEGNTEYFGYLDMYLMSRC
KHHIVANSSFSWWGAWLGRDADGMVIAPDPWFNCSNCADIHTDRMILI
DPKGELLTDDKGVRNESEE
FutC 5: DNA
(SEQ ID NO: 30)
ATGATCATATTACAGATGAGCGGCGGACTCGGGAATCAGATGTTCCAG
TACGCTTTATATCTGAAACTTAAGAAGCTCGGCAGAGAAGTCAAATTC
GATGATGAGACGAGCTATGAACTTGATAATGCGAGACCGGTACAGCTT
GCCGTTTTTGACATAACCTATCCTCGTGCGACGAGACAGGAAGTCACC
GACATGCGCGATTCTTCCCCCGCATGGAAGGACAGGATCAGACGTAAG
TTAAAAGGCCGGAACCTGAAGCAGTACACCGAAGCAAACTACAGTTAC
GATGAACATGTATTCGAGCTGGACGATACGTATCTTCGGGGATATTTT
CAGACCGAGAAGTATTTTTCCGATATCAGGGATGAGATCTACAAGACA
TACACGATGCGTAAGGATCTGATCACCGAACAGACTACGCAGTATGAG
GAAGACATATTAAGTCATGAAAACAGTGTGAGCATCCATATACGCCGC
GGCGATTACATGACCATAGAGGGCGGAGAGATATATGCCGGCATCTGT
ACGGACGAATTTTATGACTCAGCCATAAAGTATGTTCTTGAGAGACAT
CCGGATGCTGTATTTTATCTTTTTACCAATGACAGTTCATGGGCGGAG
TATTTCTGTAACATACATTCCGATGTGAACATTCATGTCGTCGAAGGC
AATACCGAATATTTCGGATACCTGGACATGTACCTGATGAGCAGGTGT
AAGCATCATATCGTGGCAAACAGTTCTTTTTCATGGTGGGGAGCATGG
CTCGGCAGGGATGCGGACGGTATGGTCATAGCACCGGATCCGTGGTTT
AACTGCAGCAACTGTGCGGACATCCACACCGACAGGATGATCCTGATC
GATCCCAAGGGTGAGCTGTTGACAGATGATAAGGGCGTAAGAAATGAG
TCAGAAGAATAA
FutC 6: AA
(SEQ ID NO: 31)
MIIARLFGGLGNQMFQYAAGKSLAERLGAELALDFRIIDERGTRRLTD
VEDLDIVPATNLPATKHENLLRYGLWRAFGQSPKFRRETGLGYNAAFA
EWSDDTYLHGYWQSEQYFSAISDHLRRVFQAVPAPSKENGAIADDIRD
CSAISLHVRRGDYLALGAHGVCDEAYYNAALSHIAPQLNQDPRVFVFS
DDPQWAKDNLPLPFEKIVVDLNGPTTDYEDLRLMSLCDHNIIANSSFS
WWGAWLNANPDKIVTAPANWFADAKLDNPDILPEGWQRITP
FutC 6: DNA
(SEQ ID NO: 32)
ATGATCATTGCAAGACTGTTCGGGGGTCTGGGAAACCAGATGTTCCAA
TATGCCGCAGGAAAGTCACTCGCTGAACGATTGGGTGCTGAGCTTGCA
CTCGATTTTAGAATAATTGATGAACGTGGCACCCGCCGCCTGACAGAC
GTGTTTGACCTCGACATTGTGCCGGCAACAAACCTTCCCGCCACCAAA
CATGAAAATCTTCTGAGATATGGGCTATGGCGTGCATTCGGTCAGTCC
CCAAAATTTCGACGCGAGACAGGTCTTGGATACAATGCCGCCTTCGCG
GAATGGAGCGACGACACTTATCTGCATGGCTATTGGCAGTCAGAGCAG
TATTTTTCGGCAATCTCCGACCATTTACGCCGCGTGTTTCAAGCGGTG
CCTGCACCGTCGAAAGAGAATGGTGCAATTGCAGATGACATTCGCGAC
TGCAGCGCGATCTCGCTGCATGTGCGCCGCGGGGACTACCTTGCCCTT
GGGGCGCATGGCGTCTGTGATGAAGCCTATTACAATGCGGCGTTGTCT
CATATCGCACCCCAATTGAACCAAGATCCACGTGTCTTTGTGTTTTCC
GACGATCCGCAATGGGCCAAAGACAACCTTCCCCTGCCGTTTGAAAAG
ATTGTCGTCGATCTGAACGGCCCGACAACCGACTATGAAGACCTGCGA
TTGATGAGCCTGTGCGACCACAACATCATCGCAAACAGTTCATTTTCC
TGGTGGGGCGCATGGTTAAACGCAAACCCTGACAAGATTGTCACCGCG
CCAGCAAACTGGTTCGCGGATGCAAAGTTGGACAACCCCGACATTCTC
CCTGAAGGCTGGCAAAGGATCACCCCCTGA
FutC 7: AA
(SEQ ID NO: 33)
MYFQKKMIIVKLSGGLGNQLFQYALGRQLSIVNHTDLKMDTTNFSQPS
GGTTRTFALGSFNIHAAQANKDEIKLLAGEPNRIFQRVRRKIGLMPIH
YFKEPHFHFYQPVLSLQDGVYLDGYWQSEKYFAEIADRIREDLKPVGS
FSNQYETFKQSIKQSVSVSVHIRRGDYTTTSKANRYLKPCEALYYQTA
VEYLTKRISNLVFFVFSDDIEWAKAHIHFGFPMQYVEGNSAQEDLLLI
ASCQHHIIANSTFSWWGAWLNPHPDKIVIAPQKWFSTERFDTKDLLPE
SWILL
FutC 7: DNA
(SEQ ID NO: 34)
ATGGCTTACTTCCAGAAAAAGATGATCATCGTTAAACTGAGTGGCGGT
CTGGGCAATCAGCTGTTTCAGTATGCCCTGGGTCGTCAGCTGAGCATT
GTTAATCATACCGATCTGAAAATGGATACCACCAATTTTAGCCAGCCG
AGCGGTGGCACCACCCGTACCTTTGCACTGGGCAGCTTTAATATTCAT
GCCGCACAGGCCAATAAGGATGAAATTAAGCTGCTGGCAGGCGAACCG
AATCGCATTTTTCAGCGTGTTCGTCGCAAAATTGGCCTGATGCCGATT
CATTATTTTAAAGAACCGCATTTTCACTTCTACCAGCCGGTGCTGAGT
CTGCAGGATGGCGTGTATCTGGATGGCTATTGGCAGAGTGAAAAATAT
TTTGCCGAAATTGCCGATCGCATTCGCGAAGATCTGAAACCGGTGGGT
AGTTTTAGCAATCAGTATGAAACCTTTAAGCAGAGCATTAAGCAGAGC
GTTAGCGTTAGCGTGCATATTCGTCGTGGTGACTATACCACCACCAGT
AAAGCCAATCGCTATCTGAAACCGTGTGAAGCACTGTATTATCAGACC
GCAGTTGAATATCTGACCAAACGTATTAGCAATCTGGTTTTCTTTGTG
TTTAGTGATGATATTGAGTGGGCCAAAGCCCATATTCATTTTGGTTTT
CCGATGCAGTATGTTGAAGGCAATAGTGCCCAGGAAGATCTGCTGCTG
ATTGCAAGCTGCCAGCATCATATTATTGCCAATAGTACCTTTAGCTGG
TGGGGTGCATGGCTGAATCCGCATCCGGATAAAATTGTGATTGCCCCG
CAGAAATGGTTTAGTACCGAACGTTTTGATACCAAAGATCTGCTGCCG
GAAAGCTGGATTCTGCTGTAA
FutC 8: AA
(SEQ ID NO: 35)
MIISNIIGGLGNQMFQYAMARSLSLELKSDLLLDISSYDSYPLHQGYE
LDRVFKVRSSLAKVEDVKSVLGWQQNLFIHRVLRRPQFSWLRKKSLAI
EPFFQYWEGVNFLPKNCYLFGYWQSEKYFNKFSEVIRQDFSFDSNMSE
ENSFYSERIRKSNSVSVHIRRGDYLNNSVYASCSLEYYRSAIAHVSAR
SGNPVFFVFSDDIEWVKDNLEFEAESYFVAHNKAGESYNDMRLMSYCK
HHVIANSSFSWWGAWLNPSPEKIVIAPKQWFTDGTNTKDLIPSEWMVL
FutC 8: DNA
(SEQ ID NO: 36)
ATGGCTATCATCAGTAACATCATCGGCGGTCTGGGTAATCAGATGTTT
CAGTATGCAATGGCTCGTAGTCTGAGTCTGGAACTGAAAAGCGATCTG
CTGCTGGATATTAGCAGTTATGATAGCTATCCGCTGCATCAGGGCTAT
GAACTGGATCGTGTTTTTAAAGTTCGTAGTAGCCTGGCCAAAGTGGAA
GATGTGAAAAGTGTGCTGGGCTGGCAGCAGAATCTGTTTATTCATCGC
GTGCTGCGTCGTCCGCAGTTTAGCTGGCTGCGTAAAAAATCTCTGGCC
ATTGAACCGTTTTTCCAGTATTGGGAAGGCGTTAATTTTCTGCCGAAA
AATTGTTATCTGTTCGGTTATTGGCAGAGCGAAAAATATTTTAATAAG
TTCAGCGAGGTTATTCGTCAGGATTTTAGTTTTGATAGTAACATGAGT
GAGGAAAATAGTTTTTACAGTGAACGTATTCGCAAAAGCAATAGCGTG
AGTGTTCATATTCGTCGTGGTGACTATCTGAATAATAGCGTTTATGCC
AGTTGTAGTCTGGAATATTATCGTAGTGCCATTGCACATGTGAGCGCC
CGCAGCGGTAATCCGGTGTTTTTCGTTTTTAGTGATGATATTGAGTGG
GTGAAAGATAATCTGGAATTTGAAGCAGAAAGTTATTTCGTTGCCCAT
AATAAGGCAGGCGAAAGTTATAATGATATGCGTCTGATGAGTTATTGT
AAACATCATGTGATTGCCAATAGTAGCTTTAGCTGGTGGGGTGCCTGG
CTGAATCCGAGCCCGGAAAAAATTGTTATTGCACCGAAACAGTGGTTT
ACCGATGGCACCAATACCAAAGATCTGATTCCGAGCGAATGGATGGTT
CTGTAA
FutC 9: AA
(SEQ ID NO: 37)
MVIIKMMGGLGNQMFQYALYKAFEQKHIDVYADLAWYKNKSVKFELYN
FGIKINVASEKDINRLSDCQADFVSRIRRKIFGKKKSFVSEKNDSCYE
NDILRMDNVYLSGYWQTEKYFSNTREKLLEDYSFALVNSQVSEWEDSI
RNKNSVSIHIRRGDYLQGELYGGICTSLYYAEAIEYIKMRVPNAKFFV
FSDDVEWVKQQEDFKGFVIVDRNEYSSALSDMYLMSLCKHNIIANSSF
SWWAAWLNRNEEKIVIAPRRWLNGKCTPDIWCKKWIRI
FutC 9: DNA
(SEQ ID NO: 38)
ATGGTTATTATCAAGATGATGGGTGGTCTGGGCAATCAGATGTTTCAG
TATGCCCTGTATAAAGCATTTGAACAGAAACATATCGACGTGTATGCC
GATCTGGCA
TGGTATAAAAATAAGAGCGTGAAATTTGAGCTGTATAATTTTGGCATT
AAGATCAATGTGGCCAGTGAAAAAGATATTAATCGTCTGAGCGATTGC
CAGGCAGATTTTGTTAGTCGCATTCGCCGCAAAATTTTTGGCAAAAAG
AAAAGTTTCGTGAGTGAAAAGAATGATAGTTGTTATGAAAACGACATC
CTGCGTATGGATAATGTGTATCTGAGCGGTTATTGGCAGACCGAAAAA
TATTTTAGCAATACCCGTGAAAAGCTGCTGGAAGATTATAGCTTTGCA
CTGGTTAATAGTCAGGTGAGCGAATGGGAAGATAGCATTCGTAATAAG
AATAGTGTTAGCATTCACATTCGCCGTGGTGACTATCTGCAGGGCGAA
CTGTATGGCGGCATTTGTACCAGTCTGTATTATGCCGAAGCCATTGAA
TATATTAAGATGCGCGTTCCGAATGCCAAATTTTTCGTTTTTAGTGAT
GACGTGGAATGGGTGAAACAGCAGGAAGATTTTAAAGGTTTTGTGATT
GTTGACCGTAATGAATATAGCAGTGCACTGAGTGATATGTATCTGATG
AGCCTGTGCAAACATAATATTATTGCCAATAGTAGCTTCAGCTGGTGG
GCAGCATGGCTGAATCGTAATGAAGAAAAAATTGTTATCGCGCCGCGC
CGTTGGCTGAATGGCAAATGTACCCCGGATATTTGGTGCAAAAAATGG
ATTCGCATTTAA
FutC 10: AA
(SEQ ID NO: 39)
MIIVRLCGGLGNQMFQYAAGLAAAHRIGSEVKFDTHWFDATCLHQGLE
LRRVFGLELPEPSSKDLRKVLGACVHPAVRRLLSRRLLRALRPKSLVI
QPHFHYWTGFEHLTDNVYLEGYWQSERYFSNIADIIRQQFRFVEPLDP
HNAALMDEMQSGVSVSLHIRRGDYFNNPQMRRVHGVDLSEYYPAAVAT
MIEKTNAERFYVFSDDPQWVLEHLKLPVSYTVVDHNRGAASYRDMQLM
SACRHHIIANSTFSWWGAWLNPRPDKVVIAPRHWFNVDVFDTRDLYCP
EWIVL
FutC 10: DNA
(SEQ ID NO: 40)
ATGGCTATCATCGTGCGTCTGTGCGGTGGTCTGGGTAATCAGATGTTT
CAGTATGCCGCAGGTCTGGCAGCCGCACATCGCATTGGTAGTGAAGTG
AAATTTGATACCCATTGGTTTGATGCAACCTGTCTGCATCAGGGCCTG
GAACTGCGTCGTGTGTTTGGTCTGGAACTGCCGGAACCGAGCAGCAAA
GATCTGCGCAAAGTTCTGGGTGCATGCGTGCATCCGGCAGTTCGCCGT
CTGCTGAGTCGCCGTCTGTTACGTGCACTGCGTCCGAAAAGTCTGGTT
ATTCAGCCGCATTTTCATTATTGGACCGGTTTTGAACATCTGACCGAT
AATGTTTATCTGGAAGGTTATTGGCAGAGCGAACGCTATTTTAGCAAT
ATTGCAGATATTATCCGCCAGCAGTTTCGCTTTGTGGAACCGCTGGAC
CCTCATAATGCCGCCCTGATGGATGAAATGCAGAGTGGCGTTAGTGTG
AGCCTGCATATTCGCCGTGGTGACTATTTTAATAATCCGCAGATGCGC
CGTGTTCATGGCGTTGATCTGAGTGAATATTATCCGGCCGCAGTGGCC
ACCATGATTGAAAAAACCAATGCCGAACGTTTTTATGTGTTTAGTGAT
GATCCGCAGTGGGTTCTGGAACATCTGAAACTGCCGGTTAGCTATACC
GTGGTTGATCATAATCGTGGTGCCGCAAGTTATCGCGATATGCAGCTG
ATGAGTGCATGTCGCCATCATATTATTGCAAATAGCACCTTTAGTTGG
TGGGGTGCATGGCTGAATCCGCGCCCGGATAAAGTGGTTATTGCCCCG
CGTCATTGGTTTAATGTGGATGTTTTTGATACCCGTGATCTGTATTGC
CCGGAATGGATTGTGCTGTAA
FutC 11: AA
(SEQ ID NO: 41)
MIISKLKGGLGNQLFQYAIGRKMALEQGVELKLELSFFERQNNKTQAR
DFGLSCFNIDASIASSEDIRMILGPHFLRPLKRRLSKMGIPLFRWNYV
RENSWAYHPEILKKKAPLILDGYWQSAAYFESIRDVLLSDFELKAECV
SDKLRLLQKQITTESSVALHVRRGDYVTNPIVAKEFGICSESYYEEAV
SYMKALEGEPVFFVFSDDIDWCKKHFGEKAGTFVFVSGNQDYEDLMLM
SACKHQIIANSSFSWWSAWLNKNPEKKVIAPKIWFADTQMYKTEHIVP
QEWIRI
FutC 11: DNA
(SEQ ID NO: 42)
ATGGCTATCATCAGCAAACTGAAAGGTGGCCTGGGCAATCAGCTGTTT
CAGTATGCCATTGGCCGCAAAATGGCCCTGGAACAGGGTGTTGAACTG
AAACTGGAACTGAGTTTCTTTGAACGTCAGAATAATAAGACCCAGGCC
CGTGATTTTGGTCTGAGTTGTTTTAATATTGACGCCAGCATTGCAAGT
AGCGAAGATATTCGTATGATTCTGGGCCCGCATTTTCTGCGTCCGCTG
AAACGTCGCCTGAGCAAAATGGGCATTCCGCTGTTTCGTTGGAATTAT
GTTCGCGAAAATAGTTGGGCCTATCATCCGGAAATTCTGAAAAAGAAA
GCACCGCTGATTCTGGATGGTTATTGGCAGAGTGCAGCCTATTTTGAA
AGCATTCGTGATGTTCTGCTGAGCGATTTTGAACTGAAAGCAGAATGC
GTTAGTGATAAACTGCGTCTGCTGCAGAAACAGATTACCACCGAAAGT
AGCGTGGCCCTGCATGTGCGCCGCGGTGACTATGTTACCAATCCGATT
GTTGCAAAAGAATTTGGTATTTGCAGTGAAAGTTACTATGAAGAAGCA
GTTAGTTATATGAAGGCACTGGAAGGTGAACCGGTTTTCTTTGTGTTT
AGCGATGATATTGATTGGTGTAAAAAGCATTTCGGCGAAAAAGCAGGT
ACCTTTGTGTTTGTGAGCGGTAATCAGGATTATGAAGATCTGATGCTG
ATGAGCGCATGTAAACATCAGATTATTGCAAATAGTAGCTTCAGCTGG
TGGAGCGCCTGGCTGAATAAGAATCCGGAAAAGAAAGTTATTGCACCG
AAAATTTGGTTTGCAGATACCCAGATGTATAAAACCGAACATATTGTT
CCGCAGGAATGGATTCGCATTTAA
FutC 12: AA
(SEQ ID NO: 43)
MITVSLIGGLGNQMFQYAAGKALAERHGVPLVLDLSGFRDYAVRSYLL
DRLHVPEAGGALGQAESFQKFAARFARAKWKGRIDRLLGQVGLPKIVA
SSQEYREPHFHYDPAFEALGPSAVLFGYFQSERYFGSISESLSDWFSA
REPFGDTAADMLARIETSPLAISVHVRRGDYLNPGTAEFHGILGESYY
RQALGRLERLCGQDSELFVFSDDPPAAEKVLDFASRSRLVHVRGDPER
PWEDMALMARCHHHIIANSSFSWWGAWLNRSPHKHVVAPRAWFAPAEL
EKTNTADLYPAEWILV
FutC 12: DNA
(SEQ ID NO: 44)
ATGGCTATCACCGTTAGTCTGATTGGCGGCCTGGGCAATCAGATGTTT
CAGTATGCAGCCGGCAAAGCCCTGGCAGAACGTCATGGTGTGCCGCTG
GTTCTGGATCTGAGTGGCTTTCGTGATTATGCAGTGCGCAGCTATCTG
CTGGATCGCCTGCATGTTCCGGAAGCCGGCGGCGCTCTGGGCCAAGCA
GAAAGCTTTCAGAAATTTGCCGCACGTTTTGCCCGCGCAAAATGGAAA
GGTCGCATTGATCGTCTGCTGGGCCAGGTGGGTCTGCCGAAAATTGTT
GCAAGCAGCCAGGAATATCGCGAACCGCATTTTCATTATGATCCGGCA
TTTGAAGCACTGGGTCCGAGCGCCGTGCTGTTTGGCTATTTTCAGAGC
GAACGTTATTTTGGTAGCATTAGTGAAAGCCTGAGTGATTGGTTTAGC
GCCCGTGAACCGTTTGGTGACACCGCCGCCGATATGCTGGCCCGTATT
GAAACCAGCCCGCTGGCCATTAGCGTGCATGTTCGCCGCGGTGACTAT
CTGAATCCGGGCACCGCCGAATTTCATGGTATTCTGGGTGAAAGCTAT
TATCGCCAGGCACTGGGTCGCCTGGAACGCCTGTGCGGTCAGGATAGT
GAACTGTTTGTGTTTAGTGATGATCCGCCGGCCGCAGAAAAAGTGCTG
GATTTTGCCAGTCGCAGCCGTCTGGTTCATGTTCGCGGCGATCCGGAA
CGCCCGTGGGAAGATATGGCACTGATGGCCCGCTGCCATCATCATATT
ATTGCAAATAGTAGTTTCAGCTGGTGGGGCGCCTGGCTGAATCGTAGT
CCGCATAAACATGTTGTGGCACCGCGTGCATGGTTTGCCCCGGCAGAA
CTGGAAAAAACCAATACCGCAGATCTGTATCCGGCCGAATGGATTCTG
GTTTAA
FutC 13: AA
(SEQ ID NO: 45)
MQLKRWPQLKPTDAAVFGSGKQTIMIIVKLMGGLGNQMFQYAAGRRLA
EKLGVKLKLDIEMFKDNTLRKYELGAFNIQECFAAVEEIERLTVVKRG
IVEKALDRVFKRPIRRPGGYVAEKYFVFDPSILQLPDQVYLDGYWQSE
KYFAEIETIIREEFTIKYPQTDKNKVLSDSIKSGNSVTVHVRRGDYVN
NPETNSLHGVCGIDYYQRCIDFIITKIANPHFFFFSDDPEWVKNNLKI
KYESTVVEHNGAEKCYEDLRLLSQGKYHIIANSTFSWWGAWLNKNPEK
MVVAPEKWFKKEDVNTKGFIPEDWIRL
FutC 13: DNA
(SEQ ID NO: 46)
ATGGCTCAGCTGAAACGTTGGCCGCAGCTGAAACCGACCGATGCAGCC
GTGTTTGGCAGTGGCAAACAGACCATTATGATTATTGTTAAACTGATG
GGTGGTCTGGGCAATCAGATGTTTCAGTATGCCGCCGGCCGCCGTCTG
GCCGAAAAACTGGGTGTTAAACTGAAACTGGATATTGAAATGTTCAAG
GATAACACCCTGCGCAAATATGAACTGGGCGCATTCAATATTCAGGAA
TGTTTTGCCGCAGTTGAAGAAATTGAACGTCTGACCGTTGTTAAACGC
GGTATTGTTGAAAAAGCCCTGGATCGCGTTTTTAAACGCCCGATTCGC
CGTCCGGGTGGTTATGTTGCCGAAAAATATTTTGTTTTCGACCCGAGT
ATTCTGCAGCTGCCGGATCAGGTTTATCTGGATGGCTATTGGCAGAGT
GAAAAATATTTCGCAGAAATTGAAACCATCATCCGCGAAGAATTCACT
ATTAAGTATCCGCAGACCGATAAAAATAAGGTTCTGAGCGATAGTATT
AAGAGCGGCAATAGCGTGACCGTGCATGTGCGTCGTGGTGACTATGTT
AATAATCCGGAAACCAATAGCCTGCATGGCGTGTGCGGTATTGATTAT
TATCAGCGCTGTATTGATTTCATTATCACCAAAATTGCGAACCCGCAT
TTCTTTTTCTTTAGTGATGATCCGGAATGGGTTAAAAATAATCTGAAA
ATTAAGTACGAGAGCACCGTGGTGGAACATAATGGCGCAGAAAAATGC
TATGAAGATCTGCGTCTGCTGAGTCAGGGTAAATATCATATTATTGCC
AATAGCACCTTCAGTTGGTGGGGTGCATGGCTGAATAAGAATCCGGAA
AAAATGGTTGTTGCCCCGGAAAAATGGTTTAAAAAAGAAGATGTGAAC
ACCAAAGGCTTTATTCCGGAAGATTGGATTCGTCTGTAA
FutC 14: AA
(SEQ ID NO: 47)
MIVIKLIGGLGNQMFQYATAKAIALHKNTTLKLDVSAFENYDLHDYSL
DHFNITAKKYQQPPKWLKKIQNKLKPKTYYNEESFRYNSFLFDSNAKT
ILLNGYFQSEQYFLKYREEIIKDFSITSPLKPETKALLQKVHKTNAVS
IHIRRGDFLKHDVHNTFKEEYYKKAMKTIESKIDNPTYYLFSDDMPWV
KLNFKSNFKTVYVDFNDAQTAFEDLVLMSNCKHNIIANSSFSWWAAWL
NTNPSKIVIAPEQWENGNKYDYTDVVPETWVKI
FutC 14: DNA
(SEQ ID NO: 48)
ATGGCTATCGTGATTAAGCTGATTGGTGGTCTGGGTAATCAGATGTTT
CAGTATGCCACCGCCAAAGCAATTGCCCTGCATAAAAATACCACCCTG
AAACTGGATGTTAGTGCCTTTGAAAATTATGATCTGCATGATTATAGC
CTGGATCATTTTAATATCACCGCAAAAAAGTACCAGCAGCCGCCGAAA
TGGCTGAAAAAGATTCAGAATAAGCTGAAACCGAAAACCTATTATAAC
GAAGAAAGTTTTCGCTATAACAGTTTTCTGTTTGATAGCAATGCCAAA
ACCATTCTGCTGAATGGTTATTTTCAGAGCGAACAGTATTTTCTGAAA
TATCGTGAAGAAATCATCAAGGATTTCAGTATTACCAGCCCGCTGAAA
CCGGAAACCAAAGCACTGCTGCAGAAAGTGCATAAAACCAATGCCGTT
AGCATTCATATTCGCCGTGGCGATTTTCTGAAACATGATGTTCATAAT
ACCTTCAAAGAGGAATATTACAAGAAGGCCATGAAAACCATTGAAAGC
AAAATTGATAACCCGACCTATTATCTGTTTAGTGATGATATGCCGTGG
GTTAAACTGAATTTTAAAAGCAATTTCAAGACCGTGTACGTGGATTTT
AATGATGCCCAGACCGCATTTGAAGATCTGGTGCTGATGAGCAATTGT
AAACATAATATTATCGCCAACAGCAGTTTTAGCTGGTGGGCCGCCTGG
CTGAATACCAATCCGAGCAAAATTGTTATTGCACCGGAACAGTGGTTT
AATGGTAATAAGTATGATTACACCGACGTTGTGCCGGAAACCTGGGTT
AAAATTTAA
FutC 15: AA
(SEQ ID NO: 49)
MIIIKFCGALGNQLFQYALYEKMRILGKDVKADISAFGDGNEKRFFYL
DELGIEFNIASADEIAEYLNRKTIRFVPGFLQHRHYYFEKKPYVYNKK
ILSYDDCYLEGYWQNYRYFDDIKDELLKHMKFPCLPLEQKKLAEKMEN
ENSVAVHVRMGDYLNLQDLYGGICDADYYDRAFSYIEGNISNPVYYGF
SDDVDKASALLAKHKINWIDYNSEKGAIYDLILMSKCKNNIIANSSFS
WWGAYLEYNNGKVVVSPNRWMNCFENSNIAYWGWISL
FutC 15: DNA
(SEQ ID NO: 50)
ATGGCTATCATCATCAAGTTCTGTGGTGCCCTGGGTAATCAGCTGTTT
CAGTATGCCCTGTATGAAAAAATGCGTATTCTGGGCAAAGATGTGAAA
GCAGATATTAGCGCCTTTGGCGATGGTAATGAAAAACGTTTCTTTTAT
CTGGATGAGCTGGGTATTGAATTCAATATTGCCAGCGCAGATGAAATT
GCAGAATATCTGAATCGTAAAACCATTCGTTTTGTTCCGGGTTTTCTG
CAGCATCGCCATTATTATTTTGAAAAGAAACCGTATGTGTACAACAAA
AAGATTCTGAGTTACGATGATTGCTATCTGGAAGGCTATTGGCAGAAT
TATCGTTATTTTGATGACATTAAGGACGAACTGCTGAAACATATGAAA
TTTCCGTGCCTGCCGCTGGAACAGAAAAAACTGGCCGAAAAAATGGAA
AATGAAAATAGCGTGGCAGTTCATGTTCGTATGGGCGATTATCTGAAT
CTGCAGGATCTGTATGGTGGTATTTGCGATGCAGATTATTATGATCGT
GCATTTTCATATATCGAGGGTAATATTAGCAACCCGGTTTATTATGGT
TTTAGCGATGATGTGGATAAAGCAAGCGCACTGCTGGCAAAACATAAA
ATTAATTGGATTGACTACAACAGCGAAAAAGGTGCAATCTATGATCTG
ATTCTGATGAGTAAATGTAAGAATAACATCATCGCCAATAGCAGCTTT
AGCTGGTGGGGTGCATATCTGGAATATAATAATGGTAAAGTGGTGGTG
AGTCCGAATCGCTGGATGAATTGCTTTGAAAATAGCAATATCGCCTAT
TGGGGCTGGATTAGCCTGTAA
FutC 16: AA
(SEQ ID NO: 51)
MSKKKPVIIEILGGIGNQMFQFALAKILAEKNDSELFIDTNFYKETSQ
NLKNFPRYFSVGIFDLQFKLATEKEKIFFKHPSLKNRLNRKLGLNYPK
VFKEKSFNFDPELLTMKAPIFLKGYFQSYKYFAGTESKIRQLYEFPDE
KLDSRNEEIKNRIITKTSVSVHIRRGDYVENRKTQDFHGNCSVEYYKK
AVEYLSATIKDFNLVFFSDDIAWVQNQFKDLPYEKKFVTGNLYENSWK
DMYLMSLCDHNIIANSSFSWWAAWLNKNPEKKVVAPKKWFADMDQEQK
SLDLLPPDWVRI
FutC 16: DNA
(SEQ ID NO: 52)
ATGGCTAGCAAAAAGAAGCCGGTTATTATTGAAATTCTGGGTGGCATT
GGCAATCAGATGTTTCAGTTTGCCCTGGCCAAAATTCTGGCAGAAAAG
AATGATAGTGAACTGTTTATTGACACCAATTTTTACAAGGAAACCAGC
CAGAATCTGAAAAATTTTCCGCGTTATTTTAGCGTGGGTATTTTTGAT
CTGCAGTTTAAACTGGCAACCGAAAAAGAAAAAATCTTTTTCAAGCAC
CCGAGCCTGAAAAATCGTCTGAATCGTAAACTGGGCCTGAATTATCCG
AAAGTGTTTAAAGAAAAGAGCTTTAATTTCGACCCGGAACTGCTGACC
ATGAAAGCCCCGATTTTTCTGAAAGGCTATTTTCAGAGCTATAAATAT
TTCGCAGGTACCGAAAGTAAAATTCGTCAGCTGTATGAATTTCCGGAT
GAAAAACTGGATAGCCGCAATGAAGAAATTAAGAATCGCATTATTACC
AAGACCAGTGTTAGCGTTCATATTCGTCGTGGCGATTATGTTGAAAAT
CGCAAAACCCAGGATTTTCATGGTAATTGCAGTGTGGAATATTATAAA
AAGGCAGTTGAATACCTGAGCGCAACCATTAAGGATTTTAATCTGGTT
TTCTTTAGCGATGATATCGCATGGGTTCAGAATCAGTTTAAAGATCTG
CCGTATGAAAAGAAATTCGTGACCGGTAATCTGTATGAAAATAGTTGG
AAAGATATGTACCTGATGAGTCTGTGCGATCATAATATTATTGCAAAT
AGTAGCTTCAGCTGGTGGGCAGCATGGCTGAATAAGAATCCGGAAAAG
AAAGTTGTTGCCCCGAAAAAATGGTTTGCAGATATGGATCAGGAACAG
AAAAGCCTGGATCTGCTGCCGCCGGATTGGGTTCGTATTTAA
FutC 17: AA
(SEQ ID NO: 53)
MIVVRIIGGLGNQMFQYAFAKSLQQKGYQVKIDITKFKTYKLHGGYQL
DKFKIDLETATTLENIISRLGFRRSTKERSLLFNKKFLEVPKREYIKG
YFQTEKYFEDIKAILLKQFVVKNEISSSTLKYLKEITIQQNACSLHIR
RGDYVSDKKANSVHGTCDLAYYKEAIKVMKNKENDTHFFIFSDDIAWV
KQNLKVKNTTYIDHEVIPHEDIHLMSLCKHNITANSSFSWWGAWLNQH
SNKVVIAPKQWYLNKENEIASKDWIKI
FutC 17: DNA
(SEQ ID NO: 54)
ATGGCTATCGTGGTGCGCATTATTGGCGGCCTGGGTAATCAGATGTTT
CAGTATGCCTTTGCCAAAAGTCTGCAGCAGAAAGGTTATCAGGTTAAA
ATTGATATCACCAAATTCAAGACCTACAAACTGCATGGTGGTTATCAG
CTGGATAAATTCAAAATTGATCTGGAAACCGCCACCACCCTGGAAAAT
ATTATTAGTCGCCTGGGTTTTCGCCGTAGTACCAAAGAACGCAGTCTG
CTGTTTAATAAGAAATTTCTGGAAGTGCCGAAACGTGAATATATTAAG
GGTTATTTTCAGACCGAAAAGTATTTTGAAGATATTAAGGCCATCCTG
CTGAAACAGTTTGTGGTGAAAAATGAAATTAGCAGCAGCACCCTGAAA
TATCTGAAAGAAATTACCATTCAGCAGAATGCCTGTAGTCTGCATATT
CGTCGCGGTGACTATGTGAGCGATAAAAAAGCCAATAGTGTGCATGGC
ACCTGTGATCTGGCATATTATAAAGAAGCAATTAAGGTTATGAAGAAC
AAGTTTAACGACACCCATTTCTTTATTTTCAGTGATGATATCGCCTGG
GTGAAACAGAATCTGAAAGTGAAAAATACCACCTATATCGATCATGAA
GTTATTCCGCATGAAGATATTCATCTGATGAGCCTGTGCAAACATAAT
ATTACCGCCAATAGCAGTTTTAGTTGGTGGGGTGCATGGCTGAATCAG
CATAGCAATAAGGTGGTTATTGCCCCGAAACAGTGGTATCTGAATAAG
GAAAATGAAATTGCAAGCAAAGACTGGATTAAGATTTAA
FutC 18: AA
(SEQ ID NO: 55)
MIVTRIVGGLGNQMFQYAVGRALSAKTGQEFKLDLSEMDRYKVHALQL
DQFNIKGVRAGRHEIPFRPRKSFFGKILTALKNRNRIPQVFETTPSFD
PSVLQRKGSCYLSGYWQSEKYFSDCSELIRADFSLKGPMSDERQAVLS
QIRDAEAPVSVHVRRGDYVTNTTANSIHGTCEPEWYRQAMRKISDRTG
DPTFFVFSDDPMWARSNLPTYEKMVFVEPRADGKDAEDMHLMSSCQSH
IIANSTFSWWGAWLNPRQDKRVIAPARWFRAEDRDSTDLVPAQWERL
FutC 18: DNA
(SEQ ID NO: 56)
ATGGCTATCGTTACCCGTATTGTGGGTGGCCTGGGTAATCAGATGTTT
CAGTATGCAGTTGGCCGTGCCCTGAGTGCAAAAACCGGTCAGGAATTC
AAACTGGATCTGAGCGAAATGGATCGCTATAAAGTTCATGCACTGCAG
CTGGATCAGTTTAATATTAAGGGTGTTCGCGCCGGCCGTCATGAAATT
CCGTTTCGTCCGCGCAAAAGTTTCTTTGGCAAAATTCTGACCGCACTG
AAAAATCGCAATCGTATTCCGCAGGTTTTTGAAACCACCCCGAGCTTT
GATCCGAGCGTGCTGCAGCGTAAAGGTAGCTGTTATCTGAGTGGTTAT
TGGCAGAGCGAAAAATATTTTAGCGATTGTAGCGAACTGATTCGTGCA
GATTTTAGCCTGAAAGGTCCGATGAGCGATGAACGTCAGGCAGTGCTG
AGTCAGATTCGTGATGCAGAAGCACCGGTGAGCGTTCATGTTCGCCGC
GGCGATTATGTTACCAATACCACCGCCAATAGCATTCATGGCACCTGT
GAACCGGAATGGTATCGTCAGGCCATGCGCAAAATTAGTGATCGTACC
GGTGACCCGACCTTTTTCGTTTTTAGCGATGATCCGATGTGGGCACGC
AGCAATCTGCCGACCTATGAAAAAATGGTTTTTGTGGAACCGCGTGCC
GATGGTAAAGATGCCGAAGATATGCATCTGATGAGCAGCTGCCAGAGT
CATATTATTGCAAATAGCACCTTTAGTTGGTGGGGTGCATGGCTGAAT
CCGCGCCAGGATAAACGCGTGATTGCACCGGCACGCTGGTTTCGCGCA
GAAGATCGCGATAGCACCGATCTGGTTCCGGCCCAGTGGGAACGTCTG
TAA
FutC 19: AA
(SEQ ID NO: 57)
MIITHINGGLGNQMFQYAAGRALALRHGEELRLDTREFDGKVQFGFGL
DHFAIAARPGAPAELPPERRRDRLRYLAWRGFRLSPRLVRENGLGYNP
GFAEIGDGAYLKGYWQSERYFRDVEATIRRDFTIITPPDPVNRAILDD
LAASPAVSLHIRRGDYVVDPRTNATHGTCSMDYYARAVDLIAERMAET
PVVYAFSDDPAWVRDNLELPCEIRVMDHNDSARNYEDLRLMSACRHHV
IANSSFSWWGAWLNPSADKIVVSPARWFADPKLVNEDIWPTSWIRLS
FutC 19: DNA
(SEQ ID NO: 58)
ATGGCTATCATCACCCATATTAACGGCGGTCTGGGCAATCAGATGTTT
CAGTATGCCGCCGGTCGTGCACTGGCCCTGCGTCATGGTGAAGAACTG
CGTCTGGATACCCGCGAATTTGATGGCAAAGTGCAGTTTGGTTTTGGT
CTGGATCATTTTGCCATTGCCGCACGCCCGGGTGCCCCGGCAGAATTA
CCGCCTGAACGTCGTCGCGATCGCCTGCGCTATCTGGCCTGGCGTGGC
TTTCGCCTGAGTCCGCGTCTGGTGCGTGAAAATGGTCTGGGCTATAAT
CCGGGTTTTGCCGAAATTGGTGACGGCGCATATCTGAAAGGTTATTGG
CAGAGTGAACGCTATTTTCGCGATGTTGAAGCAACCATTCGTCGTGAT
TTTACCATTATTACCCCGCCGGACCCTGTGAATCGCGCCATTCTGGAT
GATCTGGCCGCCAGTCCGGCAGTGAGCCTGCATATTCGTCGTGGCGAT
TATGTTGTGGACCCTCGTACCAATGCCACCCACGGTACCTGTAGCATG
GATTATTATGCCCGCGCAGTTGATCTGATTGCAGAACGTATGGCAGAA
ACCCCGGTGGTGTATGCATTTTCAGATGATCCGGCCTGGGTGCGCGAT
AATCTGGAACTGCCGTGCGAAATTCGCGTTATGGATCATAATGATAGC
GCACGCAATTATGAAGATCTGCGCCTGATGAGTGCCTGCCGTCATCAT
GTTATTGCCAATAGTAGCTTTAGCTGGTGGGGCGCATGGCTGAATCCG
AGCGCCGATAAAATTGTGGTTAGTCCGGCCCGTTGGTTTGCCGATCCG
AAACTGGTTAATGAAGATATTTGGCCGACCAGTTGGATTCGTCTGAGT
TAA
FutC 20: AA
(SEQ ID NO: 59)
MVIVRVQGGLGNQMFQYGFAKYQELSNEEVYLDITDYQTHIHHYGFEL
EKVFSNLTYKTIDGERLNKVRANPNMLLNRMLNKVLNIQIVRGSEFRE
QPAVSVSKRYTYNKDIYENGFWANNEYVDAVKDTLKKDFTFKYILEGR
NRELMDFLQGKISVGVHVRRGDYLQEKELRDVCDPDYYRKAFEIFMKR
DVKTVFIIFSDDIPWVRKNFHFSKNMVFVDWNSGGEKSHVDMQMMSLC
NHNIIANSTFSWWGAWLNANKDKCVVAPRYWRNNSKNESLIYPKNWML
L
FutC 20: DNA
(SEQ ID NO: 60)
ATGGTTATCGTTCGTGTGCAGGGCGGTCTGGGTAATCAGATGTTTCAG
TATGGTTTTGCAAAATATCAGGAACTGAGTAATGAAGAAGTTTATCTG
GATATTACCGATTATCAGACCCATATTCATCATTATGGTTTTGAACTG
GAAAAGGTGTTTAGTAATCTGACCTATAAAACCATTGACGGTGAACGT
CTGAATAAGGTTCGCGCAAATCCGAATATGCTGCTGAATCGCATGCTG
AATAAGGTGCTGAATATTCAGATTGTGCGTGGTAGTGAATTTCGCGAA
CAGCCGGCAGTGAGCGTTAGCAAACGCTATACCTATAATAAGGATATC
TATTTCAACGGCTTCTGGGCCAATAATGAATATGTGGATGCAGTGAAA
GATACCCTGAAAAAAGATTTTACCTTCAAATACATCCTGGAAGGCCGC
AATCGTGAACTGATGGATTTTCTGCAGGGCAAAATTAGTGTGGGTGTG
CATGTGCGTCGCGGCGATTATCTGCAGGAAAAAGAACTGCGCGATGTT
TGTGATCCGGATTATTATCGCAAAGCATTTGAAATTTTCATGAAGCGC
GATGTTAAAACCGTTTTTATTATTTTCAGCGACGATATTCCGTGGGTG
CGCAAAAATTTTCATTTTAGCAAAAACATGGTGTTCGTTGATTGGAAT
AGCGGCGGCGAAAAAAGCCATGTTGATATGCAGATGATGAGCCTGTGT
AATCATAATATTATCGCAAATAGCACCTTCAGCTGGTGGGGTGCATGG
CTGAATGCCAATAAGGATAAATGTGTGGTTGCACCGCGTTATTGGCGT
AATAATAGCAAAAATGAAAGCCTGATCTATCCGAAAAATTGGATGCTG
CTGTAA
FutC 21: AA
(SEQ ID NO: 61)
MAFKVVQICGGLGNQMFQYAFAKSLQKHLNTPVLLDITSFDWSNRKMQ
LELFPIDLPYASAKEIAIAKMQHLPKLVRDTLKCMGFDRVSQEIVFEY
EPGLLKPSRLTYFYGYFQDPRYFDAISPLIKQTFTLPPPENGNNKKKE
EEYHRKLALILAAKNSVFVHVRRGDYVGIGCQLGIDYQKKALEYIAKR
VPNMELFVFCEDLKFTQNLDLGYPFMDMTTRDKEEEAYWDMLLMQSCK
HGIIANSTYSWWAAYLINNPEKIIIGPKHWLFGHENILCKEWVKIESH
FEVKSKKYNA
FutC 21: DNA
(SEQ ID NO: 62)
ATGGCATTCAAGGTGGTGCAGATTTGTGGCGGTCTGGGCAACCAGATG
TTCCAGTATGCCTTCGCCAAGAGTCTGCAGAAGCATCTGAACACCCCG
GTGCTGCTGGATATTACCAGTTTTGATTGGAGCAATCGCAAGATGCAG
CTGGAGCTGTTCCCTATTGATCTGCCGTATGCCAGCGCCAAAGAGATC
GCCATCGCCAAAATGCAGCATCTGCCGAAACTGGTGCGCGATACTTTA
AAATGCATGGGCTTTGATCGTGTGAGCCAAGAAATCGTGTTTGAGTAT
GAGCCGGGTCTGCTGAAACCGAGCCGTTTAACCTATTTCTACGGCTAT
TTCCAAGATCCGCGCTACTTCGATGCCATCAGCCCGCTGATTAAGCAG
ACCTTCACTTTACCGCCGCCGGAGAATGGCAACAATAAGAAAAAAGAG
GAAGAATATCATCGCAAGCTGGCTTTAATTCTGGCAGCCAAAAACAGC
GTGTTCGTGCATGTTCGCCGCGGTGACTATGTGGGTATCGGCTGCCAG
CTGGGCATCGACTATCAGAAGAAGGCTTTAGAATATATCGCAAAACGC
GTGCCGAACATGGAGCTGTTTGTGTTTTGCGAGGATTTAAAATTTACC
CAGAATTTAGATTTAGGCTACCCGTTTATGGACATGACCACCCGTGAT
AAAGAAGAAGAAGCCTATTGGGACATGCTGCTGATGCAGAGCTGCAAG
CACGGCATCATTGCCAACAGCACCTATAGCTGGTGGGCCGCATATTTA
ATTAACAACCCGGAAAAGATCATCATCGGCCCGAAGCATTGGCTGTTC
GGCCATGAGAACATTTTATGCAAGGAATGGGTTAAAATTGAGAGCCAT
TTCGAAGTGAAAAGCAAAAAGTATAACGCC
Oc Pyruvate Kinase: AA
(SEQ ID NO: 63)
MSKSHSEAGSAFIQTQQLHAAMADTFLEHMCRLDIDSAPITARNTGII
CTIGPASRSVETLKEMIKSGMNVARMNFSHGTHEYHAETIKNVRTATE
SFASDPILYRPVAVALDTKGPEIRTGLIKGSGTAEVELKKGATLKITL
DNAYMEKCDENILWLDYKNICKVVDVGSKVYVDDGLISLQVKQKGPDF
LVTEVENGGFLGSKKGVNLPGAAVDLPAVSEKDIQDLKFGVEQDVDMV
FASFIRKAADVHEVRKILGEKGKNIKIISKIENHEGVRRFDEILEASD
GIMVARGDLGIEIPAEKVFLAQKMIIGRCNRAGKPVICATQMLESMIK
KPRPTRAEGSDVANAVLDGADCIMLSGETAKGDYPLEAVRMQHLIARE
AEAAMFHRKLFEELARSSSHSTDLMEAMAMGSVEASYKCLAAALIVLT
ESGRSAHQVARYRPRAPIIAVTRNHQTARQAHLYRGIFPVVCKDPVQE
AWAEDVDLRVNLAMNVGKARGFFKKGDVVIVLTGWRPGSGFTNTMRVV
PVP
Oc Creatine Kinase: AA
(SEQ ID NO: 64)
MPFGNTHNKYKLNYKSEEEYPDLSKHNNHMAKVLTPDLYKKLRDKETP
SGFTLDDVIQTGVDNPGHPFIMTVGCVAGDEESYTVFKDLFDPIIQDR
HGGFKPTDKHKTDLNHENLKGGDDLDPHYVLSSRVRTGRSIKGYTLPP
HCSRGERRAVEKLSVEALNSLTGEFKGKYYPLKSMTEQEQQQLIDDHF
LFDKPVSPLLLASGMARDWPDARGIWHNDNKSFLVWVNEEDHLRVISM
EKGGNMKEVFRRFCVGLQKIEEIFKKAGHPFMWNEHLGYVLTCPSNLG
TGLRGGVHVKLAHLSKHPKFEEILTRLRLQKRGTGGVDTAAVGSVFDI
SNADRLGSSEVEQVQLVVDGVKLMVEMEKKLEKGQSIDDMIPAQK
Gs AckA: AA
(SEQ ID NO: 65)
MAKVLAVNAGSSSLKFQLFDMPAETVLTKGIVERIGFDDAIFTIVVNG
EKQREVTSIPNHAVAVKLLLDKLIRYGIIRSFDEIDGIGHRVVHGGEK
FSDSVLITDEVIKQIEEVSELAPLHNPANLVGIRAFQEVLPNVPAVAV
FDTAFHQTMPEQSFLYSLPYEYYTKFGIRKYGFHGTSHKYVTQRAAEL
LGRPIEQLRLISCHLGNGASIAAVEGGKSIDTSMGFTPLAGVAMGTRS
GNIDPALIPYIMEKTGMTVNEVIEVLNKKSGMLGISGISSDLRDLEKA
AAEGNERAELALEVFANRIHKYIGSYAARMCGVDAIIFTAGIGENSEV
VRAKVLRGLEFMGVYWDPILNKVRGKEAFISYPHSPVKVLVIPTNEEV
MIARDVMRLANL
Gs AckA: DNA
(SEQ ID NO: 66)
ATGGCAAAAGTCCTGGCGGTCAATGCGGGGTCGAGCAGTTTGAAATTC
CAGCTCTTCGACATGCCGGCGGAAACTGTGCTGACCAAAGGGATTGTG
GAACGAATCGGCTTCGACGATGCTATTTTTACGATTGTGGTGAACGGC
GAAAAACAGCGTGAAGTCACAAGCATACCAAATCACGCGGTTGCCGTC
AAACTGCTGCTGGACAAATTAATTCGCTATGGGATTATTCGTAGCTTC
GATGAAATTGATGGCATCGGCCACCGCGTGGTGCACGGGGGAGAAAAA
TTCAGCGATTCTGTACTTATCACAGATGAAGTAATCAAACAGATTGAA
GAAGTCTCGGAACTCGCTCCGTTACATAACCCGGCAAACCTGGTAGGA
ATCCGCGCGTTCCAGGAGGTGCTTCCCAACGTCCCGGCGGTCGCGGTT
TTTGACACGGCGTTTCACCAGACCATGCCGGAGCAAAGCTTCTTGTAT
TCTTTGCCGTATGAGTATTATACAAAATTTGGTATCCGCAAATACGGT
TTCCACGGCACATCCCATAAATATGTGACCCAACGTGCGGCTGAGTTG
TTGGGGCGTCCTATCGAACAGCTGAGACTCATCAGTTGTCACCTGGGG
AACGGCGCATCTATTGCGGCTGTAGAAGGCGGTAAATCCATAGACACG
TCTATGGGTTTCACTCCGCTGGCTGGTGTGGCCATGGGTACGCGCTCG
GGAAATATCGACCCCGCCCTTATCCCCTACATTATGGAAAAGACCGGC
ATGACGGTGAACGAAGTTATTGAGGTCCTGAATAAAAAGTCGGGCATG
CTCGGCATATCCGGTATTAGCTCGGATCTCCGAGATCTGGAGAAAGCG
GCGGCGGAAGGTAATGAACGCGCGGAACTGGCGTTAGAGGTTTTTGCG
AATCGCATTCATAAGTATATTGGTAGCTATGCGGCACGAATGTGTGGT
GTCGATGCTATTATTTTTACGGCCGGCATTGGTGAAAATTCTGAAGTG
GTACGAGCCAAGGTGTTACGTGGTCTGGAGTTTATGGGCGTATATTGG
GACCCGATACTGAATAAAGTACGCGGTAAAGAAGCGTTTATCAGTTAT
CCGCATAGCCCTGTCAAAGTCTTGGTTATCCCAACGAACGAAGAAGTC
ATGATTGCGCGCGATGTTATGCGGTTAGCGAATTTATAA
MaeB: AA
(SEQ ID NO: 67)
MDDQLKQSALDFHEFPVPGKIQVSPTKPLATQRDLALAYSPGVAAPCL
EIEKDPLKAYKYTARGNLVAVISNGTAVLGLGNIGALAGKPVMEGKGV
LFKKFAGIDVFDIEVDELDPDKFIEVVAALEPTFGGINLEDIKAPECF
YIEQKLRERMNIPVFHDDQHGTAIISTAAILNGLRVVEKNISDVRMVV
SGAGAAAIACMNLLVALGLQKHNIVVCDSKGVIYQGREPNMAETKAAY
AVVDDGKRTLDDVIEGADIFLGCSGPKVLTQEMVKKMARAPMILALAN
PEPEILPPLAKEVRPDAIICTGRSDYPNQVNNVLCFPFIFRGALDVGA
TAINEEMKLAAVRAIAELAHAEQSEVVASAYGDQDLSFGPEYIIPKPF
DPRLIVKIAPAVAKAAMESGVATRPIADFDVYIDKLTEFVYKTNLFMK
PIFSQARKAPKRVVLPEGEEARVLHATQELVTLGLAKPILIGRPNVIE
MRIQKLGLQIKAGVDFEIVNNESDPRFKEYWTEYFQIMKRRGVTQEQA
QRALISNPTVIGAIMVQRGEADAMICGTVGDYHEHFSVVKNVFGYRDG
VHTAGAMNALLLPSGNTFIADTYVNDEPDAEELAEITLMAAETVRRFG
IEPRVALLSHSNFGSSDCPSSSKMRQALELVRERAPELMIDGEMHGDA
ALVEAIRNDRMPDSSLKGSANILVMPNMEAARISYNLLRVSSSEGVTV
GPVLMGVAKPVHVLTPIASVRRIVNMVALAVVEAQTQPL
MaeB: DNA
(SEQ ID NO: 68)
ATGGATGACCAGTTAAAACAAAGTGCACTTGATTTCCATGAATTTCCA
GTTCCAGGGAAAATCCAGGTTTCTCCAACCAAGCCTCTGGCAACACAG
CGCGATCTGGCGCTGGCCTACTCACCAGGCGTTGCCGCACCTTGTCTT
GAAATCGAAAAAGACCCGTTAAAAGCCTACAAATATACCGCCCGAGGT
AACCTGGTGGCGGTGATCTCTAACGGTACGGCGGTGCTGGGGTTAGGC
AACATTGGCGCGCTGGCAGGCAAACCGGTGATGGAAGGCAAGGGCGTT
CTGTTTAAGAAATTCGCCGGGATTGATGTATTTGACATTGAAGTTGAC
GAACTCGACCCGGACAAATTTATTGAAGTTGTCGCCGCGCTCGAACCA
ACCTTCGGCGGCATCAACCTCGAAGAtATTAAAGCGCCAGAATGTTTC
TATATTGAACAGAAACTGCGCGAGCGGATGAATATTCCGGTATTCCAC
GACGATCAGCACGGCACGGCAATTATCAGCACTGCCGCCATCCTCAAC
GGCTTGCGCGTGGTGGAGAAAAACATCTCCGACGTGCGGATGGTGGTT
TCCGGCGCGGGTGCCGCAGCAATCGCCTGTATGAACCTGCTGGTAGCG
CTGGGTCTGCAAAAACATAACATCGTGGTTTGCGATTCAAAAGGCGTT
ATCTATCAGGGCCGTGAGCCAAACATGGCGGAAACCAAAGCCGCgTAT
GCGGTGGTGGATGACGGCAAACGTACCCTCGATGATGTGATTGAAGGC
GCGGATATTTTCCTGGGCTGTTCCGGCCCGAAAGTGCTGACCCAGGAA
ATGGTGAAGAAAATGGCTCGTGCGCCAATGATCCTGGCGCTGGCGAAC
CCGGAACCGGAAATTCTGCCGCCGCTGGCGAAAGAAGTGCGTCCGGAT
GCCATCATTTGCACCGGTCGTTCTGACTATCCGAACCAGGTGAACAAC
GTCCTGTGCTTCCCGTTCATCTTCCGTGGCGCGCTGGACGTTGGCGCA
ACCGCCATCAACGAAGAGATGAAACTGGCGGCGGTACGTGCGATTGCA
GAACTCGCCCATGCGGAACAGAGCGAAGTGGTGGCTTCAGCGTATGGC
GATCAGGATCTGAGCTTTGGTCCGGAATACATCATTCCAAAACCGTTT
GATCCGCGCTTGATCGTTAAGATCGCTCCTGCGGTCGCTAAAGCCGCG
ATGGAGTCGGGCGTGGCGACTCGTCCGATTGCTGATTTCGACGTCTAC
ATCGACAAGCTGACTGAGTTCGTTTACAAAACCAACCTGTTTATGAAG
CCGATTTTCTCCCAGGCTCGCAAAGCGCCGAAGCGCGTTGTTCTGCCG
GAAGGGGAAGAGGCGCGCGTTCTGCATGCCACTCAGGAACTGGTAACG
CTGGGACTGGCGAAACCGATCCTTATCGGTCGTCCGAACGTGATCGAA
ATGCGCATTCAGAAACTGGGCTTGCAGATCAAAGCGGGCGTTGATTTT
GAGATCGTCAATAACGAATCCGATCCGCGCTTTAAAGAGTACTGGACC
GAATACTTCCAGATCATGAAGCGTCGCGGCGTCACTCAGGAACAGGCG
CAGCGGGCGCTGATCAGTAACCCGACAGTGATCGGCGCGATCATGGTT
CAGCGTGGGGAAGCCGATGCAATGATTTGCGGTACGGTGGGTGATTAT
CATGAACATTTTAGCGTGGTGAAAAATGTCTTTGGTTATCGCGATGGC
GTTCACACCGCAGGTGCCATGAACGCGCTGCTGCTGCCGAGTGGTAAC
ACCTTTATTGCCGATACCTATGTTAATGATGAACCGGATGCAGAAGAG
CTGGCGGAGATCACCTTGATGGCGGCAGAAACTGTCCGTCGTTTTGGT
ATTGAGCCGCGCGTTGCTTTGTTGTCGCACTCCAACTTTGGTTCTTCT
GACTGCCCGTCGTCGAGCAAAATGCGTCAGGCGCTGGAACTGGTCAGG
GAACGTGCACCAGAACTGATGATTGATGGTGAAATGCACGGCGATGCA
GCGCTGGTGGAAGCGATTCGCAACGACCGTATGCCGGACAGCTCTTTG
AAAGGTTCCGCCAATATTCTGGTGATGCCGAACATGGAAGCTGCCCGC
ATTAGTTACAACTTACTGCGTGTTTCCAGCTCGGAAGGTGTGACTGTC
GGCCCGGTGCTGATGGGTGTGGCGAAACCGGTTCACGTGTTAACGCCG
ATCGCATCGGTGCGTCGTATCGTCAACATGGTGGCGCTGGCCGTGGTA
GAAGCGCAAACCCAACCGCTGTAA
FDH: AA
(SEQ ID NO: 69)
MKIVLVLYDAGKHAADEEKLYGCTENKLGIANWLKDQGHELITTSDKE
GGNSVLDQHIPDADIIITTPFHPAYITKERIDKAKKLKLVVVAGVGSD
HIDLDYINQTGKKISVLEVTGSNVVSVAEHVVMTMLVLVRNFVPAHEQ
IINHDWEVAAIAKDAYDIEGKTIATIGAGRIGYRVLERLVPFNPKELL
YYQHQALPKDAEEKVGARRVENIEELVAQADIVTVNAPLHAGTKGLIN
KELLSKFKKGAWLVNTARGAICVAEDVAAALESGQLRGYGGDVWFPQP
APKDHPWRDMRNKYGAGNAMTPHYSGTTLDAQTRYAQGTKNILESFFT
GKFDYRPQDIILLNGEYVTKAYGKHDKK
FDH: DNA
(SEQ ID NO: 70)
ATGAAGATCGTTTTAGTCTTATATGATGCTGGTAAACACGCTGCCGAT
GAAGAAAAATTATACGGTTGTACTGAAAACAAATTAGGTATTGCCAAT
TGGTTGAAAGATCAAGGACATGAATTAATCACCACGTCTGATAAAGAA
GGCGGAAACAGTGTGTTGGATCAACATATACCAGATGCCGATATTATC
ATTACAACTCCTTTCCATCCTGCTTATATCACTAAGGAAAGAATCGAC
AAGGCTAAAAAATTGAAATTAGTTGTTGTCGCTGGTGTCGGTTCTGAT
CATATTGATTTGGATTATATCAACCAAACCGGTAAGAAAATCTCCGTT
TTGGAAGTTACCGGTTCTAATGTTGTCTCTGTTGCAGAACACGTTGTC
ATGACCATGCTTGTCTTGGTTAGAAATTTTGTTCCAGCTCACGAACAA
ATCATTAACCACGATTGGGAGGTTGCTGCTATCGCTAAGGATGCTTAC
GATATCGAAGGTAAAACTATCGCCACCATTGGTGCCGGTAGAATTGGT
TACAGAGTCTTGGAAAGATTAGTCCCATTCAATCCTAAAGAATTATTA
TACTACCAGCATCAAGCTTTACCAAAAGATGCTGAAGAAAAAGTTGGT
GCTAGAAGGGTTGAAAATATTGAAGAATTGGTTGCCCAAGCTGATATA
GTTACAGTTAATGCTCCATTACACGCTGGTACAAAAGGTTTAATTAAC
AAGGAATTATTGTCTAAATTCAAGAAAGGTGCTTGGTTAGTCAATACT
GCAAGAGGTGCCATTTGTGTTGCCGAAGATGTTGCTGCAGCTTTAGAA
TCTGGTCAATTAAGAGGTTATGGTGGTGATGTTTGGTTCCCACAACCA
GCTCCAAAAGATCACCCATGGAGAGATATGAGAAACAAATATGGTGCT
GGTAACGCCATGACTCCTCATTACTCTGGTACTACTTTAGATGCTCAA
ACTAGATACGCTCAAGGTACTAAAAATATCTTGGAGTCATTCTTTACT
GGTAAGTTTGATTACAGACCACAAGATATCATCTTATTAAACGGTGAA
TACGTTACCAAAGCTTACGGTAAACACGATAAGAAATAA
PTDH: AA
(SEQ ID NO: 71)
MLPKLVITHRVHEEILQLLAPHCELITNQTDSTLTREEILRRCRDAQA
MMAFMPDRVDADFLQACPELRVIGCALKGFDNFDVDACTARGVWLTFV
PDLLTVPTAELAIGLAVGLGRHLRAADAFVRSGKFRGWQPRFYGTGLD
NATVGFLGMGAIGLAMADRLQGWGATLQYHARKALDTQTEQRLGLRQV
ACSELFASSDFILLALPLNADTLHLVNAELLALVRPGALLVNPCRGSV
VDEAAVLAALERGQLGGYAADVFEMEDWARADRPQQIDPALLAHPNTL
FTPHIGSAVRAVRLEIERCAAQNILQALAGERPINAVNRLPKANPAAD
PTDH: DNA
(SEQ ID NO: 72)
ATGCTGCCGAAACTCGTTATAACTCACCGAGTACACGAAGAGATCCTG
CAACTGCTGGCGCCACATTGCGAGCTGATCACCAACCAGACCGACAGC
ACGCTGACGCGCGAGGAAATTCTGCGCCGCTGCCGCGATGCTCAGGCG
ATGATGGCGTTCATGCCCGATCGGGTCGATGCAGACTTTCTTCAAGCC
TGCCCTGAGCTGCGTGTAATCGGCTGCGCGCTCAAGGGCTTCGACAAT
TTCGATGTGGACGCCTGTACTGCCCGCGGGGTCTGGCTGACCTTCGTG
CCTGATCTGTTGACGGTCCCGACTGCCGAGCTGGCGATCGGACTGGCG
GTGGGGCTGGGGCGGCATCTGCGGGCAGCAGATGCGTTCGTCCGCTCT
GGCAAGTTCCGGGGCTGGCAACCACGGTTCTACGGCACGGGGCTGGAT
AACGCTACGGTCGGCTTCCTTGGCATGGGCGCCATCGGACTGGCCATG
GCTGATCGCTTGCAGGGATGGGGCGCGACCCTGCAGTACCACGCGCGG
AAGGCTCTGGATACACAAACCGAGCAACGGCTCGGCCTGCGCCAGGTG
GCGTGCAGCGAACTCTTCGCCAGCTCGGACTTCATCCTGCTGGCGCTT
CCCTTGAATGCCGATACCCTGCATCTGGTCAACGCCGAGCTGCTTGCC
CTCGTACGGCCGGGCGCTCTGCTTGTAAACCCCTGTCGTGGTTCGGTA
GTGGATGAAGCCGCCGTGCTCGCGGCGCTTGAGCGAGGCCAGCTCGGC
GGGTATGCGGCGGATGTATTCGAAATGGAAGATTGGGCTCGCGCGGAC
CGGCCGCAGCAGATCGATCCTGCGCTGCTCGCGCATCCGAATACGCTG
TTCACTCCGCACATAGGGTCGGCAGTGCGCGCGGTGCGCCTGGAGATT
GAACGTTGTGCAGCGCAGAACATCCTCCAGGCATTGGCAGGTGAGCGC
CCAATCAACGCTGTGAACCGTCTGCCCAAGGCCAACCCTGCCGCAGAT
TGATAA
GDH: AA
(SEQ ID NO: 73)
MYPDLKGKVVAITGAASGLGKAMAIRFGKEQAKVVINYYSNKQDPNEV
KEEVIKAGGEAVVVQGDVTKEEDVKNIVQTAIKEFGTLDIMINNAGLE
NPVPSHEMPLKDWDKVIGTNLTGAFLGSREAIKYFVENDIKGNVINMS
SVHEVIPWPLFVHYAASKGGIKLMTETLALEYAPKGIRVNNIGPGAIN
TPINAEKFADPKQKADVESMIPMGYIGEPEEIAAVAAWLASKEASYVT
GITLFADGGMTQYPSFQAGRG
GDH: DNA
(SEQ ID NO: 74)
ATGTATCCTGATCTCAAGGGAAAAGTTGTAGCCATTACAGGTGCAGCC
AGTGGACTTGGAAAAGCTATGGCGATTAGATTCGGGAAAGAACAAGCA
AAGGTCGTCATCAACTATTATTCTAATAAGCAGGACCCCAACGAAGTA
AAAGAAGAAGTAATCAAAGCAGGAGGTGAAGCCGTTGTGGTTCAGGGA
GATGTTACCAAAGAAGAGGATGTCAAGAATATAGTTCAGACCGCGATT
AAGGAATTTGGAACGTTAGATATTATGATTAATAATGCAGGTTTGGAA
AACCCCGTACCTTCTCACGAAATGCCATTGAAGGATTGGGATAAGGTA
ATAGGAACGAATCTAACCGGAGCGTTCTTAGGCAGCAGAGAAGCCATC
AAGTATTTTGTCGAGAACGATATAAAAGGAAATGTTATTAACATGTCA
TCCGTCCATGAGGTTATTCCATGGCCACTTTTCGTTCATTACGCTGCT
AGTAAAGGTGGTATCAAATTAATGACAGAAACTTTGGCTCTGGAATAT
GCACCAAAAGGTATTAGAGTTAACAACATTGGACCAGGCGCTATTAAT
ACTCCCATAAATGCTGAGAAATTTGCCGACCCAAAACAAAAAGCTGAT
GTTGAATCAATGATACCCATGGGATATATTGGAGAGCCTGAGGAAATA
GCCGCTGTTGCTGCATGGCTTGCTTCCAAGGAAGCTTCTTATGTGACT
GGGATCACTCTTTTCGCAGACGGAGGAATGACGCAATATCCATCCTTT
CAGGCCGGGGGGGCTAA
Arabidopsis thaliana, At GMD M2: AA
(SEQ ID NO: 75)
MASENNGSRSDSESITAPKADSTVVEPRKIALITGITGQDGSYLTEFL
LGKGYEVHGLIRRSSNFNTQRINHIYIDPANVNKALMKLHYADLTDAS
SLRRWIDVIKPDEVYNLAAQSHVAVSFEIPDYTADVVATGALRLLEAV
RSHTIDSGRTVKYYQAGSSEMFGSTPPPQSETTPFHPRSPYAASKCAA
HWYTVNYREAYGLFACNGILFNHESPRRGENFVTRKITRALGRIKVGL
QTKLFLGNLQASRDWGFAGDYVEAMWLMLQQEKPDDYVVATEEGHTVE
EFLDVSFGYLGLNWKDYVEIDQRYFRPAEVDNLQGDASKAKEVLGWKP
QVGFEKLVKMMVDEDLELAKREKVLVDAGYMDAKQQP
Arabidopsis thaliana, At GMD M2: DNA
(SEQ ID NO: 76)
ATGGCAAGTGAGAACAATGGTTCACGTTCTGACTCTGAAAGCATCACG
GCTCCTAAAGCGGACAGCACCGTTGTGGAACCACGGAAAATCGCTCTA
ATCACCGGCATCACGGGTCAGGACGGTAGTTACTTGACTGAATTTCTA
CTAGGCAAAGGTTACGAAGTGCATGGCCTGATCCGTAGGAGTAGCAAT
TTTAACACGCAGCGGATCAATCATATCTATATTGATCCACACAACGTG
AACAAAGCTTTAATGAAACTCCATGCCGCGGATCTCACTGACGCCTCT
TCGTTGCGTCGCTGGATCGACGTCATTAAACCTGACGAAGTGTATAAC
CTGGCGGCACAGTCTCATGTGGCCGTTTCATTCGAAATACCTGATTAT
ACGGCGGACGTGGTTGCCACCGGTGCCTTAAGACTGCTCGAGGCGGTT
CGCTCCCATACCATTGATTCCGGGCGCACGGTAAAATATTATCAGGCA
GGAAGCAGCGAAATGTTTGGAAGTACGCCGCCCCCTCAGTCTGAGACA
ACCCCGTTTCACCCGCGCAGTCCGTATGCGGCATCTAAATGTGCCGCA
CATTGGTATACAGTCAATTATCGTGAGGCTTATGGCTTGTTTGCATGC
AATGGCATTCTGTTCAATCATGAAAGCCCGCGCAGAGGCGAAAATTTT
GTTACCCGCAAAATTACGCGTGCCCTGGGCCGTATTAAAGTAGGTCTG
CAAACTAAACTGTTTCTTGGCAACCTCCAGGCTAGCCGTGACTGGGGA
TTTGCCGGTGATTATGTCGAAGCCATGTGGCTCATGTTACAGCAGGAG
AAACCGGACGATTATGTTGTTGCGACAGAAGAAGGACACACAGTGGAG
GAATTTTTGGATGTATCGTTCGGCTATTTAGGTCTAAACTGGAAAGAT
TACGTTGAGATTGATCAACGCTACTTCCGGCCGGCGGAAGTGGACAAC
CTGCAAGGAGATGCCTCCAAGGCAAAAGAAGTACTGGGTTGGAAACCG
CAGGTGGGCTTCGAGAAACTTGTCAAAATGATGGTGGATGAAGATCTG
GAATTAGCTAAACGCGAGAAGGTACTGGTAGATGCAGGATACATGGAT
GCGAAGCAGCAACCGTAA
Arabidopsis thaliana, At GMD M3: AA
(SEQ ID NO: 77)
MASENNGSRSDSESITAPKADSTVVEPRKIALITGITGQDGSYLTEFL
LGKGYEVHGLIRRSSNFNTQRINHIYIDPHNVNKALMKLHYADLTDAS
SLRRWIDVIKPDEVYNLAAQSHVAVSFEIPDYTADVVATGALRLLEAV
RSHTIDSGRTVKYYQAGSSEMFGSTPPPQSETTPFHPRSPYAASKCAA
HWYTVNYREAYGLFACNGILFNHESPRRGENFVTRAITRALGRIKVGL
QTKLFLGNLQASRDWGFAGDYVEAMWLMLQQEKPDDYVVATEEGHTVE
EFLDVSFGYLGLNWKDYVEIDQRYFRPAEVDNLQGDASKAKEVLGWKP
QVGFEKLVKMMVDEDLELAKREKVLVDAGYMDAKQQP
Arabidopsis thaliana, At GMD M3: DNA
(SEQ ID NO: 78)
ATGGCAAGTGAGAACAATGGTTCACGTTCTGACTCTGAAAGCATCACG
GCTCCTAAAGCGGACAGCACCGTTGTGGAACCACGGAAAATCGCTCTA
ATCACCGGCATCACGGGTCAGGACGGTAGTTACTTGACTGAATTTCTA
CTAGGCAAAGGTTACGAAGTGCATGGCCTGATCCGTAGGAGTAGCAAT
TTTAACACGCAGCGGATCAATCATATCTATATTGATCCACACAACGTG
AACAAAGCTTTAATGAAACTCCATTACGCGGATCTCACTGACGCCTCT
TCGTTGCGTCGCTGGATCGACGTCATTAAACCTGACGAAGTGTATAAC
CTGGCGGCACAGTCTCATGTGGCCGTTTCATTCGAAATACCTGATTAT
ACGGCGGACGTGGTTGCCACCGGTGCCTTAAGACTGCTCGAGGCGGTT
CGCTCCCATACCATTGATTCCGGGCGCACGGTAAAATATTATCAGGCA
GGAAGCAGCGAAATGTTTGGAAGTACGCCGCCCCCTCAGTCTGAGACA
ACCCCGTTTCACCCGCGCAGTCCGTATGCGGCATCTAAATGTGCCGCA
CATTGGTATACAGTCAATTATCGTGAGGCTTATGGCTTGTTTGCATGC
AATGGCATTCTGTTCAATCATGAAAGCCCGCGCAGAGGCGAAAATTTT
GTTACCCGCGCAATTACGCGTGCCCTGGGCCGTATTAAAGTAGGTCTG
CAAACTAAACTGTTTCTTGGCAACCTCCAGGCTAGCCGTGACTGGGGA
TTTGCCGGTGATTATGTCGAAGCCATGTGGCTCATGTTACAGCAGGAG
AAACCGGACGATTATGTTGTTGCGACAGAAGAAGGACACACAGTGGAG
GAATTTTTGGATGTATCGTTCGGCTATTTAGGTCTAAACTGGAAAGAT
TACGTTGAGATTGATCAACGCTACTTCCGGCCGGCGGAAGTGGACAAC
CTGCAAGGAGATGCCTCCAAGGCAAAAGAAGTACTGGGTTGGAAACCG
CAGGTGGGCTTCGAGAAACTTGTCAAAATGATGGTGGATGAAGATCTG
GAATTAGCTAAACGCGAGAAGGTACTGGTAGATGCAGGATACATGGAT
GCGAAGCAGCAACCGTAA
Arabidopsis thaliana, At GMD M4: AA
(SEQ ID NO: 79)
MASENNGSRSDSESITAPKADSTVVEPRKIALITGITGQDGSYLTEFL
LGKGYEVHGLIRRSSNFNTQRINHIYIDPHNVNKALMKLHYADLTDAS
SLRRWIDVIKPDEVYNLAAQSHVAVSFEIPDYTADVVATGALRLLEAV
RSHTIDSGRTVKYYQAGSSEMFGSTPPPQSETTPFHPRSPYAASKCAA
HWYTVNYREAYGLFACNGILFNHESPRRGENFVTRKITAALGRIKVGL
QTKLFLGNLQASRDWGFAGDYVEAMWLMLQQEKPDDYVVATEEGHTVE
EFLDVSFGYLGLNWKDYVEIDQRYFRPAEVDNLQGDASKAKEVLGWKP
QVGFEKLVKMMVDEDLELAKREKVLVDAGYMDAKQQP
Arabidopsis thaliana, At GMD M4: DNA
(SEQ ID NO: 80)
ATGGCAAGTGAGAACAATGGTTCACGTTCTGACTCTGAAAGCATCACG
GCTCCTAAAGCGGACAGCACCGTTGTGGAACCACGGAAAATCGCTCTA
ATCACCGGCATCACGGGTCAGGACGGTAGTTACTTGACTGAATTTCTA
CTAGGCAAAGGTTACGAAGTGCATGGCCTGATCCGTAGGAGTAGCAAT
TTTAACACGCAGCGGATCAATCATATCTATATTGATCCACACAACGTG
AACAAAGCTTTAATGAAACTCCATTACGCGGATCTCACTGACGCCTCT
TCGTTGCGTCGCTGGATCGACGTCATTAAACCTGACGAAGTGTATAAC
CTGGCGGCACAGTCTCATGTGGCCGTTTCATTCGAAATACCTGATTAT
ACGGCGGACGTGGTTGCCACCGGTGCCTTAAGACTGCTCGAGGCGGTT
CGCTCCCATACCATTGATTCCGGGCGCACGGTAAAATATTATCAGGCA
GGAAGCAGCGAAATGTTTGGAAGTACGCCGCCCCCTCAGTCTGAGACA
ACCCCGTTTCACCCGCGCAGTCCGTATGCGGCATCTAAATGTGCCGCA
CATTGGTATACAGTCAATTATCGTGAGGCTTATGGCTTGTTTGCATGC
AATGGCATTCTGTTCAATCATGAAAGCCCGCGCAGAGGCGAAAATTTT
GTTACCCGCAAAATTACGGCTGCCCTGGGCCGTATTAAAGTAGGTCTG
CAAACTAAACTGTTTCTTGGCAACCTCCAGGCTAGCCGTGACTGGGGA
TTTGCCGGTGATTATGTCGAAGCCATGTGGCTCATGTTACAGCAGGAG
AAACCGGACGATTATGTTGTTGCGACAGAAGAAGGACACACAGTGGAG
GAATTTTTGGATGTATCGTTCGGCTATTTAGGTCTAAACTGGAAAGAT
TACGTTGAGATTGATCAACGCTACTTCCGGCCGGCGGAAGTGGACAAC
CTGCAAGGAGATGCCTCCAAGGCAAAAGAAGTACTGGGTTGGAAACCG
CAGGTGGGCTTCGAGAAACTTGTCAAAATGATGGTGGATGAAGATCTG
GAATTAGCTAAACGCGAGAAGGTACTGGTAGATGCAGGATACATGGAT
GCGAAGCAGCAACCGTAA
Homo sapiens, Hs GMD M2: AA
(SEQ ID NO: 81)
MRNVALITGITGQDGSYLAEFLLEKGYEVHGIVRRSSSFNTGRIEHLY
KNPQAAIEGNMKLHYGDLTDSTCLVKIINEVKPTEIYNLGAQSHVKIS
FDLAEYTADVDGVGTLRLLDAVKTCGLINSVKFYQASTSELYGKVQEI
PQKETTPFYPRSPYGAAKLYAYWIVVNFREAYNLFAVNGILFNHESPR
RGANFVTRKISRSVAKIYLGQLECFSLGNLDAKRDWGHAKDYVEAMWL
MLQNDEPEDFVIATGEVHSVREFVEKSFLHIGKTIVWEGKNENEVGRC
KETGKVHVTVDLKYYRPTEVDFLQGDCTKAKQKLNWKPRVAFDELVRE
MVHADVELMRTNPNA
Homo sapiens, Hs GMD M2: DNA
(SEQ ID NO: 82)
ATGCGAAACGTTGCCTTGATCACCGGTATTACCGGCCAGGATGGCTCA
TATCTGGCAGAATTTCTGCTTGAAAAAGGCTATGAGGTTCATGGCATC
GTGCGCCGCAGCAGTAGTTTTAATACCGGCCGCATTGAACATCTGTAT
AAAAACCCACAAGCAGCCATCGAAGGAAATATGAAACTGCATTATGGC
GATTTGACAGACTCAACGTGTCTGGTTAAGATAATAAACGAAGTGAAG
CCTACCGAAATTTACAACCTGGGTGCGCAGTCTCATGTGAAAATTAGC
TTCGATTTGGCCGAATATACCGCGGATGTCGATGGTGTGGGTACGTTA
CGACTGTTGGACGCTGTTAAAACCTGCGGGCTGATCAACAGCGTGAAA
TTTTATCAGGCTAGCACGAGTGAGCTCTATGGAAAGGTCCAGGAGATT
CCCCAGAAGGAAACGACGCCTTTCTATCCACGCAGCCCGTATGGGGCA
GCAAAACTTTATGCCTATTGGATCGTAGTGAACTTTCGCGAAGCTTAT
AATCTTTTTGCGGTTAATGGCATACTGTTTAACCACGAGTCGCCACGA
CGCGGCGCAAACTTCGTGACCCGTAAAATAAGTCGTAGCGTCGCGAAG
ATCTATCTGGGTCAGCTCGAATGTTTCAGCCTTGGCAACCTGGATGCG
AAACGTGATTGGGGACACGCGAAAGATTATGTCGAAGCCATGTGGCTG
ATGTTACAAAACGATGAACCTGAGGACTTCGTTATCGCCACGGGTGAA
GTGCATAGCGTACGCGAATTTGTCGAAAAAAGCTTCCTCCATATAGGT
AAGACCATCGTGTGGGAAGGCAAAAATGAGAACGAGGTTGGTCGCTGC
AAAGAAACCGGCAAAGTTCACGTTACGGTTGATCTCAAATACTACAGA
CCCACCGAAGTGGACTTTCTGCAAGGCGATTGTACCAAAGCCAAACAG
AAACTAAATTGGAAACCTCGCGTTGCCTTCGACGAACTCGTCCGTGAA
ATGGTCCATGCAGATGTCGAACTGATGAGAACAAACCCTAACGCGTGA
Homo sapiens, Hs GMD M3: AA
(SEQ ID NO: 83)
MRNVALITGITGQDGSYLAEFLLEKGYEVHGIVRRSSSFNTGRIEHLY
KNPQAHIEGNMKLHYGDLTDSTCLVKIINEVKPTEIYNLGAQSHVKIS
FDLAEYTADVDGVGTLRLLDAVKTCGLINSVKFYQASTSELYGKVQEI
PQKETTPFYPRSPYGAAKLYAYWIVVNFREAYNLFAVNGILFNHESPR
RGANFVTRAISRSVAKIYLGQLECFSLGNLDAKRDWGHAKDYVEAMWL
MLQNDEPEDFVIATGEVHSVREFVEKSFLHIGKTIVWEGKNENEVGRC
KETGKVHVTVDLKYYRPTEVDFLQGDCTKAKQKLNWKPRVAFDELVRE
MVHADVELMRTNPNA
Homo sapiens, Hs GMD M3: DNA
(SEQ ID NO: 84)
ATGCGAAACGTGGCCTTGATCACCGGTATTACCGGCCAGGATGGCTCA
TATCTGGCAGAATTTCTGCTTGAAAAAGGCTATGAGGTTCATGGCATC
GTGCGCCGCAGCAGTAGTTTTAATACCGGCCGCATTGAACATCTGTAT
AAAAACCCACAAGCACACATCGAAGGAAATATGAAACTGCATTATGGC
GATTTGACAGACTCAACGTGTCTGGTTAAGATAATAAACGAAGTGAAG
CCTACCGAAATTTACAACCTGGGTGCGCAGTCTCATGTGAAAATTAGC
TTCGATTTGGCCGAATATACCGCGGATGTCGATGGTGTGGGTACGTTA
CGACTGTTGGACGCTGTTAAAACCTGCGGGCTGATCAACAGCGTGAAA
TTTTATCAGGCTAGCACGAGTGAGCTCTATGGAAAGGTCCAGGAGATT
CCCCAGAAGGAAACGACGCCTTTCTATCCACGCAGCCCGTATGGGGCA
GCAAAACTTTATGCCTATTGGATCGTAGTGAACTTTCGCGAAGCTTAT
AATCTTTTTGCGGTTAATGGCATACTGTTTAACCACGAGTCGCCACGA
CGCGGCGCAAACTTCGTGACCCGTGCAATAAGTCGTAGCGTCGCGAAG
ATCTATCTGGGTCAGCTCGAATGTTTCAGCCTTGGCAACCTGGATGCG
AAACGTGATTGGGGACACGCGAAAGATTATGTCGAAGCCATGTGGCTG
ATGTTACAAAACGATGAACCTGAGGACTTCGTTATCGCCACGGGTGAA
GTGCATAGCGTACGCGAATTTGTCGAAAAAAGCTTCCTCCATATAGGT
AAGACCATCGTGTGGGAAGGCAAAAATGAGAACGAGGTTGGTCGCTGC
AAAGAAACCGGCAAAGTTCACGTTACGGTTGATCTCAAATACTACAGA
CCCACCGAAGTGGACTTTCTGCAAGGCGATTGTACCAAAGCCAAACAG
AAACTAAATTGGAAACCTCGCGTTGCCTTCGACGAACTCGTCCGTGAA
ATGGTCCATGCAGATGTCGAACTGATGAGAACAAACCCTAACGCGTGA
Homo sapiens, Hs GMD M4: AA
(SEQ ID NO: 85)
MRNVALITGITGQDGSYLAEFLLEKGYEVHGIVRRSSSFNTGRIEHLY
KNPQAHIEGNMKLHYGDLTDSTCLVKIINEVKPTEIYNLGAQSHVKIS
FDLAEYTADVDGVGTLRLLDAVKTCGLINSVKFYQASTSELYGKVQEI
PQKETTPFYPRSPYGAAKLYAYWIVVNFREAYNLFAVNGILFNHESPR
RGANFVTRKISASVAKIYLGQLECFSLGNLDAKRDWGHAKDYVEAMWL
MLQNDEPEDFVIATGEVHSVREFVEKSFLHIGKTIVWEGKNENEVGRC
KETGKVHVTVDLKYYRPTEVDFLQGDCTKAKQKLNWKPRVAFDELVRE
MVHADVELMRTNPNA
Homo sapiens, Hs GMD M4: DNA
(SEQ ID NO: 86)
ATGCGAAACGTGGCCTTGATCACCGGTATTACCGGCCAGGATGGCTCA
TATCTGGCAGAATTTCTGCTTGAAAAAGGCTATGAGGTTCATGGCATC
GTGCGCCGCAGCAGTAGTTTTAATACCGGCCGCATTGAACATCTGTAT
AAAAACCCACAAGCACACATCGAAGGAAATATGAAACTGCATTATGGC
GATTTGACAGACTCAACGTGTCTGGTTAAGATAATAAACGAAGTGAAG
CCTACCGAAATTTACAACCTGGGTGCGCAGTCTCATGTGAAAATTAGC
TTCGATTTGGCCGAATATACCGCGGATGTCGATGGTGTGGGTACGTTA
CGACTGTTGGACGCTGTTAAAACCTGCGGGCTGATCAACAGCGTGAAA
TTTTATCAGGCTAGCACGAGTGAGCTCTATGGAAAGGTCCAGGAGATT
CCCCAGAAGGAAACGACGCCTTTCTATCCACGCAGCCCGTATGGGGCA
GCAAAACTTTATGCCTATTGGATCGTAGTGAACTTTCGCGAAGCTTAT
AATCTTTTTGCGGTTAATGGCATACTGTTTAACCACGAGTCGCCACGA
CGCGGCGCAAACTTCGTGACCCGTAAAATAAGTGCTAGCGTCGCGAAG
ATCTATCTGGGTCAGCTCGAATGTTTCAGCCTTGGCAACCTGGATGCG
AAACGTGATTGGGGACACGCGAAAGATTATGTCGAAGCCATGTGGCTG
ATGTTACAAAACGATGAACCTGAGGACTTCGTTATCGCCACGGGTGAA
GTGCATAGCGTACGCGAATTTGTCGAAAAAAGCTTCCTCCATATAGGT
AAGACCATCGTGTGGGAAGGCAAAAATGAGAACGAGGTTGGTCGCTGC
AAAGAAACCGGCAAAGTTCACGTTACGGTTGATCTCAAATACTACAGA
CCCACCGAAGTGGACTTTCTGCAAGGCGATTGTACCAAAGCCAAACAG
AAACTAAATTGGAAACCTCGCGTTGCCTTCGACGAACTCGTCCGTGAA
ATGGTCCATGCAGATGTCGAACTGATGAGAACAAACCCTAACGCGTGA
ASR 1: AA
(SEQ ID NO: 87)
MAFKVVQICGGLGNQMFQYAFAKSLQKHLNIPVLLDVTSFDWSNRKLQ
LELFPIDLPYASAKEIAMAKMQHLPKLVRDALKRMGFDRVSQEIVFEY
EPKLLKPNRLTYFHGYFQDPRYFDGISPLIKQTFTLPPPPPENGNNKK
KEEEYQRKLSLILAAKNSVFVHIRRGDYVGIGCQLGIDYQKKAVEYMA
KRVPNMELFVFCEDLEFTQNLDLGYPFMDMTTRDKEEEAYWDMMLMQS
CKHGIIANSTYSWWAAYLINNPEKIIIGPKHWLFGHENILCKDWVKIE
SHFEVKSEKYNA
ASR 1: DNA
(SEQ ID NO: 88)
ATGGCGTTTAAAGTCGTCCAGATTTGTGGAGGCTTAGGTAATCAAATG
TTTCAGTATGCTTTTGCTAAGTCACTGCAAAAACACCTTAACATTCCT
GTGCTTCTGGACGTTACCTCGTTTGATTGGTCGAATCGCAAATTACAG
CTGGAGTTGTTTCCAATTGACTTGCCGTATGCCTCAGCCAAAGAAATC
GCAATGGCGAAAATGCAGCATCTTCCGAAACTGGTGCGCGATGCGCTG
AAACGCATGGGATTCGATCGCGTGTCCCAGGAAATCGTCTTTGAATAT
GAACCAAAGCTCCTGAAACCAAACCGCTTGACCTACTTTCATGGCTAC
TTTCAGGACCCCCGCTATTTCGACGGCATCTCTCCCTTAATTAAACAG
ACCTTCACACTCCCTCCTCCGCCGCCTGAAAACGGGAATAATAAAAAG
AAAGAGGAGGAATATCAACGCAAACTGAGTCTGATTCTGGCGGCGAAA
AACTCTGTTTTCGTCCACATCCGTCGCGGCGATTACGTCGGTATTGGT
TGCCAGTTGGGCATTGATTACCAGAAAAAAGCGGTGGAATATATGGCG
AAACGAGTGCCGAATATGGAACTATTTGTGTTTTGTGAGGATCTGGAG
TTCACGCAGAACCTAGACTTGGGGTATCCATTTATGGATATGACCACG
CGGGACAAGGAAGAGGAAGCCTACTGGGATATGATGCTGATGCAGTCA
TGCAAGCACGGTATTATCGCCAATAGCACCTACTCGTGGTGGGCCGCC
TACTTAATTAACAATCCTGAGAAGATTATTATTGGTCCGAAACACTGG
TTATTTGGCCACGAAAACATCCTCTGCAAGGATTGGGTTAAAATTGAA
TCGCACTTTGAAGTCAAATCTGAAAAATACAACGCA
ASR 2: AA
(SEQ ID NO: 89)
MIIIRMSGGLGNQMFQYALYLKLKAMGKEVKIDDITEYEGDNARPIML
DVFGIDYDRATKEEVTELTDGSMDFLSRIRRKLFGRKSKEYREKSCNF
DPQVLEMDPAYLEGYFQSEKYFQDVREQVRKAFRFRGIESGSIPLSEK
TRELQKQIEDSESVSIHIRRGDYLENGHGEVYGGICTDAYYKKAIEYM
KEKFPDAKFYIFSNDTEWAKQHFKGENFVVVEGSTENTGYLDMFLMSK
CRHHIIANSSFSWWGAWLNENPEKIVIAPSKWLNNRECKDIYTERMIR
INPEV
ASR 2: DNA
(SEQ ID NO: 90)
ATGATTATCATTCGCATGAGCGGGGGTCTGGGCAATCAGATGTTCCAG
TATGCCCTCTATCTGAAGCTGAAAGCGATGGGCAAGGAAGTAAAAATC
GATGATATAACCGAATACGAGGGCGATAATGCTCGCCCGATAATGCTG
GACGTGTTTGGAATCGATTATGATCGTGCGACCAAAGAAGAAGTTACC
GAACTCACCGACGGTTCTATGGACTTTCTGTCGCGCATCCGCCGTAAA
CTTTTCGGCCGCAAATCGAAAGAATACCGTGAAAAAAGCTGCAATTTT
GACCCGCAAGTTTTGGAGATGGACCCGGCGTACCTGGAGGGCTATTTC
CAGAGCGAAAAATATTTTCAAGATGTGCGCGAACAGGTTCGAAAAGCG
TTCCGATTTCGTGGTATTGAATCAGGGTCCATTCCGCTGTCAGAAAAA
ACCCGCGAATTGCAGAAACAGATCGAAGATAGCGAGTCCGTTAGCATT
CATATCCGTCGTGGTGACTATCTGGAGAACGGCCACGGCGAAGTGTAC
GGCGGAATCTGCACCGATGCCTATTACAAAAAAGCCATCGAATACATG
AAGGAGAAATTCCCTGATGCCAAATTTTACATTTTTAGCAATGATACG
GAGTGGGCAAAACAACATTTCAAGGGAGAGAACTTTGTGGTGGTTGAG
GGCTCCACTGAAAATACTGGTTATCTTGATATGTTCCTGATGAGCAAA
TGTCGCCACCACATCATTGCGAATAGTTCGTTTAGCTGGTGGGGGGCG
TGGTTGAACGAAAACCCGGAAAAAATCGTGATTGCCCCGAGCAAATGG
CTGAATAACCGTGAATGTAAAGACATCTATACCGAACGCATGATCCGT
ATCAACCCCGAGGTG
ASR 3: AA
(SEQ ID NO: 91)
MIIIRIMGGLGNQMFQYALYRKLKSMGKEVKLDISWYDDHNQTHRSFE
LDVFGIDYDVASKEEISKFSNRSANFLSRIRRKLFGRKNKIYKEEDFN
YDPEILELDDVYLEGYWQSEKYFEDIREQLRKEFTFPEELNEKNRELL
EQMENENSVSIHIRRGDYLNNENADVYGGICTDDYYKKAIEYIRERIP
DPKFYIFSDDIEWAKQQFKGDDFTIVDWNNGKDSYYDMYLMSKCKHNI
IANSTFSWWGAWLNQNPEKIVISPKKWLNNHETSDIVCESWIRIDGQG
EIR
ASR 3: DNA
(SEQ ID NO: 92)
ATGATCATCATTCGCATTATGGGCGGCCTGGGTAATCAGATGTTTCAA
TACGCGCTGTATCGCAAACTGAAATCGATGGGAAAAGAAGTGAAACTG
GACATCAGTTGGTACGATGATCATAATCAAACTCACCGCAGCTTTGAA
CTCGACGTCTTTGGTATTGATTATGATGTGGCATCCAAAGAGGAAATT
AGCAAGTTTTCCAACCGCTCCGCGAATTTCCTGAGTAGAATTAGGCGA
AAACTGTTTGGCCGAAAAAACAAAATTTATAAAGAGGAGGACTTTAAC
TACGATCCAGAAATCCTTGAATTAGATGATGTTTATCTGGAGGGCTAT
TGGCAAAGTGAGAAGTATTTCGAAGATATTCGCGAACAACTGCGTAAA
GAGTTTACCTTTCCCGAAGAGCTGAACGAAAAGAATCGTGAGCTGCTG
GAACAAATGGAAAACGAAAACTCGGTATCGATTCACATTCGTCGCGGA
GATTATCTGAACAACGAGAACGCAGATGTATATGGTGGCATCTGCACA
GATGATTACTATAAAAAAGCTATCGAATATATTCGTGAGCGCATTCCC
GATCCAAAGTTTTATATATTCTCAGATGACATCGAATGGGCAAAACAA
CAGTTTAAAGGTGATGACTTCACCATCGTAGATTGGAACAATGGCAAA
GACAGCTATTATGATATGTATCTGATGTCAAAGTGTAAACACAACATC
ATTGCTAATTCCACCTTTTCCTGGTGGGGCGCCTGGCTGAATCAAAAT
CCCGAGAAAATCGTGATTTCCCCTAAGAAATGGCTTAACAACCATGAA
ACCTCAGACATAGTATGCGAAAGTTGGATTAGGATTGACGGTCAAGGT
GAAATTCGC
ASR 4: AA
(SEQ ID NO: 93)
MIIVRLTGGLGNQMFQYAMGRRLAEKHNTELKLDISGFENYKLRKYSL
NHFNIQENFATPEEISRLTSVKQGRIEKLLRRILRKRPKKPNTYIREK
HFHFDPEILNLPDNVYLDGYWQSEKYFKDIEDIIRREFTIKNPQTGKN
KEIAEQIQSCNSVSLHVRRGDYVTNPTTNQVHGVCGLDYYQRCVDYIA
KKVENPHFFVFSDDPEWVKENLKIDYPTTFVDHNGADKDYEDLRLMSQ
CKHHIIANSTFSWWGAWLNSNPDKIVIAPKKWFNTSDMDTKDLIPENW
IKL
ASR 4: DNA
(SEQ ID NO: 94)
ATGATTATTGTCCGGCTTACGGGCGGCTTAGGCAACCAAATGTTTCAG
TACGCAATGGGGCGCCGCTTAGCTGAAAAACATAATACCGAGCTGAAA
TTAGACATCAGCGGGTTTGAAAACTATAAACTGCGTAAATACAGCTTG
AATCACTTTAATATTCAGGAAAATTTTGCCACACCGGAAGAGATTTCG
CGGCTGACATCAGTTAAACAGGGCCGTATTGAAAAGTTGTTGCGCAGG
ATTCTGAGGAAGCGCCCAAAAAAACCGAATACGTATATCCGCGAGAAA
CACTTCCACTTTGATCCTGAAATTCTGAACCTCCCGGACAACGTTTAC
TTGGACGGTTACTGGCAGAGTGAGAAATACTTTAAGGACATTGAGGAC
ATCATTCGCCGTGAGTTTACCATAAAAAATCCGCAGACCGGCAAAAAC
AAAGAGATCGCGGAACAGATCCAGAGTTGCAATAGTGTCTCACTGCAT
GTTCGTCGCGGTGATTACGTTACGAACCCCACTACCAACCAAGTCCAC
GGCGTCTGTGGGCTAGATTACTATCAACGTTGCGTGGATTATATCGCA
AAAAAGGTTGAAAACCCACACTTCTTTGTTTTTAGCGATGATCCCGAG
TGGGTGAAAGAAAACCTTAAAATCGATTATCCTACTACCTTCGTGGAC
CACAACGGTGCGGATAAAGACTATGAAGATTTACGTCTGATGTCACAA
TGCAAACATCATATCATTGCAAACTCTACCTTTAGTTGGTGGGGTGCC
TGGCTCAATTCTAACCCTGACAAAATTGTGATTGCGCCGAAGAAGTGG
TTCAACACTAGCGATATGGATACCAAAGATTTGATTCCAGAGAATTGG
ATCAAACTA
ASR 5: AA
(SEQ ID NO: 95)
MIVVKLIGGLGNQMFQYAAAKALALEKNQKLRLDVSAFESYKLHNYGL
NHFNITAKIYKKENKWLRKIKSFFKKNTYYKEQDFGYNPDLFDLKADN
IFLEGYFQSEKYFLKYEKEIRKDFEIISPLKKQTKEMIEQIQSVNSVS
IHIRRGDYLTNPIHNTSKEEYYKKAMEFIESKIENPVFFVFSDDMDWV
KENFKTNHETVFVDFNDASTNFEDLKLMSSCKHNIIANSSFSWWGAWL
NKNPNKIVIAPKQWFNDDSINTSDIIPESWIKI
ASR 5: DNA
(SEQ ID NO: 96)
ATGATCGTTGTAAAACTGATTGGTGGTCTTGGCAACCAGATGTTCCAG
TACGCGGCGGCGAAAGCTCTGGCGCTCGAAAAAAACCAGAAGCTGCGT
CTTGATGTCAGTGCTTTCGAATCATACAAACTGCACAATTATGGACTG
AATCATTTCAACATAACCGCCAAAATCTATAAAAAAGAAAATAAGTGG
TTACGCAAAATCAAAAGTTTCTTCAAAAAGAATACCTACTACAAAGAA
CAAGACTTTGGCTATAACCCGGATCTGTTTGATTTGAAAGCGGACAAT
ATTTTTCTGGAGGGTTATTTCCAAAGCGAGAAATATTTTCTAAAGTAC
GAAAAAGAAATACGTAAAGATTTCGAGATCATCTCACCATTAAAAAAA
CAGACCAAAGAAATGATTGAACAAATTCAGTCTGTGAATAGTGTCTCG
ATACATATAAGGCGCGGTGATTATCTGACCAATCCGATTCATAATACG
TCAAAAGAAGAATACTATAAGAAGGCAATGGAGTTTATTGAATCCAAA
ATTGAAAACCCGGTATTCTTCGTGTTTAGTGATGACATGGACTGGGTC
AAAGAAAACTTTAAAACGAACCATGAGACTGTGTTCGTAGATTTCAAT
GATGCCAGCACCAACTTTGAGGACCTAAAGCTGATGTCCTCATGTAAA
CACAATATTATTGCGAACAGCTCTTTTAGCTGGTGGGGTGCTTGGCTG
AATAAAAATCCGAACAAAATTGTTATCGCGCCAAAACAGTGGTTTAAC
GACGATAGCATTAATACTTCAGACATCATCCCGGAGTCCTGGATTAAA
ATA
ASR 6: AA
(SEQ ID NO: 97)
MAFKVVQICGGLGNQMFQYAFAKSLQKHLNIPVLLDVTSFDSSNRKLQ
LELFPIDLPYASAKEIAMAKMQHLPKLVRDALKRMGFDRVSQEIVFEY
EPKLLKPNRLTYFHGYFQDPRYFDGISPLIKQTFTLPPPPPENGNNKK
KEEEYQRKLSLILAAKNSVFVHIRRGDYVGIGCQLGIDYQKKAVEYMA
KRVPNMELFVFCEDLEFTQNLDLGYPFMDMTTRDKEEEAYWDMMLMQS
CKHGIIANSTYSWWAAYLINNPEKIIIGPKHWLFGHENILCKDWVKIE
SHFEVKSEKYNA
ASR 6: DNA
(SEQ ID NO: 98)
ATGGCGTTTAAAGTGGTTCAGATTTGCGGCGGCTTAGGTAATCAGATG
TTCCAGTATGCTTTTGCGAAAAGCCTGCAAAAACATCTGAATATTCCT
GTCCTTTTAGACGTCACGAGCTTTGACTCCTCTAATAGAAAACTCCAA
TTAGAACTGTTCCCAATTGATCTGCCGTATGCAAGTGCAAAAGAGATT
GCGATGGCAAAAATGCAGCACCTCCCAAAACTGGTTCGAGATGCCTTA
AAGCGAATGGGATTCGACCGCGTCAGCCAGGAGATTGTTTTTGAATAC
GAACCTAAACTTCTTAAGCCAAACCGCCTGACGTACTTCCACGGTTAC
TTTCAAGATCCGCGCTATTTCGACGGAATCAGTCCGCTGATCAAGCAG
ACGTTCACCTTGCCGCCGCCGCCCCCTGAAAACGGTAATAATAAGAAA
AAAGAAGAGGAATATCAGCGGAAGCTGAGCTTGATCCTGGCAGCCAAA
AACAGTGTCTTTGTGCACATTCGTCGCGGCGACTATGTGGGCATTGGT
TGTCAATTGGGGATTGATTACCAGAAAAAAGCGGTCGAGTACATGGCG
AAACGAGTGCCCAATATGGAGCTGTTTGTTTTCTGCGAGGACTTAGAA
TTTACCCAGAATTTGGATCTGGGCTATCCGTTTATGGACATGACGACA
CGCGATAAAGAAGAGGAAGCCTACTGGGATATGATGCTGATGCAGAGC
TGCAAGCACGGTATTATCGCTAACTCAACATATTCCTGGTGGGCCGCA
TATCTGATTAATAACCCCGAAAAGATTATCATCGGACCAAAACACTGG
CTCTTCGGTCACGAAAATATCCTGTGCAAAGATTGGGTAAAGATTGAA
AGCCACTTTGAAGTGAAAAGCGAAAAATATAACGCC
ASR 7: AA
(SEQ ID NO: 99)
MIIIRMSGGLGNQMFQYALYLKLKSMGKEVKIDDITAYEGDNARPIML
DVFGIDYDRATKEEITEMTDSSMDFLSRIRRKLFGRKSKEYREKDFNF
DPQVLEMDPAYLEGYFQSEKYFQDVREQVRKAFRFRKGSVPKELSEQT
KELQKQIENSNSVSIHIRRGDYLENSHGEIYGGICTDAYYKKAIEYMK
EKFPDAKFYIFSNDTEWAKQHFKGENFVIVEGSTENTGYLDMYLMSKC
KHHIIANSSFSWWGAWLNDNPEKIVIAPSKWLNNRECKDIYTDRMIRI
DAKGEVRSDDYGVRTNSTVK
ASR 7: DNA
(SEQ ID NO: 100)
ATGATTATCATCCGCATGAGCGGCGGACTGGGCAACCAAATGTTCCAG
TATGCCTTGTATCTGAAACTGAAAAGTATGGGTAAAGAAGTGAAAATC
GATGATATAACAGCCTATGAAGGGGATAACGCCCGCCCGATCATGCTG
GACGTTTTCGGCATCGATTATGACCGTGCTACGAAAGAGGAGATTACC
GAAATGACCGATTCCTCGATGGATTTTCTGTCACGCATTCGTCGCAAA
CTGTTTGGACGTAAAAGTAAAGAATATCGCGAAAAAGATTTCAATTTC
GATCCGCAGGTCCTGGAGATGGACCCGGCGTACTTGGAAGGCTACTTC
CAGTCCGAGAAATACTTTCAGGATGTGCGCGAACAGGTCCGCAAGGCG
TTCCGGTTCCGCAAGGGAAGCGTACCGAAAGAATTGTCCGAACAGACC
AAGGAACTGCAAAAACAGATTGAAAACTCGAACTCAGTGTCAATTCAT
ATCCGTCGCGGCGACTATCTGGAAAACTCACACGGTGAGATTTATGGG
GGGATTTGCACCGATGCTTACTATAAAAAAGCGATTGAATACATGAAA
GAAAAATTCCCGGATGCCAAATTCTATATTTTCAGCAACGACACTGAA
TGGGCCAAGCAGCATTTTAAAGGCGAAAACTTTGTCATCGTTGAGGGC
TCAACTGAAAATACCGGGTACTTAGACATGTATCTGATGTCCAAATGT
AAACACCACATTATTGCAAACTCTAGCTTTAGCTGGTGGGGTGCCTGG
CTGAACGATAACCCGGAAAAAATTGTAATCGCCCCGTCAAAATGGTTA
AACAATCGCGAGTGCAAGGACATTTATACTGACCGCATGATTCGTATA
GATGCAAAAGGCGAAGTCCGTAGCGATGATTATGGGGTTCGTACGAAC
AGCACGGTGAAA
ASR 8: AA
(SEQ ID NO: 101)
MIIIRIMGGLGNQMFQYALYRKLKSMGKEVKLDISWYDDHNTHRSFEL
DVFGIEYDVASKKEISKFSNRSSNFLSRIRRKLFGKKNKIYQEEDFNY
DPEILEMDDVYLEGYWQSEKYFEDIREQLRKEFTFPKEMNKQNKELLE
QMENENSVSIHIRRGDYLNKENASIYGGICTDDYYKKAIEYIREKVSN
PKFYIFSDDIEWAKQHFKGDDMTIVDWNNGKDSYYDMYLMSSCKHNII
ANSTFSWWGAWLNQNPEKIVIAPKKWLNNHETSDIVCDNWIRIDGNGE
IRSEEYGVRTGSTVK
ASR 8: DNA
(SEQ ID NO: 102)
ATGATTATTATCCGCATTATGGGGGGCTTGGGCAACCAGATGTTCCAA
TATGCTCTGTATCGCAAACTAAAGTCAATGGGTAAAGAGGTTAAATTG
GATATTTCGTGGTATGACGATCATAATACCCATCGCTCATTTGAATTA
GATGTTTTTGGCATTGAATATGACGTCGCATCCAAAAAAGAAATCTCG
AAATTCTCTAACCGCTCAAGCAACTTTTTGTCTCGAATCCGCCGGAAG
TTGTTCGGAAAAAAGAATAAAATCTATCAGGAGGAGGACTTCAACTAT
GACCCGGAGATCCTGGAAATGGATGATGTGTACCTGGAAGGGTACTGG
CAGTCGGAAAAATATTTTGAGGATATTCGTGAACAGTTACGTAAAGAA
TTTACCTTCCCGAAAGAGATGAACAAACAGAACAAGGAACTGCTGGAA
CAGATGGAAAACGAAAATTCCGTGTCCATCCATATTCGTCGTGGAGAT
TATTTAAACAAAGAAAACGCAAGCATTTATGGAGGAATCTGCACCGAT
GATTATTATAAAAAGGCAATTGAGTATATTCGCGAGAAAGTTAGTAAC
CCGAAGTTCTATATTTTTTCGGATGATATAGAGTGGGCAAAACAGCAT
TTCAAAGGGGACGATATGACCATCGTGGACTGGAATAACGGCAAAGAT
TCCTATTACGATATGTACCTGATGTCGAGTTGTAAACACAACATTATT
GCCAACTCCACGTTTTCATGGTGGGGCGCCTGGCTGAACCAAAACCCG
GAAAAGATTGTGATCGCTCCGAAAAAATGGCTTAACAATCATGAAACT
AGCGATATTGTTTGCGATAACTGGATTCGTATCGATGGTAATGGAGAA
ATTCGGTCGGAGGAATATGGGGTCCGCACCGGAAGCACCGTGAAA
ASR 9: AA
(SEQ ID NO: 103)
MIIVRLTGGLGNQMFQYAMGRRLAEKHNTELKLDISAFENYKLRKYSL
HHFNIQENFATPEEISRLTSVKQNKIEKLLHKILRKKPKKSNTYIKEK
HFHFDPNILNLPDNVYLDGYWQSEKYFKDIEDIIRKEFTIKYPQTGKN
KEIAEKIQSCNSVSIHIRRGDYVTNPTTNQVHGVCGLDYYQRCIDYIA
KKVENPHFFVFSDDPEWVKENLKIQYPTTYVDHNNTDKDYEDLRLMSQ
CKHHIIANSTFSWWGAWLNSNPDKIVIAPKKWFNTSDYNTKDLIPENW
IKL
ASR 9: DNA
(SEQ ID NO: 104)
ATGATTATTGTCCGACTCACCGGCGGTCTGGGCAATCAAATGTTCCAA
TATGCAATGGGTCGCCGTTTAGCGGAAAAACACAATACAGAACTCAAA
CTGGACATTAGCGCGTTCGAGAATTATAAACTGCGAAAGTATAGTCTG
CACCATTTTAATATCCAAGAAAATTTTGCAACCCCAGAAGAGATTAGT
CGTTTAACGAGCGTAAAACAAAACAAGATCGAAAAACTGTTGCACAAA
ATCCTTCGCAAGAAACCGAAAAAATCAAACACCTACATTAAGGAGAAA
CATTTTCATTTTGATCCGAATATACTGAATCTGCCGGATAATGTATAC
TTAGATGGATACTGGCAAAGCGAAAAATACTTCAAGGATATTGAAGAT
ATTATTCGTAAAGAATTTACAATCAAATATCCACAGACGGGTAAAAAC
AAGGAAATTGCGGAGAAAATTCAGTCTTGCAACTCTGTAAGTATACAC
ATTCGTCGCGGTGATTATGTAACCAACCCGACCACTAACCAGGTTCAT
GGTGTTTGTGGCCTGGATTATTATCAGAGGTGCATCGACTATATTGCG
AAAAAGGTGGAGAACCCGCACTTTTTTGTTTTCTCTGATGATCCTGAA
TGGGTAAAAGAAAATCTTAAAATCCAGTATCCAACCACGTATGTGGAC
CATAATAACACAGATAAAGATTACGAAGATTTGCGTCTGATGTCGCAG
TGTAAACACCACATCATCGCGAACTCTACCTTTAGCTGGTGGGGTGCC
TGGCTGAATAGTAATCCAGATAAAATAGTGATTGCTCCGAAAAAATGG
TTTAATACGAGCGACTACAATACCAAAGACTTAATACCTGAAAATTGG
ATCAAACTG
ASR 10: AA
(SEQ ID NO: 105)
MIVVKLIGGLGNQMFQYAAAKALALEKNQKLRLDVSAFETYKLHNYGL
NHFNITAKIYKKENKWLRKIKSFFKKNTYYKEQDFGYNPDLFNLKADN
IFLEGYFQSEKYFLKYEKEIRKDFEIISPLKKQTKEMIEKIQSVNSVS
IHIRRGDYLTNPIHNTSKEEYYKKAMKFIESKIENPVFFVFSDDMDWV
KENFKTNHETVFVDENDASTNFEDIKLMSSCKHNIIANSSFSWWGAWL
NQNPNKIVIAPKQWFNDDSINTSDIIPESWIKI
ASR 10: DNA
(SEQ ID NO: 106)
ATGATCGTCGTTAAACTTATCGGTGGTCTGGGGAACCAAATGTTTCAG
TATGCCGCGGCGAAGGCTCTGGCGCTCGAAAAAAACCAAAAACTGCGC
TTGGACGTTAGTGCATTTGAAACTTATAAATTACACAACTATGGCCTC
AATCATTTCAATATCACGGCGAAAATTTACAAAAAGGAAAACAAGTGG
TTACGCAAAATAAAATCATTCTTTAAAAAAAACACCTATTATAAAGAG
CAGGACTTCGGATACAATCCTGACCTGTTTAACTTGAAAGCTGATAAC
ATCTTTCTTGAAGGGTATTTCCAATCGGAAAAATATTTCCTCAAATAT
GAAAAAGAGATTCGAAAAGACTTCGAAATTATTAGTCCTCTGAAAAAA
CAAACGAAAGAAATGATCGAAAAAATCCAATCCGTGAACTCTGTCTCT
ATCCATATCCGTCGCGGCGACTACCTCACGAATCCCATACATAACACC
TCCAAGGAGGAATACTATAAAAAAGCAATGAAATTTATTGAGTCGAAA
ATCGAAAACCCCGTGTTCTTTGTATTTTCGGATGATATGGACTGGGTG
AAAGAAAACTTTAAAACGAACCATGAGACTGTATTCGTGGATTTCAAT
GATGCGAGCACAAATTTCGAAGATATTAAGCTGATGTCATCGTGTAAA
CACAATATCATTGCGAACAGTTCCTTCTCTTGGTGGGGGGCCTGGCTG
AATCAGAATCCAAATAAAATTGTGATCGCTCCGAAGCAATGGTTTAAT
GATGATTCGATTAATACCTCGGATATTATTCCTGAGAGTTGGATCAAA
ATC
ASR 11: AA
(SEQ ID NO: 107)
MIIIRMSGGLGNQMFQYALYRKLKAMGKEVKIDDVTGYEDDNQRPIML
DVFGIDYDRATKEEVTELTDSSMDFLSRIRRKLFGRKSKEYREEDCNF
DPQVLEMDDAYLEGYFQSEKYFQDVREQLRKEFRFRSGSVPLSEKTRE
LQKQIENSNSVSIHIRRGDYLENGHAEVYGGICTDDYYKKAIEYMKEK
FPDAKFYIFSNDVEWAKQHFKGENFVVVEGSEENTGYLDMFLMSKCRH
HIIANSSFSWWGAWLNENPEKIVIAPSKWLNNRECKDIYTERMIRISA
EV
ASR 11: DNA
(SEQ ID NO: 108)
ATGATCATTATTCGCATGTCAGGCGGGCTGGGCAACCAGATGTTTCAG
TATGCCCTCTATCGCAAGTTGAAAGCTATGGGCAAAGAGGTTAAAATT
GACGACGTAACGGGATATGAAGATGACAATCAACGTCCGATCATGCTG
GACGTGTTTGGTATCGATTACGACCGTGCGACCAAAGAAGAAGTGACC
GAACTCACCGACTCCTCAATGGACTTTCTGTCCCGTATCCGCCGTAAG
CTGTTTGGCCGCAAATCTAAAGAATATCGTGAAGAAGATTGTAATTTT
GATCCGCAGGTGCTTGAAATGGATGACGCATACCTGGAGGGTTATTTC
CAGAGCGAAAAATACTTTCAGGATGTTAGGGAACAGCTGCGCAAAGAG
TTTCGATTTCGTTCAGGTTCAGTGCCGCTGTCGGAAAAGACGCGGGAA
TTACAGAAACAGATTGAGAACAGCAACTCTGTGAGTATCCATATCAGA
CGTGGTGACTACCTGGAAAATGGTCATGCAGAAGTTTATGGTGGCATC
TGTACGGACGACTACTATAAAAAAGCCATCGAATACATGAAAGAGAAA
TTCCCGGATGCGAAGTTCTACATTTTTTCTAATGATGTCGAATGGGCT
AAGCAGCATTTTAAAGGCGAAAATTTTGTGGTTGTGGAAGGTTCGGAA
GAAAATACCGGCTATTTAGATATGTTTCTTATGAGCAAGTGTCGCCAT
CATATAATTGCCAACTCTAGTTTTAGCTGGTGGGGCGCATGGCTCAAT
GAAAACCCAGAAAAGATTGTAATCGCGCCGTCTAAATGGCTGAACAAC
CGTGAATGCAAAGATATTTATACCGAACGTATGATTCGTATTTCCGCA
GAAGTA
ASR 12: AA
(SEQ ID NO: 109)
MIIIRMSGGLGNQMFQYALYRKLKSMGKEVKIDDITGYEDDNQRSIML
DVFGIDYDKATKEEITKLTDSSMDFLSRIRRKLFGRKSKEYQEEDFNF
DPQVLEMDDAYLEGYFQSEKYFQDVREQLRKEFTFRKNSVPELSEQTK
ELRKQIENSNSVSIHIRRGDYLENSHAEIYGGICTDDYYKKAIEYMKE
KFPDAKFYIFSNDIEWAKQHFKGENFVIVDASEENTGYADMYLMSKCK
HHIIANSSFSWWGAWLNDNPEKIVIAPSKWLNNKECKDIYTDRMIKID
AKGEVRSEDYGVRTNSTVK
ASR 12: DNA
(SEQ ID NO: 110)
ATGATTATTATACGTATGAGTGGCGGCCTGGGTAATCAAATGTTTCAG
TATGCCCTGTACCGCAAATTGAAATCGATGGGGAAAGAGGTGAAAATA
GACGACATCACCGGGTATGAGGACGATAACCAGCGTTCTATCATGCTC
GATGTGTTTGGGATTGATTACGACAAAGCAACCAAAGAAGAGATAACC
AAGCTGACCGACAGTAGCATGGACTTTCTGTCTCGCATTCGTCGCAAA
CTGTTTGGCCGCAAATCGAAGGAGTACCAGGAAGAAGATTTTAATTTT
GACCCACAAGTCCTGGAAATGGATGATGCCTACCTCGAAGGGTACTTC
CAAAGTGAAAAGTATTTCCAGGATGTGCGGGAGCAGCTGCGAAAAGAA
TTTACCTTTCGAAAAAACAGCGTGCCGGAACTGTCGGAACAGACGAAA
GAACTGCGCAAACAAATTGAAAATAGCAACAGCGTGTCGATTCACATT
CGCCGTGGTGACTATTTGGAAAACTCCCACGCCGAGATTTATGGCGGT
ATTTGTACTGACGATTACTACAAGAAAGCGATTGAGTACATGAAAGAG
AAATTCCCGGATGCAAAGTTTTACATTTTCTCGAATGATATTGAATGG
GCGAAACAGCACTTTAAAGGGGAGAATTTTGTAATTGTTGACGCATCA
GAAGAGAACACTGGCTATGCGGATATGTACCTGATGAGCAAATGCAAA
CACCACATTATTGCCAATTCCTCCTTCTCGTGGTGGGGTGCCTGGCTG
AACGATAACCCGGAAAAAATCGTGATTGCTCCGAGTAAATGGCTCAAT
AATAAAGAGTGCAAAGATATTTACACCGACCGCATGATTAAAATTGAC
GCCAAAGGTGAGGTCCGTTCAGAGGATTACGGCGTACGTACCAACTCT
ACCGTGAAA

1. A method for producing 2′-fucosyllactose comprising:
(i) incubating GDP-L-fucose with an α-1,2-fucosyltransferase in a culture medium comprising lactose for a sufficient time to convert said GDP-L-fucose and lactose into 2′-fucosyllactose and GDP; wherein said α-1,2-fucosyltransferase is selected from the group consisting of a polypeptide comprising an amino acid sequence having at least 90% sequence identity to SEQ ID NO: 109, SEQ ID NO: 29, SEQ ID NO: 107, SEQ ID NO: 89, SEQ ID NO: 91, SEQ ID NO: 93, SEQ ID NO: 95, SEQ ID NO: 99, SEQ ID NO: 101, SEQ ID NO: 103, or SEQ ID NO: 105; or
(ii) incubating GDP-mannose and/or GDP-L-galactose with a dehydratase and a reductase in the presence of NADPH and/or NADP+ in a culture medium for a sufficient time to convert said GDP-mannose and/or GDP-L-galactose into GDP-L-fucose; and incubating said GDP-L-fucose with an α-1,2-fucosyltransferase and lactose for a sufficient time to convert said GDP-L-fucose and lactose into 2′-fucosyllactose and GDP: wherein said dehydratase is selected from the group consisting of a polypeptide comprising the amino acid sequence of SEQ ID NO: 79, SEQ ID NO: 77, SEQ ID NO: 75, or SEQ ID NO: 5; and a polypeptide comprising the amino acid sequence of SEQ ID NO: 85, SEQ ID NO: 83, SEQ ID NO: 81, or SEQ ID NO: 9; or
(iii) incubating GDP-L-galactose with a dehydratase and a reductase in the presence of NADPH and/or NADP+ for a sufficient time to convert said GDP-L-galactose into GDP-L-fucose; and incubating said GDP-L-fucose with lactose and an α-1,2-fucosyltransferase for a sufficient time to convert said GDP-L-fucose and lactose into 2′-fucosyllactose and GDP; or
(iv) incubating GDP-L-fucose with lactose and an α-1,2-fucosyltransferase for a sufficient time to convert said GDP-L-fucose and lactose into 2′-fucosyllactose, wherein the α-1,2-fucosyltransferase is an enzyme comprising an amino acid sequence having at least 90% sequence identity to any one of SEQ ID NOs: 21, 23, 25, 27, 29, 31, 33, 35, 37, 39, 41, 43, 45, 47, 49, 51, 53, 55, 57, 59, and 61; or
(v) providing a reaction mixture comprising a fucokinase/guanylyltransferase, a dehydratase, a reductase, an α-1,2-fucosyltransferase, ATP, GTP, NADP+, and NADPH; and adding L-galactose to the reaction mixture; and incubating said reaction mixture for a sufficient time to produce 2′-fucosyllactose; wherein the reaction mixture further comprises: (a) a first regenerating enzyme and a first substrate for said first regenerating enzyme, wherein said first regenerating enzyme is capable of catalyzing a first regeneration reaction involving the first substrate that uses NADP+ as a co-factor, thereby regenerating NADPH: (b) a second regenerating enzyme and a second substrate for said second regenerating enzyme, wherein said second regenerating enzyme is capable of catalyzing a second regeneration reaction involving the second substrate that uses ADP as a co-factor, thereby regenerating ATP; and (c) a third regenerating enzyme and a third substrate for said third regenerating enzyme, wherein said third regenerating enzyme is capable of catalyzing a third regeneration reaction involving the third substrate that uses GDP as a co-factor, thereby regenerating GTP.

2. The method of claim 1, wherein the α-1,2-fucosyltransferase is a polypeptide comprising the amino acid sequence of SEQ ID NO: 109.

3. The method of claim 1, wherein the α-1,2-fucosyltransferase is a polypeptide comprising to the amino acid sequence of SEQ ID NO: 29.

4. The method of claim 2, wherein said GDP-L-fucose is generated in situ in the culture medium from GDP-mannose or GDP-L-galactose in a reaction catalyzed by a dehydratase enzyme.

5. The method of claim 4, where said dehydratase enzyme is a polypeptide comprising the amino acid sequence of SEQ ID NO: 5 or SEQ ID NO: 9.

6. The method of claim 4, wherein said dehydratase enzyme is a polypeptide comprising the amino acid sequence of SEQ ID NO: 79, SEQ ID NO: 77, or SEQ ID NO: 75.

7. The method of claim 4, wherein said dehydratase enzyme is a polypeptide comprising the amino acid sequence of SEQ ID NO: 85, SEQ ID NO: 83, or SEQ ID NO: 81.

8. (canceled)

9. The method of claim 1, wherein the dehydratase is a polypeptide comprising the amino acid of SEQ ID NO: 79, SEQ ID NO: 77, or SEQ ID NO: 75.

10. The method of claim 1, wherein the α-1,2-fucosyltransferase is a polypeptide comprising an amino acid sequence having at least 90% sequence identity to SEQ ID NO: 109, SEQ ID NO: 29, SEQ ID NO: 107, SEQ ID NO: 89, SEQ ID NO: 91, SEQ ID NO: 93, SEQ ID NO: 95, SEQ ID NO: 99, SEQ ID NO: 101, SEQ ID NO: 103, or SEQ ID NO: 105.

11. The method of claim 1, wherein the reductase is a polypeptide comprising an amino acid having at least 90% sequence identity to SEQ ID NO: 7, SEQ ID NO: 15, or SEQ ID NO: 17.

12. An engineered microorganism for enhanced production of 2′-fucosyllactose, said microorganism comprising at least the following heterologous genes for producing 2′-fucosyllactose:
(i) a first heterologous gene that encodes a mutant dehydratase for producing GDP-L-fucose, said mutant dehydratase being a polypeptide comprising the amino sequence selected from SEQ ID NO: 79, SEQ ID NO: 77, SEQ ID NO: 75, SEQ ID NO: 85, SEQ ID NO: 83, and SEQ ID NO: 81; and
(ii) a second heterologous gene that encodes a mutant α-1,2-fucosyltransferase for converting GDP-L-fucose to 2′-fucosyllactose, said mutant α-1,2-fucosyltransferase being a polypeptide comprising an amino acid sequence having at least 90% sequence identity to SEQ ID NO: 109.

13. The microorganism of claim 12, wherein the microorganism further comprises a heterologous gene for exporting 2′-fucosyllactose extracellularly.

14. A method for producing 2′-fucosyllactose comprising culturing the microorganism of claim 12 in a culture medium comprising at least one carbon source.

15. The method of claim 14, further comprising separating the culture medium from the microorganism.

16. The method of claim 15, further comprising isolating 2′-fucosyllactose from the culture medium.

17. A polypeptide comprising:
a. mutant dehydratase for producing GDP-L-fucose, said mutant dehydratase being a polypeptide comprising the amino sequence selected from the group consisting of SEQ ID NO: 79, SEQ ID NO: 77, SEQ ID NO: 75, SEQ ID NO: 85, SEQ ID NO: 83, and SEQ ID NO: 81; or
b. a mutant α-1,2-fucosyltransferase for producing 2′-fucosyllactose, said mutant α-1,2-fucosyltransferase being a polypeptide comprising an amino acid sequence having at least 90% sequence identity to SEQ ID NO: 109, SEQ ID NO: 29, SEQ ID NO: 107, SEQ ID NO: 89, SEQ ID NO: 91, SEQ ID NO: 93, SEQ ID NO: 95, SEQ ID NO: 99, SEQ ID NO: 101, SEQ ID NO: 103, or SEQ ID NO: 105.

18. (canceled)

19. A nucleic acid construct comprising a nucleic acid sequence that encodes at least one of the mutant enzymes of claim 17.

20. A microorganism comprising the nucleic acid construct of claim 19.

21.-58. (canceled)
